(19) United States
(12) Patent Application Publication
(10) Pub. No.: US 2009/0124598 A1
(43) Pub. Date: May 14, 2009

(54) PHARMACEUTICAL USE OF SUBSTITUTED AMIDES

(76) Inventors: Henrik Sune Andersen, Lyngby (DK); Anker Steen Jorgensen, Copenhagen (DK); John Paul Kilburn, Haslev (DK); Gita Camilla Tejlgaard Kampen, Naerum (DK); Soren Ebdrup, Roskilde (DK)

Correspondence Address: TRANSTECH PHARMA, INC., 4170 MENDENHALL OAKS PKWY, HIGH POINT, NC, 27265, US

(21) Appl. No.: 12/092,230

(22) PCT Filed: Nov. 1, 2006
(86) PCT No.: PCT/EP2006/068015

§ 371 Date: Oct. 23, 2008

(30) Foreign Application Priority Data
Nov. 1, 2005 (EP) 05110228.3
Jul. 7, 2006 (EP) 06116808.4

(51) Int. Cl. A61K 31/55; C07D 209/52; A61K 31/403; C07D 223/14; A61P 3/10; A61K 31/5377; C07D 413/12

(52) U.S. Cl. 514/216; 548/452; 514/412; 514/235.2; 544/143; 540/583

(57) ABSTRACT

The use of substituted amides for modulating the activity of 11β-hydroxysteroid dehydrogenase type 1 (11βHSD1) and the use of these compounds as pharmaceutical compositions, are described. Also a novel class of substituted amides, their use in therapy, pharmaceutical compositions comprising the compounds, as well as their use in the manufacture of medicaments are described. The present compounds are modulators and more specifically inhibitors of the activity of 11βHSD1 and may be useful in the treatment of a range of medical disorders where a decreased intracellular concentration of active glucocorticoid is desirable.

### FIELD OF INVENTION

The present invention relates to use of substituted amides and pharmaceutical compositions comprising the same for treating disorders where it is desirable to modulate the activity of 11β-hydroxysteroid dehydrogenase type 1 (11βHSD1). The present invention also relates to novel substituted amides, to their use in therapy, to pharmaceutical compositions comprising the same, to the use of said compounds in the manufacture of medicaments, and to therapeutic methods comprising the administration of the compounds. The present compounds modulate the activity of 11β-hydroxysteroid dehydrogenase type 1 (11βHSD1) and are accordingly useful in the treatment of diseases in which such a modulation is beneficial, such as the metabolic syndrome.

### BACKGROUND OF THE INVENTION

The metabolic syndrome is a major global health problem. In the US, the prevalence in the adult population is currently estimated to be approximately 25%, and it continues to increase both in the US and worldwide. The metabolic syndrome is characterised by a combination of insulin resistance, dyslipidemia, obesity and hypertension leading to increased morbidity and mortality of cardiovascular diseases. People with the metabolic syndrome are at increased risk of developing frank type 2 diabetes, the prevalence of which is equally escalating.

In type 2 diabetes, obesity and dyslipidemia are also highly prevalent and around 70% of people with type 2 diabetes additionally have hypertension once again leading to increased mortality of cardiovascular diseases.

In the clinical setting, it has long been known that glucocorticoids are able to induce all of the cardinal features of the metabolic syndrome and type 2 diabetes.

11β-hydroxysteroid dehydrogenase type 1 (11βHSD1) catalyses the local generation of active glucocorticoid in several tissues and organs including predominantly the liver and adipose tissue, but also e.g., skeletal muscle, bone, pancreas, endothelium, ocular tissue and certain parts of the central nervous system. Thus, 11βHSD1 serves as a local regulator of glucocorticoid actions in the tissues and organs where it is expressed (Tannin et al., J. Biol. Chem., 266, 16653 (1991); Bujalska et al., Endocrinology, 140, 3188 (1999); Whorwood et al., J. Clin Endocrinol Metab., 86, 2296 (2001); Cooper et al., Bone, 27, 375 (2000); Davani et al., J. Biol. Chem., 275, 34841 (2000); Brem et al., Hypertension, 31, 459 (1998); Rauz et al., Invest. Ophthalmol. Vis. Sci., 42, 2037 (2001); Moisan et al., Endocrinology, 127, 1450 (1990)).

The role of 11βHSD1 in the metabolic syndrome and type 2 diabetes is supported by several lines of evidence. In humans, treatment with the non-specific 11βHSD1 inhibitor carbenoxolone improves insulin sensitivity in lean healthy volunteers and people with type 2 diabetes. Likewise, 11βHSD1 knock-out mice are resistant to insulin resistance induced by obesity and stress. Additionally, the knock-out mice present with an anti-atherogenic lipid profile of decreased VLDL triglycerides and increased HDL-cholesterol. Conversely, mice that overexpress 11βHSD1 in adipocytes develop insulin resistance, hyperlipidemia and visceral obesity, a phenotype that resembles the human metabolic syndrome (Andrews et al., J. Clin. Endocrinol. Metab., 88, 285 (2003); Walker et al., J. Clin. Endocrinol. Metab., 80, 3155 (1995); Morton et al., J. Biol. Chem., 276, 41293 (2001); Kotelevtsev et al., Proc. Natl. Acad. Sci. USA, 94, 14924 (1997); Masuzaki et al., Science, 294, 2166 (2001)).

The more mechanistic aspects of 11βHSD1 modulation and thereby modulation of intracellular levels of active glucocorticoid have been investigated in several rodent models and different cellular systems. 11βHSD1 promotes the features of the metabolic syndrome by increasing hepatic expression of the rate-limiting enzymes in gluconeogenesis, namely phosphoenolpyuvate carboxykinase and glucose-6-phosphatase, promoting the differentiation of preadipocytes into adipocytes thus facilitating obesity, directly and indirectly stimulating hepatic VLDL secretion, decreasing hepatic LDL uptake and increasing vessel contractility (Kotelevtsev et al., Proc. Natl. Acad. Sci. USA, 94, 14924 (1997); Morton et al., J. Biol. Chem. 276, 41293 (2001); Bujalska et al., Endocrinology, 140, 3188 (1999); Souness et al., Steroids, 67, 195 (2002), Brindley & Salter, Prog. Lipid Res., 30, 349 (1991)).

WO 01/90090, WO 01/90091, WO 01/90092, WO 01/90093, and WO 01/90094 discloses various thiazol-sulfonamides as inhibitors of the human 11β-hydroxysteroid dehydrogenase type 1 enzyme, and further states that said compounds may be useful in treating diabetes, obesity, glaucoma, osteoporosis, cognitive disorders, immune disorders and depression.

We have now found substituted amides that modulate the activity of 11βHSD1 leading to altered intracellular concentrations of active glucocorticoid. More specifically, the present compounds inhibit the activity of 11βHSD1 leading to decreased intracellular concentrations of active glucocorticoid. Thus, the present compounds can be used to treat disorders where a decreased level of active intracellular glucocorticoid is desirable, such as e.g., the metabolic syndrome, type 2 diabetes, impaired glucose tolerance (IGT), impaired fasting glucose (IFG), dyslipidemia, obesity, hypertension, diabetic late complications, cardiovascular diseases, arteriosclerosis, atherosclerosis, myopathy, muscle wasting, osteoporosis, neurodegenerative and psychiatric disorders, and adverse effects of treatment or therapy with glucocorticoid receptor agonists.

One object of the present invention is to provide compounds, pharmaceutical compositions and use of compounds that modulate the activity of 11βHSD1.

### DEFINITIONS

In the following structural formulas and throughout the present specification, the following terms have the indicated meaning. The examples provided in the definitions present in this application are non-inclusive unless otherwise stated. They include but are not limited to the recited examples.

The term “halo” includes fluorine, chlorine, bromine, and iodine.

The term “trihalomethyl” includes trifluoromethyl, trichloromethyl, tribromomethyl, and triiodomethyl.

The term “trihalomethoxy” includes trifluorometoxy, trichlorometoxy, tribromometoxy, and triiodometoxy.

The term “alkyl” includes C₁-C₈ straight chain saturated and methylene aliphatic hydrocarbon groups and C₃-C₈ branched saturated hydrocarbon groups having the specified number of carbon atoms. For example, this definition includes methyl (Me), ethyl (Et), propyl (Pr), butyl (Bu), pentyl, hexyl, isopropyl (i-Pr), isobutyl (i-Bu), tert-butyl (t-Bu), sec-butyl (s-Bu), isopentyl, and neopentyl.

The term “alkenyl” includes C₂-C₆ straight chain unsaturated aliphatic hydrocarbon groups and branched C₃-C₆ unsaturated aliphatic hydrocarbon groups having the specified number of carbon atoms. For example, this definition includes ethenyl, propenyl, butenyl, pentenyl, hexenyl, methylpropenyl, and methylbutenyl.

The term “alkynyl” includes C₂-C₆ straight chain unsaturated aliphatic hydrocarbon groups and C₄-C₆ branched unsaturated aliphatic hydrocarbon groups having the specified number of carbon atoms. For example, this definition includes ethynyl, propynyl, butynyl, pentynyl, hexynyl, and methylbutynyl.

The term “saturated or partially saturated monocyclic, bicyclic, or tricyclic ring system” represents but is not limited to aziridinyl, azepanyl, azocanyl, pyrrolinyl, pyrrolidinyl, 2-imidazolinyl, imidazolidinyl, 2-pyrazolinyl, morpholinyl, piperidinyl, thiomorpholinyl, piperazinyl, phthalimide, 1,2,3,4-tetrahydro-quinolinyl, 1,2,3,4-tetrahydro-isoquinolinyl, 1,2,3,4-tetrahydro-quinoxalinyl, indolinyl, 1,6-aza-bicyclo[3.2.1]octane, 2-aza-bicyclo[4.1.1]octane, 2-aza-bicyclo[3.2.1]octanyl, 7-aza-bicyclo[4.1.1]octanyl, 9-aza-bicyclo[3.3.2]decanyl, 4-aza-tricyclo[4.3.1.1^{3,8}]undecanyl, 9-aza-tricyclo[3.3.2.0^{3,7}]decanyl.

The term “saturated or partially saturated ring” represents cyclopropyl, cyclobutyl, cyclopentyl, cyclohexyl, cyclobutenyl, cyclopentenyl, cyclohexenyl, cycloheptenyl, cyclooctenyl, cyclononenyl, cyclodecenyl, tetrahydrofuranyl, and tetrahydropyranyl.

The term “saturated or partially saturated aromatic ring” represents cyclopentyl, cyclohexyl, cyclobutenyl, cyclopentenyl, cyclohexenyl, cycloheptenyl, cyclooctenyl, cyclononenyl, cyclodecenyl, tetrahydrofuranyl, tetrahydropyranyl, phenyl, pyridyl, and pyrimidinyl.

The term “cycloalkyl” represents a saturated, mono-, bi-, tri- or spirocarbocyclic group having the specified number of carbon atoms (e.g., cyclopropyl, cyclobutyl, cyclopentyl, cyclohexyl, cycloheptyl, cyclooctyl, cyclononyl, cyclodecyl, bicyclo[3.2.1]octyl, spiro[4.5]decyl, norpinyl, norbonyl, norcaryl, and adamantyl).

The term “cycloalkylalkyl” represents a cycloalkyl group as defined above attached through an alkyl group having the indicated number of carbon atoms or substituted alkyl group as defined above (e.g., cyclopropylmethyl, cyclobutylethyl, and adamantylmethyl).

The term “cycloalkenyl” represents a partially saturated, mono-, bi-, tri- or spirocarbocyclic group having the specified number of carbon atoms (e.g., cyclobutenyl, cyclopentenyl, cyclohexenyl, cycloheptenyl, cyclooctenyl, cyclononenyl, and cyclodecenyl).

The term “cycloalkylcarbonyl” represents a cycloalkyl group as defined above having the indicated number of carbon atoms attached through a carbonyl group (e.g., cyclopropylcarbonyl and cyclohexylcarbonyl).

The term “cycloalkylalkylcarbonyl” represents a cycloalkyl group as defined above attached through an alkyl group having the indicated number of carbon atoms or substituted alkyl group as defined above (e.g., cyclohexylmethylcarbonyl and cycloheptylethylcarbonyl).

The term “hetcycloalkyl” represents a saturated mono-, bi-, tri-, or spirocarbocyclic group having the specified number of atoms with 1-4 of the specified number being heteroatoms or groups selected from nitrogen, oxygen, sulphur, and S(O)ₘ (m=0-2)(e.g., tetrahydrofuranyl, tetrahydropyranyl, tertahydrothiopyranyl, piperidine, and pyridzine).

The term “hetcycloalkylalkyl” represents a hetcycloalkyl group as defined above attached through an alkyl group having the indicated number of carbon atoms (e.g., tetrahydrofuranylmethyl, tetrahydropyranylethyl, and tertahydrothiopyranylmethyl).

The term “hetcycloalkylcarbonyl” represents a hetcycloalkyl group as defined above having the indicated number of carbon atoms attached through a carbonyl group (e.g., 1-piperidin-4-yl-carbonyl and 1-(1,2,3,4-tetrahydro-isoquinolin-6-yl)carbonyl).

The term “alkyloxy” represents an alkyl group having the indicated number of carbon atoms attached through an oxygen bridge (e.g., methoxy, ethoxy, propyloxy, allyloxy, and cyclohexyloxy).

The term “alkyloxyalkyl” represents an alkyloxy group as defined above attached through an alkyl group having the indicated number of carbon atoms (e.g., methyloxymethyl).

The term “aryl” includes a carbocyclic aromatic ring that is monocyclic, bicyclic, or polycyclic, such as phenyl, biphenyl, naphthyl, anthracenyl, phenanthrenyl, fluorenyl, indenyl, pentalenyl, azulenyl, and biphenylenyl. Aryl also includes the partially hydrogenated derivatives of the carbocyclic aromatic enumerated above. Examples of partially hydrogenated derivatives include 1,2,3,4-tetrahydronaphthyl and 1,4-dihydronaphthyl.

The term “hetaryl” includes pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), isoxazolyl (3-isoxazolyl, 4-isoxazolyl, 5-isoxazolyl), thiazolyl (2-thiazolyl, 4-thiazolyl, 5-thiazolyl), thiophenyl (2-thiophenyl, 3-thiophenyl, 4-thiophenyl, 5-thiophenyl), furanyl (2-furanyl, 3-furanyl, 4-furanyl, 5-furanyl), pyridyl (2-pyridyl, 3-pyridyl, 4-pyridyl, 5-pyridyl), 5-tetrazolyl, pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo-[b]furanyl), 6-(2,3-dihydro-benzo-[b]furanyl), 7-(2,3-dihydro-benzo[b]furanyl)), 1,4-benzodioxin (2-(1,4-benzodioxin), 3-(1,4-benzodioxin), 5-(1,4-benzodioxin), 6-(1,4-benzodioxin), 7-(1,4-benzodioxin), 8-(1,4-benzodioxin)), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydrobenzo[b]thiophenyl), 4-(2,3-dihydrobenzo[b]thiophenyl), 5-(2,3-dihydro-benzo[b]thiophenyl), 6-(2,3-dihydro-benzo[b]thiophenyl), 7-(2,3-dihydro-benzo[b]thiophenyl)), 4,5,6,7-tetrahydrobenzo[b]thiophenyl (2-(4,5,6,7-tetrahydro-benzo[b]thiophenyl), 3-(4,5,6,7-tetrahydrobenzo[b]thiophenyl), 4-(4,5,6,7-tetrahydro-benzo[b]thiophenyl), 5-(4,5,6,7-tetrahydrobenzo[b]thiophenyl), 6-(4,5,6,7-tetrahydro-benzo[b]thiophenyl), 7-(4,5,6,7-tetrahydrobenzo[b]thiophenyl)), thieno[2,3-b]thiophenyl, 4,5,6,7-tetrahydro-thieno[2,3-c]pyridyl (4-(4,5,6,7-tetrahydro-thieno[2,3-c]pyridyl), 5-4,5,6,7-tetrahydro-thieno[2,3-c]pyridyl), 6-(4,5,6,7-tetrahydro-thieno[2,3-c]pyridyl), 7-(4,5,6,7-tetrahydro-thieno[2,3-c]pyridyl)), indolyl (1-indolyl, 2-indolyl, 3-indolyl, 4-indolyl, 5-indolyl, 6-indolyl, 7-indolyl), isoindolyl (1-isoindolyl, 2-isoindolyl, 3-isoindolyl, 4-isoindolyl, 5-isoindolyl, 6-isoindolyl, 7-isoindolyl), 1,3-dihydroisoindolyl (1-(1,3-dihydro-isoindolyl), 2-(1,3-dihydro-isoindolyl), 3-(1,3-dihydro-isoindolyl), 4-(1,3-dihydro-isoindolyl), 5-(1,3-dihydro-isoindolyl), 6-(1,3-dihydro-isoindolyl), 7-(1,3-dihydroisoindolyl)), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benz-oxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), benzo-[1,2,5]oxadiazolyl, (4-benzo[1,2,5]oxadiazole, 5-benzo[1,2,5]oxadiazole), carbazolyl (1-carbazolyl, 2-carbazolyl, 3-carbazolyl, 4-carbazolyl), piperidinyl (2-piperidinyl, 3-piperidinyl, 4-piperidinyl), and pyrrolidinyl (1-pyrrolidinyl, 2-pyrrolidinyl, 3-pyrrolidinyl).

The term “arylalkyl” represents an aryl group as defined above attached through an alkyl group having the indicated number of carbon atoms (e.g., benzyl, phenylethyl, 3-phenylpropyl, 1-naphthylmethyl, and 2-(1-naphtyl)ethyl).

The term “hetarylalkyl” or “hetaralkyl” represents a hetaryl group as defined above attached through an alkyl group having the indicated number of carbon atoms (e.g., (2-furyl)methyl, (3-furyl)methyl, (2-thienyl)methyl, (3-thienyl)methyl, (2-pyridyl)methyl, and 1-methyl-1-(2-pyrimidyl)ethyl).

The term “aryloxyhetaryl” represents an aryloxy group as defined above attached through a hetaryl group (e.g., 2-phenoxy-pyridyl).

The term “aryloxy” represents an aryl group as defined above attached through an oxygen bridge (e.g., phenoxy and naphthyloxy).

The term “hetaryloxy” represents a hetaryl group as defined above attached through an oxygen bridge (e.g., 2-pyridyloxy).

The term “arylalkyloxy” represents an arylalkyl group as defined above attached through an oxygen bridge (e.g., phenethyloxy and naphthylmethyloxy).

The term “hetarylalkyloxy” represents a hetarylalkyl group as defined above attached through an oxygen bridge (e.g., 2-pyridylmethyloxy).

The term “alkyloxycarbonyl” represents an alkyloxy group as defined above attached through a carbonyl group (e.g., methylformiat and ethylformiat).

The term “aryloxycarbonyl” represents an aryloxy group as defined above attached through a carbonyl group (e.g., phenylformiat and 2-thiazolylformiat).

The term “arylalkyloxycarbonyl” represents an “arylalkyloxy” group as defined above attached through a carbonyl group (e.g., benzylformiat and phenyletylformiat).

The term “alkylthio” represents an alkyl group having the indicated number of carbon atoms attached through a sulphur bridge (e.g., methylthio and ethylthio).

The term “arylthio” represents an aryl group as defined above attached through a sulphur bridge (e.g., benzenthiol and naphthylthiol).

The term “hetarylthio” represents a hetaryl group as defined above attached through a sulphur bridge (e.g., pyridine-2-thiol and thiazole-2-thiol).

The term “arylthioalkyl” represents an arylthio group as defined above attached through an alkyl group having the indicated number of carbon atoms (e.g., methylsulfanyl benzene, and ethylsulfanyl naphthalene).

The term “hetarylthioalkyl” represents a hetarylthio group as defined above attached through an alkyl group having the indicated number of carbon atoms (e.g., 2-methylsulfanyl-pyridine and 1-ethylsulfanyl-isoquinoline).

The term “hetaryloxyaryl” represents a hetaryloxy group as defined above attached through an aryl group as defined above (e.g., 1-phenoxy-isoquinolyl and 2-phenoxypyridyl).

The term “hetaryloxyhetaryl” represents a hetaryloxy group as defined above attached through a hetaryl group as defined above (e.g., 1-(2-pyridyloxy-isoquinoline) and 2-(imidazol-2-yloxy-pyridine)).

The term “aryloxyalkyl” represents an aryloxy group as defined above attached through an alkyl group having the indicated number of carbon atoms (e.g., phenoxymethyl and naphthyloxyethyl).

The term “aryloxyaryl” represents an aryloxy group as defined above attached through an aryl group as defined above (e.g., 1-phenoxy-naphthalene and phenyloxyphenyl).

The term “arylalkyloxyalkyl” represents an arylalkyloxy group as defined above attached through an alkyl group having the indicated number of carbon atoms (e.g., ethoxy-methyl-benzene and 2-methoxymethyl-naphthalene).

The term “hetaryloxyalkyl” represents a hetaryloxy group as defined above attached through an alkyl group having the indicated number of carbon atoms (e.g., 2-pyridyloxymethyl and 2-quinolyloxyethyl).

The term “hetarylalkyloxyalkyl” represents a hetarylalkyloxy group as defined above attached through an alkyl group having the indicated number of carbon atoms (e.g., 4-methoxymethyl-pyrimidine and 2-methoxymethyl-quinoline).

The term “alkylcarbonyl” represents an alkyl group as defined above having the indicated number of carbon atoms attached through a carbonyl group (e.g., octylcarbonyl, pentylcarbonyl, and 3-hexenylcarbonyl).

The term “arylcarbonyl” represents an aryl group as defined above attached through a carbonyl group (e.g., benzoyl).

The term “hetarylcarbonyl” represents a hetaryl group as defined above attached through a carbonyl group (e.g., 2-thiophenylcarbonyl, 3-methoxy-anthrylcarbonyl, and oxazolylcarbonyl).

The term “carbonylalkyl” represents a carbonyl group attached through an alkyl group having the indicated number of carbon atoms (e.g., acetyl).

The term “alkylcarbonylalkyl” represents an alkylcarbonyl group as defined above attached through an alkyl group having the indicated number of carbon atoms (e.g., propan-2-one and 4,4-dimethyl-pentan-2-one).

The term “arylcarbonylalkyl” represents a arylcarbonyl group as defined above attached through an alkyl group having the indicated number of carbon atoms (e.g., 1-phenyl-propan-1-one and 1-(3-chloro-phenyl)-2-methyl-butan-1-one).

The term “hetarylcarbonylalkyl” represents a hetarylcarbonyl group as defined above attached through an alkyl group having the indicated number of carbon atoms (e.g., 1-pyridin-2-yl-propan-1-one and 1-(1-H-imidazol-2-yl)-propan-1-one).

The term “arylalkylcarbonyl” represents an arylalkyl group as defined above having the indicated number of carbon atoms attached through a carbonyl group (e.g., phenylpropylcarbonyl and phenylethylcarbonyl).

The term “hetarylalkylcarbonyl” represents a hetarylalkyl group as defined above wherein the alkyl group is in turn attached through a carbonyl (e.g., imidazolylpentylcarbonyl).

The term “alkylcarbonylamino” represents an “alkylcarbonyl” group as defined above wherein the carbonyl is in turn attached through the nitrogen atom of an amino group (e.g., methylcarbonylamino, cyclopentylcarbonyl-aminomethyl, and methylcarbonylaminophenyl). The nitrogen atom may itself be substituted with an alkyl or aryl group.

The term “alkylcarbonylaminoalkyl” represents an “alkylcarbonylamino” group attached through an alkyl group having the indicated number of carbon atoms (e.g.N-propyl-acetamide and N-butyl-propionamide).

The term “arylalkylcarbonylamino” represents an “arylalkylcarbonyl” group as defined above attached through an amino group (e.g., phenylacetamide and 3-phenyl-propionamide).

The term “arylalkylcarbonylaminoalkyl” represents an “arylalkylcarbonylamino” group attached through an alkyl group having the indicated number of carbon atoms (e.g., N-ethyl-phenylacetamide and N-butyl-3-phenyl-propionamide).

The term “arylcarbonylamino” represents an “arylcarbonyl” group as defined above attached through an amino group (e.g., benzamide and naphthalene-1-carboxylic acid amide).

The term “arylcarbonylaminoalkyl” represents an “arylcarbonylamino” group attached through an alkyl group having the indicated number of carbon atoms (e.g., N-propyl-benzamide and N-butyl-naphthalene-1-carboxylic acid amide).

The term “alkylcarboxy” represents an alkylcarbonyl group as defined above wherein the carbonyl is in turn attached through an oxygen bridge (e.g., heptylcarboxy, cyclopropyl-carboxy, and 3-pentenylcarboxy).

The term “arylcarboxy” represents an arylcarbonyl group as defined above wherein the carbonyl is in turn attached through an oxygen bridge (e.g., benzoic acid).

The term “alkylcarboxyalkyl” represents an alkylcarboxy group as defined above wherein the oxygen is attached via an alkyl bridge (e.g., heptylcarboxymethyl, propylcarboxy tert-butyl, and 3-pentylcarboxyethyl).

The term “arylalkylcarboxy” represents an arylalkylcarbonyl group as defined above wherein the carbonyl is in turn attached through an oxygen bridge (e.g., benzylcarboxy and phenylpropylcarboxy).

The term “arylalkylcarboxyalkyl” represents an arylalkylcarboxy group as defined above wherein the carboxy group is in turn attached through an alkyl group having the indicated number of carbon atoms (e.g., benzylcarboxymethyl and phenylpropylcarboxypropyl).

The term “hetarylcarboxy” represents a hetarylcarbonyl group as defined above wherein the carbonyl is in turn attached through an oxygen bridge (e.g., pyridine-2-carboxylic acid).

The term “hetarylalkylcarboxy” represents a hetarylalkylcarbonyl group as defined above wherein the carbonyl is in turn attached through an oxygen bridge (e.g., (1-H-imidazol-2-yl)-acetic acid and 3-pyrimidin-2-yl-propionic acid).

The term “alkylS(O)ₘ” represents an alkyl group having the number of indicated carbon atoms, wherein the alkyl group is in turn attached through a sulphur bridge wherein the sulphur is substituted with m oxygen atoms (m=0-2)(e.g., ethylsulfonyl and ethylsulfinyl).

The term “arylS(O)ₘ” represents an aryl group as defined above, wherein the aryl group is in turn attached through a sulphur bridge wherein the sulphur is substituted with m oxygen atoms (m=0-2)(e.g., phenylsulfinyl and naphthyl-2-sulfonyl).

The term “hetarylS(O)ₘ” represents a hetaryl group as defined above, wherein the hetaryl group is in turn attached through a sulphur bridge wherein the sulphur is substituted with m oxygen atoms (m=0-2)(e.g., thiazol-2-sulfinyl and pyridine-2-sulfonyl).

Certain of the above defined terms may occur more than once in the structural formulae, and upon such occurrence each term shall be defined independently of the other.

The term “optionally substituted” as used herein means that the groups in question are either unsubstituted or substituted with one or more of the substituents specified. When the groups in question are substituted with more than one substituent, the substituents may be the same or different.

The term “treatment” or “treating” is defined as the management and care of a patient for the purpose of combating or alleviating the disease, condition, or disorder, and the term includes the administration of the active compound to prevent or delay the onset of the symptoms or complications; alleviating (both temporary and permanent) the symptoms or complications; and/or eliminating the disease, condition, or disorder. Thus, “treatment” or “treating” includes prevention and/or prophylaxis of the disease, condition, or disorder.

The term “pharmaceutically acceptable” is defined as being suitable for administration to humans without adverse events.

The term “prodrug” is defined as a chemically modified form of the active drug, said prodrug being administered to the patient and subsequently being converted to the active drug. Techniques for development of prodrugs are well known in the art.

### DETAILED DESCRIPTION OF THE INVENTION

Thus, in an embodiment, the present invention provides a novel substituted amide, a prodrug thereof, or a salt thereof with a pharmaceutically acceptable acid or base, or any optical isomer or mixture of optical isomers, including a racemic mixture or any tautomeric forms, wherein the compound is of formula I:

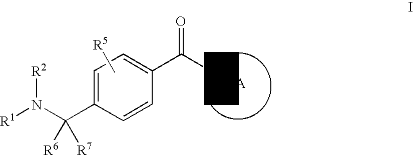

wherein:

R¹ is selected from H, R⁸(C═O)—, R⁹S(O)ₙ—, R¹⁰R¹¹NC(═Y)—, and R¹⁰R¹¹NS(O)ₙ—;

R² is selected from H, C₁-C₆alkyl, and C₁-C₆cycloalkyl;

alternatively, R¹ and R², together with the nitrogen to which they are attached, form a 3-12 membered saturated or partially saturated monocyclic or bicyclic ring consisting of the shown nitrogen, 2-10 carbon atoms, and 0-2 additional heteroatoms selected from nitrogen, oxygen, and S(O)ₘ, wherein this ring is substituted with 0-3 groups selected from C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)R¹², —S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸;

Ring A is a 5-12 membered saturated or partially saturated bicyclic or tricyclic ring consisting of the shown nitrogen, 4-10 carbon atoms and from 0 to 2 additional heteroatoms selected from nitrogen, oxygen, and S(O)ₘ;

Ring A is substituted with 0-3 groups selected from C₁-C₈alkyl, halo, OH, oxo, cyano, C₁-C₆alkyloxy, C₁-C₆alkyloxyC₁-C₆alkyl or C₁-C₆alkylcarbonyl, wherein each alkyl group is substituted with 0-3 R¹⁸;

R⁵ is selected from H, C₁-C₆alkyl, C₃-C₆cycloalkyl, halo, OH, and cyano;

R⁶ and R⁷ are independently selected from H, C₁-C₆alkyl, F, trihalomethyl, and trihalomethoxy;

alternatively, R⁶ and R⁷, together with the carbon atom to which they are attached, form a 3-8 membered saturated or partially saturated monocyclic ring consisting of the shown carbon atom, 2-5 additional carbon atoms, and 0-2 heteroatoms selected from nitrogen, oxygen, and S(O)ₘ, wherein this ring is substituted with 0-3 groups selected from halo, trihalomethyl, OH, C₁-C₆alkyl, oxo, and C₁-C₆alkyloxy;

R⁸ is selected from C₁-C₈alkyl, C₂-C₈alkenyl, aryl, hetaryl, arylC₁-C₆alkyl, hetaryl-C₁-C₆alkyl, C₃-C₁₀cycloalkyl, 3-10 membered hetcycloalkyl, aryloxyC₁-C₆alkyl, hetaryloxyC₁-C₆alkyl, arylC₁-C₆alkyloxyC₁-C₆alkyl, and hetarylC₁-C₆alkyloxyC₁-C₆alkyl, wherein each of the alkyl/alkyl, alkenyl, aryl, hetaryl, cycloalkyl, and hetcycloalkyl groups are independently substituted with 0-3 R¹⁹;

R⁹ is selected from C₁-C₈alkyl, C₂-C₈alkenyl, aryl, hetaryl, arylC₁-C₆alkyl, hetaryl-C₁-C₆alkyl, C₃-C₁₀cycloalkyl, 3-10 membered hetcycloalkyl, aryloxyC₁-C₆alkyl, and arylC₁-C₆alkyloxyC₁-C₆alkyl, wherein each of the alkyl/alkyl, alkenyl, aryl, hetaryl, cycloalkyl, and hetcycloalkyl groups are independently substituted with 0-3 R²⁰;

R¹⁰ and R¹¹ are independently selected from H, C₁-C₈alkyl, C₃-C₁₀cycloalkyl, 3-10 membered hetcycloalkyl, aryl, hetaryl, arylC₁-C₆alkyl, and hetarylC₁-C₆alkyl, wherein each of the alkyl/alkyl, cycloalkyl, hetcycloalkyl, aryl, and hetaryl groups are independently substituted with 0-3 R²¹;

alternatively, R¹⁰ and R¹¹, together with the nitrogen to which they are attached, form a 5-12 membered saturated or partially saturated monocyclic, bicyclic, or tricyclic ring consisting of the shown nitrogen atom, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkyl, hetarylC₁-C₆alkyl, hydroxy, oxo, COOH, C₁-C₆alkyloxy, arylC₁-C₆alkyloxy, hetarylC₁-C₆alkyloxy, C₁-C₆alkyloxyC₁-C₆alkyl, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkyl-carboxy, and hetarylC₁-C₆alkylcarboxy;

R¹² is selected from OH, C₁-C₈alkyl, C₃-C₁₀cycloalkyl, 3-10 membered hetcycloalkyl, trihalomethyl, C₁-C₈alkyloxy, aryl, arylC₁-C₆alkyl, hetaryl, hetarylC₁-C₆alkyl, aryloxy, hetaryloxy, and NR¹³R¹⁴;

R¹³ and R¹⁴ are independently selected from H, C₁-C₈alkyl, C₃-C₁₀cycloalkyl, aryl, hetaryl, arylC₁-C₆alkyl, and hetarylC₁-C₆alkyl, wherein each of the alkyl/alkyl, cycloalkyl, aryl, and hetaryl groups are independently substituted with 0-3 R²²;

alternatively, R¹³ and R¹⁴, together with the nitrogen to which they are attached, form a 5-12 membered saturated or partially saturated monocyclic, bicyclic, or tricyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkyl, hetarylC₁-C₆alkyl, OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkylcarboxy, and hetarylC₁-C₆alkylcarboxy;

R¹⁵ is selected from H, C₁-C₆alkyl, and C₃-C₆cycloalkyl;

R¹⁶ and R¹⁷ are independently selected from H, C₁-C₈alkyl, C₃-C₁₀cycloalkyl, halo, OH, cyano, —C(═O)R¹², —S(O)ₙR¹², —S(O)ₙNR¹³R¹⁴, —N(R¹³)S(O)ₙR¹², C₁-C₈alkyl, aryl, and hetaryl, wherein the alkyl and cycloalkyl groups are independently substituted with 0-3 R²²;

R¹⁸ is selected from halo, OH, oxo, COOH, cyano C₁-C₆alkyloxy, C₃-C₁₀cycloalkyloxy, aryloxy, hetaryloxy, hetarylthio and arylC₁-C₆alkyloxy;

R¹⁹, R²⁰ and R²¹ are independently selected from H, halo, OH, oxo, cyano, C₁-C₈alkyl, C₃-C₁₀cycloalkyl, 3-10 membered hetcycloalkyl, trihalomethyl, trihalomethyloxy, methylendioxo, dihalo-methylenedioxo, C₃-C₆spirocycloalkyl, C₁-C₆alkyloxy, aryl, hetaryl, arylC₁-C₆alkyl, hetarylC₁-C₆alkyl, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, and —C(═NR¹⁶)NR¹⁷;

R²² is selected from H, OH, oxo, halo, cyano, nitro, C₁-C₆alkyl, C₁-C₆alkyloxy, NR²³R²⁴, methylendioxo, dihalomethylendioxo, trihalomethyl, and trihalomethyloxy;

R²³ and R²⁴ are independently selected from H, C₁-C₈alkyl, and arylC₁-C₆alkyl;

m is selected from 0, 1, and 2;

n is selected from 1 and 2;

Y is selected from O and S;

or a salt thereof with a pharmaceutically acceptable acid or base, or any optical isomer or mixture of optical isomers, including a racemic mixture, or any tautomeric forms.

In another embodiment, the present invention provides the novel substituted amides of formula I, wherein:

R¹ is selected from R⁸(C═O)—, R⁹S(O)₂—, R¹⁰R¹¹NC(═O)—, and R¹¹R¹¹NS(O)₂—;

R² is C₁-C₄alkyl;

alternatively, R¹ and R², together with the nitrogen to which they are attached, form a 5-6 membered saturated ring consisting of the shown nitrogen, 2-4 carbon atoms, and 0-2 additional heteroatoms selected from nitrogen, oxygen, and S(O)ₘ, wherein this ring is substituted with 0-2 groups selected from C₁-C₈alkyl, aryl, hetaryl, arylC₁-C₆alkyl, hetarylC₁-C₆alkyl, —C(═O)R¹², —S(O)ₙR¹², —S(═O)ₙNR¹³R¹⁴, —N(R¹³)S(O)ₙR¹², OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹³;

Ring A is an 8-11 membered saturated or partially saturated bicyclic or tricyclic ring consisting of the shown nitrogen, 5-10 carbon atoms and from 0 to 1 additional heteroatoms selected from nitrogen, oxygen, and S(O)ₘ;

Ring A is substituted with 0-3 groups selected from C₁-C₄alkyl, halo, OH, oxo, cyano, C₁-C₄alkyloxy, C₁-C₄alkyloxyC₁-C₄alkyl or C₁-C₄alkylcarbonyl, wherein each alkyl/alkyl group is substituted with 0-1 R¹⁸;

R⁵ is H;

R⁶ and R⁷ are independently selected from H and C₁-C₄alkyl; and,

n is 2.

In another embodiment, the present invention provides the novel substituted amides of formula I, wherein:

R¹ is selected from R⁸(C═O)—, R⁹S(O)₂—, R¹⁰R¹¹NC(═O)—, and R¹¹R¹¹NS(O)₂—;

R² is C₁-C₄alkyl;

alternatively, R¹ and R², together with the nitrogen to which they are attached, form a 5-6 membered saturated ring consisting of the shown nitrogen, 2-4 carbon atoms, and 0-2 additional heteroatoms selected from nitrogen, oxygen, and S(O)ₘ, wherein this ring is substituted with 1-2 groups selected from C₁-C₈alkyl, aryl, hetaryl, arylC₁-C₆alkyl, hetarylC₁-C₆alkyl, —C(═O)R¹², —S(O)ₙR¹², —S(═O)ₙNR¹³R¹⁴, —N(R¹³)S(O)ₙR¹², OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸;

Ring A is an 8-11 membered saturated or partially saturated bicyclic or tricyclic ring consisting of the shown nitrogen, 5-10 carbon atoms and from 0 to 1 additional heteroatoms selected from nitrogen, oxygen, and S(O)ₘ;

Ring A is substituted with 0-3 groups selected from C₁-C₄alkyl, halo, OH, oxo, cyano, C₁-C₄alkyloxy, C₁-C₄alkyloxyC₁-C₄alkyl or C₁-C₄alkylcarbonyl, wherein each alkyl/alkyl group is substituted with 0-1 R¹⁸;

R⁵ is H;

R⁶ and R⁷ are independently selected from H and C₁-C₄alkyl; and,

n is 2.

In another embodiment, the present invention provides the novel substituted amides of formula I, wherein:

R¹ is selected from R⁸(C═O)—, R⁹S(O)₂—, R¹⁰R¹¹NC(═O)—, and R¹¹R¹¹NS(O)₂—;

R² is selected from H, C₁-C₄alkyl and C₃-C₆cycloalkyl;

Ring A is an 8-11 membered saturated or partially saturated bicyclic or tricyclic ring consisting of the shown nitrogen, 5-10 carbon atoms and from 0 to 1 additional heteroatoms selected from nitrogen, oxygen, and S(O)ₘ;

Ring A is substituted with 0-3 groups selected from C₁-C₄alkyl, halo, OH, oxo, cyano, C₁-C₄alkyloxy, C₁-C₄alkyloxyC₁-C₄alkyl or C₁-C₄alkylcarbonyl, wherein each alkyl/alkyl group is substituted with 0-1 R¹⁸;

R⁵ is H;

R⁶ and R⁷ are independently selected from H and C₁-C₄alkyl; and,

n is 2.

In another embodiment, the present invention provides the novel substituted amides of formula I, wherein:

R¹ is selected from R⁸(C═O)—, R⁹S(O)₂—, R¹⁰R¹¹NC(═O)—, and R¹¹R¹¹NS(O)₂—;

R² is C₁-C₄alkyl.

[3] In another embodiment, the present invention provides the novel substituted amides of formula I novel use of compounds of formula I, wherein:

R⁸ is selected from C₁-C₆alkyl, C₂-C₆alkenyl, aryl, hetaryl, arylC₁-C₄alkyl, hetarylC₁-C₄alkyl, C₃-C₆cycloalkyl, 3-6 membered hetcycloalkyl, aryloxyC₁-C₄alkyl, and hetaryloxyC₁-C₄alkyl, wherein each of the alkyl/alkyl, alkenyl, aryl, hetaryl, cycloalkyl, and hetcycloalkyl groups are independently substituted with 0-2 R¹⁹;

R⁹ is selected from C₁-C₆alkyl, C₂-C₆alkenyl, aryl, hetaryl, arylC₁-C₄alkyl, hetarylC₁-C₄alkyl, C₃-C₆cycloalkyl, 3-6 membered hetcycloalkyl, and aryloxyC₁-C₄alkyl, wherein each of the alkyl/alkyl, alkenyl, aryl, hetaryl, cycloalkyl, and hetcycloalkyl groups are independently substituted with 0-2 R²⁰;

R¹⁰ and R¹¹ are independently selected from H, C₃-C₆cycloalkyl, 3-6 membered hetcycloalkyl, aryl, and hetaryl, wherein each of the cycloalkyl, hetcycloalkyl, aryl, and hetaryl groups are independently substituted with 0-3 R²¹;

alternatively, R¹⁰ and R¹¹, together with the nitrogen to which they are attached, form a 5-6 membered saturated or partially saturated monocyclic ring consisting of the shown nitrogen atom, 4-5 carbon atoms, and 0-1 additional heteroatoms selected from nitrogen, oxygen, and S(O)ₘ, wherein this ring is substituted with 0-2 groups selected from C₁-C₈alkyl, aryl, hetaryl, hydroxy, oxo, COOH, C₁-C₆alkyloxy, arylC₁-C₆alkyloxy, hetarylC₁-C₆alkyloxy, and C₁-C₆alkylcarbonyl;

R¹² is selected from OH, C₁-C₄alkyl, C₃-C₆cycloalkyl, 3-10 membered hetcycloalkyl, trihalomethyl, C₁-C₄alkyloxy, aryl, arylC₁-C₄alkyl, hetaryl, hetarylC₁-C₄alkyl, aryloxy, and hetaryloxy;

R¹⁹, R²⁰ and R²¹ are independently selected from H, halo, OH, oxo, cyano, C₁-C₆alkyl, C₃-C₆cycloalkyl, 3-6 membered hetcycloalkyl, trihalomethyl, trihalomethyloxy, dihalo-methylenedioxo, C₁-C₄alkyloxy, aryl, hetaryl, arylC₁-C₄alkyl, hetarylC₁-C₄alkyl, —C(═O)R¹², —S(O)ₙR¹², and —S(O)ₙNR¹³R¹⁴; and,

n is 2.

In another embodiment, the present invention provides the novel substituted amide or prodrug thereof of formula Ia:

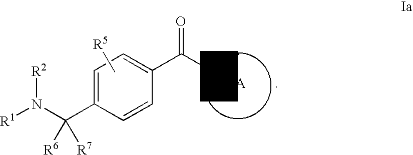

In another embodiment, the present invention provides the novel substituted amide or prodrug thereof of formula Ib:

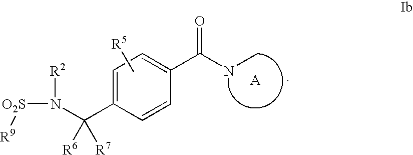

In another embodiment, the present invention provides the novel substituted amide or prodrug thereof of formula Ic:

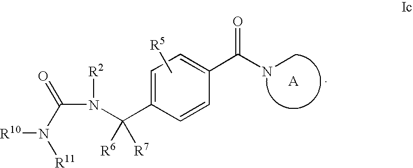

In another embodiment, the present invention provides the novel substituted amide or prodrug thereof of formula Id:

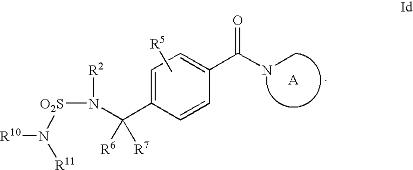

In another embodiment, the present invention provides the novel substituted amide or prodrug thereof of formula Ie:

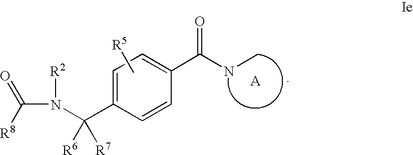

In another embodiment, the present invention provides the novel substituted amide formula I, wherein:

R¹ and R², together with the nitrogen to which they are attached, form a 5-12 membered saturated or partially saturated monocyclic or bicyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹²—S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸.

In another embodiment, the present invention provides the novel substituted amide formula I, wherein R¹ and R², together with the nitrogen to which they are attached, form a 5-12 membered saturated or partially saturated monocyclic or bicyclic ring consisting of the shown nitrogen, 4-10 carbon atoms, and 0-2 additional heteroatoms selected from nitrogen, oxygen, and S(O)ₘ, wherein this ring is substituted with 1-3 groups selected from C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹²—S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸.

In another embodiment, the present invention provides the novel substituted amide formula I, wherein R¹ and R², together with the nitrogen to which they are attached, form a 5 membered saturated ring consisting of the shown nitrogen, 2-3 carbon atoms, and 0-2 additional heteroatoms selected from nitrogen, oxygen, and S(O)ₘ, wherein this ring is substituted with 0-2 groups selected from C₁-C₈alkyl, aryl, hetaryl, arylC₁-C₆alkyl, hetarylC₁-C₆alkyl, —C(═O)R¹², —S(O)ₙR¹², —S(═O)ₙNR¹³R¹⁴, —N(R¹³)S(O)ₙR¹², OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸.

In another embodiment, the present invention provides the novel substituted amide formula I, wherein R¹ and R², together with the nitrogen to which they are attached, form a 5 membered saturated ring consisting of the shown nitrogen, 2-3 carbon atoms, and 1-2 additional heteroatoms selected from nitrogen, oxygen, and S(O)ₘ, wherein this ring is substituted with 0-2 groups selected from C₁-C₈alkyl, aryl, hetaryl, arylC₁-C₆alkyl, hetarylC₁-C₆alkyl, —C(═O)R¹², —S(O)ₙR¹², —S(═O)ₙNR¹³R¹⁴, —N(R¹³)S(O)ₙR¹², OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹³.

In another embodiment, the present invention provides the novel substituted amide formula I, wherein R¹ and R², together with the nitrogen to which they are attached, form a 5 membered saturated ring consisting of the shown nitrogen, 2-3 carbon atoms, and 1-2 additional heteroatoms selected from nitrogen and S(O)ₘ, wherein this ring is substituted with 0-2 groups selected from C₁-C₈alkyl, aryl, hetaryl, arylC₁-C₆alkyl, hetarylC₁-C₆alkyl, —C(═O)R¹², —S(O)ₙR¹², OH, oxo, C₁-C₆alkyloxy, arylC₁-C₆alkyloxy, hetarylC₁-C₆alkyloxy, and C₁-C₆alkyloxyC₁-C₆alkyl, wherein each alkyl and aryl/hetaryl group is substituted with 0-3 R¹⁸.

In another embodiment, the present invention provides the novel substituted amide formula I, wherein R¹ and R², together with the nitrogen to which they are attached, are:

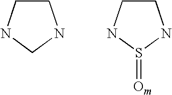

wherein this ring is substituted with 0-2 groups selected from C₁-C₈alkyl, aryl, hetaryl, arylC₁-C₆alkyl, hetarylC₁-C₆alkyl, —C(═O)R¹², —S(O)ₙR¹², OH, oxo, C₁-C₆alkyloxy, arylC₁-C₆alkyloxy, hetarylC₁-C₆alkyloxy, and C₁-C₆alkyloxyC₁-C₆alkyl, wherein each alkyl and aryl/hetaryl group is substituted with 0-3 R¹⁸.

In another embodiment, the present invention provides the novel substituted amide of formula I, wherein Ring A is selected from:

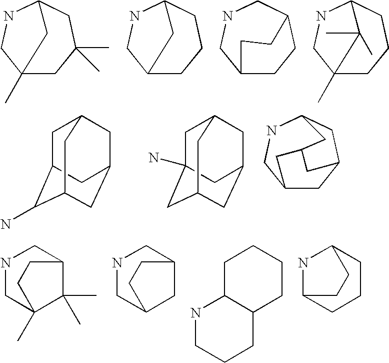

Ring A is substituted with 0-2 R²⁵; and, R²⁵ is selected from C₁-C₈alkyl, halo, hydroxy, oxo, cyano, C(═O)R¹², and C₁-C₆alkyloxy, wherein R¹² is as defined above.

In another embodiment, the present invention provides the novel substituted amide or prodrug thereof of formula I, wherein Ring A is selected from:

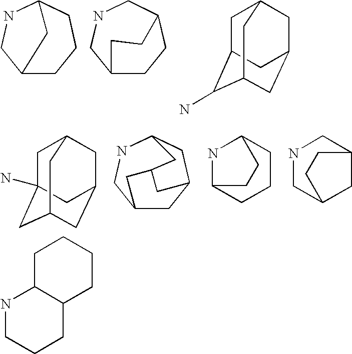

Ring A is substituted with 0-2 R²⁵; and, R²⁵ is selected from C₁-C₈alkyl, halo, hydroxy, oxo, cyano, and C₁-C₆alkyloxy.

In another embodiment, the present invention provides the novel substituted amide or prodrug thereof of formula I, wherein ring A is

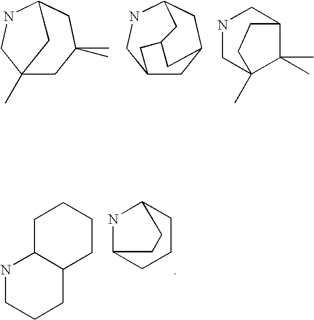

Ring A is substituted with 0-2 R²⁵; and, R²⁵ is selected from C₁-C₈alkyl, halo, hydroxy, oxo, cyano, and C₁-C₆alkyloxy.

In another embodiment, the present invention provides the novel substituted amide or prodrug thereof of formula I, wherein ring A is

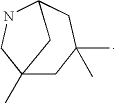

Ring A is substituted with 0-2 R²⁵; and, R²⁵ is selected from C₁-C₈alkyl, halo, hydroxy, oxo, cyano, and C₁-C₆alkyloxy.

In another embodiment, the present invention provides the novel substituted amide or prodrug thereof of formula I, wherein ring A is

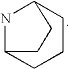

Ring A is substituted with 0-2 R²⁵; and, R²⁵ is selected from C₁-C₈alkyl, halo, hydroxy, oxo, cyano, and C₁-C₆alkyloxy.

In another embodiment, the present invention provides the novel substituted amide or prodrug thereof of formula I, wherein ring A is

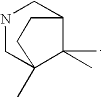

Ring A is substituted with 0-2 R²⁵; and, R²⁵ is selected from C₁-C₈alkyl, halo, hydroxy, oxo, cyano, and C₁-C₆alkyloxy.

In another embodiment, the present invention provides the novel substituted amide or prodrug thereof of formula I, wherein ring A is

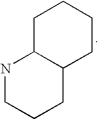

Ring A is substituted with 0-2 R²⁵; and, R²⁵ is selected from C₁-C₈alkyl, halo, hydroxy, oxo, cyano, and C₁-C₆alkyloxy.

In another embodiment, the present invention provides the novel substituted amide or prodrug thereof of formula I, wherein ring A is

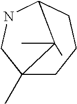

Ring A is substituted with 0-2 R²⁵; and, R²⁵ is selected from C₁-C₈alkyl, halo, hydroxy, oxo, cyano, and C₁-C₆alkyloxy.

In another embodiment, the present invention provides the novel substituted amide or prodrug thereof of formula I, wherein ring A is

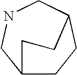

Ring A is substituted with 0-2 R²⁵; and, R²⁵ is selected from C₁-C₈alkyl, halo, hydroxy, oxo, cyano, and C₁-C₆alkyloxy.

In another embodiment, the present invention provides the novel substituted amide or prodrug thereof of formula I, wherein ring A is

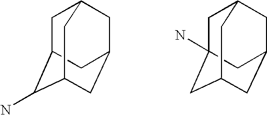

Ring A is substituted with 0-2 R²⁵; and, R²⁵ is selected from C₁-C₈alkyl, halo, hydroxy, oxo, cyano, and C₁-C₆alkyloxy.

In another embodiment, the present invention provides the novel substituted amide or prodrug thereof of formula I, wherein ring A is

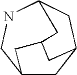

Ring A is substituted with 0-2 R²⁵; and, R²⁵ is selected from C₁-C₈alkyl, halo, hydroxy, oxo, cyano, and C₁-C₆alkyloxy.

In another embodiment, the present invention provides the novel substituted amide or prodrug thereof of formula I, wherein ring A is azepane.

In another embodiment, the present invention provides the novel compounds of formula I, wherein the substituted amide or a prodrug thereof is of the selected from the group:
N-Methyl-N-[4-(1,3,3-trimethyl-6-aza-bicyclo[3.2.1]octane-6-carbonyl)-benzyl]-acetamide
N-Methyl-N-[4-(1,3,3-trimethyl-6-aza-bicyclo[3.2.1]octane-6-carbonyl)-benzyl]-isobutyramide
Cyclopentanecarboxylic acid methyl-[4-(1,3,3-trimethyl-6-aza-bicyclo[3.2.1]octane-6-carbonyl)-benzyl]-amide
Cyclohexanecarboxylic acid methyl-[4-(1,3,3-trimethyl-6-aza-bicyclo[3.2.1]octane-6-carbonyl)-benzyl]-amide
Piperidine-1-carboxylic acid methyl-[4-(1,3,3-trimethyl-6-aza-bicyclo[3.2.1]octane-6-carbonyl)-benzyl]-amide
N-Methyl-N-[4-(1,3,3-trimethyl-6-aza-bicyclo[3.2.1]octane-6-carbonyl)-benzyl]-benzamide
1-Acetyl-piperidine-4-carboxylic acid methyl-[4-(1,3,3-trimethyl-6-azabicyclo[3.2.1]octane-6-carbonyl)-benzyl]-amide
1-Acetyl-piperidine-3-carboxylic acid methyl-[4-(1,3,3-trimethyl-6-azabicyclo[3.2.1]octane-6-carbonyl)-benzyl]-amide
Cyclopentanecarboxylic acid ethyl-[4-(1,3,3-trimethyl-6-aza-bicyclo[3.2.1]octane-6-carbonyl)-benzyl]-amide
Morpholine-4-carboxylic acid methyl-[4-(1,3,3-trimethyl-6-aza-bicyclo[3.2.1]octane-6-carbonyl)-benzyl]-amide
2,2-N-Trimethyl-N-[4-(1,3,3-trimethyl-6-aza-bicyclo[3.2.1]octane-6-carbonyl)-benzyl]-propionamide
Tetrahydro-furan-3-carboxylic acid methyl-[4-(1,3,3-trimethyl-6-azabicyclo[3.2.1]octane-6-carbonyl)-benzyl]-amide
N-Methyl-4-trifluoromethoxy-N-[4-(1,3,3-trimethyl-6-aza-bicyclo[3.2.1]octane-6-carbonyl)-benzyl]-benzamide
Thiophene-2-carboxylic acid methyl-[4-(1,3,3-trimethyl-6-aza-bicyclo[3.2.1]octane-6-carbonyl)-benzyl]-amide
Furan-2-carboxylic acid methyl-[4-(1,3,3-trimethyl-6-aza-bicyclo[3.2.1]octane-6-carbonyl)-benzyl]-amide
3-Chloro-4-(propane-2-sulfonyl)-thiophene-2-carboxylic acid methyl-[4-(1,3,3-trimethyl-6-aza-bicyclo[3.2.1]octane-6-carbonyl)-benzyl]-amide
6-Chloro-N-methyl-N-[4-(1,3,3-trimethyl-6-aza-bicyclo[3.2.1]octane-6-carbonyl)-benzyl]-nicotinamide
5-Methyl-isoxazole-3-carboxylic acid methyl-[4-(1,3,3-trimethyl-6-azabicyclo[3.2.1]octane-6-carbonyl)-benzyl]-amide
3,3,N-Trimethyl-N-[4-(1,3,3-trimethyl-6-aza-bicyclo[3.2.1]octane-6-carbonyl)-benzyl]-butyramide
3-Cyano-N-methyl-N-[4-(1,3,3-trimethyl-6-aza-bicyclo[3.2.1]octane-6-carbonyl)-benzyl]-benzamide
N-Methyl-2-phenoxy-N-[4-(1,3,3-trimethyl-6-aza-bicyclo[3.2.1]octane-6-carbonyl)-benzyl]-acetamide
N-Methyl-N-[4-(1,3,3-trimethyl-6-aza-bicyclo[3.2.1]octane-6-carbonyl)-benzyl]-malonamic acid methyl ester
3-Methyl-but-2-enoic acid methyl-[4-(1,3,3-trimethyl-6-aza-bicyclo[3.2.1]octane-6-carbonyl)-benzyl]-amide
N-Methyl-2-phenyl-N-[4-(1,3,3-trimethyl-6-aza-bicyclo[3.2.1]octane-6-carbonyl)-benzyl]-acetamide
1-Trifluoromethyl-cyclobutanecarboxylic acid methyl-[4-(1,3,3-trimethyl-6-azabicyclo[3.2.1]octane-6-carbonyl)-benzyl]-amide
3,5-Dimethoxy-N-methyl-N-[4-(1,3,3-trimethyl-6-aza-bicyclo[3.2.1]octane-6-carbonyl)-benzyl]-benzamide
4-Methanesulfonyl-N-methyl-N-[4-(1,3,3-trimethyl-6-aza-bicyclo[3.2.1]octane-6-carbonyl)-benzyl]-benzamide
N-Methyl-3-trifluoromethoxy-N-[4-(1,3,3-trimethyl-6-aza-bicyclo[3.2.1]octane-6-carbonyl)-benzyl]-benzamide
2,2-Difluoro-1,3-benzodioxole-4-carboxylic acid methyl-[4-(1,3,3-trimethyl-6-azabicyclo[3.2.1]octane-6-carbonyl)-benzyl]-amide
N-Methyl-6-morpholin-4-yl-N-[4-(1,3,3-trimethyl-6-aza-bicyclo[3.2.1]octane-6-carbonyl)-benzyl]-nicotinamide
N-Methyl-4-(2,2,2-trifluoro-acetyl)-N-[4-(1,3,3-trimethyl-6-aza-bicyclo[3.2.1]octane-6-carbonyl)-benzyl]-benzamide
N-[4-(1,3,3-Trimethyl-6-aza-bicyclo[3.2.1]octane-6-carbonyl)-benzyl]-benzamide
N-Methyl-N-[4-(1,3,3-trimethyl-6-aza-bicyclo[3.2.1]octane-6-carbonyl)-benzyl]-isophthalamic acid
2,3-Dihydro-benzofuran-7-carboxylic acid methyl-[4-(1,3,3-trimethyl-6-azabicyclo[3.2.1]octane-6-carbonyl)-benzyl]-amide
3-Acetyl-N-methyl-N-[4-(1,3,3-trimethyl-6-aza-bicyclo[3.2.1]octane-6-carbonyl)-benzyl]-benzamide
1,1,3-Trimethyl-3-[4-(1,3,3-trimethyl-bicyclo[3.2.1]octane-6-carbonyl)-benzyl]-sulphonylurea
N-Methyl-N-[4-(1,3,3-trimethyl-6-aza-bicyclo[3.2.1]octane-6-carbonyl)-benzyl]-methanesulfonamide
2,2,2-Trifluoro-ethanesulfonic acid methyl-[4-(1,3,3-trimethyl-6-azabicyclo[3.2.1]octane-6-carbonyl)-benzyl]-amide
N-Methylphenyl-N-[4-(1,3,3-trimethyl-6-aza-bicyclo[3.2.1]octane-6-carbonyl)-benzyl]-methanesulfonamide
Trifluoro-N-isopropyl-N-[4-(1,3,3-trimethyl-6-aza-bicyclo[3.2.1]octane-6-carbonyl)-benzyl]-methanesulfonamide
N-Cyclopropyl-trifluoro-N-[4-(1,3,3-trimethyl-6-aza-bicyclo[3.2.1]octane-6-carbonyl)-benzyl]-methanesulfonamide
N-Ethyl-trifluoro-N-[4-(1,3,3-trimethyl-6-aza-bicyclo[3.2.1]octane-6-carbonyl)-benzyl]-methanesulfonamide
Trifluoro-N-methyl-N-[4-(1,3,3-trimethyl-6-aza-bicyclo[3.2.1]octane-6-carbonyl)-benzyl]-methanesulfonamide
3-Benzoyl-1-methyl-1-[4-(1,3,3-trimethyl-6-aza-bicyclo[3.2.1]octane-6-carbonyl)-benzyl]-urea
3-Cyclohexyl-1-methyl-1-[4-(1,3,3-trimethyl-6-aza-bicyclo[3.2.1]octane-6-carbonyl)-benzyl]-urea
3-(4-methyl-phenyl)sulfonyl-1-methyl-1-[4-(1,3,3-tri-methyl-6-aza-bicyclo[3.2.1]octane-6-carbonyl)-benzyl]-urea
1,3-Dimethyl-3-phenyl-1-[4-(1,3,3-trimethyl-6-aza-bicyclo[3.2.1]octane-6-carbonyl)-benzyl]-urea
3-(2,3-Dihydro-1,4-benzodioxin-2-ylmethyl)-1-methyl-1-[4-(1,3,3-trimethyl-6-aza-bicyclo[3.2.1]octane-6-carbonyl)-benzyl]-urea
3-(3-Methoxy-benzyl)-1-methyl-1-[4-(1,3,3-trimethyl-6-aza-bicyclo[3.2.1]octane-6-carbonyl)-benzyl]-urea
3-(1,1-Dioxo-tetrahydro-thiophen-3-yl)-1-methyl-1-[4-(1,3,3-trimethyl-6-azabicyclo[3.2.1]octane-6-carbonyl)-benzyl]-urea
1-Methyl-3-(tetrahydro-pyran-4-yl)-1-[4-(1,3,3-trimethyl-6-aza-bicyclo[3.2.1]-octane-6-carbonyl)-benzyl]-urea
{1,3-Dimethyl-3-[4-(1,3,3-trimethyl-6-aza-bicyclo[3.2.1]octane-6-carbonyl)-benzyl]-ureido}-acetic acid methyl ester
1-Methyl-3-(5-trifluoromethyl-1,3,4-thiadiazol-2-yl)-1-[4-(1,3,3-trimethyl-6-azabicyclo[3.2.1]octane-6-carbonyl)-benzyl]-urea
2-{3-Methyl-3-[4-(1,3,3-trimethyl-6-aza-bicyclo[3.2.1]octane-6-arbonyl)-benzyl]-ureido}-benzoic acid methyl ester
3-{3-Methyl-3-[4-(1,3,3-trimethyl-6-aza-bicyclo[3.2.1]octane-6-carbonyl)-benzyl]-ureido}-benzoic acid ethyl ester
1-Methyl-3-(3-ethylsulfanyl-phenyl)-1-[4-(1,3,3-trimethyl-6-aza-bicyclo-[3.2.1]-octane-6-carbonyl)-benzyl]-urea
1-Methyl-3-(4-ethylsulfanyl-phenyl)-1-[4-(1,3,3-trimethyl-6-aza-bicyclo-[3.2.1]-octane-6-carbonyl)-benzyl]-urea
3-(4-Benzyloxy-phenyl)-1-methyl-1-[4-(1,3,3-trimethyl-6-aza-bicyclo[3.2.1]octane-6-carbonyl)-benzyl]-urea
1-Methyl-3-(4-trifluoro-methylsulfanyl-phenyl)-1-[4-(1,3,3-trimethyl-6-azabicyclo[3.2.1]octane-6-carbonyl)-benzyl]-urea
3-(4-Acetyl-phenyl)-1-methyl-1-[4-(1,3,3-tri-methyl-6-aza-bicyclo[3.2.1]octane-6-carbonyl)-benzyl]-urea
3-(3-Acetyl-phenyl)-1-methyl-1-[4-(1,3,3-tri-methyl-6-aza-bicyclo[3.2.1]octane-6-carbonyl)-benzyl]-urea
3-(3-Cyano-phenyl)-1-methyl-1-[4-(1,3,3-tri-methyl-6-aza-bicyclo[3.2.1]octane-6-carbonyl)-benzyl]-urea
1-Methyl-3-(4-trifluoro-methyl-phenyl)-1-[4-(1,3,3-trimethyl-6-aza-bicyclo-[3.2.1]octane-6-carbonyl)-benzyl]-urea
3-(4-Methoxy-benzyl)-1-methyl-1-[4-(1,3,3-tri-methyl-6-aza-bicyclo[3.2.1]octane-6-carbonyl)-benzyl]-urea
1-Methyl-3-(2,2,4,4-tetrafluoro-4H-benzo-[1,3]dioxin-6-yl)-1-[4-(1,3,3-trimethyl-6-aza-bicyclo-[3.2.1]octane-6-carbonyl)-benzyl]-urea
1-Methyl-3-(4-trifluoro-methoxy-phenyl)-1-[4-(1,3,3-trimethyl-6-aza-bicyclo[3.2.1]-octane-6-carbonyl)-benzyl]-urea
1-Methyl-3-[4-(2,2,2-trifluoro-acetyl)-cyclohexyl]-1-[4-(1,3,3-trimethyl-6-aza-bicyclo[3.2.1]octane-6-carbonyl)-benzyl]-urea
1-(4-Acetyl-phenyl)-1,3-dimethyl-3-[4-(1,3,3-tri-methyl-6-aza-bicyclo[3.2.1]octane-6-carbonyl)-benzyl]-urea
1-Phenyl-3-{1-[4-(1,3,3-trimethyl-6-aza-bicyclo-[3.2.1]octane-6-carbonyl)-phenyl]-cyclopropyl}-urea
Piperidine-1-carboxylic acid methyl-[4-(octahydro-quinoline-1-carbonyl)-benzyl]-amide
Piperidine-1-carboxylic acid [4-(3-aza-bicyclo-[3.2.2]nonane-3-carbonyl)-benzyl]-methyl-amide
Morpholine-4-carboxylic acid methyl-[4-(octahydro-quinoline-1-carbonyl)-benzyl]-amide
Morpholine-4-carboxylic acid [4-(3-aza-bicyclo-[3.2.2]nonane-3-carbonyl)-benzyl]-methyl-amide
1,3-Dimethyl-3-[4-(octahydro-quinoline-1-carbonyl)-benzyl]-1-phenyl-urea
1-[4-(3-Aza-bicyclo-[3.2.2]nonane-3-carbonyl)-benzyl]-1,3-dimethyl-3-phenyl-urea
Piperidine-1-carboxylic acid [4-(6-aza-bicyclo-[3.2.1]octane-6-carbonyl)-benzyl]-methyl-amide
Piperidine-1-carboxylic acid methyl-[4-(1,8,8-trimethyl-3-aza-bicyclo[3.2.1]octane-3-carbonyl)-benzyl]-amide
Morpholine-4-carboxylic acid [4-(6-aza-bicyclo-[3.2.1]octane-6-carbonyl)-benzyl]-methyl-amide
Morpholine-4-carboxylic acid methyl-[4-(1,8,8-trimethyl-3-aza-bicyclo[3.2.1]-octane-3-carbonyl)-benzyl]-amide
1-[4-(6-Aza-bicyclo-[3.2.1]octane-6-carbonyl)-benzyl]-1,3-dimethyl-3-phenyl-urea
1,3-Dimethyl-1-phenyl-3-[4-(1,8,8-trimethyl-3-aza-bicyclo[3.2.1]octane-3-carbonyl)-benzyl]-urea
Piperidine-1-carboxylic acid [4-(3-hydroxy-8-aza-bicyclo[3.2.1]octane-8-carbonyl)-benzyl]-methyl-amide
Morpholine-4-carboxylic acid [4-(3-hydroxy-8-aza-bicyclo[3.2.1]octane-8-carbonyl)-benzyl]-methyl-amide
1-[4-(3-Hydroxy-8-aza-bicyclo[3.2.1]octane-8-carbonyl)-benzyl]-1,3-di-methyl-3-phenyl-urea
1-[4-(3-Fluoro-8-aza-bicyclo[3.2.1]octane-8-carbonyl)-benzyl]-1,3-di-methyl-3-phenyl-urea
Piperidine-1-carboxylic acid [4-(3-fluoro-8-aza-bicyclo[3.2.1]octane-8-carbonyl)-benzyl]-methyl-amide
Morpholine-4-carboxylic acid [4-(3-fluoro-8-aza-bicyclo[3.2.1]octane-8-carbonyl)-benzyl]-methyl-amide
N-Adamantan-2-yl-4-(1,3-dimethyl-3-pyridin-2-yl-ureidomethyl)-benzamide
1-[4-(3-Aza-bicyclo[3.2.2]nonane-3-carbonyl)-benzyl]-1,3-dimethyl-3-pyridin-2-yl-urea
1-[4-(3-Hydroxy-8-aza-bicyclo[3.2.1]octane-8-carbonyl)-benzyl]-1,3-dimethyl-3-pyridin-2-yl-urea
Morpholine-4-carboxylic acid [4-(adamantan-2-ylcarbamoyl)-benzyl]-methyl-amide
1-[4-(3-Aza-bicyclo[3.2.2]nonane-3-carbonyl)-benzyl]-1,3-dimethyl-3-thiazol-2-yl-urea
1,3-Dimethyl-3-[4-(2-oxa-5-aza-bicyclo[2.2.1]heptane-5-carbonyl)-benzyl]-1-thiazol-2-yl-urea
4-[3-(1-Acetyl-piperidin-4-yl)-1,3-dimethyl-ureidomethyl]-N-adamantan-2-yl-benzamide
1-(1-Acetyl-piperidin-4-yl)-3-[4-(3-aza-bicyclo[3.2.2]nonane-3-carbonyl)-benzyl]-1,3-dimethyl-urea
N-Adamantan-2-yl-4-(1,3-dimethyl-3-pyrimidin-2-yl-ureidomethyl)-benzamide
1-[4-(3-Aza-bicyclo[3.2.2]nonane-3-carbonyl)-benzyl]-1,3-dimethyl-3-pyrimidin-2-yl-urea
N-Adamantan-2-yl-4-(1,3-dimethyl-3-thiazol-2-yl-ureidomethyl)-benzamide
N-Adamantan-2-yl-4-(1,3-dimethyl-3-phenyl-ureidomethyl)-benzamide
1-[4-(3-Hydroxy-8-aza-bicyclo[3.2.1]octane-8-carbonyl)-benzyl]-1,3-dimethyl-3-pyrimidin-2-yl-urea
N-Adamantan-2-yl-4-[3-(4-hydroxy-cyclohexyl)-1,3-dimethyl-ureidomethyl]-benzamide
1-[4-(3-Aza-bicyclo[3.2.2]nonane-3-carbonyl)-benzyl]-3-(4-hydroxy-cyclohexyl)-1,3-dimethyl-urea
1-(4-Hydroxy-cyclohexyl)-1,3-dimethyl-3-[4-(2-oxa-5-aza-bicyclo[2.2.1]heptane-5-carbonyl)-benzyl]-urea
1-Methyl-3-[4-(1,3,3-trimethyl-6-aza-bicyclo[3.2.1]octane-6-carbonyl)-benzyl]-imidazo-lidin-2-one
[4-(1,1-Dioxo-isothiazolidin-2-ylmethyl)-phenyl]-(1,3,3-trimethyl-6-aza-bicyclo[3.2.1]-oct-6-yl)-methanone
[4-(1,1-Dioxo-1,2,5-thiadiazolidin-2-ylmethyl)-phenyl]-(1,3,3-trimethyl-bicyclo[3.2.1]oct-6-yl)-methanone
[4-(5-Methyl-1,1-dioxo-1,2,5-thiadiazolidin-2-ylmethyl)-phenyl]-(1,3,3-trimethyl-bicyclo[3.2.1]oct-6-yl)-methanone
(Octahydro-quinolin-1-yl)-[4-(1,1-dioxo-1,2,5-thiadiazolidin-2-ylmethyl)-phenyl]-methanone
(4-Aza-tricyclo[4.3.1.1^{3,8}]-undec-4-yl)-[4-(1,1-dioxo-1,2,5-thiadiazolidin-2-ylmethyl)-phenyl]-methanone
(Octahydro-isoquinolin-2-yl)-[4-(1,1-dioxo-1,2,5-thiadiazolidin-2-ylmethyl)-phenyl]-methanone
(3-Aza-bicyclo[3.2.2]non-3-yl)-[4-(1,1-dioxo-1,2,5-thiadiazolidin-2-ylmethyl)-phenyl]-methanone
(6-Aza-bicyclo[3.2.1]oct-6-yl)-[4-(1,1-dioxo-1,2,5-thiadiazolidin-2-ylmethyl)-phenyl]-methanone
[4-(5-Benzyl-1,1-dioxo-[1,2,5]thiadiazolidin-2-ylmethyl)-phenyl]-(1,3,3-trimethyl-6-aza-bicyclo[3.2.1]oct-6-yl)-methanone

or a salt thereof with a pharmaceutically acceptable acid or base, or any optical isomer or mixture of optical isomers, including a racemic mixture, or any tautomeric forms.

In another embodiment, the present invention provides the novel compounds of for mula I, wherein the substituted amide or a prodrug thereof is the selected from the group:
[4-(1-Amino-cyclopropyl)-phenyl]-(1,3,3-trimethyl-6-aza-bicyclo[3.2.1]oct-6-yl)-methanone
Piperidine-3-carboxylic acid methyl-[4-(1,3,3-trimethyl-6-aza-bicyclo[3.2.1]octane-6-carbonyl)-benzyl]-amide
N-Methyl-N-[4-(1,3,3-tri-methyl-6-aza-bicyclo[3.2.1]-octane-6-carbonyl)-benzyl]-butyramide
N-Methyl-N-[4-(octahydro-quinoline-1-carbonyl)-benzyl]-benzamide
N-[4-(3-Aza-bicyclo[3.2.2]-nonane-3-carbonyl)-benzyl]-N-methyl-benzamide

### 3-Cyano-N-methyl-N-[4-(octahydro-quinoline-1-carbonyl)-benzyl]-benzamide

N-[4-(3-Azabicyclo[3.2.2]-nonane-3-carbonyl)-benzyl]-3-cyano-N-methyl-benzamide
3-Fluoro-N-methyl-N-[4-(octahydro-quinoline-1-carbonyl)-benzyl]-benzamide
N-[4-(3-Aza-bicyclo[3.2.2]-nonane-3-carbonyl)-benzyl]-3-fluoro-N-methyl-benzamide
N-[4-(Azepane-1-carbonyl)-benzyl]-3-fluoro-N-methyl-benzamide
N-[4-(Azepane-1-carbonyl)-benzyl]-N-methyl-benzamide
N-[4-(Azepane-1-carbonyl)-benzyl]-3-cyano-N-methyl-benzamide
Piperidine-1-carboxylic acid [4-(azepane-1-carbonyl)-benzyl]-methyl-amide
Morpholine-4-carboxylic acid [4-(azepane-1-carbonyl)-benzyl]-methyl-amide
N-[4-(Octahydro-quinoline-1-carbonyl)-benzyl]-benzamide
N-[4-(3-Azabicyclo[3.2.2]-nonane-3-carbonyl)-benzyl]-benzamide
N-[4-(Azepane-1-carbonyl)-benzyl]-benzamide
N-[4-(6-Aza-bicyclo[3.2.1]-octane-6-carbonyl)-benzyl]-N-methyl-benzamide
4-[(Benzoyl-methyl-amino)-methyl]-N-(3-hydroxy-adamantan-1-yl)-benzamide
N-[4-(6-Aza-bicyclo[3.2.1]-octane-6-carbonyl)-benzyl]-3-cyano-N-methyl-benzamide
4-[(3-Cyano-benzoyl-methyl-amino)-methyl]-N-(3-hydroxy-adamantan-1-yl)-benzamide
N-[4-(6-Aza-bicyclo[3.2.1]-octane-6-carbonyl)-benzyl]-3-fluoro-N-methyl-benzamide
4-[(3-Fluoro-benzoyl-methyl-amino)-methyl]-N-(3-hydroxy-adamantan-1-yl)-benzamide
1-Acetyl-piperidine-4-carboxylic acid methyl-[4-(octahydro-quinoline-1-carbonyl)-benzyl]-amide
N-[4-(3-Hydroxy-8-aza-bicyclo[3.2.1]octane-8-carbonyl)-benzyl]-N-methyl-benzamide
3-Fluoro-N-methyl-N-[4-(1,8,8-trimethyl-3-aza-bicyclo[3.2.1]octane-3-carbonyl)-benzyl]-benzamide
3-Fluoro-N-[4-(3-hydroxy-8-aza-bicyclo[3.2.1]octane-8-carbonyl)-benzyl]-N-methyl-benzamide
N-Methyl-N-[4-(1,8,8-trimethyl-3-aza-bicyclo[3.2.1]-octane-3-carbonyl)-benzyl]-benzamide
N-[4-(6-Aza-bicyclo[3.2.1]-octane-6-carbonyl)-benzyl]-benzamide
N-[4-(1,8,8-Trimethyl-3-aza-bicyclo[3.2.1]octane-3-carbonyl)-benzyl]-benzamide
N-[4-(3-Hydroxy-8-aza-bicyclo[3.2.1]octane-8-carbonyl)-benzyl]-benzamide
1-Acetyl-piperidine-4-carboxylic acid [4-(azepane-1-carbonyl)-benzyl]-methyl-amide
4-(Benzoylamino-methyl)-N-(3-hydroxy-adamantan-1-yl)-benzamide
3-Cyano-N-methyl-N-[4-(1,8,8-trimethyl-3-aza-bi-cyclo[3.2.1]octane-3-car-bonyl)-benzyl]-benzamide
3-Cyano-N-[4-(3-fluoro-8-aza-bicyclo[3.2.1]octane-8-carbonyl)-benzyl]-N-methyl-benzamide
3-Fluoro-N-[4-(3-fluoro-8-aza-bicyclo[3.2.1]octane-8-carbonyl)-benzyl]-N-methyl-benzamide
4-(3-Fluoro-benzoylamino-methyl)-N-methyl-N-(3-fluoro-adamantan-1-yl)-benzamide
4-(3-Cyano-benzoylamino-methyl)-N-methyl-N-(3-fluoro-adamantan-1-yl)-benzamide
1-Acetyl-piperidine-4-carboxylic acid methyl-[4-(1,8,8-trimethyl-3-aza-bicyclo[3.2.1]-octane-3-carbonyl)-benzyl]-amide
N-[4-(3-Fluoro-8-aza-bicyclo[3.2.1]octane-8-carbonyl)-benzyl]-N-methyl-benzamide
4-[(Benzoyl-methyl-amino)-methyl]-N-(3-fluoro-adamantan-1-yl)-benzamide
3-Cyano-N-[4-(3-hydroxy-8-aza-bicyclo[3.2.1]octane-8-carbonyl)-benzyl]-N-methyl-benzamide
4-(Benzoylamino-methyl)-N-(3-fluoro-adamantan-1-yl)-benzamide
1-Acetyl-piperidine-4-carboxylic acid [4-(3-aza-bicyclo[3.2.2]nonane-3-carbonyl)-benzyl]-methyl-amide
N-[4-(3-Fluoro-8-aza-bicyclo[3.2.1]octane-8-carbonyl)-benzyl]-benzamide
4-(3-Cyano-benzoylamino-methyl)-N-(adamantan-2-yl)-benzamide
4-(3-Fluoro-benzoylamino-methyl)-N-(adamantan-2-yl)-benzamide
N-[4-(4-Azatricyclo-[4.3.1.1*3,8*]undecane-4-carbonyl)-benzyl]-3-fluoro-N-methyl-benzamide
N-{1-[4-(1,3,3-Trimethyl-6-aza-bicyclo[3.2.1]octane-6-carbonyl)-phenyl]-cyclo-propyl}-benzamide
N-{1-[4-(1,3,3-Trimethyl-6-aza-bicyclo[3.2.1]octane-6-carbonyl)-phenyl]-cyclo-propyl}-acetamide
4-Methanesulfonyl-N-{1-[4-(1,3,3-trimethyl-6-aza-bicyclo[3.2.1]octane-6-carbonyl)-phenyl]-cyclo-propyl}-benzamide
N-Methyl-N-{1-[4-(1,3,3-trimethyl-6-aza-bicyclo-[3.2.1]octane-6-carbonyl)-phenyl]-cyclo-propyl}-benzamide
N-Methyl-N-{1-[4-(1,3,3-trimethyl-6-aza-bicyclo-[3.2.1]octane-6-carbonyl)-phenyl]-cyclo-propyl}-acetamide
4-Methanesulfonyl-N-methyl-N-{1-[4-(1,3,3-trimethyl-6-aza-bicyclo[3.2.1]octane-6-carbonyl)-phenyl]-cyclo-propyl}-benzamide
N-[4-(Azepane-1-carbonyl)-benzyl]-N-methyl-methane-sulfonamide
N-[4-(3-Aza-bicyclo[3.2.2]-nonane-3-carbonyl)-benzyl]-N-methyl-methane-sulfonamide
N-Methyl-N-[4-(1,8,8-tri-methyl-3-aza-bicyclo[3.2.1]-octane-3-carbonyl)-benzyl]-methane-sulfonamide
N-[4-(6-Aza-bicyclo[3.2.1]-octane-6-carbonyl)-benzyl]-N-methyl-methane-sulfonamide
N-[4-(3-Hydroxy-8-aza-bicyclo[3.2.1]octane-8-carbonyl)-benzyl]-N-methyl-methanesulfonamide
N-Methyl-N-[4-(octahydro-quinoline-1-carbonyl)-benzyl]-methanesulfon-amide
N-(3-Hydroxy-adamantan-1-yl)-4-[(methanesulfonyl-methyl-amino)-methyl]-benzamide
N-(3-Fluoro-adamantan-1-yl)-4-[(methanesulfonyl-methyl-amino)-methyl]-benzamide
N-[4-(3-Fluoro-8-aza-bicyclo[3.2.1]octane-8-carbonyl)-benzyl]-N-methyl-methane-sulfonamide
N-Adamantan-2-yl-4-[(methanesulfonyl-methyl-amino)-methyl]-benzamide
N-(4-{1-[4-(1,3,3-Trimethyl-6-aza-bicyclo[3.2.1]octane-6-carbonyl)-phenyl]-cyclo-propylsulfamoyl}-phenyl)-acetamide
4-Chloro-N-{1-[4-(1,3,3-tri-methyl-6-aza-bicyclo-[3.2.1]octane-6-carbonyl)-phenyl]-cyclo-propyl}-benzene-sulfonamide
1-Methyl-1H-imidazole-4-sulfonic acid {1-[4-(1,3,3-trimethyl-6-aza-bicyclo-[3.2.1]-octane-6-carbonyl)-phenyl]-cyclopropyl}-amide
N-{1-[4-(1,3,3-Trimethyl-6-aza-bicyclo[3.2.1]octane-6-carbonyl)-phenyl]-cyclo-propyl}-ethanesulfonamide
1-[4-(Azepane-1-carbonyl)-benzyl]-1,3-dimethyl-3-phenyl-urea
Piperidine-1-carboxylic acid [4-(3-hydroxy-adamantan-1-yl-carbamoyl)-benzyl]-methyl-amide
Morpholine-4-carboxylic acid [4-(3-hydroxy-adamantan-1-yl-carbamoyl)-benzyl]-methyl-amide
4-(1,3-Dimethyl-3-phenyl-ureidomethyl)-N-(3-hydroxy-adamantan-1-yl)-benzamide
Piperidine-1-carboxylic acid [4-(3-fluoro-adamantan-1-yl-carbamoyl)-benzyl]-methyl-amide
Morpholine-4-carboxylic acid [4-(3-fluoro-adamantan-1-yl-carbamoyl)-benzyl]-methyl-amide
4-(1,3-Dimethyl-3-phenyl-ureidomethyl)-N-(3-fluoro-adamantan-1-yl)-benzamide
N-Adamantan-2-yl-4-(1,3-dimethyl-3-pyridin-2-yl-ureidomethyl)-benzamide
1,3-Dimethyl-3-[4-(2-oxa-5-aza-bicyclo[2.2.1]heptane-5-carbonyl)-benzyl]-1-pyridin-2-yl-urea
1-[4-(3-Hydroxy-8-aza-bicyclo[3.2.1]octane-8-carbonyl)-benzyl]-1,3-dimethyl-3-thiazol-2-yl-urea
1-(1-Acetyl-piperidin-4-yl)-1,3-dimethyl-3-[4-(2-oxa-5-aza-bicyclo[2.2.1]heptane-5-carbonyl)-benzyl]-urea
1-(1-Acetyl-piperidin-4-yl)-3-[4-(3-hydroxy-8-aza-bicyclo[3.2.1]-octane-8-carbonyl)-benzyl]-1,3-dimethyl-urea
1,3-Dimethyl-3-[4-(2-oxa-5-aza-bicyclo[2.2.1]heptane-5-arbonyl)-benzyl]-1-pyrimidin-2-yl-urea
Morpholine-4-carboxylic acid [4-(4-aza-tricyclo[4.3.1.1*3,8*]-undecane-4-carbonyl)-benzyl]-methyl-amide
1-[4-(3-Hydroxy-8-aza-bicyclo[3.2.1]octane-8-carbonyl)-benzyl]-3-(4-hydroxy-cyclohexyl)-1,3-dimethyl-urea
1-[4-(3-Aza-bicyclo[3.2.2]nonane-3-carbonyl)-benzyl]-3-(4-fluoro-cyclohexyl)-1,3-dimethyl-urea
N-Adamantan-2-yl-4-[3-(1-cyclopropyl-piperidin-4-yl)-1,3-dimethyl-ureidomethyl]-benzamide
1-[4-(3-Methoxy-8-aza-bicyclo[3.2.1]octane-8-carbonyl)-benzyl]-1,3-dimethyl-3-phenyl-urea
N-Adamantan-2-yl-4-[3-(4-fluoro-phenyl)-2-oxo-imidazolidin-1-ylmethyl]-benzamide
1-[4-(3-Aza-bicyclo[3.2.2]nonane-3-carbonyl)-benzyl]-3-(4-fluoro-phenyl)-imidazolidin-2-one
1-(4-Fluoro-phenyl)-3-[4-(3-hydroxy-8-aza-bicyclo[3.2.1]octane-8-carbonyl)-benzyl]-imidazolidin-2-one
N-Adamantan-2-yl-4-(2-oxo-3-phenyl-imidazolidin-1-ylmethyl)-benzamide
N-Adamantan-1-yl-4-(1,1-dioxo-[1,2,5]thiadiazolidin-2-ylmethyl)-benzamide
N-Adamantan-2-yl-4-(1,1-dioxo-[1,2,5]thiadiazolidin-2-ylmethyl)-benzamide
(4-Azatricyclo[4.3.1.1*3,8*]-undec-4-yl)-[4-(1,1-dioxo-[1,2,5]thiadiazolidin-2-ylmethyl)-phenyl]-methanone
Azepan-1-yl-[4-(1,1-dioxo-[1,2,5]thiadiazolidin-2-ylmethyl)-phenyl]-methanone
Azepan-1-yl-[4-(5-methyl-1,1-dioxo-[1,2,5]thiadiazolidin-2-ylmethyl)-phenyl]-methanone
N-Adamantan-1-yl-4-(5-methoxymethyl-1,1-dioxo-[1,2,5]thiadiazolidin-2-ylmethyl)-benzamide
4-(1,1-Dioxo-[1,2,5]thiadiazolidin-2-ylmethyl)-N-(3-hydroxy-adamantan-1-yl)-benzamide
{5-[4-(Adamantan-1-yl-carbamoyl)-benzyl]-1,1-dioxo-[1,2,5]thiadiazolidin-2-yl}-acetic acid tert-butyl ester
   or a salt thereof with a pharmaceutically acceptable acid or base, or any optical isomer or mixture of optical isomers, including a racemic mixture, or any tautomeric forms.

[1u] In another embodiment, the present invention provides for the novel use of a substituted amide, a prodrug thereof, or a salt thereof with a pharmaceutically acceptable acid or base, or any optical isomer or mixture of optical isomers, including a racemic mixture or any tautomeric forms, wherein the substituted amide or a prodrug thereof is of formula I:

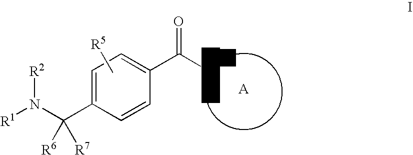

wherein:

R¹ is selected from H, R⁸(C═O)—, R⁹S(O)ₙ—, R¹⁰R¹¹NC(═Y)—, and R¹⁰R¹¹NS(O)ₙ—;

R² is selected from H, C₁-C₆alkyl, and C₃-C₆cycloalkyl;

alternatively, R¹ and R², together with the nitrogen to which they are attached, form a 3-12 membered saturated or partially saturated monocyclic or bicyclic ring consisting of the shown nitrogen, 2-10 carbon atoms, and 0-2 additional heteroatoms selected from nitrogen, oxygen, and S(O)ₘ, wherein this ring is substituted with 0-3 groups selected from C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)R¹², —S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸;

Ring A is a 5-12 membered saturated or partially saturated bicyclic or tricyclic ring consisting of the shown nitrogen, 4-10 carbon atoms and from 0 to 2 additional heteroatoms selected from nitrogen, oxygen, and S(O)ₘ;

Ring A is substituted with 0-3 groups selected from C₁-C₈alkyl, halo, OH, oxo, cyano, C₁-C₆alkyloxy, C₁-C₆alkyloxyC₁-C₆alkyl or C₁-C₆alkylcarbonyl, wherein each alkyl group is substituted with 0-3 R¹⁸;

R⁵ is selected from H, C₁-C₆alkyl, C₃-C₆cycloalkyl, halo, OH, and cyano;

R⁶ and R⁷ are independently selected from H, C₁-C₆alkyl, F, trihalomethyl, and trihalomethoxy;

alternatively, R⁶ and R⁷, together with the carbon atom to which they are attached, form a 3-8 membered saturated or partially saturated monocyclic ring consisting of the shown carbon atom, 2-5 additional carbon atoms, and 0-2 heteroatoms selected from nitrogen, oxygen, and S(O)ₘ, wherein this ring is substituted with 0-3 groups selected from halo, trihalomethyl, OH, C₁-C₆alkyl, oxo, and C₁-C₆alkyloxy;

R⁸ is selected from C₁-C₈alkyl, C₂-C₈alkenyl, aryl, hetaryl, arylC₁-C₆alkyl, hetarylC₁-C₆alkyl, C₃-C₁₀cycloalkyl, 3-10 membered hetcycloalkyl, aryloxyC₁-C₆alkyl, hetaryloxyC₁-C₆alkyl, arylC₁-C₆alkyloxyC₁-C₆alkyl, and hetarylC₁-C₆alkyloxyC₁-C₆alkyl, wherein each of the alkyl/alkyl, alkenyl, aryl, hetaryl, cycloalkyl, and hetcycloalkyl groups are independently substituted with 0-3 R¹⁹;

R⁹ is selected from C₁-C₈alkyl, C₂-C₈alkenyl, aryl, hetaryl, arylC₁-C₆alkyl, hetarylC₁-C₆alkyl, C₃-C₁₀cycloalkyl, 3-10 membered hetcycloalkyl, aryloxyC₁-C₆alkyl, and arylC₁-C₆alkyloxyC₁-C₆alkyl, wherein each of the alkyl/alkyl, alkenyl, aryl, hetaryl, cycloalkyl, and hetcycloalkyl groups are independently substituted with 0-3 R²⁰;

R¹⁰ and R¹¹ are independently selected from H, C₁-C₈alkyl, C₃-C₁₀cycloalkyl, 3-10 membered hetcycloalkyl, aryl, hetaryl, arylC₁-C₆alkyl, and hetarylC₁-C₆alkyl, wherein each of the alkyl/alkyl, cycloalkyl, hetcycloalkyl, aryl, and hetaryl groups are independently substituted with 0-3 R²¹.

alternatively, R¹⁰ and R¹¹, together with the nitrogen to which they are attached, form a 5-12 membered saturated or partially saturated monocyclic, bicyclic, or tricyclic ring consisting of the shown nitrogen atom, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkyl, hetarylC₁-C₆alkyl, hydroxy, oxo, COOH, C₁-C₆alkyloxy, arylC₁-C₆alkyloxy, hetarylC₁-C₆alkyloxy, C₁-C₆alkyloxyC₁-C₆alkyl, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkyl-carboxy, and hetarylC₁-C₆alkylcarboxy;

R¹² is selected from OH, C₁-C₈alkyl, C₃-C₁₀cycloalkyl, 3-10 membered hetcycloalkyl, trihalomethyl, C₁-C₈alkyloxy, aryl, arylC₁-C₆alkyl, hetaryl, hetarylC₁-C₆alkyl, aryloxy, hetaryloxy, and NR¹³R¹⁴;

R¹³ and R¹⁴ are independently selected from H, C₁-C₈alkyl, C₃-C₁₀cycloalkyl, aryl, hetaryl, arylC₁-C₆alkyl, and hetarylC₁-C₆alkyl, wherein each of the alkyl/alkyl, cycloalkyl, aryl, and hetaryl groups are independently substituted with 0-3 R²²;

alternatively, R¹³ and R¹⁴, together with the nitrogen to which they are attached, form a 5-12 membered saturated or partially saturated monocyclic, bicyclic, or tricyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkyl, hetarylC₁-C₆alkyl, OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkylcarboxy, and hetarylC₁-C₆alkylcarboxy;

R¹⁵ is selected from H, C₁-C₆alkyl, and C₃-C₆cycloalkyl;

R¹⁶ and R¹⁷ are independently selected from H, C₁-C₈alkyl, C₃-C₁₀cycloalkyl, halo, OH, cyano, —C(═O)R¹², —S(O)ₙR¹², —S(O)ₙNR¹³R¹⁴, —N(R¹³)S(O)ₙR¹², C₁-C₈alkyl, aryl, and hetaryl, wherein the alkyl and cycloalkyl groups are independently substituted with 0-3 R²²;

R¹⁸ is selected from halo, OH, oxo, COOH, cyano C₁-C₆alkyloxy, C₃-C₁₀cycloalkyloxy, aryloxy, hetaryloxy, hetarylthio and arylC₁-C₆alkyloxy;

R¹⁹, R²⁰ and R²¹ are independently selected from H, halo, OH, oxo, cyano, C₁-C₈alkyl, C₃-C₁₀cycloalkyl, 3-10 membered hetcycloalkyl, trihalomethyl, trihalomethyloxy, methylendioxo, dihalo-methylenedioxo, C₃-C₆spirocycloalkyl, C₁-C₆alkyloxy, aryl, hetaryl, arylC₁-C₆alkyl, hetarylC₁-C₆alkyl, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, and —C(═NR¹⁶)NR¹⁷;

R²² is selected from H, OH, oxo, halo, cyano, nitro, C₁-C₆alkyl, C₁-C₆alkyloxy, NR²³R²⁴, methylendioxo, dihalomethylendioxo, trihalomethyl, and trihalomethyloxy;

R²³ and R²⁴ are independently selected from H, C₁-C₈alkyl, and arylC₁-C₆alkyl;

m is selected from 0, 1, and 2;

n is selected from 1 and 2;

Y is selected from O and S;

or a salt thereof with a pharmaceutically acceptable acid or base, or any optical isomer or mixture of optical isomers, including a racemic mixture, or any tautomeric forms.

[1] In another embodiment, the present invention provides the novel use of compounds of formula I, wherein:

R¹ is selected from H, R⁸(C═O)—, R⁹S(O)ₙ—, R¹⁰R¹¹NC(═Y)—, and R¹⁰R¹¹NS(O)ₙ—;

R² is selected from H, C₁-C₆alkyl, and C₃-C₆cycloalkyl;

alternatively, R¹ and R², together with the nitrogen to which they are attached, form a 3-12 membered saturated or partially saturated monocyclic or bicyclic ring consisting of the shown nitrogen, 2-10 carbon atoms, and 0-2 additional heteroatoms selected from nitrogen, oxygen, and S(O)ₘ, wherein this ring is substituted with 0-3 groups selected from C₁-C₈alkyl, C₃-C₆spirocycloalkyl, 3-6 membered spirohetcycloalkyl, aryl, hetaryl, arylC₁-C₆alkylene, hetarylC₁-C₆alkylene, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each aryl/hetaryl group is substituted with 0-3 R¹⁸;

Ring A is a 5-12 membered saturated or partially saturated bicyclic or tricyclic ring consisting of the shown nitrogen, 4-10 carbon atoms and from 0 to 2 additional heteroatoms selected from nitrogen, oxygen, and S(O)ₘ;

Ring A is substituted with 0-3 groups selected from C₁-C₈alkyl, halo, OH, oxo, cyano, C₁-C₆alkyloxy, C₁-C₆alkyloxyC₁-C₆alkylene or C₁-C₆alkylcarbonyl, wherein each alkyl/alkylene group is substituted with 0-3 R¹⁸;

R⁵ is selected from H, C₁-C₆alkyl, C₃-C₆cycloalkyl, halo, OH, and cyano;

R⁶ and R⁷ are independently selected from H, C₁-C₆alkyl, F, trihalomethyl, and trihalomethoxy;

alternatively, R⁶ and R⁷, together with the carbon atom to which they are attached, form a 3-8 membered saturated or partially saturated monocyclic ring consisting of the shown carbon atom, 2-5 additional carbon atoms, and 0-2 heteroatoms selected from nitrogen, oxygen, and S(O)ₘ, wherein this ring is substituted with 0-3 groups selected from halo, trihalomethyl, OH, C₁-C₆alkyl, oxo, and C₁-C₆alkyloxy;

R⁸ is selected from C₁-C₈alkyl, C₂-C₈alkenyl, aryl, hetaryl, arylC₁-C₆alkylene, hetarylC₁-C₆alkylene, C₃-C₁₀cycloalkyl, 3-10 membered hetcycloalkyl, aryloxyC₁-C₆alkylene, hetaryloxyC₁-C₆alkylene, arylC₁-C₆alkyloxyC₁-C₆alkylene, and hetaryl-C₁-C₆alkyloxyC₁-C₆alkylene, wherein each of the alkyl/alkylene, alkenyl, aryl, hetaryl, cycloalkyl, and hetcycloalkyl groups are independently substituted with 0-3 R¹⁹;

R⁹ is selected from C₁-C₈alkyl, C₂-C₈alkenyl, aryl, hetaryl, arylC₁-C₆alkylene, hetarylC₁-C₆alkylene, C₃-C₁₀cycloalkyl, 3-10 membered hetcycloalkyl, aryloxyC₁-C₆alkylene, and arylC₁-C₆alkyloxyC₁-C₆alkylene, wherein each of the alkyl/alkylene, alkenyl, aryl, hetaryl, cycloalkyl, and hetcycloalkyl groups are independently substituted with 0-3 R²⁰;

R¹⁰ and R¹¹ are independently selected from H, C₁-C₈alkyl, C₁-C₁₀cycloalkyl, 3-10 membered hetcycloalkyl, aryl, hetaryl, arylC₁-C₆alkylene, and hetarylC₁-C₆alkylene, wherein each of the alkyl/alkylene, cycloalkyl, hetcycloalkyl, aryl, and hetaryl groups are independently substituted with 0-3 R²¹;

alternatively, R¹⁰ and R¹¹, together with the nitrogen to which they are attached, form a 5-12 membered saturated or partially saturated monocyclic, bicyclic, or tricyclic ring consisting of the shown nitrogen atom, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkylene, hetarylC₁-C₆alkylene, hydroxy, oxo, COOH, C₁-C₆alkyloxy, arylC₁-C₆alkyloxy, hetarylC₁-C₆alkyloxy, C₁-C₆alkyloxyC₁-C₆alkylene, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkyl-carboxy, and hetarylC₁-C₆alkylcarboxy;

R¹² is selected from OH, C₁-C₈alkyl, C₃-C₁₀cycloalkyl, 3-10 membered hetcycloalkyl, trihalomethyl, C₁-C₈alkyloxy, aryl, arylC₁-C₆alkylene, hetaryl, hetarylC₁-C₆alkylene, aryloxy, hetaryloxy, and NR¹³R¹⁴;

R¹³ and R¹⁴ are independently selected from H, C₁-C₈alkyl, C₃-C₁₀cycloalkyl, aryl, hetaryl, arylC₁-C₆alkylene, and hetarylC₁-C₆alkylene, wherein each of the alkyl/alkylene, cycloalkyl, aryl, and hetaryl groups are independently substituted with 0-3 R²²;

alternatively, R¹³ and R¹⁴, together with the nitrogen to which they are attached, form a 5-12 membered saturated or partially saturated monocyclic, bicyclic, or tricyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkylene, hetarylC₁-C₆alkylene, OH, oxo, C₁-C₆alkyloxy, arylC₁-C₆alkyloxy, hetarylC₁-C₆alkyloxy, C₁-C₆alkyloxyC₁-C₆alkylene, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkylcarboxy, and hetarylC₁-C₆alkylcarboxy;

R¹⁵ is selected from H, C₁-C₆alkyl, and C₃-C₆cycloalkyl;

R¹⁶ and R¹⁷ are independently selected from H, C₁-C₈alkyl, C₁-C₁₀cycloalkyl, halo, OH, cyano, —C(═O)R¹², —S(O)ₙR¹², —S(O)ₙNR¹³R¹⁴, —N(R¹³)S(O)ₙR¹², C₁-C₈alkyl, aryl, and hetaryl, wherein the alkyl and cycloalkyl groups are independently substituted with 0-3 R²²;

R¹⁸ is selected from halo, OH, oxo, and cyano;

R¹⁹, R²⁰ and R²¹ are independently selected from H, halo, OH, oxo, cyano, C₁-C₈alkyl, C₃-C₁₀cycloalkyl, 3-10 membered hetcycloalkyl, trihalomethyl, trihalomethyloxy, methylendioxo, dihalo-methylenedioxo, C₃-C₆spirocycloalkyl, C₁-C₆alkyloxy, aryl, hetaryl, arylC₁-C₆alkylene, hetarylC₁-C₆alkylene, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, and —C(═NR¹⁶)NR¹⁷;

R²² is selected from H, OH, oxo, halo, cyano, nitro, C₁-C₆alkyl, C₁-C₆alkyloxy, NR²³R²⁴, methylendioxo, dihalomethylendioxo, trihalomethyl, and trihalomethyloxy;

R²³ and R²⁴ are independently selected from H, C₁-C₈alkyl, and arylC₁-C₆alkylene;

m is selected from 0, 1, and 2;

n is selected from 1 and 2;

Y is selected from O and S;
[2u] In another embodiment, the present invention provides the novel use of compounds of formula I, wherein:

R¹ is selected from R⁸(C═O)—, R⁹S(O)₂—, R¹⁰R¹¹NC(═O)—, and R¹⁰R¹¹NS(O)₂—;

R² is C₁-C₄alkyl;

alternatively, R¹ and R², together with the nitrogen to which they are attached, form a 5-6 membered saturated ring consisting of the shown nitrogen, 2-4 carbon atoms, and 0-2 additional heteroatoms selected from nitrogen, oxygen, and S(O)ₘ, wherein this ring is substituted with 0-2 groups selected from C₁-C₈alkyl, aryl, hetaryl, arylC₁-C₆alkyl, hetarylC₁-C₆alkyl, —C(═O)R¹², —S(O)ₙR¹², —S(═O)ₙNR¹³R¹⁴, —N(R¹³)S(O)ₙR¹², OH, oxo, C₁-C₆alkyloxy, arylC₁-C₆alkyloxy, hetarylC₁-C₆alkyloxy, C₁-C₆alkyloxyC₁-C₆alkyl, C₁-C₆alkyl-carboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸;

Ring A is an 8-11 membered saturated or partially saturated bicyclic or tricyclic ring consisting of the shown nitrogen, 5-10 carbon atoms and from 0 to 1 additional heteroatoms selected from nitrogen, oxygen, and S(O)ₘ;

Ring A is substituted with 0-3 groups selected from C₁-C₄alkyl, halo, OH, oxo, cyano, C₁-C₄alkyloxy, C₁-C₄alkyloxyC₁-C₄alkyl or C₁-C₄alkylcarbonyl, wherein each alkyl/alkyl group is substituted with 0-1 R¹⁸;

R⁵ is H;

R⁶ and R⁷ are independently selected from H and C₁-C₄alkyl; and,

n is 2.

[2] In another embodiment, the present invention provides the novel use of compounds of formula I, wherein:

R¹ is selected from R⁸(C═O)—, R⁹S(O)₂—, R¹⁰R¹¹NC(═O)—, and R¹¹R¹¹NS(O)₂—;

R² is C₁-C₄alkyl;

alternatively, R¹ and R², together with the nitrogen to which they are attached, form a 5-6 membered saturated ring consisting of the shown nitrogen, 2-4 carbon atoms, and 0-2 additional heteroatoms selected from nitrogen, oxygen, and S(O)ₘ, wherein this ring is substituted with 0-2 groups selected from C₁-C₈alkyl, aryl, hetaryl, arylC₁-C₆alkylene, hetarylC₁-C₆alkylene, —C(═O)R¹², —S(O)ₙR¹², —S(═O)ₙNR¹³R¹⁴, —N(R¹³)S(O)ₙR¹², OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetaryl-C₁-C₆alkylcarboxy, wherein each aryl/hetaryl group is substituted with 0-3 R¹⁸;

Ring A is an 8-11 membered saturated or partially saturated bicyclic or tricyclic ring consisting of the shown nitrogen, 5-10 carbon atoms and from 0 to 1 additional heteroatoms selected from nitrogen, oxygen, and S(O)ₘ;

Ring A is substituted with 0-3 groups selected from C₁-C₄alkyl, halo, OH, oxo, cyano, C₁-C₄alkyloxy, C₁-C₄alkyloxyC₁-C₄alkylene or C₁-C₄alkylcarbonyl, wherein each alkyl/alkylene group is substituted with 0-1 R¹⁸;

R⁵ is H;

R⁶ and R⁷ are independently selected from H and C₁-C₄alkyl; and,

n is 2.

[3u] In another embodiment, the present invention provides the novel use of compounds of formula I, wherein:

R⁸ is selected from C₁-C₆alkyl, C₂-C₆alkenyl, aryl, hetaryl, arylC₁-C₄alkyl, hetarylC₁-C₄alkyl, C₃-C₆cycloalkyl, 3-6 membered hetcycloalkyl, aryloxyC₁-C₄alkyl, and hetaryloxyC₁-C₄alkyl, wherein each of the alkyl/alkyl, alkenyl, aryl, hetaryl, cycloalkyl, and hetcycloalkyl groups are independently substituted with 0-2 R¹⁹;

R⁹ is selected from C₁-C₆alkyl, C₂-C₆alkenyl, aryl, hetaryl, arylC₁-C₄alkyl, hetarylC₁-C₄alkyl, C₃-C₆cycloalkyl, 3-6 membered hetcycloalkyl, and aryloxyC₁-C₄alkyl, wherein each of the alkyl/alkyl, alkenyl, aryl, hetaryl, cycloalkyl, and hetcycloalkyl groups are independently substituted with 0-2 R²⁰;

R¹⁰ and R¹¹ are independently selected from H, C₃-C₆cycloalkyl, 3-6 membered hetcycloalkyl, aryl, and hetaryl, wherein each of the cycloalkyl, hetcycloalkyl, aryl, and hetaryl groups are independently substituted with 0-3 R²¹;

alternatively, R¹⁰ and R¹¹, together with the nitrogen to which they are attached, form a 5-6 membered saturated or partially saturated monocyclic ring consisting of the shown nitrogen atom, 4-5 carbon atoms, and 0-1 additional heteroatoms selected from nitrogen, oxygen, and S(O)ₘ, wherein this ring is substituted with 0-2 groups selected from C₁-C₈alkyl, aryl, hetaryl, hydroxy, oxo, COOH, C₁-C₆alkyloxy, arylC₁-C₆alkyloxy, hetarylC₁-C₆alkyloxy, and C₁-C₆alkylcarbonyl;

R¹² is selected from OH, C₁-C₄alkyl, C₁-C₆cycloalkyl, 3-10 membered hetcycloalkyl, trihalomethyl, C₁-C₄alkyloxy, aryl, arylC₁-C₄alkyl, hetaryl, hetarylC₁-C₄alkyl, aryloxy, and hetaryloxy;

R¹⁹, R²⁰ and R²¹ are independently selected from H, halo, OH, oxo, cyano, C₁-C₆alkyl, C₃-C₆cycloalkyl, 3-6 membered hetcycloalkyl, trihalomethyl, trihalomethyloxy, dihalo-methylenedioxo, C₁-C₄alkyloxy, aryl, hetaryl, arylC₁-C₄alkyl, hetarylC₁-C₄alkyl, —C(═O)R¹², —S(O)ₙR¹², and —S(O)ₙNR¹³R¹⁴; and,

n is 2.

[3] In another embodiment, the present invention provides the novel use of compounds of formula I, wherein:

R⁸ is selected from C₁-C₆alkyl, C₂-C₆alkenyl, aryl, hetaryl, arylC₁-C₄alkylene, hetarylC₁-C₄alkylene, C₃-C₆cycloalkyl, 3-6 membered hetcycloalkyl, aryloxyC₁-C₄alkylene, and hetaryloxyC₁-C₄alkylene, wherein each of the alkyl/alkylene, alkenyl, aryl, hetaryl, cycloalkyl, and hetcycloalkyl groups are independently substituted with 0-2 R¹⁹;

R⁹ is selected from C₁-C₆alkyl, C₂-C₆alkenyl, aryl, hetaryl, arylC₁-C₄alkylene, hetarylC₁-C₄alkylene, C₃-C₆cycloalkyl, 3-6 membered hetcycloalkyl, and aryloxyC₁-C₄alkylene, wherein each of the alkyl/alkylene, alkenyl, aryl, hetaryl, cycloalkyl, and hetcycloalkyl groups are independently substituted with 0-2 R²⁰;

R¹⁰ and R¹¹ are independently selected from H, C₃-C₆cycloalkyl, 3-6 membered hetcycloalkyl, aryl, and hetaryl, wherein each of the cycloalkyl, hetcycloalkyl, aryl, and hetaryl groups are independently substituted with 0-3 R²¹;

alternatively, R¹⁰ and R¹¹, together with the nitrogen to which they are attached, form a 5-6 membered saturated or partially saturated monocyclic ring consisting of the shown nitrogen atom, 4-5 carbon atoms, and 0-1 additional heteroatoms selected from nitrogen, oxygen, and S(O)ₘ, wherein this ring is substituted with 0-2 groups selected from C₁-C₈alkyl, aryl, hetaryl, hydroxy, oxo, COOH, C₁-C₆alkyloxy, arylC₁-C₆alkyloxy, hetarylC₁-C₆alkyloxy, and C₁-C₆alkylcarbonyl;

R¹² is selected from OH, C₁-C₄alkyl, C₃-C₆cycloalkyl, 3-10 membered hetcycloalkyl, trihalomethyl, C₁-C₄alkyloxy, aryl, arylC₁-C₄alkylene, hetaryl, hetarylC₁-C₄alkylene, aryloxy, and hetaryloxy;

R¹⁹, R²⁰ and R²¹ are independently selected from H, halo, OH, oxo, cyano, C₁-C₆alkyl, C₃-C₆cycloalkyl, 3-6 membered hetcycloalkyl, trihalomethyl, trihalomethyloxy, dihalo-methylenedioxo, C₁-C₄alkyloxy, aryl, hetaryl, arylC₁-C₄alkylene, hetarylC₁-C₄alkylene, —C(═O)R¹², —S(O)ₙR¹², and —S(O)ₙNR¹³R¹⁴; and,

n is 2.

[4u] In another embodiment, the present invention provides the novel use of compounds wherein the substituted amide or prodrug thereof is of formula Ia:

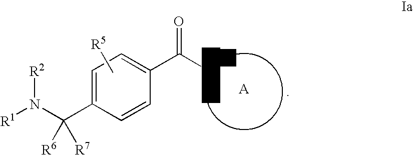

[5] In another embodiment, the present invention provides the novel use of compounds wherein the substituted amide or prodrug thereof is of formula Ib:

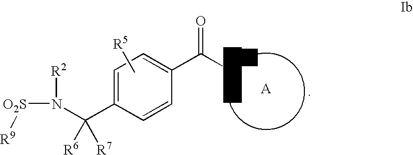

[6] In another embodiment, the present invention provides the novel use of compounds wherein the substituted amide or prodrug thereof is of formula Ic:

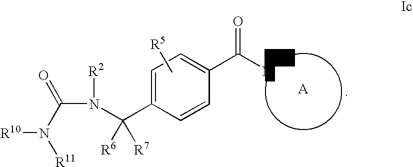

[7] In another embodiment, the present invention provides the novel use of compounds wherein the substituted amide or prodrug thereof is of formula Id:

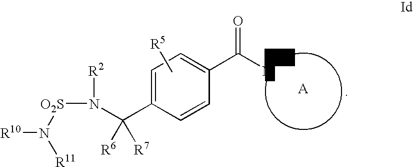

[8] In another embodiment, the present invention provides the novel use of compounds wherein the substituted amide or prodrug thereof is of formula Ie:

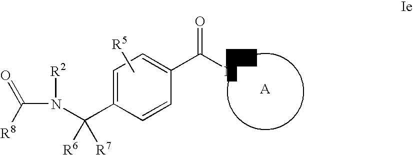

[9u] In another embodiment, the present invention provides the novel use of compounds of formula I, wherein:

R¹ and R², together with the nitrogen to which they are attached, form a 5-12 membered saturated or partially saturated monocyclic or bicyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹²—S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹³.

[9] In another embodiment, the present invention provides the novel use of compounds of formula I, wherein:

R¹ and R², together with the nitrogen to which they are attached, form a 5-12 membered saturated or partially saturated monocyclic or bicyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, C₃-C₆spirocycloalkyl, 3-6 membered spirohetcycloalkyl, aryl, hetaryl, arylC₁-C₆alkylene, hetarylC₁-C₆alkylene, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each aryl/hetaryl group is substituted with 0-3 R¹³.

[10u] In another embodiment, the present invention provides the novel use of compounds of formula I, wherein:

Ring A is selected from:

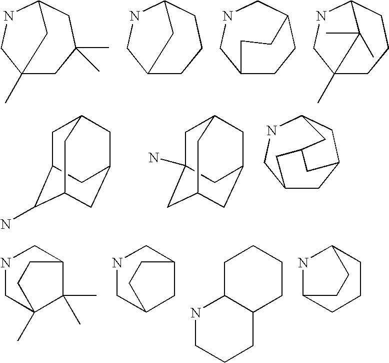

Ring A is substituted with 0-2 R²⁵; and,

R²⁵ is selected from C₁-C₈alkyl, halo, hydroxy, oxo, cyano, C(═O)R¹², and C₁-C₆alkyloxy, wherein R¹² is as defined above.

[10] In another embodiment, the present invention provides the novel use of compounds of formula I, wherein:

Ring A is selected from:

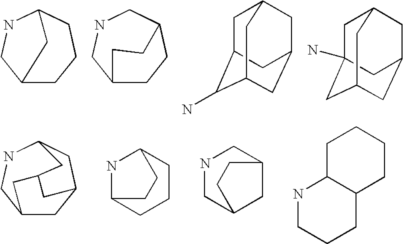

Ring A is substituted with 0-2 R²⁵; and,

R²⁵ is selected from C₁-C₈alkyl, halo, hydroxy, oxo, cyano, and C₁-C₆alkyloxy.

[11] In another embodiment, the present invention provides the novel use of compounds of formula I, wherein:

ring A is

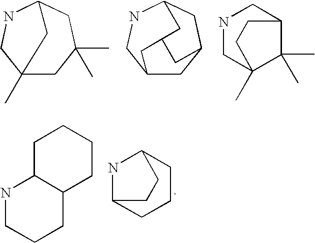

Ring A is substituted with 0-2 R²⁵; and,

R²⁵ is selected from C₁-C₈alkyl, halo, hydroxy, oxo, cyano, and C₁-C₆alkyloxy.

In another embodiment, the present invention provides the novel use of compounds of formula I, wherein the substituted amide or a prodrug thereof is of the selected from the group:
N-Methyl-N-[4-(1,3,3-trimethyl-6-aza-bicyclo[3.2.1]octane-6-carbonyl)-benzyl]-acetamide
N-Methyl-N-[4-(1,3,3-trimethyl-6-aza-bicyclo[3.2.1]octane-6-carbonyl)-benzyl]-isobutyramide
Cyclopentanecarboxylic acid methyl-[4-(1,3,3-trimethyl-6-aza-bicyclo[3.2.1]octane-6-carbonyl)-benzyl]-amide
Cyclohexanecarboxylic acid methyl-[4-(1,3,3-trimethyl-6-aza-bicyclo[3.2.1]octane-6-carbonyl)-benzyl]-amide
Piperidine-1-carboxylic acid methyl-[4-(1,3,3-trimethyl-6-aza-bicyclo[3.2.1]octane-6-carbonyl)-benzyl]-amide
N-Methyl-N-[4-(1,3,3-trimethyl-6-aza-bicyclo[3.2.1]octane-6-carbonyl)-benzyl]-benzamide
1-Acetyl-piperidine-4-carboxylic acid methyl-[4-(1,3,3-trimethyl-6-aza-bicyclo[3.2.1]octane-6-carbonyl)-benzyl]-amide
1-Acetyl-piperidine-3-carboxylic acid methyl-[4-(1,3,3-trimethyl-6-aza-bicyclo[3.2.1]octane-6-carbonyl)-benzyl]-amide
Cyclopentanecarboxylic acid ethyl-[4-(1,3,3-trimethyl-6-aza-bicyclo[3.2.1]octane-6-carbonyl)-benzyl]-amide
Morpholine-4-carboxylic acid methyl-[4-(1,3,3-trimethyl-6-aza-bicyclo[3.2.1]octane-6-carbonyl)-benzyl]-amide
2,2-N-Trimethyl-N-[4-(1,3,3-trimethyl-6-aza-bicyclo[3.2.1]octane-6-carbonyl)-benzyl]-propionamide
Tetrahydro-furan-3-carboxylic acid methyl-[4-(1,3,3-trimethyl-6-aza-bicyclo[3.2.1]octane-6-carbonyl)-benzyl]-amide
N-Methyl-4-trifluoromethoxy-N-[4-(1,3,3-trimethyl-6-aza-bicyclo[3.2.1]octane-6-carbonyl)-benzyl]-benzamide
Thiophene-2-carboxylic acid methyl-[4-(1,3,3-trimethyl-6-aza-bicyclo[3.2.1]octane-6-carbonyl)-benzyl]-amide
Furan-2-carboxylic acid methyl-[4-(1,3,3-trimethyl-6-aza-bicyclo[3.2.1]octane-6-carbonyl)-benzyl]-amide
3-Chloro-4-(propane-2-sulfonyl)-thiophene-2-carboxylic acid methyl-[4-(1,3,3-trimethyl-6-aza-bicyclo[3.2.1]octane-6-carbonyl)-benzyl]-amide
6-Chloro-N-methyl-N-[4-(1,3,3-trimethyl-6-aza-bicyclo[3.2.1]octane-6-carbonyl)-benzyl]-nicotinamide
5-Methyl-isoxazole-3-carboxylic acid methyl-[4-(1,3,3-trimethyl-6-aza-bicyclo[3.2.1]octane-6-carbonyl)-benzyl]-amide
3,3,N-Trimethyl-N-[4-(1,3,3-trimethyl-6-aza-bicyclo[3.2.1]octane-6-carbonyl)-benzyl]-butyramide
3-Cyano-N-methyl-N-[4-(1,3,3-trimethyl-6-aza-bicyclo[3.2.1]octane-6-carbonyl)-benzyl]-benzamide
N-Methyl-2-phenoxy-N-[4-(1,3,3-trimethyl-6-aza-bicyclo[3.2.1]octane-6-carbonyl)-benzyl]-acetamide
N-Methyl-N-[4-(1,3,3-trimethyl-6-aza-bicyclo[3.2.1]octane-6-carbonyl)-benzyl]-malonamic acid methyl ester
3-Methyl-but-2-enoic acid methyl-[4-(1,3,3-trimethyl-6-aza-bicyclo[3.2.1]octane-6-carbonyl)-benzyl]-amide
N-Methyl-2-phenyl-N-[4-(1,3,3-trimethyl-6-aza-bicyclo[3.2.1]octane-6-carbonyl)-benzyl]-acetamide
1-Trifluoromethyl-cyclobutanecarboxylic acid methyl-[4-(1,3,3-trimethyl-6-aza-bicyclo[3.2.1]octane-6-carbonyl)-benzyl]-amide
3,5-Dimethoxy-N-methyl-N-[4-(1,3,3-trimethyl-6-aza-bicyclo[3.2.1]octane-6-carbonyl)-benzyl]-benzamide
4-Methanesulfonyl-N-methyl-N-[4-(1,3,3-trimethyl-6-aza-bicyclo[3.2.1]octane-6-carbonyl)-benzyl]-benzamide
N-Methyl-3-trifluoromethoxy-N-[4-(1,3,3-trimethyl-6-aza-bicyclo[3.2.1]octane-6-carbonyl)-benzyl]-benzamide
2,2-Difluoro-1,3-benzodioxole-4-carboxylic acid methyl-[4-(1,3,3-trimethyl-6-aza-bicyclo[3.2.1]octane-6-carbonyl)-benzyl]-amide
N-Methyl-6-morpholin-4-yl-N-[4-(1,3,3-trimethyl-6-aza-bicyclo[3.2.1]octane-6-carbonyl)-benzyl]-nicotinamide
N-Methyl-4-(2,2,2-trifluoro-acetyl)-N-[4-(1,3,3-trimethyl-6-aza-bicyclo[3.2.1]octane-6-carbonyl)-benzyl]-benzamide
N-[4-(1,3,3-Trimethyl-6-aza-bicyclo[3.2.1]octane-6-carbonyl)-benzyl]-benzamide
N-Methyl-N-[4-(1,3,3-trimethyl-6-aza-bicyclo[3.2.1]octane-6-carbonyl)-benzyl]-isophthalamic acid
2,3-Dihydro-benzofuran-7-carboxylic acid methyl-[4-(1,3,3-trimethyl-6-aza-bicyclo[3.2.1]octane-6-carbonyl)-benzyl]-amide
3-Acetyl-N-methyl-N-[4-(1,3,3-trimethyl-6-aza-bicyclo[3.2.1]octane-6-carbonyl)-benzyl]-benzamide
1,1,3-Trimethyl-3-[4-(1,3,3-trimethyl-bicyclo[3.2.1]octane-6-carbonyl)-benzyl]-sulphonylurea
N-Methyl-N-[4-(1,3,3-trimethyl-6-aza-bicyclo[3.2.1]octane-6-carbonyl)-benzyl]-methanesulfonamide
2,2,2-Trifluoro-ethanesulfonic acid methyl-[4-(1,3,3-trimethyl-6-aza-bicyclo[3.2.1]octane-6-carbonyl)-benzyl]-amide
N-Methylphenyl-N-[4-(1,3,3-trimethyl-6-aza-bicyclo[3.2.1]octane-6-carbonyl)-benzyl]-methanesulfonamide
Trifluoro-N-isopropyl-N-[4-(1,3,3-trimethyl-6-aza-bicyclo[3.2.1]octane-6-carbonyl)-benzyl]-methanesulfonamide
N-Cyclopropyl-trifluoro-N-[4-(1,3,3-trimethyl-6-aza-bicyclo[3.2.1]octane-6-carbonyl)-benzyl]-methanesulfonamide
N-Ethyl-trifluoro-N-[4-(1,3,3-trimethyl-6-aza-bicyclo[3.2.1]octane-6-carbonyl)-benzyl]-methanesulfonamide
Trifluoro-N-methyl-N-[4-(1,3,3-trimethyl-6-aza-bicyclo[3.2.1]octane-6-carbonyl)-benzyl]-methanesulfonamide
3-Benzoyl-1-methyl-1-[4-(1,3,3-trimethyl-6-aza-bicyclo[3.2.1]octane-6-carbonyl)-benzyl]-urea
3-Cyclohexyl-1-methyl-1-[4-(1,3,3-trimethyl-6-aza-bicyclo[3.2.1]octane-6-carbonyl)-benzyl]-urea
3-(4-methyl-phenyl)sulfonyl-1-methyl-1-[4-(1,3,3-tri-methyl-6-aza-bicyclo[3.2.1]octane-6-carbonyl)-benzyl]-urea
1,3-Dimethyl-3-phenyl-1-[4-(1,3,3-trimethyl-6-aza-bicyclo[3.2.1]octane-6-carbonyl)-benzyl]-urea
3-(2,3-Dihydro-1,4-benzodioxin-2-ylmethyl)-1-methyl-1-[4-(1,3,3-trimethyl-6-aza-bicyclo[3.2.1]octane-6-carbonyl)-benzyl]-urea
3-(3-Methoxy-benzyl)-1-methyl-1-[4-(1,3,3-trimethyl-6-aza-bicyclo[3.2.1]octane-6-carbonyl)-benzyl]-urea
3-(1,1-Dioxo-tetrahydro-thiophen-3-yl)-1-methyl-1-[4-(1,3,3-trimethyl-6-aza-bicyclo[3.2.1]octane-6-carbonyl)-benzyl]-urea
1-Methyl-3-(tetrahydro-pyran-4-yl)-1-[4-(1,3,3-trimethyl-6-aza-bicyclo[3.2.1]-octane-6-carbonyl)-benzyl]-urea
{1,3-Dimethyl-3-[4-(1,3,3-trimethyl-6-aza-bicyclo[3.2.1]octane-6-carbonyl)-benzyl]-ureido}-acetic acid methyl ester
1-Methyl-3-(5-trifluoromethyl-1,3,4-thiadiazol-2-yl)-1-[4-(1,3,3-trimethyl-6-aza-bicyclo[3.2.1]octane-6-carbonyl)-benzyl]-urea
2-{3-Methyl-3-[4-(1,3,3-trimethyl-6-aza-bicyclo[3.2.1]octane-6-carbonyl)-benzyl]-ureido}-benzoic acid methyl ester
3-{3-Methyl-3-[4-(1,3,3-trimethyl-6-aza-bicyclo[3.2.1]octane-6-carbonyl)-benzyl]-ureido}-benzoic acid ethyl ester
1-Methyl-3-(3-ethylsulfanyl-phenyl)-1-[4-(1,3,3-trimethyl-6-aza-bicyclo-[3.2.1]-octane-6-carbonyl)-benzyl]-urea
1-Methyl-3-(4-ethylsulfanyl-phenyl)-1-[4-(1,3,3-trimethyl-6-aza-bicyclo-[3.2.1]-octane-6-carbonyl)-benzyl]-urea
3-(4-Benzyloxy-phenyl)-1-methyl-1-[4-(1,3,3-trimethyl-6-aza-bicyclo[3.2.1]octane-6-carbonyl)-benzyl]-urea
1-Methyl-3-(4-trifluoro-methylsulfanyl-phenyl)-1-[4-(1,3,3-trimethyl-6-aza-bicyclo[3.2.1]octane-6-carbonyl)-benzyl]-urea
3-(4-Acetyl-phenyl)-1-methyl-1-[4-(1,3,3-tri-methyl-6-aza-bicyclo[3.2.1]octane-6-carbonyl)-benzyl]-urea
3-(3-Acetyl-phenyl)-1-methyl-1-[4-(1,3,3-tri-methyl-6-aza-bicyclo[3.2.1]octane-6-carbonyl)-benzyl]-urea
3-(3-Cyano-phenyl)-1-methyl-1-[4-(1,3,3-tri-methyl-6-aza-bicyclo[3.2.1]octane-6-carbonyl)-benzyl]-urea
1-Methyl-3-(4-trifluoro-methyl-phenyl)-1-[4-(1,3,3-trimethyl-6-aza-bicyclo-[3.2.1]octane-6-carbonyl)-benzyl]-urea
3-(4-Methoxy-benzyl)-1-methyl-1-[4-(1,3,3-tri-methyl-6-aza-bicyclo[3.2.1]octane-6-carbonyl)-benzyl]-urea
1-Methyl-3-(2,2,4,4-tetrafluoro-4H-benzo-[1,3]dioxin-6-yl)-1-[4-(1,3,3-trimethyl-6-aza-bicyclo-[3.2.1]octane-6-carbonyl)-benzyl]-urea
1-Methyl-3-(4-trifluoro-methoxy-phenyl)-1-[4-(1,3,3-trimethyl-6-aza-bicyclo[3.2.1]-octane-6-carbonyl)-benzyl]-urea
1-Methyl-3-[4-(2,2,2-trifluoro-acetyl)-cyclohexyl]-1-[4-(1,3,3-trimethyl-6-aza-bicyclo[3.2.1]octane-6-carbonyl)-benzyl]-urea
1-(4-Acetyl-phenyl)-1,3-dimethyl-3-[4-(1,3,3-tri-methyl-6-aza-bicyclo[3.2.1]octane-6-carbonyl)-benzyl]-urea
1-Phenyl-3-{1-[4-(1,3,3-trimethyl-6-aza-bicyclo-[3.2.1]octane-6-carbonyl)-phenyl]-cyclopropyl}-urea
Piperidine-1-carboxylic acid methyl-[4-(octahydro-quinoline-1-carbonyl)-benzyl]-amide
Piperidine-1-carboxylic acid [4-(3-aza-bicyclo-[3.2.2]nonane-3-carbonyl)-benzyl]-methyl-amide
Morpholine-4-carboxylic acid methyl-[4-(octahydro-quinoline-1-carbonyl)-benzyl]-amide
Morpholine-4-carboxylic acid [4-(3-aza-bicyclo-[3.2.2]nonane-3-carbonyl)-benzyl]-methyl-amide
1,3-Dimethyl-3-[4-(octahydro-quinoline-1-carbonyl)-benzyl]-1-phenyl-urea
1-[4-(3-Aza-bicyclo-[3.2.2]nonane-3-carbonyl)-benzyl]-1,3-dimethyl-3-phenyl-urea
Piperidine-1-carboxylic acid [4-(6-aza-bicyclo-[3.2.1]octane-6-carbonyl)-benzyl]-methyl-amide
Piperidine-1-carboxylic acid methyl-[4-(1,8,8-trimethyl-3-aza-bicyclo[3.2.1]octane-3-carbonyl)-benzyl]-amide
Morpholine-4-carboxylic acid [4-(6-aza-bicyclo-[3.2.1]octane-6-carbonyl)-benzyl]-methyl-amide
Morpholine-4-carboxylic acid methyl-[4-(1,8,8-trimethyl-3-aza-bicyclo[3.2.1]-octane-3-carbonyl)-benzyl]-amide
1-[4-(6-Aza-bicyclo-[3.2.1]octane-6-carbonyl)-benzyl]-1,3-dimethyl-3-phenyl-urea
1,3-Dimethyl-1-phenyl-3-[4-(1,8,8-trimethyl-3-aza-bicyclo[3.2.1]octane-3-carbonyl)-benzyl]-urea
Piperidine-1-carboxylic acid [4-(3-hydroxy-8-aza-bicyclo[3.2.1]octane-8-carbonyl)-benzyl]-methyl-amide
Morpholine-4-carboxylic acid [4-(3-hydroxy-8-aza-bicyclo[3.2.1]octane-8-carbonyl)-benzyl]-methyl-amide
1-[4-(3-Hydroxy-8-aza-bicyclo[3.2.1]octane-8-carbonyl)-benzyl]-1,3-di-methyl-3-phenyl-urea
1-[4-(3-Fluoro-8-aza-bicyclo[3.2.1]octane-8-carbonyl)-benzyl]-1,3-di-methyl-3-phenyl-urea
Piperidine-1-carboxylic acid [4-(3-fluoro-8-aza-bicyclo[3.2.1]octane-8-carbonyl)-benzyl]-methyl-amide
Morpholine-4-carboxylic acid [4-(3-fluoro-8-aza-bicyclo[3.2.1]octane-8-carbonyl)-benzyl]-methyl-amide
N-Adamantan-2-yl-4-(1,3-dimethyl-3-pyridin-2-yl-ureidomethyl)-benzamide
1-[4-(3-Aza-bicyclo[3.2.2]nonane-3-carbonyl)-benzyl]-1,3-dimethyl-3-pyridin-2-yl-urea
1-[4-(3-Hydroxy-8-aza-bicyclo[3.2.1]octane-8-carbonyl)-benzyl]-1,3-dimethyl-3-pyridin-2-yl-urea
Morpholine-4-carboxylic acid [4-(adamantan-2-ylcarbamoyl)-benzyl]-methyl-amide
1-[4-(3-Aza-bicyclo[3.2.2]nonane-3-carbonyl)-benzyl]-1,3-dimethyl-3-thiazol-2-yl-urea
1,3-Dimethyl-3-[4-(2-oxa-5-aza-bicyclo[2.2.1]heptane-5-carbonyl)-benzyl]-1-thiazol-2-yl-urea
4-[3-(1-Acetyl-piperidin-4-yl)-1,3-dimethyl-ureidomethyl]-N-adamantan-2-yl-benzamide
1-(1-Acetyl-piperidin-4-yl)-3-[4-(3-aza-bicyclo[3.2.2]nonane-3-carbonyl)-benzyl]-1,3-dimethyl-urea
N-Adamantan-2-yl-4-(1,3-dimethyl-3-pyrimidin-2-yl-ureidomethyl)-benzamide
1-[4-(3-Aza-bicyclo[3.2.2]nonane-3-carbonyl)-benzyl]-1,3-dimethyl-3-pyrimidin-2-yl-urea
N-Adamantan-2-yl-4-(1,3-dimethyl-3-thiazol-2-yl-ureidomethyl)-benzamide
N-Adamantan-2-yl-4-(1,3-dimethyl-3-phenyl-ureidomethyl)-benzamide
1-[4-(3-Hydroxy-8-aza-bicyclo[3.2.1]octane-8-carbonyl)-benzyl]-1,3-dimethyl-3-pyrimidin-2-yl-urea
N-Adamantan-2-yl-4-[3-(4-hydroxy-cyclohexyl)-1,3-dimethyl-ureidomethyl]-benzamide
1-[4-(3-Aza-bicyclo[3.2.2]nonane-3-carbonyl)-benzyl]-3-(4-hydroxy-cyclohexyl)-1,3-dimethyl-urea
1-(4-Hydroxy-cyclohexyl)-1,3-dimethyl-3-[4-(2-oxa-5-aza-bicyclo[2.2.1]heptane-5-carbonyl)-benzyl]-urea
1-Methyl-3-[4-(1,3,3-trimethyl-6-aza-bicyclo[3.2.1]octane-6-carbonyl)-benzyl]-imidazo-lidin-2-one
[4-(1,1-Dioxo-isothiazolidin-2-ylmethyl)-phenyl]-(1,3,3-trimethyl-6-aza-bicyclo[3.2.1]-oct-6-yl)-methanone
[4-(1,1-Dioxo-1,2,5-thiadiazolidin-2-ylmethyl)-phenyl]-(1,3,3-trimethyl-bicyclo[3.2.1]oct-6-yl)-methanone
[4-(5-Methyl-1,1-dioxo-1,2,5-thiadiazolidin-2-ylmethyl)-phenyl]-(1,3,3-trimethyl-bicyclo[3.2.1]oct-6-yl)-methanone
(Octahydro-quinolin-1-yl)-[4-(1,1-dioxo-1,2,5-thiadiazolidin-2-ylmethyl)-phenyl]-methanone
(4-Aza-tricyclo[4.3.1.1^{3,8}]-undec-4-yl)-[4-(1,1-dioxo-1,2,5-thiadiazolidin-2-ylmethyl)-phenyl]-methanone
(Octahydro-isoquinolin-2-yl)-[4-(1,1-dioxo-1,2,5-thiadiazolidin-2-ylmethyl)-phenyl]-methanone
(3-Aza-bicyclo[3.2.2]non-3-yl)-[4-(1,1-dioxo-1,2,5-thiadiazolidin-2-ylmethyl)-phenyl]-methanone
(6-Aza-bicyclo[3.2.1]oct-6-yl)-[4-(1,1-dioxo-1,2,5-thiadiazolidin-2-ylmethyl)-phenyl]-methanone
[4-(5-Benzyl-1,1-dioxo-[1,2,5]thiadiazolidin-2-ylmethyl)-phenyl]-(1,3,3-trimethyl-6-aza-bicyclo[3.2.1]oct-6-yl)-methanone

or a salt thereof with a pharmaceutically acceptable acid or base, or any optical iso-mer or mixture of optical isomers, including a racemic mixture, or any tautomeric forms.

In another embodiment, the present invention provides for the novel preparation of a pharmaceutical composition for the treatment of conditions, disorders, or diseases wherein a modulation or an inhibition of the activity of 11βHSD1 is beneficial.

In another embodiment, the present invention provides for the novel preparation of a pharmaceutical composition, wherein: the conditions, disorders, and diseases that are influenced by intracellular glucocorticoid levels.

In another embodiment, the present invention provides for the novel preparation of a pharmaceutical composition, wherein: the conditions, disorders, or diseases are selected from metabolic syndrome, insulin resistance, dyslipidemia, hypertension, obesity, type 2 diabetes, impaired glucose tolerance (IGT), impaired fasting glucose (IFG), the progression from IGT to type 2 diabetes, the progression of the metabolic syndrome into type 2 diabetes, diabetic late complications, neurodegenerative and psychiatric disorders, and the adverse effects of glucocorticoid receptor agonist treatment or therapy.

In another embodiment, the present invention provides for the novel preparation of a pharmaceutical composition, wherein: the pharmaceutical composition is suitable for a route of administration selected from oral, nasal, buccal, transdermal, pulmonal, and parenteral.

In another embodiment, the present invention provides a novel method for the treatment of conditions, disorders, or diseases wherein a modulation or an inhibition of the activity of 11βHSD1 is beneficial, the method comprising administering to a subject in need thereof an effective amount of a compound of the present invention.

In another embodiment, the present invention provides a novel method wherein the conditions, disorders, and diseases that are influenced by intracellular glucocorticoid levels.

In another embodiment, the present invention provides a novel method wherein the conditions, disorders, or diseases are selected from metabolic syndrome, insulin resistance, dyslipidemia, hypertension, obesity, type 2 diabetes, impaired glucose tolerance (IGT), impaired fasting glucose (IFG), progression from IGT to type 2 diabetes, progression of metabolic syndrome into type 2 diabetes, diabetic late complications, neurodegenerative and psychiatric disorders, and the adverse effects of glucocorticoid receptor agonist treatment or therapy.

In another embodiment, the present invention provides a novel method wherein the administering is via a route selected from oral, nasal, buccal, transdermal, pulmonal, and parenteral.

In another embodiment, the present invention provides a novel compound, which is an agent useful for the treatment of conditions, disorders, or diseases wherein a modulation or an inhibition of the activity of 11βHSD1 is beneficial.

In another embodiment, the present invention provides a novel method wherein the conditions, disorders, and diseases that are influenced by intracellular glucocorticoid levels.

In another embodiment, the present invention provides a novel method wherein the conditions, disorders, or diseases are selected from metabolic syndrome, insulin resistance, dyslipidemia, hypertension, obesity, type 2 diabetes, impaired glucose tolerance (IGT), impaired fasting glucose (IFG), progression from IGT to type 2 diabetes, progression of metabolic syndrome into type 2 diabetes, diabetic late complications, neurodegenerative and psychiatric disorders, and the adverse effects of glucocorticoid receptor agonist treatment or therapy.

In another embodiment, the present invention provides a novel method pharmaceutical composition comprising, as an active ingredient, at least one compound according of the present invention together with one or more pharmaceutically acceptable carriers or excipients.

In another embodiment, the present invention provides a novel pharmaceutical composition, which is suitable for oral, nasal, buccal, transdermal, pulmonal, or parenteral administration.

The compounds of the present invention have asymmetric centers and may occur as racemates, racemic mixtures, and as individual enantiomers or diastereoisomers, with all isomeric forms being included in the present invention as well as mixtures thereof.

The present invention also encompasses pharmaceutically acceptable salts of the present compounds. Such salts include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, and nitric acids. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, and ketoglutarates. Further examples of pharmaceutically acceptable inorganic or organic acid addition salts include the pharmaceutically acceptable salts listed in J. Pharm. Sci., 66, 2 (1977), which is incorporated herein by reference. Examples of metal salts include lithium, sodium, potassium, barium, calcium, magnesium, zinc, and calcium salts. Examples of amines and organic amines include ammonium, methylamine, dimethylamine, trimethylamine, ethylamine, diethylamine, propylamine, butylamine, tetramethylamine, ethanolamine, diethanolamine, triethanolamine, meglumine, ethylenediamine, choline, N,N′-dibenzylethylene-diamine, N-benzylphenylethylamine, N-methyl-D-glucamine, and guanidine. Examples of cationic amino acids include lysine, arginine, and histidine.

Further, some of the compounds of the present invention may form solvates with water or common organic solvents. Such solvates are encompassed within the scope of the invention.

The pharmaceutically acceptable salts are prepared by reacting a compound of the present invention with 1 to 4 equivalents of a base such as sodium hydroxide, sodium methoxide, sodium hydride, potassium tert-butoxide, calcium hydroxide, and magnesium hydroxide, in solvents such as ether, THF, methanol, tert-butanol, dioxane, and isopropanol, ethanol. Mixtures of solvents may be used. Organic bases such as lysine, arginine, diethanolamine, choline, guandine and their derivatives etc. may also be used. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid salicylic acid, hydroxynaphthoic acid, ascorbic acid, palmitic acid, succinic acid, benzoic acid, benzenesulfonic acid, and tartaric acid in solvents such as ethyl acetate, ether, alcohols, acetone, THF, and dioxane. Mixture of solvents may also be used.

The stereoisomers of the compounds forming part of this invention may be pre-pared by using reactants in their single enantiomeric form in the process wherever possible or by conducting the reaction in the presence of reagents or catalysts in their single enanti-omer form or by resolving the mixture of stereoisomers by conventional methods. Some of the preferred methods include use of microbial resolution, enzymatic resolution, resolving the diastereomeric salts formed with chiral acids such as mandelic acid, camphorsulfonic acid, tartaric acid, and lactic acid, wherever applicable or chiral bases such as brucine, (R)- or (S)-phenylethylamine, cinchona alkaloids and their derivatives. Commonly used methods are compiled by Jaques et al. in “Enantiomers, Racemates and Resolution” (Wiley Interscience, 1981). More specifically the compound of the present invention may be converted to a 1:1 mixture of diastereomeric amides by treating with chiral amines, aminoacids, aminoalcohols derived from aminoacids; conventional reaction conditions may be employed to convert acid into an amide; the diastereomers may be separated either by fractional crystallization or chromatography and the stereoisomers of compound of formula I may be prepared by hydrolysing the pure diastereomeric amide.

Various polymorphs of the compounds forming part of this invention may be pre-pared by crystallization of said compounds under different conditions. For example, using different solvents commonly used or their mixtures for recrystallization; crystallizations at different temperatures; various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, ir spectroscopy, differential scanning calorimetry, powder X-ray diffraction or such other techniques.

The invention also encompasses prodrugs of the present compounds, which on administration undergo chemical conversion by metabolic processes before becoming active pharmacological substances. In general, such prodrugs will be functional derivatives of the present compounds, which are readily convertible in vivo into the required compound of the present invention. Conventional procedures for the selection and preparation of suitable prodrug derivatives are described, for example, in “Design of Prodrugs”, ed. H. Bundgaard, Elsevier, 1985.

It is a well known problem in drug discovery that compounds, such as enzyme inhibitors, may be very potent and selective in biochemical assays, yet be inactive in vivo. This lack of so-called bioavailability may be ascribed to a number of different factors such as lack of or poor absorption in the gut, first pass metabolism in the liver and/or poor uptake in cells. Although the factors determining bioavailability are not completely understood, there are many examples in the scientific literature—well known to those skilled in the art—of how to modify compounds, which are potent and selective in biochemical assays but show low or no activity in vivo, into drugs that are biologically active.

It is within the scope of the invention to modify the compounds of the present invention, termed the ‘original compound’, by attaching chemical groups that will improve the bioavailability of said compounds in such a way that the uptake in cells or mammals is facilitated.

Examples of said modifications, which are not intended in any way to limit the scope of the invention, include changing of one or more carboxy groups to esters (for instance methyl esters, ethyl esters, tert-butyl, acetoxymethyl, pivaloyloxymethyl esters or other acyloxymethyl esters). Compounds of the invention, original compounds, such modified by attaching chemical groups are termed ‘modified compounds’.

The invention also encompasses active metabolites of the present compounds.

The compounds according to the invention alter, and more specifically, reduce the level of active intracellular glucocorticoid and are accordingly useful for the treatment of conditions, disorders, and diseases in which such a modulation or reduction is beneficial.

Accordingly, the present compounds may be applicable for the treatment of the metabolic syndrome, insulin resistance, dyslipidemia, hypertension, obesity, type 2 diabetes, impaired glucose tolerance (IGT), impaired fasting glucose (IFG), Latent Autoimmune Diabetes in the Adult (LADA), type 1 diabetes, diabetic late complications including cardiovascular diseases, cardiovascular disorders, disorders of lipid metabolism, neurodegenerative and psychiatric disorders, dysregulation of intraocular pressure including glaucoma, immune disorders, inappropriate immune responses, musculo-skeletal disorders, gastrointestinal disorders, polycystic ovary syndrome (PCOS), reduced hair growth or other diseases, disorders or conditions that are influenced by intracellular glucocorticoid levels, adverse effects of increased blood levels of active endogenous or exogenous glucocorticoid, and any combination thereof, adverse effects of increased plasma levels of endogenous active glucocorticoid, Cushing's disease, Cushing's syndrome, adverse effects of glucocorticoid receptor agonist treatment of autoimmune diseases, adverse effects of glucocorticoid receptor agonist treatment of inflammatory diseases, adverse effects of glucocorticoid receptor agonist treatment of diseases with an inflammatory component, adverse effects of glucocorticoid receptor agonist treatment as a part of cancer chemotherapy, adverse effects of glucocorticoid receptor agonist treatment for surgical/post-surgical or other trauma, adverse effects of glucocorticoid receptor agonist therapy in the context of organ or tissue transplantation or adverse effects of glucocorticoid receptor agonist treatment in other diseases, disorders or conditions where glucocorticoid receptor agonists provide clinically beneficial effects.

More specifically the present compounds may be applicable for the treatment of the metabolic syndrome, type 2 diabetes, diabetes as a consequence of obesity, insulin resistance, hyperglycemia, prandial hyperglycemia, hyperinsulinemia, inappropriately low insulin secretion, impaired glucose tolerance (IGT), impaired fasting glucose (IFG), increased hepatic glucose production, type 1 diabetes, LADA, pediatric diabetes, dyslipidemia, diabetic dyslipidemia, hyperlipidemia, hypertriglyceridemia, hyperlipoproteinemia, hypercholesterolemia, decreased HDL cholesterol, impaired LDL/HDL ratio, other disorders of lipid metabolism, obesity, visceral obesity, obesity as a consequence of diabetes, increased food intake, hypertension, diabetic late complications, micro-/macroalbuminuria, nephropathy, retinopathy, neuropathy, diabetic ulcers, cardiovascular diseases, arteriosclerosis, atherosclerosis, coronary artery disease, cardiac hypertrophy, myocardial ischemia, heart insufficiency, congestional heart failure, stroke, myocardial infarction, arrythmia, decreased blood flow, erectile dysfunction (male or female), myopathy, loss of muscle tissue, muscle wasting, muscle catabolism, osteoporosis, decreased linear growth, neurodegenerative and psychiatric disorders, Alzheimers disease, neuronal death, impaired cognitive function, depression, anxiety, eating disorders, appetite regulation, migraine, epilepsia, addiction to chemical substances, disorders of intraocular pressure, glaucoma, polycystic ovary syndrome (PCOS), inappropriate immune responses, inappropriate T helper-1/T helper-2 polarisation, bacterial infections, mycobacterial infections, fungal infections, viral infections, parasitic infestations, suboptimal responses to immunizations, immune dysfunction, partial or complete baldness, or other diseases, disorders or conditions that are influenced by intracellular glucocorticoid levels and any combination thereof, adverse effects of glucocorticoid receptor agonist treatment of allergic-inflammatory diseases such as asthma and atopic dermatitis, adverse effects of glucocorticoid receptor agonist treatment of disorders of the respiratory system e.g., asthma, cystic fibrosis, emphysema, bronchitis, hypersensitivity, pneumonitis, eosinophilic pneumonias, pulmonary fibrosis, adverse effects of glucocorticoid receptor agonist treatment of inflammatory bowel disease such as Crohn's disease and ulcerative colitis; adverse effects of glucocorticoid receptor agonist treatment of disorders of the immune system, connective tissue and joints e.g., reactive arthritis, rheumatoid arthritis, Sjögren's syndrome, systemic lupus erythematosus, lupus nephritis, Henoch-Schönlein purpura, Wegener's granulomatosis, temporal arteritis, systemic sclerosis, vasculitis, sarcoidosis, dermatomyositis-polymyositis, pemphigus vulgaris; adverse effects of glucocorticoid receptor agonist treatment of endocrinological diseases such as hyperthyroidism, hypoaldosteronism, hypopituitarism; adverse effects of glucocorticoid receptor agonist treatment of hematological diseases e.g., hemolytic anemia, thrombocytopenia, paroxysmal nocturnal hemoglobinuria; adverse effects of glucocorticoid receptor agonist treatment of cancer such as spinal cord diseases, neoplastic compression of the spinal cord, brain tumours, acute lymphoblastic leukemia, Hodgkin's disease, chemotherapy-induced nausea, adverse effects of glucocorticoid receptor agonist treatment of diseases of muscle and at the neuro-muscular joint e.g., myasthenia gravis and hereditary myopathies (e.g., Duchenne muscular dystrophy), adverse effects of glucocorticoid receptor agonist treatment in the context of surgery & transplantation e.g., trauma, post-surgical stress, surgical stress, renal transplantation, liver transplantation, lung transplantation, pancreatic islet transplantation, blood stem cell transplantation, bone marrow transplantation, heart transplantation, adrenal gland transplantation, tracheal transplantation, intestinal transplantation, corneal transplantation, skin grafting, keratoplasty, lens implantation and other procedures where immunosuppression with glucocorticoid receptor agonists is beneficial; adverse effects of glucocorticoid receptor agonist treatment of brain abscess, nausea/vomiting, infections, hypercalcemia, adrenal hyperplasia, autoimmune hepatitis, spinal cord diseases, saccular aneurysms or adverse effects to glucocorticoid receptor agonist treatment in other diseases, disorders and conditions where glucocorticoid receptor agonists provide clinically beneficial effects.

Accordingly, in a further aspect the invention relates to a compound according to the invention for use as a pharmaceutical composition.

The invention also relates to pharmaceutical compositions comprising, as an active ingredient, at least one compound according to the invention together with one or more pharmaceutically acceptable carriers or diluents.

The pharmaceutical composition is preferably in unit dosage form, comprising from about 0.05 mg/day to about 2000 mg/day, preferably from about 0.1 mg/day to about 1000 mg/day, and more preferably from about 0.5 mg/day to about 500 mg/day of a compound according to the invention.

In another embodiment, the patient is treated with a compound according to the invention for at least about 1 week, for at least about 2 weeks, for at least about 4 weeks, for at least about 2 months or for at least about 4 months.

In yet another embodiment, the pharmaceutical composition is for oral, nasal, buccal, transdermal, pulmonal or parenteral administration.

Furthermore, the invention relates to the use of a compound according to the invention for the preparation of a pharmaceutical composition for the treatment of disorders and diseases wherein a modulation or an inhibition of the activity of 11βHSD1 is beneficial.

The invention also relates to a method for the treatment of disorders and diseases wherein a modulation or an inhibition of the activity of 11βHSD1 is beneficial, the method comprising administering to a subject in need thereof an effective amount of a compound according to the invention.

In a preferred embodiment of the invention the present compounds are used for the preparation of a medicament for the treatment of any diseases and conditions that are influenced by intracellular glucocorticoid levels as mentioned above.

Thus, in a preferred embodiment of the invention the present compounds are used for the preparation of a medicament for the treatment of conditions and disorders where a decreased level of active intracellular glucocorticoid is desirable, such as the conditions and diseases mentioned above.

In yet a preferred embodiment of the invention the present compounds are used for the preparation of a medicament for the treatment of metabolic syndrome, insulin resistance, dyslipidemia, hypertension obesity, type 2 diabetes, impaired glucose tolerance (IGT), impaired fasting glucose (IFG), progression from IGT to type 2 diabetes, progression of the metabolic syndrome into type 2 diabetes, diabetic late complications (e.g., cardiovascular diseases, arteriosclerosis, and atherosclerosis), neurodegenerative and psychiatric disorders, and, the adverse effects of glucocorticoid receptor agonist treatment or therapy.

In another embodiment of the present invention, the route of administration may be any route which effectively transports a compound according to the invention to the appropriate or desired site of action, such as oral, nasal, buccal, transdermal, pulmonal, or parenteral.

In still a further aspect of the invention the present compounds are administered in combination with one or more further active substances in any suitable ratios. Such further active substances may e.g., be selected from antiobesity agents, antidiabetics, agents modifying the lipid metabolism, antihypertensive agents, glucocorticoid receptor agonists, agents for the treatment and/or prevention of complications resulting from or associated with diabetes and agents for the treatment and/or prevention of complications and disorders resulting from or associated with obesity.

Thus, in a further aspect of the invention the present compounds may be administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator-activated receptor) modulators, RXR (retinoid X receptor) modulators, TR β agonists, AGRP (Agouti related protein) inhibitors, H3 histamine antagonists, opioid antagonists (such as naltrexone), exendin-4, GLP-1 and ciliary neurotrophic factor.

In one embodiment of the invention the antiobesity agent is leptin; dexamphetamine or amphetamine; fenfluramine or dexfenfluramine; sibutramine; orlistat; mazindol or phentermine.

Suitable antidiabetic agents include insulin, insulin analogues and derivatives such as those disclosed in EP 792 290 (Novo Nordisk A/S), e.g., N^{εB29}-tetradecanoyl des (B30) human insulin, EP 214 826 and EP 705 275 (Novo Nordisk A/S), e.g., Asp^{B28} human insulin, U.S. Pat. No. 5,504,188 (Eli Lilly), e.g., Lys^{B28} Pro^{B29} human insulin, EP 368 187 (Aventis), eg Lantus, which are all incorporated herein by reference, GLP-1 (glucagon like peptide-1) and GLP-1 derivatives such as those disclosed in WO 98/08871 to Novo Nordisk A/S, which is incorporated herein by reference as well as orally active hypoglycaemic agents.

The orally active hypoglycaemic agents preferably comprise sulphonylureas, biguanides, meglitinides, glucosidase inhibitors, glucagon antagonists such as those disclosed in WO 99/01423 to Novo Nordisk A/S and Agouron Pharmaceuticals, Inc., GLP-1 agonists, potassium channel openers such as those disclosed in WO 97/26265 and WO 99/03861 to Novo Nordisk A/S which are incorporated herein by reference, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents and antilipidemic agents as PPARα modulators, PPARδ modulators, cholesterol absorption inhibitors, HSL (hormone-sensitive lipase) inhibitors and HMG CoA inhibitors (statins), nicotinic acid, fibrates, anion exchangers, compounds lowering food intake, bile acid resins, RXR agonists and agents acting on the ATP-dependent potassium channel of the β-cells.

In one embodiment, the present compounds are administered in combination with insulin or an insulin analogue or derivative, such as N^{εB29}-tetradecanoyl des (B30) human insulin, Asp^{B28} human insulin, Lys^{B28} Pro^{B29} human insulin, Lantus®, or a mix-preparation comprising one or more of these.

In a further embodiment the present compounds are administered in combination with a sulphonylurea e.g., tolbutamide, glibenclamide, glipizide or glicazide.

In another embodiment the present compounds are administered in combination with a biguanide e.g., metformin.

In yet another embodiment the present compounds are administered in combination with a meglitinide e.g., repaglinide or senaglinide.

In still another embodiment the present compounds are administered in combination with a thiazolidinedione e.g., troglitazone, ciglitazone, pioglitazone, rosiglitazone or compounds disclosed in WO 97/41097 such as 5-[[4-[3-Methyl-4-oxo-3,4-dihydro-2-quinazolinyl]methoxy]phenyl-methyl]thiazolidine-2,4-dione or a pharmaceutically acceptable salt thereof, preferably the potassium salt.

In yet another embodiment the present compounds may be administered in combination with the insulin sensitizers disclosed in WO 99/19313 such as (−) 3-[4-[2-Phenoxazin-10-yl)ethoxy]phenyl]-2-ethoxypropanoic acid or a pharmaceutically acceptable salts thereof, preferably the arginine salt.

In a further embodiment the present compounds are administered in combination with an α-glucosidase inhibitor e.g., miglitol or acarbose.

In another embodiment the present compounds are administered in combination with an agent acting on the ATP-dependent potassium channel of the β-cells e.g., tolbutamide, glibenclamide, glipizide, glicazide or repaglinide.

Furthermore, the present compounds may be administered in combination with nateglinide.

In still another embodiment the present compounds are administered in combination with an antihyperlipidemic agent or antilipidemic agent e.g., cholestyramine, colestipol, clofibrate, gemfibrozil, fenofibrate, bezafibrate, tesaglitazar, EML-4156, LY-818, MK-767, atorvastatin, fluvastatin, lovastatin, pravastatin, simvastatin, acipimox, probucol, ezetimibe or dextrothyroxine.

In a further embodiment the present compounds are administered in combination with more than one of the above-mentioned compounds e.g., in combination with a sulphonylurea and metformin, a sulphonylurea and acarbose, repaglinide and metformin, insulin and a sulphonylurea, insulin and metformin, insulin, insulin and lovastatin, etc.

Further, the present compounds may be administered in combination with one or more antihypertensive agents. Examples of antihypertensive agents are β-blockers such as alprenolol, atenolol, timolol, pindolol, propranolol, metoprolol, bisoprololfumerate, esmolol, acebutelol, metoprolol, acebutolol, betaxolol, celiprolol, nebivolol, tertatolol, oxprenolol, amusolalul, carvedilol, labetalol, β2-receptor blockers e.g., S-atenolol, OPC-1085, ACE (angiotensin converting enzyme) inhibitors such as quinapril, lisinopril, enalapril, captopril, benazepril, perindopril, trandolapril, fosinopril, ramipril, cilazapril, delapril, imidapril, moexipril, spirapril, temocapril, zofenopril, S-5590, fasidotril, Hoechst-Marion Roussel: 100240 (EP 00481522), omapatrilat, gemopatrilat and GW-660511, calcium channel blockers such as nifedipine, felodipine, nicardipine, isradipine, nimodipine, diltiazem, amlodipine, nitrendipine, verapamil, lacidipine, lercanidipine, aranidipine, cilnidipine, clevidipine, azelnidipine, barnidipine, efonodipine, iasidipine, iemildipine, iercanidipine, manidipine, nilvadipine, pranidipine, furnidipine, α-blockers such as doxazosin, urapidil, prazosin, terazosin, bunazosin and OPC-28326, diuretics such as thiazides/sulphonamides (e.g., bendroflumetazide, chlorothalidone, hydrochlorothiazide and clopamide), loop-diuretics (e.g., bumetanide, furosemide and torasemide) and potassium sparing diuretics (e.g., amiloride, spironolactone), endothelin ET-A antagonists such as ABT-546, ambrisetan, atrasentan, SB-234551, CI-1034, S-0139 and YM-598, endothelin antagonists e.g., bosentan and J-104133, renin inhibitors such as aliskiren, vasopressin V1 antagonists e.g., OPC-21268, vasopressin V2 antagonists such as tolvaptan, SR-121463 and OPC-31260, B-type natriuretic peptide agonists e.g., Nesiritide, angiotensin II antagonists such as irbesartan, candesartancilexetil, losartan, valsartan, telmisartan, eprosartan, candesartan, CL-329167, eprosartan, iosartan, olmesartan, pratosartan, TA-606, and YM-358, 5-HT2 agonists e.g., fenoldopam and ketanserin, adenosine A1 antagonists such as naftopidil, N-0861 and FK-352, thromboxane A2 antagonists such as KT2-962, endopeptidase inhibitors e.g., ecadotril, nitric oxide agonists such as LP-805, dopamine D1 antagonists e.g., MYD-37, dopamine D2 agonists such as nolomirole, n-3 fatty acids e.g., omacor, prostacyclin agonists such as treprostinil, beraprost, PGE1 agonists e.g., ecraprost, Na+/K+ ATPase modulators e.g., PST-2238, Potassium channel activators e.g., KR-30450, vaccines such as PMD-3117, Indapamides, CGRP-unigene, guanylate cyclase stimulators, hydralazines, methyldopa, docarpamine, moxonidine, CoAprovel, MondoBiotech-811.

Further reference can be made to Remington: The Science and Practice of Pharmacy, 19^{th} Edition, Gennaro, Ed., Mack Publishing Co., Easton, Pa., 1995.

Furthermore, the present compounds may be administered in combination with one or more glucocorticoid receptor agonists. Examples of such glucocorticoid receptor agonists are betametasone, dexamethasone, hydrocortisone, methylprednisolone, prednisolone, prednisone, beclomethasone, butixicort, clobetasol, flunisolide, flucatisone (and analogues), momethasone, triamcinolonacetonide, triamcinolonhexacetonide GW-685698, NXC-1015, NXC-1020, NXC-1021, NS-126, P-4112, P-4114, RU-24858 and T-25 series.

It should be understood that any suitable combination of the compounds according to the invention with one or more of the above-mentioned compounds and optionally one or more further pharmacologically active substances are considered to be within the scope of the present invention.

### Pharmaceutical Compositions

The compounds of the present invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as hard or soft capsules, tablets, troches, dragees, pills, lozenges, powders and granules. Where appropriate, they can be prepared with coatings such as enteric coatings or they can be formulated so as to provide controlled release of the active ingredient such as sustained or prolonged release according to methods well-known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, crèmes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 2000 mg, e.g., from about 0.1 to about 1000 mg, from about 0.5 mg to about 500 mg., from about 1 mg to about 200 mg, e.g., about 100 mg.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. Examples are an acid addition salt of a compound having the utility of a free base and a base addition salt of a compound having the utility of a free acid. The term “pharmaceutically acceptable salts” refers to non-toxic salts of the compounds for use according to the present invention which are generally prepared by reacting the free base with a suitable organic or inorganic acid or by reacting the acid with a suitable organic or inorganic base. When a compound for use according to the present invention, contains a free base such salts are prepared in a conventional manner by treating a solution or suspension of the compound with a chemical equivalent of a pharmaceutically acceptable acid. When a compounds for use according to the present invention, contains a free acid such salts are prepared in a conventional manner by treating a solution or suspension of the compound with a chemical equivalent of a pharmaceutically acceptable base. Physiologically acceptable salts of a compound with a hydroxy group include the anion of said compound in combination with a suitable cation such as sodium or ammonium ion. Other salts which are not pharmaceutically acceptable may be useful in the preparation of compounds for use according to the present invention and these form a further aspect of the present invention.

For parenteral administration, solutions of the present compounds in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of suitable carriers are water, salt solutions, alcohols, polyethylene glycols, polyhydroxyethoxylated castor oil, peanut oil, olive oil, syrup, phospholipids, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents.

The pharmaceutical compositions formed by combining the compounds of the invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

Compositions intended for oral use may be prepared according to any known method, and such compositions may contain one or more agents selected from the group consisting of sweetening agents, flavouring agents, colouring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsule wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinylpyrrolidone, gum tragacanth and gum acacia; dispersing or wetting agents may be a naturally-occurring phosphatide such as lecithin, or condensation products of an alkyl oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be pre-served by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions comprising a compound for use according to the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, preservative and flavouring and colouring agent. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the present invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the present invention are contemplated. For the purpose of this application, topical applications shall include mouth washes and gargles.

The compounds for use according to the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In addition, some of the compounds for use according to the present invention may form solvates with water or common organic solvents. Such solvates are also encompassed within the scope of the present invention.

Thus, in a further embodiment, there is provided a pharmaceutical composition comprising a compound for use according to the present invention, or a pharmaceutically acceptable salt, solvate, or prodrug thereof, and one or more pharmaceutically acceptable carriers, excipients, or diluents.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet which may be prepared by conventional tabletting techniques may contain:

Core:

| | | |
|---|---|---|
| Active compound (as free compound or salt thereof) | 5.0 | mg |
| Lactosum PH. Eur. | 67.8 | mg |
| Cellulose, microcryst. (Avicel) | 31.4 | mg |
| Amberlite ® IRP88* | 1.0 | mg |

| | |
|---|---|
| Magnesii stearas PH. Eur. | q.s. |

Coating:

| | | |
|---|---|---|
| Hydroxypropyl methylcellulose | approx. 9 | mg |
| Mywacett 9-40 T** | approx. 0.9 | mg |

Polacrillin potassium NF, tablet disintegrant, Rohm and Haas.
**Acylated monoglyceride used as plasticizer for film coating.

The compounds of the invention may be administered to a patient which is a mammal, especially a human in need thereof. Such mammals include also animals, both domestic animals, e.g., household pets, and non-domestic animals such as wildlife.

Any novel feature or combination of features described herein is considered essential to this invention.

The present invention also relate to the below methods of preparing the compounds of the invention.

The present invention is further illustrated in the following representative examples which are, however, not intended to limit the scope of the invention in any way.

### EXAMPLES

### Compounds of General Formula (I)

The following examples and general procedures refer to intermediate compounds and final products for general formula (I) identified in the specification and in the synthesis schemes. The preparation of the compounds of general formula (I) of the present invention is described in detail using the following examples. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, which is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials. The structures of the compounds are confirmed by either elemental analysis or nuclear magnetic resonance (NMR), where peaks assigned to characteristic protons in the title compounds are presented where appropriate. ¹H NMR shifts (6H) are given in parts per million (ppm) down field from tetramethylsilane as internal reference standard. M.p.: is melting point and is given in IC and is not corrected. Column chromatography was carried out using the technique described by W. C. Still et al., J. Org. Chem. 43:2923 (1978) on Merck silica gel 60 (Art. 9385). HPLC analyses are performed using 5 μm C18 4×250 mm column eluted with various mixtures of water and acetonitrile, flow=1 ml/min, as described in the experimental section.

Microwave oven synthesis: The reaction was heated by microwave irradiation in sealed microwave vessels in a single mode Emrys Optimizer EXP from PersonalChemistry®.

Preparative HPLC: Column: 1.9×15 cm Waters XTerra RP-18. Buffer: linear gradient 5-95% in 15 min, MeCN, 0.1% TFA, flow rate of 15 ml/min. The pooled fractions are either evaporated to dryness in vacuo, or evaporated in vacuo until the MeCN is removed, and then frozen and freeze dried.

The abbreviations as used in the examples have the following meaning:

TLC: Thin layer chromatography

CDCl₃: Deuterio chloroform

CD₃OD: Tetradeuterio methanol

DCM: Dichloromethane

DMF: N,N-dimethylformamide

DMSO-d₆: Hexadeuterio dimethylsulfoxide

DMSO: Dimethylsulfoxide

DIPEA: Diisopropylethylamine

EDAC: 1-(3-Dimethylaminopropyl)-3-ethylcarbodiimide hydrochloride

EtOAc: Ethyl acetate

THF: Tetrahydrofuran

HOBT: 1-Hydroxy-benzotriazole

MeCN: Acetonitrile

NMP: N-Methylpyrrolidinone

TFA: Trifluoroacetic acid

min: minutes

hrs: hours

General Method A:

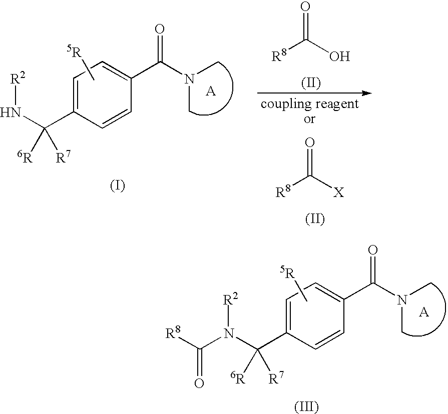

By allowing a benzyl amine (I) wherein R², R⁵, R⁶, R⁷ and A are defined as above to be coupled with an acid (II) wherein R⁸ is defined as above under standard amide forming conditions using a coupling reagent (III) (e.g. HOBT, EDAC and DIPEA in dry THF) affording amide (IV) wherein R², R⁵, R⁶, R⁷, R⁸ and A are defined as above; or by allowing a benzyl amine (I) wherein R², R⁵, R⁶, R⁷ and A are defined as above to be reacted with an acid derivative (II) wherein X is halo, R⁸(C═O)O—, C₁-C₆alkyloxy or arylC₁-C₆alkyloxy and R⁸ is defined as above under basic conditions (e.g. triethylamine, K₂CO₃, NaH and the like) in a solvent (e.g. THF, DCM, DMF, NMP and the like) affording amide (III); wherein R², R⁵, R⁶, R⁷, R⁸ and A are defined as above.

General Method B:

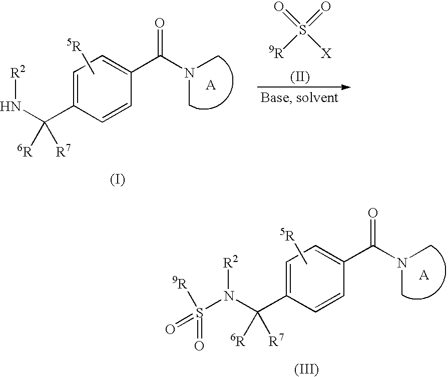

By allowing a benzyl amine (I) wherein R², R⁵, R⁶, R⁷ and A are defined as above to be reacted with a sulphonyl halide (II) wherein X is halo and R⁹ is defined as above under basic conditions (e.g. triethylamine, K₂CO₃, NaH and the like) in a solvent (e.g. THF, DCM, DMF, NMP and the like) affording sulphone amide (III); wherein R², R⁵, R⁶, R⁷, R⁹ and A are defined as above.

General Method C:

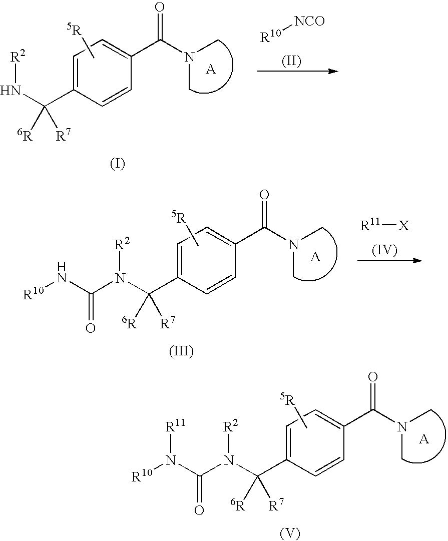

By allowing a benzyl amine (I) wherein R², R⁵, R⁶, R⁷ and A are defined as above to be reacted with an isocyanate (II) wherein R¹⁰ is defined as above in a solvent (e.g. THF, DCM, DMF, NMP and the like) affording urea (III); wherein R², R⁵, R⁶, R⁷, R¹⁰ and A are defined as above. Tri-substituted urea (III) can further be reacted with an alkyl halide or mesylate (IV); wherein X is halide or OSO₂Me and R¹¹ is defined above to react under basic condition (e.g. triethylamine, K₂CO₃, NaH and the like) in a solvent (e.g. THF, DCM, DMF, NMP and the like) affording tetra-substituted urea (V); wherein R², R⁵, R⁶, R⁷, R¹⁰, R¹¹ and A are defined as above.

General Method D:

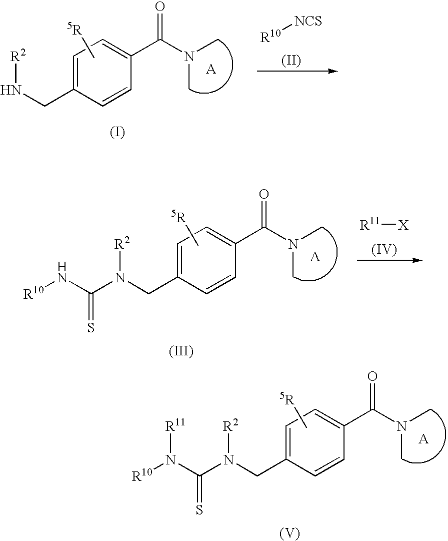

By allowing a benzyl amine (I) wherein R², R⁵, R⁶, R⁷ and A are defined as above to be reacted with an isothiocyanate (II) wherein R¹⁰ is defined as above in a solvent (e.g. THF, DCM, DMF, NMP and the like) affording thiourea (III); wherein R², R⁵, R⁶, R⁷, R¹⁰ and A are defined as above. Tri-substituted thiourea (III) can further be reacted with an alkyl halide or mesylate (IV); wherein X is halide or OSO₂Me and R¹¹ is defined above to react under basic condition (e.g. triethylamine, K₂CO₃, NaH and the like) in a solvent (e.g. THF, DCM, DMF, NMP and the like) affording tetra-substituted thiourea (V); wherein R², R⁵, R⁶, R⁷, R¹⁰, R¹¹ and A are defined as above.

General Method E:

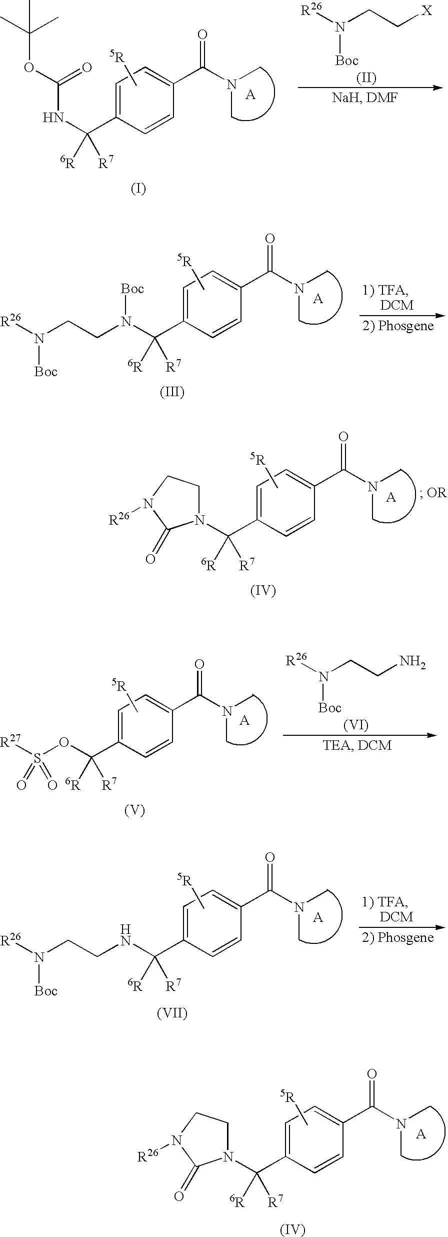

By allowing a benzyl amine (I); wherein R⁵, R⁶, R⁷ and A are defined as above to be reacted with an protected ethyl amine (II); wherein X is halo, C₁-C₆alkylOS(O)₂—, aryl-OS(O)₂— or arylC₁-C₆alkylOS(O)₂— and R²⁶ is C₁-C₈alkyl, C₃-C₁₀cycloalkyl, C₃-C₁₀het-cycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)R¹², —S(O)ₙNR¹³R¹⁴ and C₁-C₆alkyloxyC₁-C₆alkyl to react under basic condition (e.g. triethylamine, K₂CO₃, NaH and the like) in a solvent (e.g. THF, DCM, DMF, NMP and the like) affording ethylene diamine (III); wherein R⁵, R⁶, R⁷ and A are defined as above and R²⁶ is C₁-C₈alkyl, C₃-C₁₀cycloalkyl, C₃-C₁₀het-cycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)ₙNR¹³R¹⁴ and C₁-C₆alkyloxyC₁-C₆alkyl. Deprotection of ethylene diamine (III); wherein R⁵, R⁶, R⁷ and A are defined as above and R²⁶ is C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)ₙNR¹³R¹⁴ and C₁-C₆alkyloxyC₁-C₆alkyl, in a mixtyre of e.g. TFA/DCM followed by reaction with phosgene under basic conditions (e.g. triethylamine, DIPEA, DBU ad the like) in a solvent (e.g. THF, DCM, toluene and the like) affords 2-oxo-imidazolidine (IV); wherein R⁵, R⁶, R⁷ and A are defined as above and R²⁶ is C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)ₙNR¹³R¹⁴ and C₁-C₆alkyloxyC₁-C₆alkyl; or

by allowing a benzyl sulphonate (V); wherein R⁵, R⁶, R⁷, A are defined as above and R²⁷ is C₁-C₆alkyl and aryl, to be reacted with an protected ethylene di-amine (II); wherein R²⁶ is C₁-C₈alkyl, C₃-C₁₀cycloalkyl, C₃-C₁₀het-cycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)ₙNR¹³R¹⁴ and C₁-C₆alkyloxyC₁-C₆alkyl to react under basic condition (e.g. triethylamine, K₂CO₃, NaH and the like) in a solvent (e.g. THF, DCM, DMF, NMP and the like) affording ethylene di-amine (III); wherein R⁵, R⁶, R⁷ and A are defined as above and R²⁶ is C₁-C₈alkyl, C₃-C₁₀cycloalkyl, C₃-C₁₀het-cycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)ₙNR¹³R¹⁴ and C₁-C₆alkyloxyC₁-C₆alkyl. Deprotection of ethylene diamine (III); wherein R⁵, R⁶, R⁷ and A are defined as above and R²⁶ is C₁-C₈alkyl, C₃-C₁₀cycloalkyl, C₃-C₁₀het-cycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)NR¹³R¹⁴ and C₁-C₆alkyloxyC₁-C₆alkyl in a mixtyre of e.g. TFA/DCM followed by reaction with phosgene under basic conditions (e.g. triethylamine, DIPEA, DBU ad the like) in a solvent (e.g. THF, DCM, toluene and the like) affords 2-oxo-imidazolidine (IV); wherein R⁵, R⁶, R⁷ and A are defined as above and R²⁶ is C₁-C₈alkyl, C₃-C₁₀cycloalkyl, C₃-C₁₀het-cycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)ₙNR¹³R¹⁴ and C₁-C₆alkyloxyC₁-C₆alkyl.

R²⁶ is C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)ₙNR¹³R¹⁴ and C₁-C₆alkyloxyC₁-C₆alkyl wherein R¹², R¹³, and R¹⁴ are defined above and each alkyl, aryl/hetaryl group is substituted with 0-3 R¹⁸ which is defined above.

General Method F:

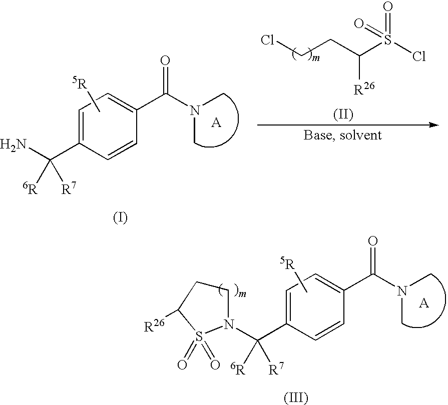

By allowing a benzyl amine (I); wherein R⁵, R⁶, R⁷ and A are defined as above to be reacted with a sulphonyl halide (II); wherein m is 1, 2 or 3 and R²⁶ is defined below under basic conditions (e.g. triethylamine, K₂CO₃, NaH and the like) in a solvent (e.g. THF, DCM, DMF, NMP and the like) affording cyclic sulphone amide (III); wherein m is 1, 2 or 3 and R⁵, R⁶, R⁷ and A are defined as above and R²⁶ is defined below.

R²⁶ is C₁-C₆alkyl, aryl, hetaryl, arylC₁-C₆alkyl, hetarylC₁-C₆alkyl, —C(═O)R¹², —S(O)ₙR¹², —S(O)ₙNR¹³R¹⁴, C₁-C₆alkyloxy, arylC₁-C₆alkyloxy, hetarylC₁-C₆alkyloxy, C₁-C₆alkyloxyC₁-C₆alkyl; wherein R¹², R¹³ and R¹⁴ are defined above and each alkyl, aryl/hetaryl group is substituted with 0-3 R¹⁸ which is defined above.

General Method G:

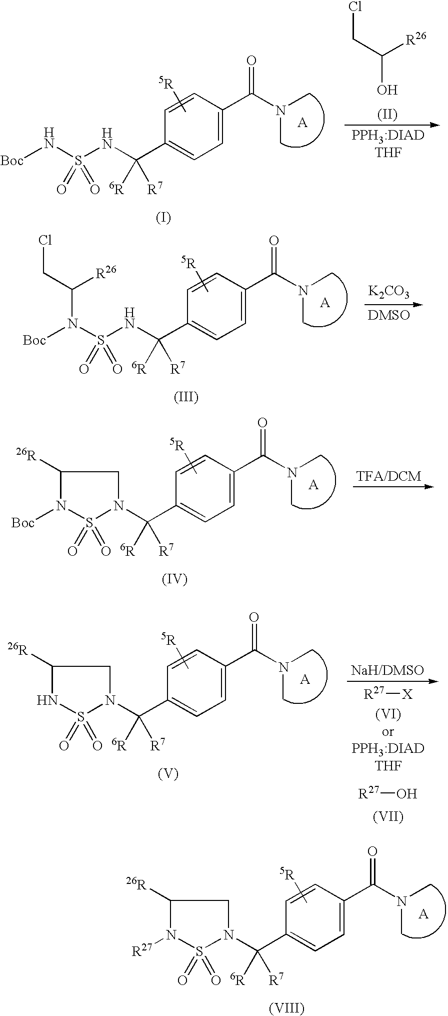

By allowing a sulfamide (I); wherein R⁵, R⁶, R⁷ and A are defined as above to be reacted with a hydroxyl ethyl halide (II); wherein R²⁶ is as defined below under Mitsunobu conditions (e.g. PPh₃ and DIAD) in a solvent (e.g. THF and the like) affording substituted sulfamide (III); wherein R⁵, R⁶, R⁷ and A are defined as above and R²⁶ is as defined below. Substituted sulfamide (III); wherein R⁵, R⁶, R⁷ and A are defined as above and R²⁶ is as defined below is cyclised under basic conditions (e.g. K₂CO₃ in DMSO) affording substituted [1,2,5]thiadiazolidine 1,1-dioxide (IV); wherein R⁵, R⁶, R⁷ and A are defined as above and R²⁶ is as defined below. Introduction of further substituents can be accomplished when allowing a substituted [1,2,5]thiadiazolidine 1,1-dioxide (IV); wherein R⁵, R⁶, R⁷ and A are defined as above and R²⁶ is as defined below to undergo deprotection (e.g. TFA/DCM) affording substituted [1,2,5]thiadiazolidine 1,1-dioxide (V); wherein R⁵, R⁶, R⁷ and A are defined as above and R²⁶ is as defined below which can be alkylated with (VI); wherein R²⁷ is as defined below under basic conditions (e.g. NaH in DMSO or DMF) or via a Mitsunobu reaction (e.g. PPh₃ and DIAD) in a solvent (e.g. THF and the like) with alcohol (VII); wherein R²⁷ is as defined below affording substituted 1,2,5]thiadiazolidine 1,1-dioxide (VIII); wherein R⁵, R⁶, R⁷ and A are defined as above and R²⁶ and R²⁷ are defined below.

R²⁶ is C₁-C₆alkyl, aryl, hetaryl, arylC₁-C₆alkyl, hetarylC₁-C₆alkyl and C₁-C₆alkyloxyC₁-C₆alkyl; wherein each alkyl, aryl/hetaryl group is substituted with 0-3 R¹⁸ which is defined above.

R²⁷ is C₁-C₆alkyl, aryl, hetaryl, arylC₁-C₆alkyl, hetarylC₁-C₆alkyl, —C(═O)R¹², —S(O)ₙR¹², —S(O)ₙNR¹³R¹⁴, C₁-C₆alkyloxyC₁-C₆alkyl; wherein R¹², R¹³ and R¹⁴ are defined above and each alkyl, aryl/hetaryl group is substituted with 0-3 R¹³ which is defined above.

### Specific Examples

### Example 1-1

### General Procedure (A)

### N-Methyl-N-[4-(1,3,3-trimethyl-6-aza-bicyclo[3.2.1]octane-6-carbonyl)-benzyl]-acetamide

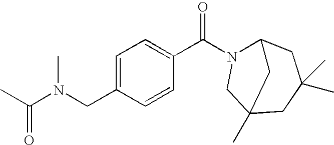

### Step A

### [4-(1,3,3-Trimethyl-6-aza-bicyclo[3.2.1]octane-6-carbonyl)-benzyl]-carbamic Acid Tert-butyl Ester

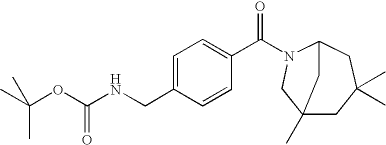

To a solution of 4-(tert-butoxycarbonylamino-methyl)-benzoic acid (15.0 g, 59.69 mmol) in THF (200 mL) was added with stirring HOBt (8.87 g, 65.66 mmol) followed by EDAC (12.59 g, 65.66 mmol) and the mixture was stirred for 30 min. at ambient temperature. To the resulting mixture was added 1,3,3-trimethyl-6-aza-bicyclo[3.2.1]octane, hydrochloride (12.46 g, 65.66 mmol) and DIPEA (21.84 mL, 125.36 mmol). The reaction mixture was stirred for 16 hrs. at ambient temperature. The solvent was evaporated and to the residue was added water (100 mL). The mixture was extracted with EtOAc (3×50 mL) and the combined organic phases were washed with saturated aqueous ammonium chloride (3×50 mL). The organic phase was dried (MgSO₄) and the solvent evaporated affording crude amide which was dissolved in EtOAc (50 mL) and filtered through a patch of silica gel using EtOAc as eluent. The combined fractions were evaporated which afforded 23 g (99%) of [4-(1,3,3-trimethyl-6-azabicyclo[3.2.1]octane-6-carbonyl)-benzyl]-carbamic acid tert-butyl ester.

¹H NMR (400 MHz, CDCl₃) δ 0.94 (d, 3H), 1.02 (d, 3H), 1.12 (d, 3H), 1.17-1.59 (m, 14.5H), 1.75 (m, 1H), 2.23 (m, 0.5H), 3.23 (q, 0.5H), 3.26 (d, 0.5H), 3.58 (d, 0.5H), 3.96 (m, 0.5H), 4.33 (bs, 2H), 4.60 (m, 0.5H), 5.02 (bs, 0.5H), 7.29 (m, 2H), 7.40 (t, 2H). HPLC-MS (Method Z1): m/z=387 (M+1); tᵣ═x.xx min (yy % ELS).

### Step B

### Methyl-[4-(1,3,3-trimethyl-6-aza-bicyclo[3.2.1]octane-6-carbonyl)-benzyl]-carbamic Acid Tert-butyl Ester

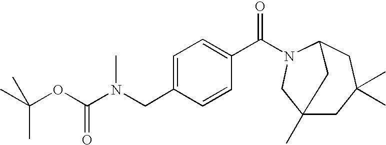

To a solution of the above carbamate (560 mg, 1.45 mmol) in THF (30 mL) was added with stirring sodium hydride (151 mg, 3.77 mmol, 60% in mineral oil) and the mixture was stirred for 1 h. at ambient temperature. To the resulting mixture was added methyl iodine (514 mg, 3.62 mmol) dissolved in THF (1 mL). The reaction mixture was stirred for 16 h. at ambient temperature. The solvent was evaporated and to the residue was added water (30 mL). The mixture was extracted with EtOAc (3×25 mL) and the combined organic phases were washed with water (3×25 mL), brine (25 mL), dried (MgSO₄) and the solvent evaporated affording 560 mg (97%) of methyl-[4-(1,3,3-trimethyl-6-aza-bicyclo[3.2.1]octane-6-carbonyl)-benzyl]-carbamic acid tert-butyl ester as a solid.

¹H NMR (400 MHz, DMSO-d₆) δ 0.89 (d, 3H), 0.96 (d, 3H), 1.05 (d, 3H), 1.15-1.50 (m, 13.5H), 1.74 (m, 1H), 2.04 (m, 0.5H), 2.78 (s, 3H), 3.11 (m, 1H), 3.28 (d, 0.5H), 3.42 (d, 0.5H), 3.92 (m, 0.5H), 4.39 (m, 2.5H), 7.26 (m, 2H), 7.38 (d, 1H), 7.44 (d, 1H). HPLC-MS (Method Z1): m/z=401 (M+1); tᵣ=x.xx min (yy % ELS).

### Step C

### (4-Methylaminomethyl-phenyl)-(1,3,3-trimethyl-6-aza-bicyclo[3.2.1]oct-6-yl)-methanone

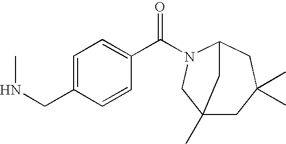

To a solution of the above amide (560 mg, 1.4 mmol) in DCM (9 mL) was added with stirring TFA (3 mL) and the mixture was stirred for 16 h. at ambient temperature. The solvent was evaporated and to the residue was added water (10 mL) and the pH adjusted to 11. The mixture was extracted with DCM (3×15 mL) and the combined organic phases were washed with brine (15 mL), dried (MgSO₄) and the solvent evaporated affording 410 mg (97%) of (4-methylaminomethyl-phenyl)-(1,3,3-trimethyl-6-aza-bicyclo[3.2.1]oct-6-yl)-methanone as an oil.

¹H NMR (400 MHz, MeOD) δ 0.95 (d, 3H), 1.02 (d, 3H), 1.12 (d, 3H), 1.2-1.68 (m, 5H), 1.84 (m, 1H), 2.16 (m, 0.5H), 2.72 (s, 3H), 3.19 (m, 1H), 3.59 (d, 0.5H), 4.01 (t, 0.5H), 4.20 (s, 2H), 4.52 (t, 0.5H), 7.50-7.58 (m, 4H). HPLC-MS (Method Z1): m/z=301 (M+1); tᵣ=1.44 min (100% ELS).

### Step D

To a solution of the above benzyl amine (155 mg, 0.516 mmol) in DCM (3 mL) was added with stirring TEA (107 μL, 0.774 mmol) followed by acetyl chloride (41 μL, 0.568 mmol) and the mixture was stirred for 16 h. at ambient temperature. The mixture was washed with water (3×1 mL), dried (MgSO₄) and the solvent evaporated. The residue was purified using preparative HPLC (Method Z4): Amount isolated=47 mg; tᵣ=10.48 min (27%) of the title compound as an oil.

¹H NMR (400 MHz, MeOD) δ 0.95 (d, 3H), 1.02 (d, 3H), 1.12 (d, 3H), 1.2-1.67 (m, 5H), 1.82 (m, 1H), 2.17 (m, 3.5H), 2.93+3.02 (2×s, 3H, rotamers), 3.19 (m, 1H), 3.57 (d, 0.5H), 4.03 (m, 0.5H), 4.50 (m, 0.5H), 4.62-4.68 (m, 2H), 7.3-7.52 (m, 4H). HPLC-MS (Method Z1): m/z=343 (M+1); tᵣ=1.78 min (100% ELS).

### Example 1-2

### General Procedure (A)

### N-Methyl-N-[4-(1,3,3-trimethyl-6-aza-bicyclo[3.2.1]octane-6-carbonyl)-benzyl]-isobutyramide

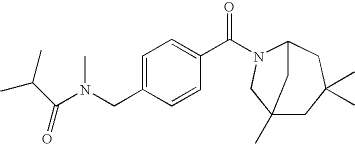

The title compound was prepared by a similar procedure as that described in Example 1, starting from (4-methylaminomethyl-phenyl)-(1,3,3-trimethyl-6-aza-bicyclo[3.2.1]oct-6-yl)-methanone and isobutyryl chloride.

HPLC-MS (Method Z1): m/z=371 (M+1); tᵣ=1.89 min (100% ELS).

### Example 1-3

### General Procedure (A)

### Cyclopentanecarboxylic acid methyl-[4-(1,3,3-trimethyl-6-aza-bicyclo[3.2.1]octane-6-carbonyl)-benzyl]-amide

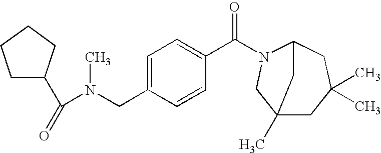

The title compound was prepared by a similar procedure as that described in Example 1, starting from (4-methylaminomethyl-phenyl)-(1,3,3-trimethyl-6-aza-bicyclo[3.2.1]oct-6-yl)-methanone and cyclopentanecarbonyl chloride.

HPLC-MS (Method Z1): m/z=397 (M+1); tᵣ=2.08 min (100% TIC).

### Example 1-4

### General Procedure (A)

### Cyclohexanecarboxylic acid methyl-[4-(1,3,3-trimethyl-6-aza-bicyclo[3.2.1]octane-6-carbonyl)-benzyl]-amide

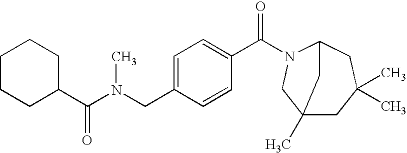

The title compound was prepared by a similar procedure as that described in Example 1, starting from (4-methylaminomethyl-phenyl)-(1,3,3-trimethyl-6-aza-bicyclo[3.2.1]oct-6-yl)-methanone and cyclohexanecarbonyl chloride.

HPLC-MS (Method Z1): m/z=411 (M+1); tᵣ=2.16 min (100% ELS).

### Example 1-5

### General Procedure (A)

### Piperidine-1-carboxylic acid methyl-[4-(1,3,3-trimethyl-6-aza-bicyclo[3.2.1]octane-6-carbonyl)-benzyl]-amide

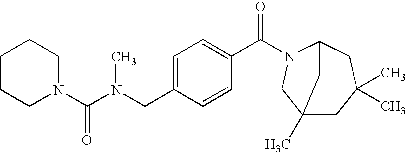

The title compound was prepared by a similar procedure as that described in Example 1, starting from (4-methylaminomethyl-phenyl)-(1,3,3-trimethyl-6-aza-bicyclo[3.2.1]oct-6-yl)-methanone and piperidine-1-carbonyl chloride.

HPLC-MS (Method Z1): m/z=412 (M+1); tᵣ=2.09 min.

### Example 1-6

### General Procedure (A)

### 1,3-Dimethyl-3-phenyl-1-[4-(1,3,3-trimethyl-6-aza-bicyclo[3.2.1]octane-6-carbonyl)-benzyl]-urea

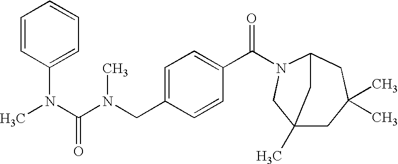

The title compound was prepared by a similar procedure as that described in Example 1, starting from (4-methylaminomethyl-phenyl)-(1,3,3-trimethyl-6-aza-bicyclo[3.2.1]oct-6-yl)-methanone and N-methyl-N-phenylcarbamoyl chloride.

HPLC-MS (Method Z1): m/z=435 (M+1); tᵣ=2.19 min.

### Example 1-7

### General Procedure (A)

### N-Methyl-N-[4-(1,3,3-trimethyl-6-aza-bicyclo[3.2.1]octane-6-carbonyl)-benzyl]-benzamide

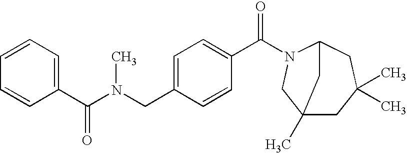

The title compound was prepared by a similar procedure as that described in Example 1, starting from (4-methylaminomethyl-phenyl)-(1,3,3-trimethyl-6-aza-bicyclo[3.2.1]oct-6-yl)-methanone and benzoyl chloride.

HPLC-MS (Method Z1): m/z=405 (M+1); tᵣ=1.98 min.

### Example 1-8

### General Procedure (A)

### 1-Acetyl-piperidine-4-carboxylic acid methyl-[4-(1,3,3-trimethyl-6-aza-bicyclo[3.2.1]octane-6-carbonyl)-benzyl]-amide

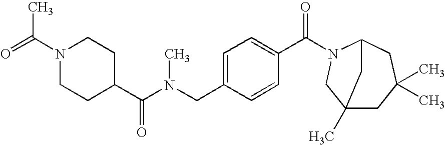

The title compound was prepared by a similar procedure as that described in Example 1, starting from (4-methylaminomethyl-phenyl)-(1,3,3-trimethyl-6-aza-bicyclo[3.2.1]oct-6-yl)-methanone and 1-acetyl-piperidine-4-carbonyl chloride.

HPLC-MS (Method Z1): m/z=454 (M+1); tᵣ=1.61 min.

### Example 1-9

### General Procedure (A)

### 1-Acetyl-piperidine-3-carboxylic acid methyl-[4-(1,3,3-trimethyl-6-aza-bicyclo[3.2.1]octane-6-carbonyl)-benzyl]-amide

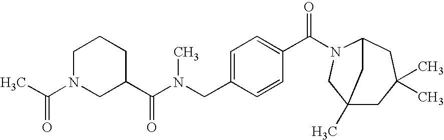

The title compound was prepared by a similar procedure as that described in Example 1, starting from (4-methylaminomethyl-phenyl)-(1,3,3-trimethyl-6-aza-bicyclo[3.2.1]oct-6-yl)-methanone and 1-acetyl-piperidine-3-carbonyl chloride.

HPLC-MS (Method Z1): m/z=454 (M+1); tᵣ=1.65 min.

### Example 1-10

### General Procedure (A)

### Cyclopentanecarboxylic acid ethyl-[4-(1,3,3-trimethyl-6-aza-bicyclo[3.2.1]octane-6-carbonyl)-benzyl]-amide

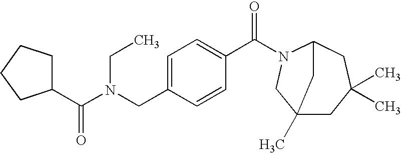

The title compound was prepared by a similar procedure as that described in Example 1, starting from (4-ethylaminomethyl-phenyl)-(1,3,3-trimethyl-6-aza-bicyclo[3.2.1]oct-6-yl)-methanone and cyclopentanecarbonyl chloride.

HPLC-MS (Method Z1): m/z=411 (M+1); tᵣ=2.17 min.

### Example 1-11

### General Procedure (A)

### Morpholine-4-carboxylic acid methyl-[4-(1,3,3-trimethyl-6-aza-bicyclo[3.2.1]octane-6-carbonyl)-benzyl]-amide

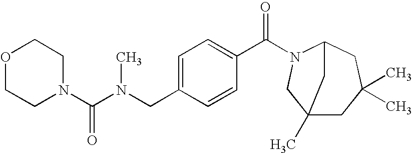

The title compound was prepared by a similar procedure as that described in Example 1, starting from (4-methylaminomethyl-phenyl)-(1,3,3-trimethyl-6-aza-bicyclo[3.2.1]oct-6-yl)-methanone and morpholine-4-carbonyl chloride.

HPLC-MS (Method Z1): m/z=414 (M+1); tᵣ=1.74 min.

### Example 1-12

### General Procedure (A)

### 2,2-N-Trimethyl-N-[4-(1,3,3-trimethyl-6-aza-bicyclo[3.2.1]octane-6-carbonyl)-benzyl]-propionamide

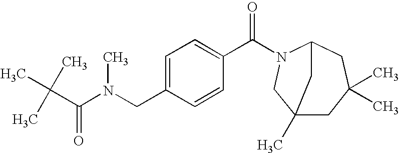

The title compound was prepared by a similar procedure as that described in Example 1, starting from (4-methylaminomethyl-phenyl)-(1,3,3-trimethyl-6-aza-bicyclo[3.2.1]oct-6-yl)-methanone and 2,2-dimethyl-propionyl chloride.

HPLC-MS (Method Z1): m/z=385 (M+1); tᵣ=2.04 min.

### Example 1-13

### General Procedure (A)

### Tetrahydro-furan-3-carboxylic acid methyl-[4-(1,3,3-trimethyl-6-aza-bicyclo[3.2.1]octane-6-carbonyl)-benzyl]-amide

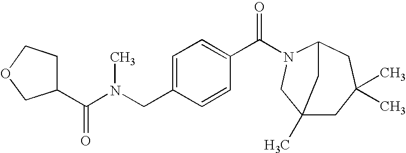

The title compound was prepared by a similar procedure as that described in Example 1, starting from (4-methylaminomethyl-phenyl)-(1,3,3-trimethyl-6-aza-bicyclo[3.2.1]oct-6-yl)-methanone and tetrahydro-furan-3-carbonyl chloride.

HPLC-MS (Method Z1): m/z=399 (M+1); tᵣ=1.68 min.

### Example 1-14

### General Procedure (A)

### N-Methyl-4-trifluoromethoxy-N-[4-(1,3,3-trimethyl-6-aza-bicyclo[3.2.1]octane-6-carbonyl)-benzyl]-benzamide

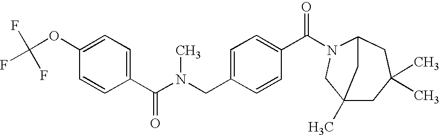

The title compound was prepared by a similar procedure as that described in Example 1, starting from (4-methylaminomethyl-phenyl)-(1,3,3-trimethyl-6-aza-bicyclo[3.2.1]oct-6-yl)-methanone and 4-trifluoromethoxy-benzoyl chloride.

HPLC-MS (Method Z1): m/z=489 (M+1); tᵣ=2.24 min.

### Example 1-15

### General Procedure (A)

### Thiophene-2-carboxylic acid methyl-[4-(1,3,3-trimethyl-6-aza-bicyclo[3.2.1]octane-6-carbonyl)-benzyl]-amide

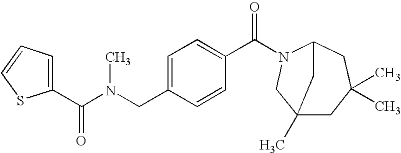

The title compound was prepared by a similar procedure as that described in Example 1, starting from (4-methylaminomethyl-phenyl)-(1,3,3-trimethyl-6-aza-bicyclo[3.2.1]oct-6-yl)-methanone and thiophene-2-carbonyl chloride.

HPLC-MS (Method Z1): m/z=411 (M+1); tᵣ=1.97 min.

### Example 1-16

### General Procedure (A)

### Furan-2-carboxylic acid methyl-[4-(1,3,3-trimethyl-6-aza-bicyclo[3.2.1]octane-6-carbonyl)-benzyl]-amide

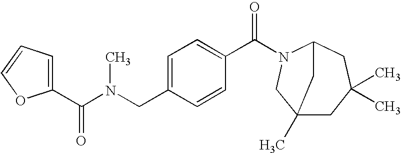

The title compound was prepared by a similar procedure as that described in Example 1, starting from (4-methylaminomethyl-phenyl)-(1,3,3-trimethyl-6-aza-bicyclo[3.2.1]oct-6-yl)-methanone and furane-2-carbonyl chloride.

HPLC-MS (Method Z1): m/z=395 (M+1); tᵣ=1.96 min.

### Example 1-17

### General Procedure (A)

### 3-Chloro-4-(propane-2-sulfonyl)-thiophene-2-carboxylic acid methyl-[4-(1,3,3-trimethyl-6-aza-bicyclo[3.2.1]octane-6-carbonyl)-benzyl]-amide

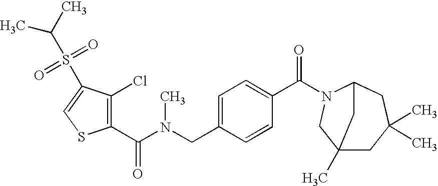

The title compound was prepared by a similar procedure as that described in Example 1, starting from (4-methylaminomethyl-phenyl)-(1,3,3-trimethyl-6-aza-bicyclo[3.2.1]oct-6-yl)-methanone and 3-chloro-4-(propane-2-sulfonyl)-thiophene-2-carbonyl chloride.

HPLC-MS (Method Z1): m/z=551 (M+1); tᵣ=2.0 min.

### Example 1-18

### General Procedure (A)

### 6-Chloro-N-methyl-N-[4-(1,3,3-trimethyl-6-aza-bicyclo[3.2.1]octane-6-carbonyl)-benzyl]-nicotinamide

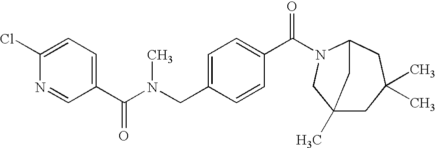

The title compound was prepared by a similar procedure as that described in Example 1, starting from (4-methylaminomethyl-phenyl)-(1,3,3-trimethyl-6-aza-bicyclo[3.2.1]oct-6-yl)-methanone and 6-chloro-nicotinoyl chloride.

HPLC-MS (Method Z1): m/z=440 (M+1); tᵣ=1.90 min.

### Example 1-19

### General Procedure (A)

### 5-Methyl-isoxazole-3-carboxylic acid methyl-[4-(1,3,3-trimethyl-6-aza-bicyclo[3.2.1]octane-6-carbonyl)-benzyl]-amide

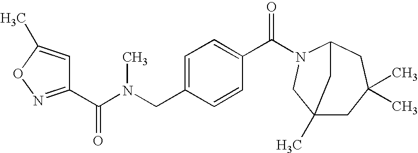

The title compound was prepared by a similar procedure as that described in Example 1, starting from (4-methylaminomethyl-phenyl)-(1,3,3-trimethyl-6-aza-bicyclo[3.2.1]oct-6-yl)-methanone and 5-methyl-isoxazole-3-carbonyl chloride.

HPLC-MS (Method Z1): m/z=410 (M+1); tᵣ=1.91 min.

### Example 1-20

### General Procedure (A)

### 3,3,N-Trimethyl-N-[4-(1,3,3-trimethyl-6-aza-bicyclo[3.2.1]octane-6-carbonyl)-benzyl]-butyramide

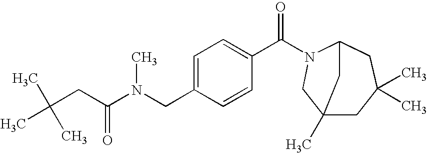

The title compound was prepared by a similar procedure as that described in Example 1, starting from (4-methylaminomethyl-phenyl)-(1,3,3-trimethyl-6-aza-bicyclo[3.2.1]oct-6-yl)-methanone and 3,3-dimethyl-butyryl chloride.

HPLC-MS (Method Z1): m/z=399 (M+1); tᵣ=2.12 min.

### Example 1-21

### General Procedure (A)

### 3-Cyano-N-methyl-N-[4-(1,3,3-trimethyl-6-aza-bicyclo[3.2.1]octane-6-carbonyl)-benzyl]-benzamide

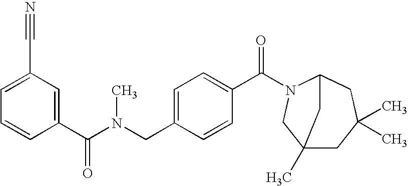

The title compound was prepared by a similar procedure as that described in Example 1, starting from (4-methylaminomethyl-phenyl)-(1,3,3-trimethyl-6-aza-bicyclo[3.2.1]oct-6-yl)-methanone and 3-cyano-benzoyl chloride.

HPLC-MS (Method Z1): m/z=430 (M+1); tᵣ=1.92 min.

### Example 1-22

### General Procedure (A)

### N-Methyl-2-phenoxy-N-[4-(1,3,3-trimethyl-6-aza-bicyclo[3.2.1]octane-6-carbonyl)-benzyl]-acetamide

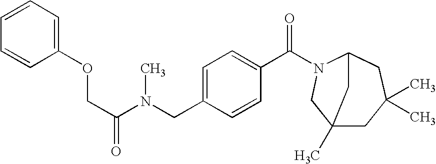

The title compound was prepared by a similar procedure as that described in Example 1, starting from (4-methylaminomethyl-phenyl)-(1,3,3-trimethyl-6-aza-bicyclo[3.2.1]oct-6-yl)-methanone and phenoxy-acetyl chloride.

HPLC-MS (Method Z1): m/z=435 (M+1); tᵣ=2.02 min.

### Example 1-23

### General Procedure (A)

### N-Methyl-N-[4-(1,3,3-trimethyl-6-aza-bicyclo[3.2.1]octane-6-carbonyl)-benzyl]-malonamic Acid Methyl Ester

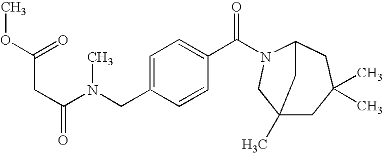

The title compound was prepared by a similar procedure as that described in Example 1, starting from (4-methylaminomethyl-phenyl)-(1,3,3-trimethyl-6-aza-bicyclo[3.2.1]oct-6-yl)-methanone and chlorocarbonyl-acetic acid methyl ester.

HPLC-MS (Method Z1): m/z=401 (M+1); tᵣ=1.69 min.

### Example 1-24

### General Procedure (A)

### 3-Methyl-but-2-enoic acid methyl-[4-(1,3,3-trimethyl-6-aza-bicyclo[3.2.1]octane-6-carbonyl)-benzyl]-amide

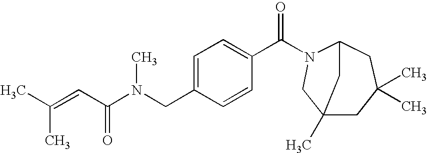

The title compound was prepared by a similar procedure as that described in Example 1, starting from (4-methylaminomethyl-phenyl)-(1,3,3-trimethyl-6-aza-bicyclo[3.2.1]oct-6-yl)-methanone and 3-methyl-but-2-enoyl chloride.

HPLC-MS (Method Z1): m/z=383 (M+1); tᵣ=1.92 min.

### Example 1-25

### General Procedure (A)

### N-Methyl-2-phenyl-N-[4-(1,3,3-trimethyl-6-aza-bicyclo[3.2.1]octane-6-carbonyl)-benzyl]-acetamide

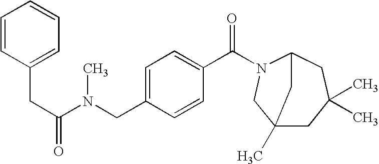

The title compound was prepared by a similar procedure as that described in Example 1, starting from (4-methylaminomethyl-phenyl)-(1,3,3-trimethyl-6-aza-bicyclo[3.2.1]oct-6-yl)-methanone and phenyl-acetyl chloride.

HPLC-MS (Method Z1): m/z=419 (M+1); tᵣ=2.03 min.

### Example 1-26

### General Procedure (A)

### 1-Trifluoromethyl-cyclobutanecarboxylic Acid Methyl-[4-(1,3,3-trimethyl-6-aza-bicyclo[3.2.1]octane-6-carbonyl)-benzyl]-amide

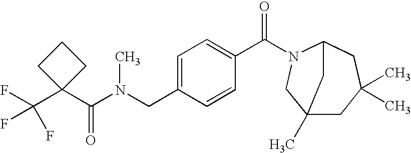

To a solution of 1-trifluoromethyl-cyclobutanecarboxylic acid (33.6 mg, 0.2 mmol) in THF (5 mL) was added with stirring HOBt (27 mg, 0.2 mmol) followed by EDAC (38 mg, 0.2 mmol) and the mixture was stirred for 30 min. at ambient temperature. To the resulting mixture was added (4-methylaminomethyl-phenyl)-(1,3,3-trimethyl-6-aza-bicyclo[3.2.1]oct-6-yl)-methanone (50 mg, 0.17 mmol, Example 1) and DIPEA (35 μL, 0.2 mmol). The reaction mixture was stirred for 16 h. at ambient temperature. The solvent was evaporated and the residue purified using preparative HPLC (Method Z4): Amount isolated=40 mg (53%) of the title compound as an oil.

¹H NMR (400 MHz, CDCl₃) δ 0.94 (d, 3H), 1.04 (d, 3H), 1.13 (d, 3H), 1.17-1.60 (m, 4.5H), 1.75-1.90 (m, 2H), 2.10 (m, 1H), 2.24 (m, 0.5H), 2.55 (m, 2H), 2.73 (m, 2H), 2.83+2.86 (2×s, 3H, rotamers), 3.15 (d, 0.5H), 3.26 (t, 1H), 3.60 (d, 0.5H), 3.98 (bs, 0.5H), 4.46 (bs, 0.5H), 4.63 (m, 2H), 7.29 (m, 2H), 7.40 (t, 2H).

HPLC-MS (Method Z1): m/z=451 (M+1); tᵣ=2.18 min.

### Example 1-27

### General Procedure (A)

### 3,5-Dimethoxy-N-methyl-N-[4-(1,3,3-trimethyl-6-aza-bicyclo[3.2.1]octane-6-carbonyl)-benzyl]-benzamide

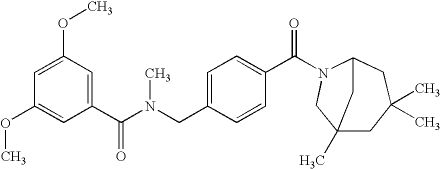

The title compound was prepared by a similar procedure as that described in Example 1, starting from (4-methylaminomethyl-phenyl)-(1,3,3-trimethyl-6-aza-bicyclo[3.2.1]oct-6-yl)-methanone and 3,5-dimethoxy-benzoyl chloride.

HPLC-MS (Method Z1): m/z=465 (M+1); tᵣ=2.05 min.

### Example 1-28

### General Procedure (A)

### 4-Methanesulfonyl-N-methyl-N-[4-(1,3,3-trimethyl-6-aza-bicyclo[3.2.1]octane-6-carbonyl)-benzyl]-benzamide

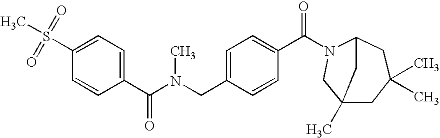

The title compound was prepared by a similar procedure as that described in Example 23, starting from (4-methylaminomethyl-phenyl)-(1,3,3-trimethyl-6-aza-bicyclo[3.2.1]oct-6-yl)-methanone and 4-methanesulfonyl-benzoic acid.

HPLC-MS (Method Z1): m/z=483 (M+1); tᵣ=1.78 min.

### Example 1-29

### General Procedure (A)

### N-Methyl-3-trifluoromethoxy-N-[4-(1,3,3-trimethyl-6-aza-bicyclo[3.2.1]octane-6-carbonyl)-benzyl]-benzamide

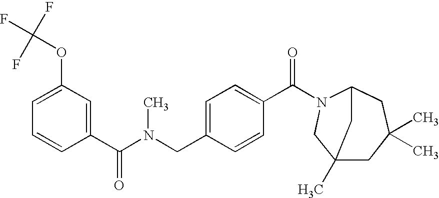

The title compound was prepared by a similar procedure as that described in Example 23, starting from (4-methylaminomethyl-phenyl)-(1,3,3-trimethyl-6-aza-bicyclo[3.2.1]oct-6-yl)-methanone and 3-trifluoromethoxy-benzoic acid.

HPLC-MS (Method Z1): m/z=489 (M+1); tᵣ=2.23 min.

### Example 1-30

### General Procedure (A)

### 2,2-Difluoro-1,3-benzodioxole-4-carboxylic acid methyl-[4-(1,3,3-trimethyl-6-aza-bicyclo[3.2.1]octane-6-carbonyl)-benzyl]-amide

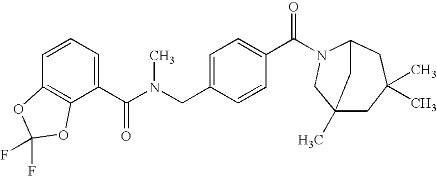

The title compound was prepared by a similar procedure as that described in Example 23, starting from (4-methylaminomethyl-phenyl)-(1,3,3-trimethyl-6-aza-bicyclo[3.2.1]oct-6-yl)-methanone and 2,2-difluoro-benzo[1,3]dioxole-4-carboxylic acid.

HPLC-MS (Method Z1): m/z=485 (M+1); tᵣ=2.21 min.

### Example 1-31

### General Procedure (A)

### N-Methyl-6-morpholin-4-yl-N-[4-(1,3,3-trimethyl-6-aza-bicyclo[3.2.1]octane-6-carbonyl)-benzyl]-nicotinamide

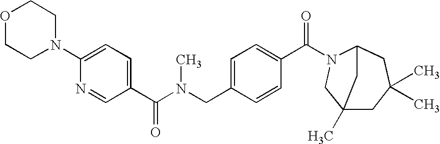

The title compound was prepared by a similar procedure as that described in Example 23, starting from (4-methylaminomethyl-phenyl)-(1,3,3-trimethyl-6-aza-bicyclo[3.2.1]oct-6-yl)-methanone and 6-morpholin-4-yl-nicotinic acid.

HPLC-MS (Method Z1): m/z=491 (M+1); tᵣ=1.52 min.

### Example 1-32

### General Procedure (A)

### N-Methyl-4-(2,2,2-trifluoro-acetyl)-N-[4-(1,3,3-trimethyl-6-aza-bicyclo[3.2.1]octane-6-carbonyl)-benzyl]-benzamide

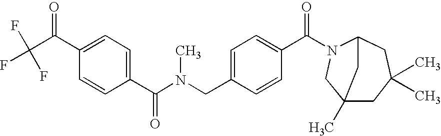

The title compound was prepared by a similar procedure as that described in Example 23, starting from (4-methylaminomethyl-phenyl)-(1,3,3-trimethyl-6-aza-bicyclo[3.2.1]oct-6-yl)-methanone and 4-(2,2,2-trifluoro-acetyl)-benzoic acid.

HPLC-MS (Method Z1): m/z=519 (M+18); tᵣ=1.84 min.

### Example 1-33

### General Procedure (A)

### 3-Acetyl-N-methyl-N-[4-(1,3,3-trimethyl-6-aza-bicyclo[3.2.1]octane-6-carbonyl)-benzyl]-benzamide

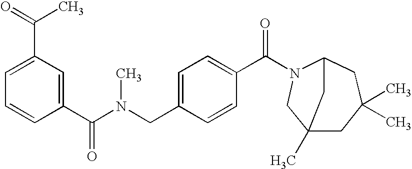

The title compound was prepared by a similar procedure as that described in Example 23, starting from (4-methylaminomethyl-phenyl)-(1,3,3-trimethyl-6-aza-bicyclo[3.2.1]oct-6-yl)-methanone and 3-acetyl-benzoic acid.

HPLC-MS (Method Z1): m/z=447 (M+1); tᵣ=1.90 min.

### Example 1-34

### General Procedure (A)

### N-Methyl-N-[4-(1,3,3-trimethyl-6-aza-bicyclo[3.2.1]octane-6-carbonyl)-benzyl]-isophthalamic Acid

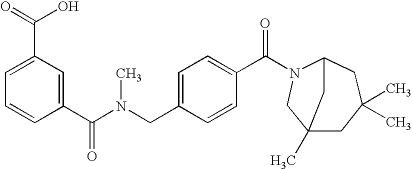

To a solution of isophthalic acid monomethyl ester (72 mg, 0.4 mmol) in THF (10 mL) was added with stirring HOBt (54 mg, 0.4 mmol) followed by EDAC (77 mg, 0.4 mmol) and the mixture was stirred for 30 min. at ambient temperature. To the resulting mixture was added (4-methylaminomethyl-phenyl)-(1,3,3-trimethyl-6-aza-bicyclo[3.2.1]oct-6-yl)-methanone (100 mg, 0.33 mmol, Example 1) and DIPEA (70 μL, 0.4 mmol). The reaction mixture was stirred for 16 h. at ambient temperature. The solvent was evaporated and the residue purified using preparative HPLC (Method Z4): Amount isolated=100 mg (65%) of N-Methyl-N-[4-(1,3,3-trimethyl-6-aza-bicyclo[3.2.1]octane-6-carbonyl)-benzyl]-isophthalamic acid methyl ester as an oil.

To a solution of the ester (100 mg) in EtOH (5 mL) was added water (2 mL) and 1N NaOH (0.5 mL). The mixture was stirred at ambient temperature for 6 h and the volatiles evaporated. The residue was dissolved in water (5 mL) and washed with Et₂O (2×10 mL) and pH adjusted to 1 by 1N HCl. The aqueous phase was extracted with EtOAc (3×10 mL), the combined organic phases dried (MgSO₄) and evaporated which afforded 73 mg (49%) of the title compound as a solid.

¹H NMR (400 MHz, CDCl₃) δ 0.95 (d, 3H), 1.05 (s, 3H), 1.14 (d, 3H), 1.33-1.49 (m, 3.5H), 1.58 (m, 1H), 1.79 (m, 1H), 2.27 (m, 0.5H), 2.88+3.08 (2×s, 3H, rotamers), 3.28 (m, 1.5H), 3.63 (d, 0.5H), 4.02 (m, 0.5H), 4.53-4.79 (m, 2.5H), 7.21-7.70 (m, 6H), 8.15 (m, 2H). HPLC-MS (Method Z1): m/z=449 (M+1); tᵣ=1.76 min.

### Example 1-35

### General Procedure (A)

### 2,3-Dihydro-benzofuran-7-carboxylic Acid Methyl-[4-(1,3,3-trimethyl-6-aza-bicyclo[3.2.1]octane-6-carbonyl)-benzyl]-amide

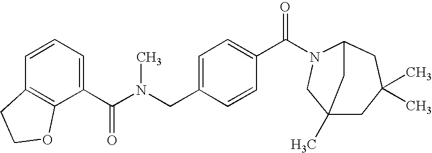

The title compound was prepared by a similar procedure as that described in Example 23, starting from (4-methylaminomethyl-phenyl)-(1,3,3-trimethyl-6-aza-bicyclo[3.2.1]oct-6-yl)-methanone and 2,3-dihydro-benzofuran-7-carboxylic acid.

HPLC-MS (Method Z1): m/z=447 (M+1); tᵣ=2.02 min.

### Example 1-36

### General Procedure (A)

### N-[4-(1,3,3-Trimethyl-6-aza-bicyclo[3.2.1]octane-6-carbonyl)-benzyl]-benzamide

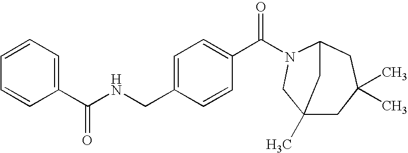

To a solution of [4-(1,3,3-trimethyl-6-aza-bicyclo[3.2.1]octane-6-carbonyl)-benzyl]-carbamic acid tert-butyl ester (11 g, 28.5 mmol, Example 1) in DCM (40 mL) was added with stirring TFA (20 mL) and the mixture was stirred for 16 h. at ambient temperature. The solvent was evaporated and to the residue was added water (50 mL) and the pH adjusted to 11. The mixture was extracted with DCM (3×20 mL) and the combined organic phases were washed with brine (20 mL), dried (MgSO₄) and the solvent evaporated affording 7.7 g (94%) of (4-aminomethyl-phenyl)-(1,3,3-trimethyl-6-aza-bicyclo[3.2.1]oct-6-yl)-methanone as an oil.

¹H NMR (400 MHz, CDCl₃) δ0.97 (d, 3H), 1.02 (d, 3H), 1.12 (d, 3H), 1.15-1.44 (m, 4.5H), 1.56 (t, 1H), 1.75 (m, 1H), 2.23 (m, 0.5H), 3.01 (bs, 2H, NH₂), 3.15 (d, 0.5H), 3.57 (d, 0.5H), 3.89 (d, 2H), 3.97 (t, 0.5H), 4.58 (t, 0.5H), 7.38 (m, 4H). HPLC-MS (Method Z1): m/z=287 (M+1); tᵣ=1.2 min (100% ELS).

To a solution of the above benzyl amine (100 mg, 0.35 mmol) in DCM (5 mL) was added with stirring TEA (150 μL, 1.05 mmol) followed by benzoyl chloride (60 μL, 0.52 mmol) and the mixture was stirred for 16 h. at ambient temperature. The mixture was washed with water (3×1 mL), dried (MgSO₄) and the solvent evaporated. The residue was purified using preparative HPLC (Method Z4): Amount isolated=95 mg (70%) of the title compound as an oil.

¹H NMR (400 MHz, CDCl₃) δ 0.92 (d, 3H), 0.95 (d, 3H), 1.01 (d, 3H), 1.13-1.58 (m, 4.5H), 1.74 (m, 1H), 2.21 (m, 0.5H), 3.12 (d, 0.5H), 3.22 (t, 1H), 3.56 (d, 0.5H), 3.94 (t, 0.5H), 4.58 (m, 2.5H), 7.22-7.32 (m, 4H), 7.40 (t, 2H), 7.49 (m, 2H), 7.87 (d, 2H). HPLC-MS (Method Z1): m/z=391 (M+1); tᵣ=1.91 min (100% ELS).

### Example 1

### General Procedure (A)

### [4-(1-Amino-cyclopropyl)-phenyl]-(1,3,3-trimethyl-6-aza-bicyclo[3.2.1]oct-6-yl)-methanone

To a solution of NaH (1.95 g, 0.049 mol, 60% in mineral oil, washed twice with dry THF) in dry DMF (50 mL) was added dropwise a solution of [4-(1,3,3-Trimethyl-6-aza-bicyclo-[3.2.1]octane-6-carbonyl)-phenyl]-acetonitrile (7.0 g, 0.024 mol) in dry DMF (180 ml) at 0° C. To the resulting mixture was added dropwise a solution of 1,2-dibromoethan (8.14 mL, 0.094 mol) in dry DMF (25 mL) and the mixture was stirred for 16 hrs at room temperature at which time it was quinced by addition of crushed ice. The aqueous phase was extracted with AcOEt (3×250 mL) and the combined organic phases were washed with water (2×100 mL), brine (1×00 mL), dried (MgSO₄) and filtered followed by evaporation of the volatiles. This afforded crud 5.28 g (69%) of 1-[4-(1,3,3-trimethyl-6-aza-bicyclo[3.2.1]octane-6-carbonyl)-phenyl]-cyclopropanecarbonitrile as an oil.

HPLC-MS (Method Z1): m/z=323 (M+1); tᵣ=2.02 min (100% ELS).

The above nitrile (5.25 g, 16.28 mmol) was added to a mixture of conc. HCl (120 mL) and AcOH (30 mL) and stirred at 80° C. for 18 hrs. The reaction mixture is diluted with ice-water (300 mL) and the pH adjusted to 3 by addition of 4 N NaOH. The oily precipitate was extracted with diethyl ether (3×200 mL) and the combined organic phases were washed with water (2×100 mL), brine (1×80 mL), dried (MgSO₄), filtered and the volatiles evaporated in vacuo affording 4.7 g (85%) of 1-[4-(1,3,3-trimethyl-6-aza-bicyclo[3.2.1]octane-6-carbonyl)-phenyl]-cyclopropane-carboxylic acid as a solid.

HPLC-MS (Method Z1): m/z=342 (M+1); tᵣ=1.83 min (100% ELS).

To a solution of the above carboxylic acid (3.00 g, 8.79 mmol) in DCM (600 mL) was added H₂SO₄ (7.2 mL) followed by NaN₃ (1.38 g, 21.23 mmol). The mixture was stirred at 45° C. for 16 hrs cooled to room temperature and quenched by addition of ice-water (300 mL). The pH was adjusted to 11 by addition of 4N NaOH and the mixture was extracted with DCM (2×150 mL). The volatiles were evaporated and the residue was subjected to preparative HPLC purification affording 2.7 g (98%) of the [4-(1-amino-cyclopropyl)-phenyl]-(1,3,3-trimethyl-6-azabicyclo[3.2.1]oct-6-yl)-methanoneas an oil.

HPLC-MS (Method Z1): m/z=313 (M+1); tᵣ=1.30 min (100% ELS). ¹H NMR (400 MHz, CDCl₃) δ 0.93 (d, 3H), 0.99-1.03 (m, 5H), 1.13 (m, 5H), 1.17-1.5 (m, 3.5H), 1.58 (d, 1H), 1.75 (m, 1H), 2.17 (bs, 2H, NH₂), 2.24 (dd, 0.5H), 3.17 (d, 0.5H), 3.28 (t, 1H), 3.58 (d, 0.5H), 3.99 (t, 0.5H), 4.60 (m, 0.5H), 7.29-7.33 (m, 2H), 7.37-7.42 (m, 2H).

To a solution of [4-(1-amino-cyclopropyl)-phenyl]-(1,3,3-trimethyl-6-aza-bicyclo[3.2.1]oct-6-yl)-methanone (150 mg, 0.48 mmol) in DCM (4 mL) was added with stirring TEA (100 μL, 0.72 mmol) followed by benzoyl chloride (61 μL, 0.53 mmol) and the mixture was stirred for 16 h. at ambient temperature. The mixture was washed with water (3×1 mL), dried (MgSO₄) and the solvent evaporated. The residue was purified using preparative HPLC (Method Z4): Amount isolated=116 mg (58%) of the title compound as an oil.

¹H NMR (400 MHz, CDCl₃) δ 0.92 (d, 3H), 1.00 (s, 3H), 1.11 (s, 3H), 1.13-1.44 (m, 5.5H), 1.57 (m, 1H), 1.71 (m, 2H), 2.20 (m, 0.5H), 3.11 (d, 0.5H), 3.25 (m, 1H), 3.55 (d, 1H), 3.96 (m, 0.5H), 4.58 (m, 0.5H), 7.09 (t, 2H), 7.28 (dd, 2H), 7.44 (t, 2H), 7.51 (t, 1H), 7.63 (d, 1H), 7.90)d, 2H). HPLC-MS (Method Z1): m/z=417 (M+1); tᵣ=2.0 min (100% ELS).

The following compounds were made as outlined in general method A above.

| | | | | LC/ |
|---|---|---|---|---|
| Ex | Structure | MW | IUPAC Name | MS |
| -38 | 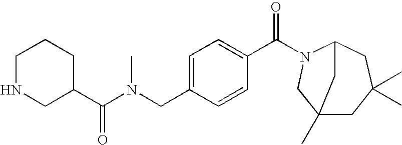 | 411.59 | Piperidine-3-carboxylic acid methyl-[4-(1,3,3-trimethyl-6- aza-bicyclo[3.2.1]octane-6- carbonyl)-benzyl]-amide | 412 |
| 1-39 | 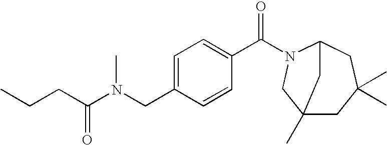 | 370.54 | N-Methyl-N-[4-(1,3,3-tri- methyl-6-aza-bicyclo[3.2.1]- octane-6-carbonyl)-benzyl]- butyramide | 371 |
| 1-40 | 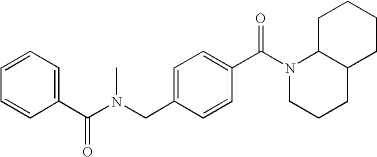 | 390.53 | N-Methyl-N-[4-(octahydro- quinoline-1-carbonyl)- benzyl]-benzamide | 391 |
| 1-41 | 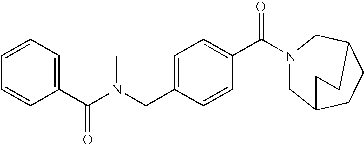 | 376.50 | N-[4-(3-Aza-bicyclo[3.2.2]- nonane-3-carbonyl)-benzyl]- N-methyl-benzamide | 377 |
| 1-42 | 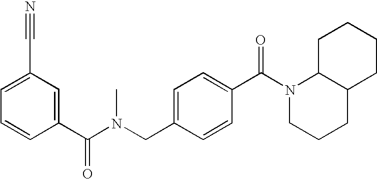 | 415.54 | 3-Cyano-N-methyl-N-[4- (octahydro-quinoline-1-car- bonyl)-benzyl]-benzamide | 416 |
| 1-43 | 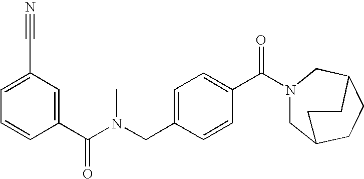 | 401.51 | N-[4-(3-Azabicyclo[3.2.2]- nonane-3-carbonyl)-benzyl]- 3-cyano-N-methyl- benzamide | 402 |
| 1-44 | 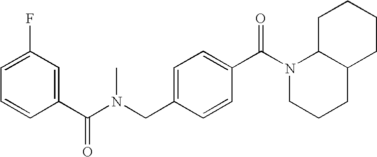 | 408.52 | 3-Fluoro-N-methyl-N-[4- (octahydro-quinoline-1-car- bonyl)-benzyl]-benzamide | 409 |
| 1-45 | 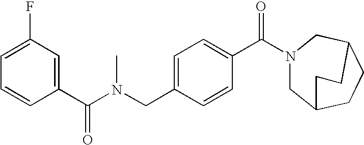 | 394.49 | N-[4-(3-Aza-bicyclo[3.2.2]- nonane-3-carbonyl)-benzyl]- 3-fluoro-N-methyl-benz- amide | 395 |
| 1-46 | 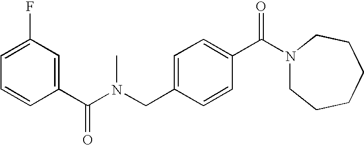 | 368.46 | N-[4-(Azepane-1-carbonyl)- benzyl]-3-fluoro-N-methyl- benzamide | 369 |
| 1-47 | 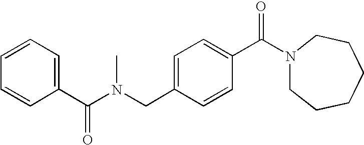 | 350.46 | N-[4-(Azepane-1-carbonyl)- benzyl]-N-methyl-benz- amide | 351 |
| 1-48 | 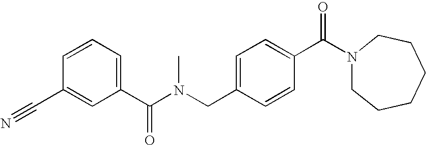 | 375.47 | N-[4-(Azepane-1-carbonyl)- benzyl]-3-cyano-N-methyl- benzamide | 376 |
| 1-49 | 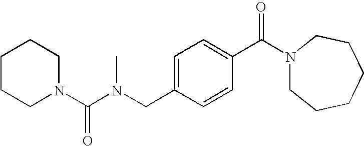 | 357.50 | Piperidine-1-carboxylic acid [4-(azepane-1-carbonyl)- benzyl]-methyl-amide | 358 |
| 1-50 | 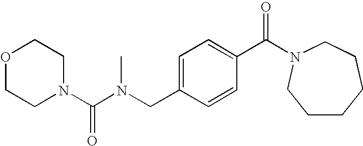 | 359.47 | Morpholine-4-carboxylic acid [4-(azepane-1-carbo- nyl)-benzyl]-methyl-amide | 360 |
| 1-51 | 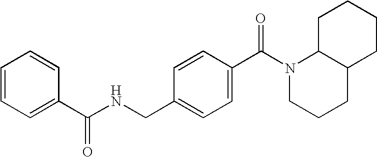 | 376.50 | N-[4-(Octahydro-quinoline- 1-carbonyl)-benzyl]-benz- amide | 377 |
| 1-52 | 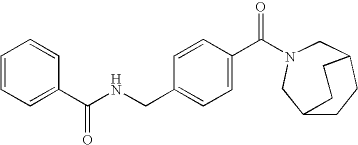 | 362.48 | N-[4-(3-Azabicyclo[3.2.2]- nonane-3-carbonyl)-benzyl]- benzamide | 363 |
| 1-53 | 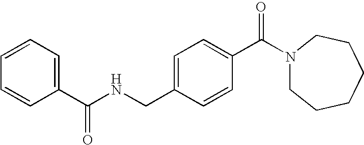 | 336.44 | N-[4-(Azepane-1-carbonyl)- benzyl]-benzamide | 337 |
| 1-54 | 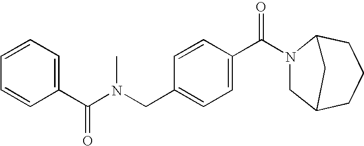 | 362.48 | N-[4-(6-Aza-bicyclo[3.2.1]- octane-6-carbonyl)-benzyl]- N-methyl-benzamide | 363 |
| 1-55 | 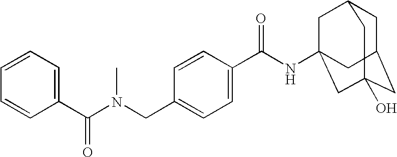 | 418.54 | 4-[(Benzoyl-methyl-amino)- methyl]-N-(3-hydroxy- adamantan-1-yl)-benzamide | 419 |
| 1-56 | 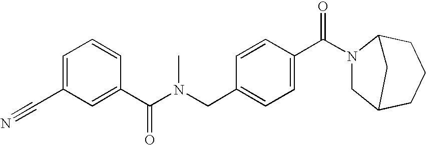 | 387.49 | N-[4-(6-Aza-bicyclo[3.2.1]- octane-6-carbonyl)-benzyl]- 3-cyano-N-methyl-benz- amide | 388 |
| 1-57 | 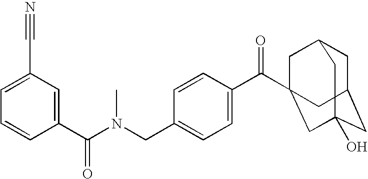 | 443.55 | 4-[(3-Cyano-benzoyl- methyl-amino)-methyl]-N-(3- hydroxy-adamantan-1-yl)- benzamide | 444 |
| 1-58 | 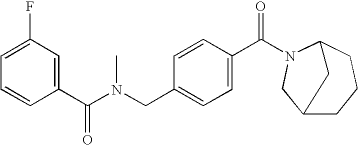 | 380.47 | N-[4-(6-Aza-bicyclo[3.2.1]- octane-6-carbonyl)-benzyl]- 3-fluoro-N-methyl- benzamide | 381 |
| 1-59 | 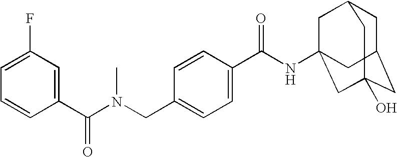 | 436.53 | 4-[(3-Fluoro-benzoyl- methyl-amino)-methyl]-N-(3- hydroxy-adamantan-1-yl)- benzamide | 437 |
| 1-60 | 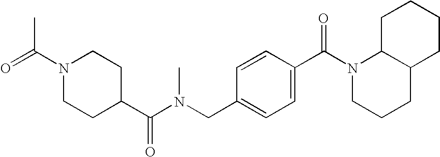 | 439.6 | 1-Acetyl-piperidine-4-car- boxylic acid methyl-[4-(octa- hydro-quinoline-1-carbonyl)- benzyl]-amide | 440 |
| 1-61 | 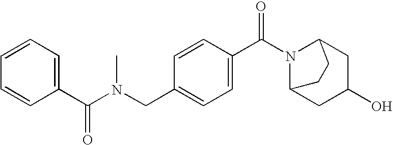 | 378.48 | N-[4-(3-Hydroxy-8-aza- bicyclo[3.2.1]octane-8-car- bonyl)-benzyl]-N-methyl- benzamide | 379 |
| 1-62 | 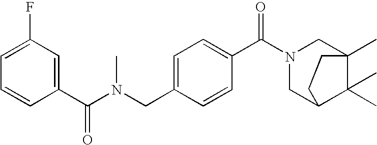 | 422.555 | 3-Fluoro-N-methyl-N-[4- (1,8,8-trimethyl-3-aza-bi- cyclo[3.2.1]octane-3-car- bonyl)-benzyl]-benzamide | 423 |
| 1-63 | 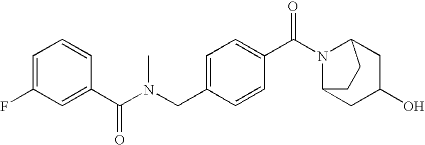 | 396.47 | 3-Fluoro-N-[4-(3-hydroxy-8- aza-bicyclo[3.2.1]octane-8- carbonyl)-benzyl]-N-methyl- benzamide | 397 |
| 1-64 | 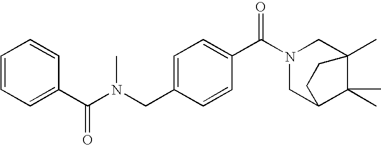 | 404.56 | N-Methyl-N-[4-(1,8,8-tri- methyl-3-aza-bicyclo[3.2.1]- octane-3-carbonyl)-benzyl]- benzamide | 405 |
| 1-65 | 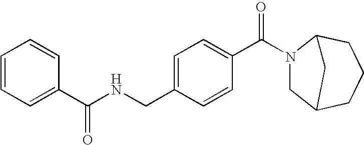 | 348.45 | N-[4-(6-Aza-bicyclo[3.2.1]- octane-6-carbonyl)-benzyl]- benzamide | 349 |
| 1-66 | 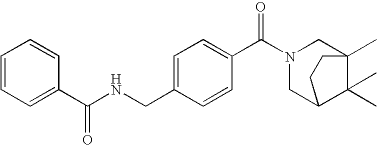 | 390.53 | N-[4-(1,8,8-trimethyl-3-aza- bicyclo[3.2.1]octane-3- carbonyl)-benzyl]- benzamide | 391 |
| 1-67 | 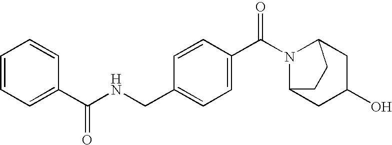 | 364.45 | N-[4-(3-Hydroxy-8-aza- bicyclo[3.2.1]octane-8- carbonyl)-benzyl]- benzamide | 365 |
| 1-68 | 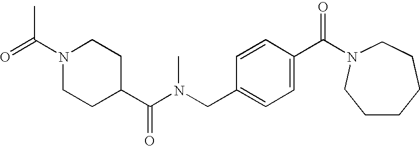 | 399.54 | 1-Acetyl-piperidine-4-carb- oxylic acid [4-(azepane-1- carbonyl)-benzyl]-methyl- amide | 400 |
| 1-69 | 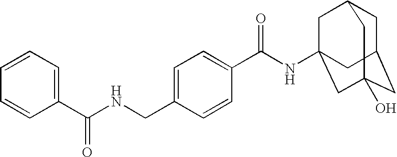 | 404.51 | 4-(Benzoylamino-methyl)-N- (3-hydroxy-adamantan-1- yl)-benzamide | 405 |
| 1-70 | 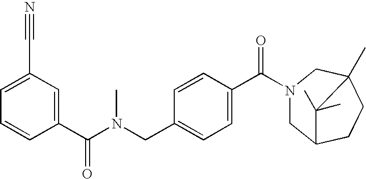 | 429.57 | 3-Cyano-N-methyl-N-[4- (1,8,8-trimethyl-3-aza-bi- cyclo[3.2.1]octane-3-car- bonyl)-benzyl]-benzamide | 430 |
| 1-71 | 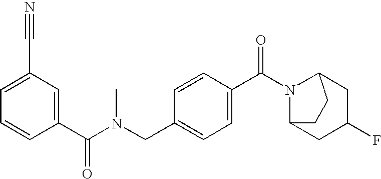 | 405.48 | 3-Cyano-N-[4-(3-fluoro-8- aza-bicyclo[3.2.1]octane-8- carbonyl)-benzyl]-N-methyl- benzamide | 406 |
| 1-72 | 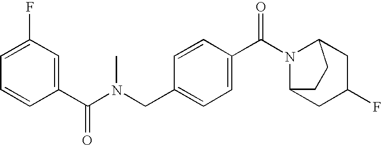 | 398.46 | 3-Fluoro-N-[4-(3-fluoro-8- aza-bicyclo[3.2.1]octane-8- carbonyl)-benzyl]-N-methyl- benzamide | 399 |
| 1-73 | 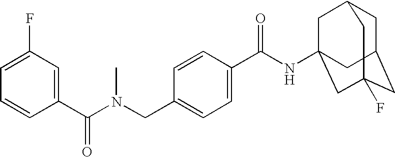 | 438.52 | 4-(3-Fluoro-benzoylamino- methyl)-N-methyl-N-(3- fluoro-adamantan-1-yl)- benzamide | 439 |
| 1-74 | 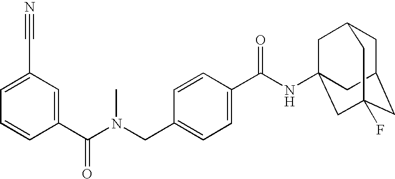 | 445.54 | 4-(3-Cyano-benzoylamino- methyl)-N-methyl-N-(3- fluoro-adamantan-1-yl)- benzamide | 446 |
| 1-75 | 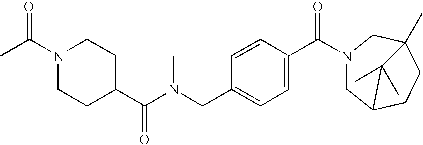 | 453.63 | 1-Acetyl-piperidine-4- carboxylic acid methyl-[4- (1,8,8-trimethyl-3-aza- bicyclo[3.2.1]octane-3- carbonyl)-benzyl]-amide | 454 |
| 1-76 | 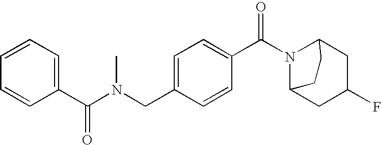 | 380.47 | N-[4-(3-Fluoro-8-aza- bicyclo[3.2.1]octane-8- carbonyl)-benzyl]-N-methyl- benzamide | 381 |
| 1-77 | 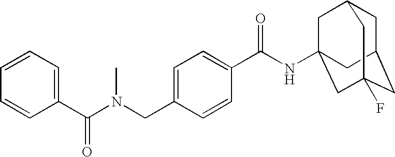 | 420.53 | 4-[(Benzoyl-methyl-amino)- methyl]-N-(3-fluoro-ada- mantan-1-yl)-benzamide | 421 |
| 1-78 | 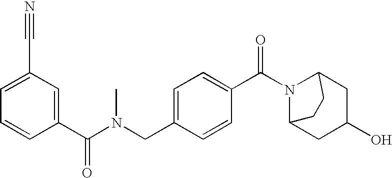 | 403.49 | 3-Cyano-N-[4-(3-hydroxy-8- aza-bicyclo[3.2.1]octane-8- carbonyl)-benzyl]-N-methyl- benzamide | 404 |
| 1-79 | 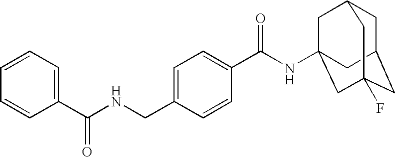 | 406.50 | 4-(Benzoylamino-methyl)-N- (3-fluoro-adamantan-1-yl)- benzamide | 407 |
| 1-80 | 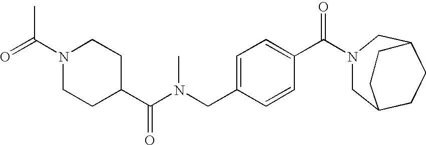 | 425.58 | 1-Acetyl-piperidine-4- carboxylic acid [4-(3-aza- bicyclo[3.2.2]nonane-3- carbonyl)-benzyl]-methyl- amide | 426 |
| 1-81 | 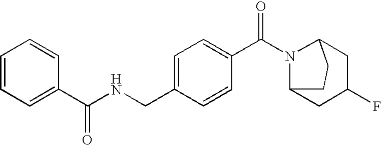 | 366.44 | N-[4-(3-Fluoro-8-aza-bi- cyclo[3.2.1]octane-8-carbo- nyl)-benzyl]-benzamide | 367 |
| 1-82 | 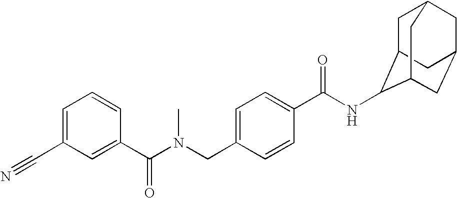 | 427.55 | 4-(3-Cyano-benzoylamino- methyl)-N-(adamantan-2- yl)-benzamide | 428 |
| 1-83 | 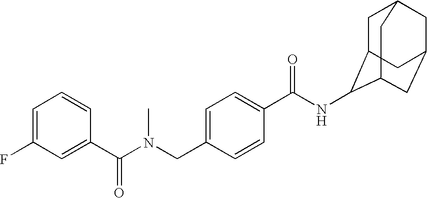 | 420.53 | 4-(3-Fluoro-benzoylamino- methyl)-N-(adamantan-2- yl)-benzamide | 421 |
| 1-84 | 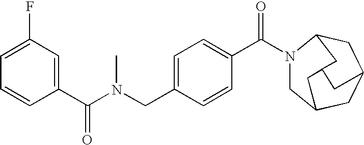 | 420.53 | N-[4-(4-Azatricyclo- [4.3.1.1*3,8*]undecane-4- carbonyl)-benzyl]-3-fluoro- N-methyl-benzamide | 421 |
| 1-85 | 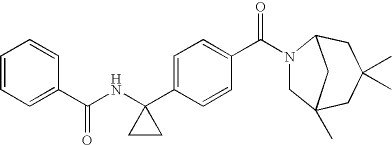 | 416.57 | N-{1-[4-(1,3,3-Trimethyl-6- aza-bicyclo[3.2.1]octane-6- carbonyl)-phenyl]-cyclo- propyl}-benzamide | 417 |
| 1-86 | 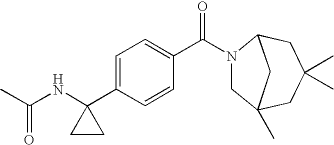 | 354.50 | N-{1-[4-(1,3,3-Trimethyl-6- aza-bicyclo[3.2.1]octane-6- carbonyl)-phenyl]-cyclo- propyl}-acetamide | 355 |
| 1-87 | 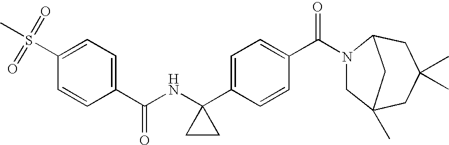 | 494.66 | 4-Methanesulfonyl-N-{1-[4- (1,3,3-trimethyl-6-aza- bicyclo[3.2.1]octane-6- carbonyl)-phenyl]-cyclo- propyl}-benzamide | 495 |
| 1-88 | 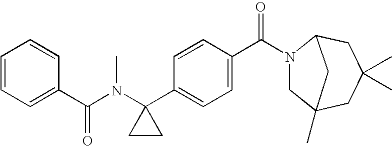 | 430.60 | N-Methyl-N-{1-[4-(1,3,3- trimethyl-6-aza-bicyclo- [3.2.1]octane-6-carbonyl)- phenyl]-cyclo-propyl}- benzamide | 431 |
| 1-89 | 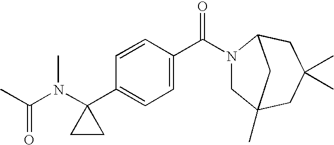 | 368.52 | N-Methyl-N-{1-[4-(1,3,3- trimethyl-6-aza-bicyclo- [3.2.1]octane-6-carbonyl)- phenyl]-cyclo-propyl}- acetamide | 369 |
| 1-90 | 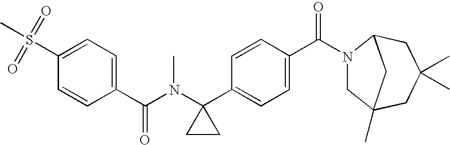 | 508.69 | 4-Methanesulfonyl-N- methyl-N-{1-[4-(1,3,3- trimethyl-6-aza- bicyclo[3.2.1]octane-6- carbonyl)-phenyl]-cyclo- propyl}-benzamide | 509 |

### Example 2-1

### General Procedure (B1)

### 1,1-Dimethyl-3-methyl-3-[4-(1,3,3-trimethyl-6-aza-bicyclo[3.2.1]octane-6-carbonyl)-benzyl]-sulfamide

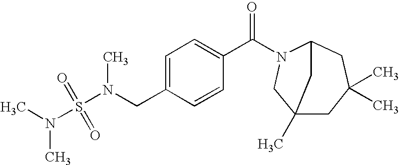

To a solution of (4-methylaminomethyl-phenyl)-(1,3,3-trimethyl-6-aza-bicyclo[3.2.1]oct-6-yl)-methanone (100 mg, 0.33 mmol, Example 1) in DCM (25 mL) was added with stirring TEA (140 μL, 1 mmol) followed by dimethylsulfamoyl chloride (54 μL, 0.5 mmol) and the mixture was stirred for 1 h. at ambient temperature. The mixture was evaporated and the residue purified using preparative HPLC (Method Z4): Amount isolated=28 mg (21%) of the title compound as an oil.

¹H NMR (400 MHz, CDCl₃) δ 0.94 (d, 3H), 1.03 (d, 3H), 1.13 (d, 3H), 1.17-1.61 (m, 4.5H), 1.77 (m, 1H), 2.24 (m, 0.5H), 2.70 (s, 3H), 2.86 (s, 6H), 3.16 (d, 0.5H), 3.26 (m, 1H), 3.60 (d, 0.5H), 3.97 (t, 0.5H), 4.34 (d, 2H), 4.61 (m, 0.5H), 7.38 (m, 2H), 7.44 (t, 2H). HPLC-MS (Method Z1): m/z=408 (M+1); tᵣ=1.98 min (100% ELS).

### Example 2-2

### General Procedure (B1)

### N-Methyl-N-[4-(1,3,3-trimethyl-6-aza-bicyclo[3.2.1]octane-6-carbonyl)-benzyl]-methanesulfonamide

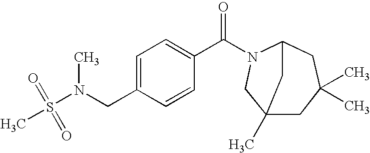

The title compound was prepared by a similar procedure as that described in Example 33, starting from (4-methylaminomethyl-phenyl)-(1,3,3-trimethyl-6-aza-bicyclo[3.2.1]oct-6-yl)-methanone and methanesulfonyl chloride.

HPLC-MS (Method Z1): m/z=379 (M+1); tᵣ=1.8 min (100% ELS).

### Example 2-3

### General Procedure (B1)

### 2,2,2-Trifluoro-ethanesulfonic Acid Methyl-[4-(1,3,3-trimethyl-6-aza-bicyclo[3.2.1]octane-6-carbonyl)-benzyl]-amide

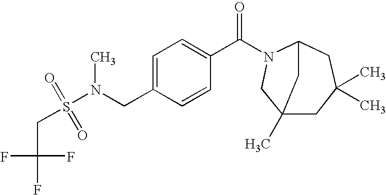

The title compound was prepared by a similar procedure as that described in Example 33, starting from (4-methylaminomethyl-phenyl)-(1,3,3-trimethyl-6-aza-bicyclo[3.2.1]oct-6-yl)-methanone and 2,2,2-trifluoro-ethanesulfonyl chloride.

HPLC-MS (Method Z1): m/z=447 (M+1); tᵣ=2.09 min.

### Example 2-4

### General Procedure (B1)

### N-Methylphenyl-N-[4-(1,3,3-trimethyl-6-aza-bicyclo[3.2.1]octane-6-carbonyl)-benzyl]-methanesulfonamide

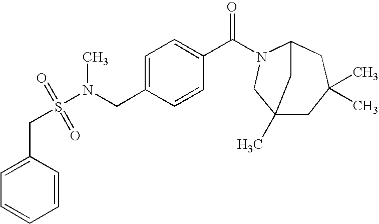

The title compound was prepared by a similar procedure as that described in Example 33, starting from (4-methylaminomethyl-phenyl)-(1,3,3-trimethyl-6-aza-bicyclo[3.2.1]oct-6-yl)-methanone and benzylsulfonyl chloride.

HPLC-MS (Method Z1): m/z=455 (M+1); tᵣ=2.17 min.

The following compounds were made as outlined in general procedure (B2) above.

### Example 2

### General procedure (B2)

### Trifluoro-N-isopropyl-N-[4-(1,3,3-trimethyl-6-aza-bicyclo[3.2.1]octane-6-carbonyl)-benzyl]-methanesulfonamide

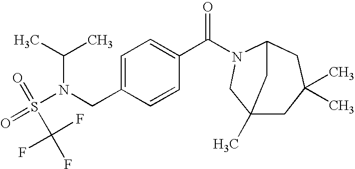

### Step A

### 4-(Tetrahydro-pyran-2-yloxymethyl)-benzoic Acid

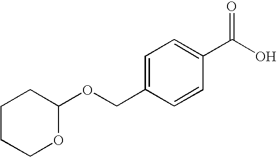

To a ice-water cooled solution of 4-hydroxymethyl-benzoic acid methyl ester (6.0 g, 36.11 mmol) and 3,4-dihydro-2H-pyran (16.47 mL, 180.53 mmol) in DCM (125 mL) was added p-toluenesulfonic acid mono hydrate (69 mg, 0.36 mmol). The mixture was stirred for 4 h. at ambient temperature. The solvent was evaporated affording crude (˜9 g) of 4-(tetrahydropyran-2-yloxymethyl)-benzoic acid methyl ester (LC/MS: 272 [M+23]) as an oil. To a solution of the ester (˜9 g) in EtOH (50 mL) was added 1 N NaOH (55 mL) and the mixture was stirred for 16 h. at ambient temperature. The volatiles were evaporated and the aqueous phase washed with Et₂O (50 mL). pH of the aqueous phase was adjusted to 3 by addition of 1N HCl. The precipitate was extracted with Et₂O (2×50 mL), dried (Na₂SO₄) and evaporated which afforded 6 g (71%) of 4-(tetrahydro-pyran-2-yloxymethyl)-benzoic acid as a solid.

¹H NMR (400 MHz, CDCl₃) δ 1.55-1.92 (m, 6H), 3.55 (m, 1H), 3.91 (t, 1H), 4.59 (d, 1H), 4.74 (t, 1H), 4.87 (d, 1H), 7.48 (d, 2H), 8.09 (d, 2H). HPLC-MS (Method Z1): m/z=259 (M+23); tᵣ=1.42 min.

### Step B

### (4-Hydroxymethyl-phenyl)-(1,3,3-trimethyl-6-aza-bicyclo[3.2.1]oct-6-yl)-methanone

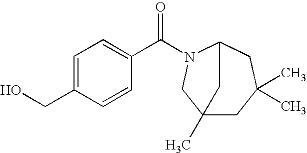

To a solution of the above benzoic acid (6.0 g, 25.40 mmol) in dry THF (100 mL) was added with stirring HOBt (3.8 g, 27.93 mmol) followed by EDAC (5.36 g, 27.93 mmol) and the mixture was stirred for 30 min. at ambient temperature. To the resulting mixture was added 1,3,3-trimethyl-6-aza-bicyclo[3.2.1]octane, hydrochloride (5.3 g, 27.93 mmol) and DIPEA (9.29 mL, 53.33 mmol). The reaction mixture was stirred for 16 h. at ambient temperature. The solvent was evaporated and to the residue was added water (100 mL). The mixture was extracted with Et₂O (3×35 mL) and the combined organic phases were dried (MgSO₄) and the solvent evaporated affording crude amide which was dissolved in MeOH (100 mL). To this mixture was added p-toluenesulfonic acid (1 g) and the mixture was stirred for 2 h at ambient temperature. The solvent was evaporated and the residue purified using silica gel column chromatography (Flash 40) using first as mixture of EtOAc-Heptane (1:2) (500 mL) followed by EtOAc-Heptane 2:1 as eluents. Pure fractions were collected, evaporated to 1/10 volume and the precipitate filtered off and washed with a Et₂O (20 mL) which afforded after drying 5.2 g (71%) of (4-hydroxymethyl-phenyl)-(1,3,3-trimethyl-6-aza-bicyclo[3.2.1]oct-6-yl)-methanone as a solid.

TLC (EtOAc-Heptane) 2:1 Rf: 0.2. ¹H NMR (400 MHz, CDCl₃) δ 0.93 (d, 3H), 1.02 (d, 3H), 1.12 (s, 3H), 1.14-1.60 (m, 5H), 1.75 (m, 1H), 2.23 (m, 0.5H), 2.54 (bs, 1H), 3.19 (q, 0.5H), 3.26 (d, 0.5H), 3.59 (d, 0.5H), 3.96 (t, 0.5H), 4.60 (m, 0.5H), 4.69 (d, 2H), 7.34-7.40 (m, 4H). HPLC-MS (Method Z1): m/z=288 (M+1); tᵣ=1.81 min.

### Step C

### [4-(Isopropylamino-methyl)-phenyl]-(1,3,3-trimethyl-6-aza-bicyclo[3.2.1]oct-6-yl)-methanone

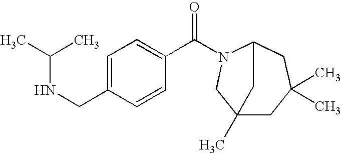

To a ice-water cooled solution of the above benzyl alcohol (500 mg, 1.74 mmol), TEA (0.5 mL, 3.48 mmol) in DCM (40 mL) was added with stirring methanesulfonyl chloride (203 μL, 2.61 mmol) and the mixture was stirred for 1 h at ambient temperature. The mixture was washed with water (20 mL), dried (MgSO₄) and the solvent evaporated. To the residue dissolved in DCM (20 mL) was added isopropyl amine (800 mL) and the mixture was stirred for for 1 h at ambient temperature followed by evaporation of the solvent. The residue was purified using silicagel column chromatography (Flash 40) and AcOEt as eluent. Pure fractions were collected and evaporated affording 300 mg (53%) of [4-(isopropylamino-methyl)-phenyl]-(1,3,3-trimethyl-6-aza-bicyclo[3.2.1]oct-6-yl)-methanone as an oil.

¹H NMR (400 MHz, CDCl₃) δ 0.92 (d, 3H), 1.02 (d, 3H), 1.12 (m, 9H), 1.18-1.45 (m, 4H), 1.56 (m, 1H), 1.75 (m, 1H), 1.94 (bs, 1H), 2.24 (dd, 0.5H), 2.87 (m, 1H), 3.20 (q, 0.5H), 3.27 (dd, 0.5H), 3.57 (d, 0.5H), 3.82 (d, 2H), 3.97 (d, 0.5H), 4.60 (m, 0.5H), 7.38 (m, 4H). HPLC-MS (Method Z1): m/z=329 (M+1); tᵣ=1.28 min (100% ELS).

### Step D

To a solution of the above isopropyl amine (100 mg, 0.304 mmol) in DCM (15 mL) cooled to −50° C. (dry ice/acetone) was added with stirring TEA (130 μL, 0.913 mmol) followed by trifluoromethansulfonic anhydride (100 μL, 0.61 mmol) and the mixture was stirred for 30 min. at −50° C. To the mixture was added water (0.2 mL), the solvent evaporated and the residue purified using preparative HPLC (Method Z4): Amount isolated=65 mg (46%) of the title compound as an oil.

¹H NMR (400 MHz, CDCl₃) δ 0.93 (d, 3H), 1.03 (d, 3H), 1.13 (bs, 9H), 1.16-1.61 (m, 4.5H), 1.77 (m, 1H), 2.24 (dd, 0.5H), 3.18 (q, 1H), 3.28 (d, 0.5H), 3.60 (d, 0.5H), 3.94 (t, 0.5H), 4.25 (m, 1H), 4.31-4.85 (bs, 2H), 4.62 (m, 0.5H), 7.45 (m, 4H). HPLC-MS (Method Z1): m/z=461 (M+1); tᵣ=2.50 min (100% ELS).

### Example 2-6

### General Procedure (B2)

### N-Cyclopropyl-trifluoro-N-[4-(1,3,3-trimethyl-6-aza-bicyclo[3.2.1]octane-6-carbonyl)-benzyl]-methanesulfonamide

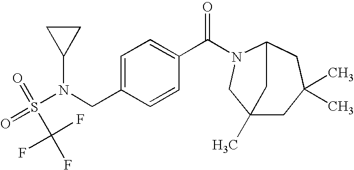

The title compound was prepared by a similar procedure as that described in Example 46, starting from (4-cyclopropylaminomethyl-phenyl)-(1,3,3-trimethyl-6-aza-bicyclo[3.2.1]oct-6-yl)-methanone and trifluoro-methanesulfonic anhydride.

HPLC-MS (Method Z1): m/z=459 (M+1); tᵣ=2.45 min.

### Example 2-7

### General Procedure (B2)

### N-Ethyl-trifluoro-N-[4-(1,3,3-trimethyl-6-aza-bicyclo[3.2.1]octane-6-carbonyl)-benzyl]-methanesulfonamide

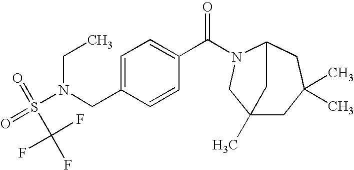

The title compound was prepared by a similar procedure as that described in Example 46, starting from (4-ethylaminomethyl-phenyl)-(1,3,3-trimethyl-6-aza-bicyclo[3.2.1]oct-6-yl)-methanone and trifluoro-methanesulfonic anhydride.

HPLC-MS (Method Z1): m/z=447 (M+1); tᵣ=2.43 min (100% ELS).

### Example 2-8

### General Procedure (B2)

### Trifluoro-N-methyl-N-[4-(1,3,3-trimethyl-6-aza-bicyclo[3.2.1]octane-6-carbonyl)-benzyl]-methanesulfonamide

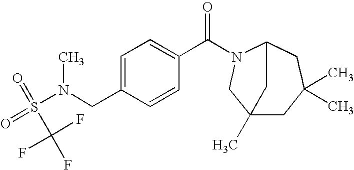

The title compound was prepared by a similar procedure as that described in Example 46, starting from (4-methylaminomethyl-phenyl)-(1,3,3-trimethyl-6-aza-bicyclo[3.2.1]oct-6-yl)-methanone and trifluoro-methanesulfonic anhydride.

HPLC-MS (Method Z1): m/z=433 (M+1); tᵣ=2.35 min.

The following compounds were made as outlined in general method B1 and B2 above.

| Ex. | Structure | MW | IUPAC Name | LC/MS |
|---|---|---|---|---|
| 2-9 | 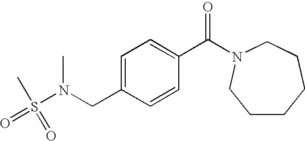 | 324.45 | N-[4-(Azepane-1- carbonyl)-benzyl]-N- methyl-methane- sulfonamide | 325 |
| 2-10 | 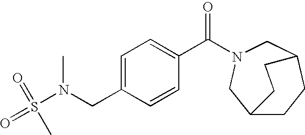 | 350.48 | N-[4-(3-Aza-bicyclo[3.2.2]- nonane-3-carbonyl)- benzyl]-N-methyl- methanesulfonamide | 351 |
| 2-11 | 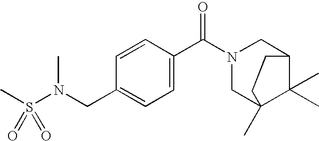 | 378.54 | N-Methyl-N-[4-(1,8,8-tri- methyl-3-aza- bicyclo[3.2.1]-octane-3- carbonyl)-benzyl]- methane-sulfonamide | 379 |
| 2-12 | 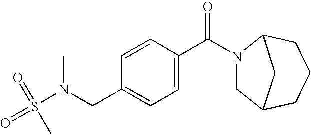 | 336.46 | N-[4-(6-Aza-bicyclo[3.2.1]- octane-6-carbonyl)- benzyl]-N-methyl- methane-sulfonamide | 337 |
| 2-13 | 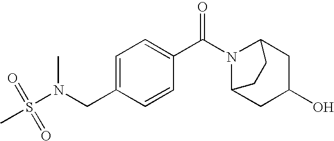 | 352.46 | N-[4-(3-Hydroxy-8-aza- bicyclo[3.2.1]octane-8- carbonyl)-benzyl]-N- methyl- methanesulfonamide | 353 |
| 2-11 | 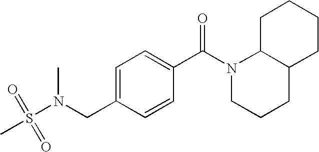 | 354.61 | N-Methyl-N-[4-(octahydro- quinoline-1-carbonyl)- benzyl]-methanesulfon- amide | 355 |
| 2-12 | 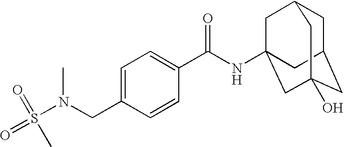 | 392.52 | N-(3-Hydroxy-adamantan- 1-yl)-4-[(methanesulfonyl- methyl-amino)-methyl]- benzamide | 393 |
| 2-13 | 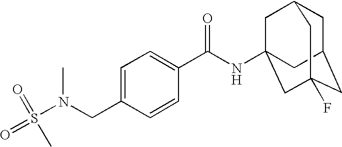 | 394.51 | N-(3-Fluoro-adamantan-1- yl)-4-[(methanesulfonyl- methyl-amino)-methyl]- benzamide | 395 |
| 2-14 | 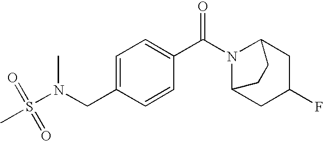 | 354.45 | N-[4-(3-Fluoro-8-aza- bicyclo[3.2.1]octane-8- carbonyl)-benzyl]-N- methyl- methanesulfonamide | 355 |
| 2-15 | 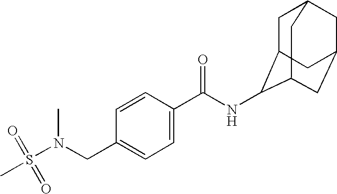 | 376.52 | N-Adamantan-2-yl-4- [(methanesulfonyl-methyl- amino)-methyl]-benzamide | 377 |
| 2-16 | 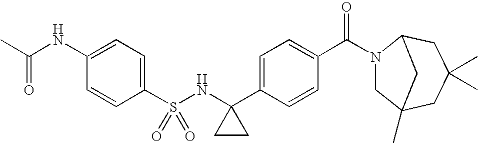 | 509.67 | N-(4-{1-[4-(1,3,3- Trimethyl-6-aza- bicyclo[3.2.1]octane-6- carbonyl)-phenyl]-cyclo- propylsulfamoyl}-phenyl)- acetamide | 510 |
| 2-17 | 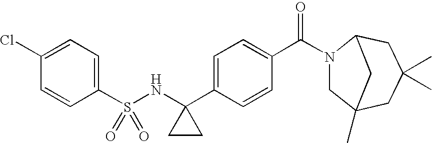 | 487.07 | 4-Chloro-N-{1-[4-(1,3,3-tri- methyl-6-aza-bicyclo- [3.2.1]octane-6-carbonyl)- phenyl]-cyclo-propyl}- benzene-sulfonamide | 488 |
| 2-18 | 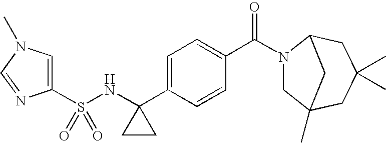 | 456.61 | 1-Methyl-1H-imidazole-4- sulfonic acid {1-[4-(1,3,3- trimethyl-6-aza-bicyclo- [3.2.1]octane-6-carbonyl)- phenyl]-cyclopropyl}- amide | 457 |
| 2-19 | 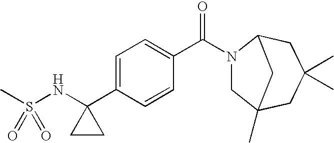 | 390.55 | N-{1-[4-(1,3,3-Trimethyl-6- aza-bicyclo[3.2.1]octane- 6-carbonyl)-phenyl]-cyclo- propyl}-ethanesulfonamide | 391 |

### Example 3-1

### General Procedure (C)

### 3-Benzoyl-1-methyl-1-[4-(1,3,3-trimethyl-6-aza-bicyclo[3.2.1]octane-6-carbonyl)-benzyl]-urea

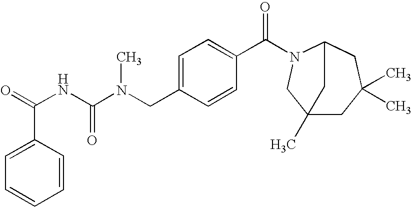

To a solution of (4-methylaminomethyl-phenyl)-(1,3,3-trimethyl-6-aza-bicyclo[3.2.1]oct-6-yl)-methanone (70 mg, 0.23 mmol, Example 1) in DCM (20 mL) was added benzoyl isocyanate (51 mg, 0.35 mmol) and the mixture was stirred for 16 h. at ambient temperature. The mixture was evaporated and the residue purified on silica gel column chromatography (Flash 40) using first a mixture of AcOEt-Heptane (1:1) as eluent followed by pure AcOEt. Pure fractions were collected and evaporated which afforded 45 mg (43%) of the title compound.

¹H NMR (400 MHz, CDCl₃) δ 0.93 (d, 3H), 1.03 (s, 3H), 1.13 (s, 3H), 1.16-1.60 (m, 4.5H), 1.76 (m, 1H), 2.23 (m, 0.5H), 2.99 (bs, 3H), 3.16 (d, 0.5H), 3.26 (m, 1H), 3.58 (d, 0.5H), 3.96 (m, 0.5H), 4.61 (m, 0.5H), 4.64 (s, 2H), 7.42 (m, 6H), 7.55 (t, 1H), 7.89 (m, 2H), 8.66 (bs, 1H). HPLC-MS (Method Z1): m/z=448 (M+1); tᵣ=1.85 min (100% ELS).

### Example 3-2

### General Procedure (C)

### 3-Cyclohexyl-1-methyl-1-[4-(1,3,3-trimethyl-6-aza-bicyclo[3.2.1]octane-6-carbonyl)-benzyl]-urea

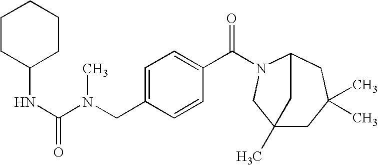

The title compound was prepared by a similar procedure as that described in Example 37, starting from (4-methylaminomethyl-phenyl)-(1,3,3-trimethyl-6-aza-bicyclo[3.2.1]oct-6-yl)-methanone and isocyanato-cyclohexane.

HPLC-MS (Method Z1): m/z=426 (M+1); tᵣ=2.06 min.

### Example 3-3

### General Procedure (C)

### 3-(4-Methyl-benzenesulfonyl)-1-methyl-1-[4-(1,3,3-trimethyl-6-aza-bicyclo[3.2.1]octane-6-carbonyl)-benzyl]-urea

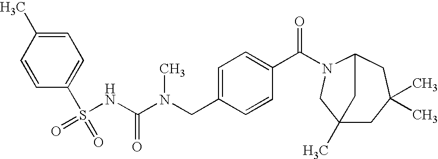

The title compound was prepared by a similar procedure as that described in Example 37, starting from (4-methylaminomethyl-phenyl)-(1,3,3-trimethyl-6-aza-bicyclo[3.2.1]oct-6-yl)-methanone and 4-methyl-benzenesulfonyl isocyanate.

HPLC-MS (Method Z1): m/z=499 (M+1); tᵣ=2.02 min.

### Example 3-4

### General Procedure (C)

### 3-(2,3-Dihydro-1,4-benzodioxin-2-ylmethyl)-1-methyl-1-[4-(1,3,3-trimethyl-6-aza-bicyclo[3.2.1]octane-6-carbonyl)-benzyl]-urea

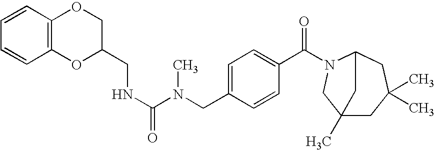

### Step A

### 1-Methyl-3-{methyl-[4-(1,3,3-trimethyl-6-aza-bicyclo[3.2.1]octane-6-carbonyl)-benzyl]-carbamoyl}-3H-imidazol-1-ium, Iodide

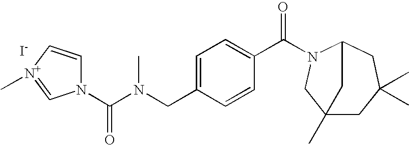

To a solution of (4-methylaminomethyl-phenyl)-(1,3,3-trimethyl-6-aza-bicyclo[3.2.1]oct-6-yl)-methanone (1.1 g, 3.66 mmol, Example 1) in DCM (40 mL) was added CDI (0.9 g, 5.49 mmol) and the mixture was stirred for 16 h. at ambient temperature. The mixture was washed with water (25 mL), dried (Na₂SO₄) and evaporated. To the residue dissolved in MeCN (40 mL) was added methyl iodine (2.5 mL, 36.61 mmol) and the resulting mixture was stirred for 16 h. at ambient temperature. The solvent was evaporated affording 2 g (˜100%) of 1-methyl-3-{methyl-[4-(1,3,3-trimethyl-6-aza-bicyclo[3.2.1]octane-6-carbonyl)-benzyl]-carbamoyl}-3H-imidazol-1-ium, iodide.

### Step B

To a solution of 2,3-dihydro-benzo[1,4]dioxin-2-yl-methylamine (22 mg, 0.134 mmol) in a mixture of DCM (2 mL) and TEA (16 μL, 0.134 mmol) was added a solution of the above imidazolium salt (60 mg, 0.112 mmol) in DCM (2 mL). The mixture was stirred for 16 h. at ambient temperature and the solvent evaporated. The residue was purified using preparative HPLC (Method Z4): Amount isolated=50 mg (91%) of the title compound as a solid.

¹H NMR (400 MHz, CDCl₃) δ 0.93 (d, 3H), 1.02 (s, 3H), 1.12 (s, 3H), 1.16-1.60 (m, 4.5H), 1.76 (m, 1H), 2.23 (m, 0.5H), 2.88 (s, 3H), 2.97 (bs, 1H), 3.14 (d, 0.5H), 3.25 (t, 1H), 3.53 (m, 0.5H), 3.59 (d, 0.5H), 3.68 (m, 0.5H), 3.97 (m, 2H), 4.30 (d, 2H), 4.53 (d, 2H), 4.60 (m, 0.5H), 4.96 (t, 0.5H), 6.85 (m, 4H), 7.27 (m, 2H), 7.41 (t, 2H). HPLC-MS (Method Z1): m/z=493 (M+1); tᵣ=2.10 min (100% ELS).

### Example 3-5

### General Procedure (C)

### 3-(3-Methoxy-benzyl)-1-methyl-1-[4-(1,3,3-trimethyl-6-aza-bicyclo[3.2.1]octane-6-carbonyl)-benzyl]-urea

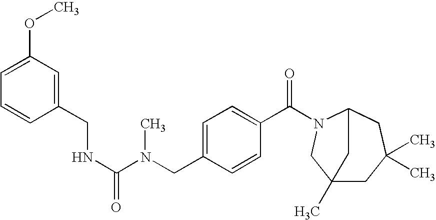

The title compound was prepared by a similar procedure as that described in Example 40, starting from (4-methylaminomethyl-phenyl)-(1,3,3-trimethyl-6-aza-bicyclo[3.2.1]oct-6-yl)-methanone and 3-methoxy-benzylamine.

HPLC-MS (Method Z1): m/z=465 (M+1); tᵣ=2.02 min (100% ELS).

### Example 3-6

### General Procedure (C)

### 3-(1,1-Dioxo-tetrahydro-thiophen-3-yl)-1-methyl-1-[4-(1,3,3-trimethyl-6-aza-bicyclo[3.2.1]octane-6-carbonyl)-benzyl]-urea

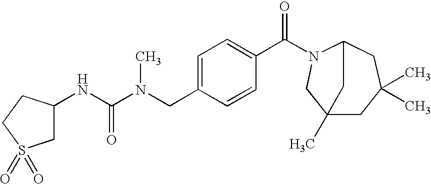

The title compound was prepared by a similar procedure as that described in Example 40, starting from (4-methylaminomethyl-phenyl)-(1,3,3-trimethyl-6-aza-bicyclo[3.2.1]oct-6-yl)-methanone and 1,1-dioxo-tetrahydro-thiophen-3-ylamine.

HPLC-MS (Method Z1): m/z=463 (M+1); tᵣ=1.63 min (100% ELS).

### Example 3-7

### General Procedure (C)

### 1-Methyl-3-(tetrahydro-pyran-4-yl)-1-[4-(1,3,3-trimethyl-6-aza-bicyclo[3.2.1]octane-6-carbonyl)-benzyl]-urea

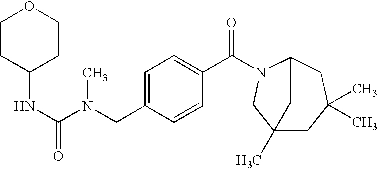

The title compound was prepared by a similar procedure as that described in Example 40, starting from (4-methylaminomethyl-phenyl)-(1,3,3-trimethyl-6-aza-bicyclo[3.2.1]oct-6-yl)-methanone and tetrahydro-pyran-4-yl-amine.

HPLC-MS (Method Z1): m/z=429 (M+1); tᵣ=1.69 min (100% ELS).

### Example 3-8

### General Procedure (C)

### {1,3-Dimethyl-3-[4-(1,3,3-trimethyl-6-aza-bicyclo[3.2.1]octane-6-carbonyl)-benzyl]-ureido}-acetic Acid Methyl Ester

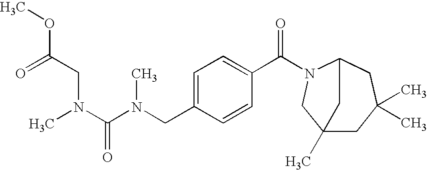

The title compound was prepared by a similar procedure as that described in Example 40, starting from (4-methylaminomethyl-phenyl)-(1,3,3-trimethyl-6-aza-bicyclo[3.2.1]oct-6-yl)-methanone and methylamino-acetic acid methyl ester.

HPLC-MS (Method Z1): m/z=431 (M+1); tᵣ=1.85 min (100% ELS).

### Example 3-9

### General Procedure (C)

### 1-Methyl-3-(5-trifluoromethyl-1,3,4-thiadiazol-2-yl)-1-[4-(1,3,3-trimethyl-6-aza-bicyclo[3.2.1]-octane-6-carbonyl)-benzyl]-urea

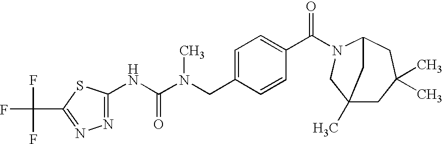

The title compound was prepared by a similar procedure as that described in Example 40, starting from (4-methylaminomethyl-phenyl)-(1,3,3-trimethyl-6-aza-bicyclo[3.2.1]oct-6-yl)-methanone and 5-trifluoromethyl-[1,3,4]thiadiazol-2-ylamine.

HPLC-MS (Method Z1): m/z=497 (M+1); tᵣ=2.19 min (100% ELS).

The following compounds were made as outlined in general method C above.

| | | | | LC/ |
|---|---|---|---|---|
| Ex. | Structure | MW | IUPAC Name | MS |
| 3-10 | 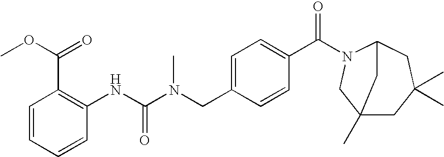 | 477.6 | 2-{3-Methyl-3-[4-(1,3,3- trimethyl-6-aza-bicyclo- [3.2.1]octane-6-arbonyl)- benzyl]-ureido}-benzoic acid methyl ester | 478 |
| 3-11 | 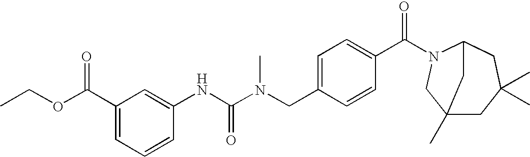 | 491.6 | 3-{3-Methyl-3-[4-(1,3,3- trimethyl-6-aza-bicyclo- [3.2.1]octane-6-carbonyl)- benzyl]-ureido}-benzoic acid ethyl ester | 492 |
| 3-12 | 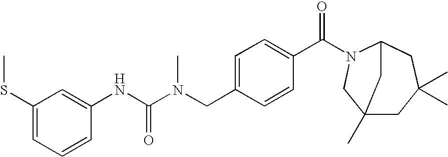 | 465.7 | 1-Methyl-3-(3-ethylsulfanyl- phenyl)-1-[4-(1,3,3-trimethyl-6- aza-bicyclo-[3.2.1]octane-6- carbonyl)-benzyl]-urea | 466 |
| 3-13 | 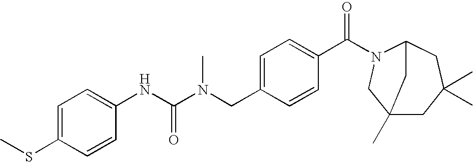 | 465.7 | 1-Methyl-3-(4-ethylsulfannyl- phenyl)-1-[4-(1,3,3-trimethyl-6- aza-bicyclo-[3.2.1]octane-6- carbonyl)-benzyl]-urea | 466 |
| 3-14 | 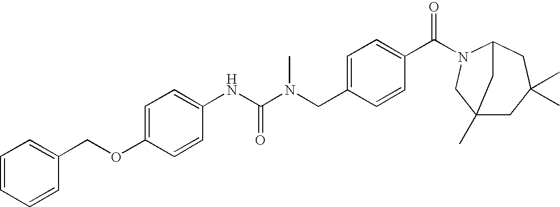 | 525.7 | 3-(4-Benzyloxy-phenyl)-1- methyl-1-[4-(1,3,3-trimethyl-6- aza-bicyclo[3.2.1]octane-6- carbonyl)-benzyl]-urea | 526 |
| 3-15 | 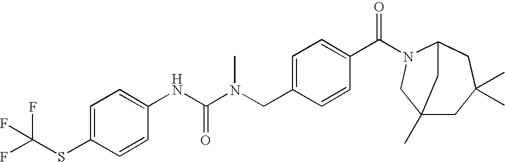 | 519.6 | 1-Methyl-3-(4-trifluoro-methyl- sulfanyl-phenyl)-1-[4-(1,3,3-tri- methyl-6-aza-bicyclo[3.2.1]- octane-6-carbonyl)-benzyl]- urea | 520 |
| 3-16 | 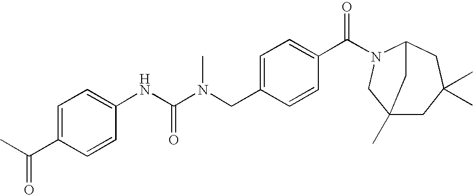 | 461.6 | 3-(4-Acetyl-phenyl)-1-methyl- 1-[4-(1,3,3-tri-methyl-6-aza- bicyclo-[3.2.1]octane-6- carbonyl)-benzyl]-urea | 462 |
| 3-17 | 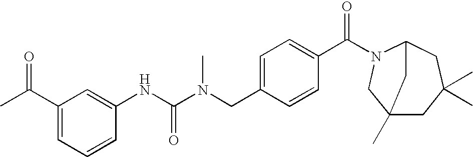 | 461.6 | 3-(3-Acetyl-phenyl)-1-methyl- 1-[4-(1,3,3-tri-methyl-6-aza- bicyclo-[3.2.1]octane-6- carbonyl)-benzyl]-urea | 462 |
| 3-18 | 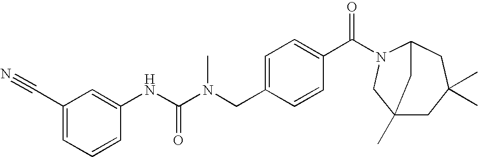 | 444.6 | 3-(3-cyano-phenyl)-1-methyl- 1-[4-(1,3,3-tri-methyl-6-aza- bicyclo-[3.2.1]octane-6- carbonyl)-benzyl]-urea | 445 |
| 3-19 | 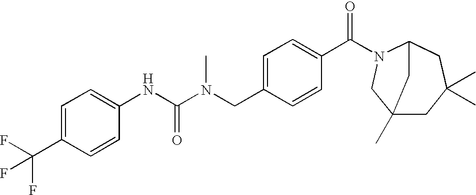 | 487.6 | 1-Methyl-3-(4-trifluoro-methyl- phenyl)-1-[4-(1,3,3-trimethyl-6- aza-bicyclo-[3.2.1]octane-6- carbonyl)-benzyl]-urea | 488 |
| 3-20 | 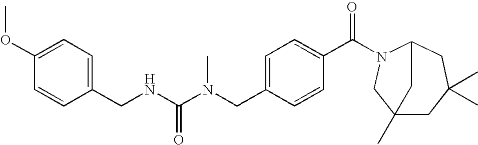 | 463.6 | 3-(4-Methoxy-benzyl)-1- methyl-1-[4-(1,3,3-tri-methyl-6- aza-bicyclo-[3.2.1]octane-6- carbonyl)-benzyl]-urea | 464 |
| 3-21 | 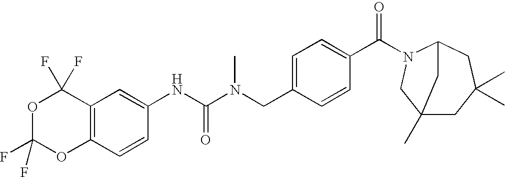 | 549.6 | 1-Methyl-3-(2,2,4,4-tetrafluoro- 4H-benzo[1,3]dioxin-6-yl)-1- [4-(1,3,3-trimethyl-6-aza- bicyclo-[3.2.1]octane-6- carbonyl)-benzyl]-urea | 550 |
| 3-22 | 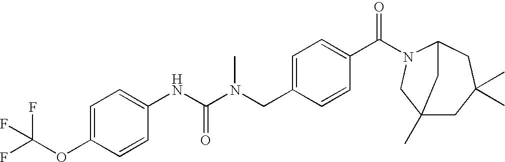 | 503.6 | 1-Methyl-3-(4-trifluoro- methoxy-phenyl)-1-[4-(1,3,3- trimethyl-6-aza- bicyclo[3.2.1]octane-6- carbonyl)-benzyl]-urea | 504 |
| 3-23 | 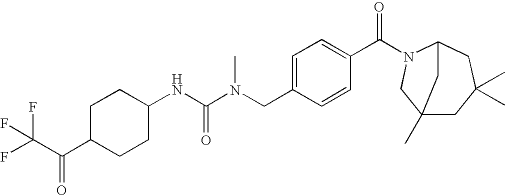 | 521.6 | 1-Methyl-3-[4-(2,2,2-trifluoro- acetyl)-cyclohexyl]-1-[4-(1,3,3- trimethyl-6-aza-bicyclo[3.2.1]- octane-6-carbonyl)-benzyl]- urea | 523 |
| 3-24 | 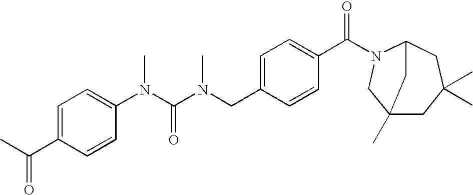 | 475.6 | 1-(4-Acetyl-phenyl)-1,3- dimethyl-3-[4-(1,3,3-tri-methyl- 6-aza-bicyclo[3.2.1]octane-6- carbonyl)-benzyl]-urea | 476 |
| 3-25 | 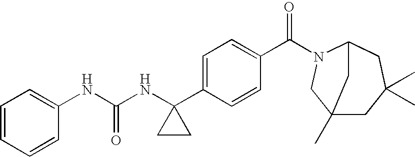 | 431.6 | 1-Phenyl-3-{1-[4-(1,3,3- trimethyl-6-aza-bicyclo- [3.2.1]octane-6-carbonyl)- phenyl]-cyclopropyl}-urea | 432 |
| 3-26 | 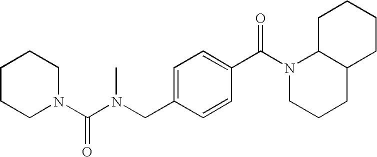 | 397.6 | Piperidine-1-carboxylic acid methyl-[4-(octahydro- quinoline-1-carbonyl)-benzyl]- amide | 398 |
| 3-27 | 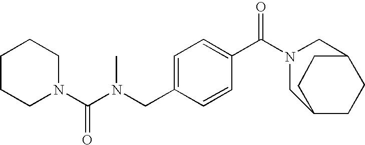 | 383.5 | Piperidine-1-carboxylic acid [4-(3-aza-bicyclo- [3.2.2]nonane-3-carbonyl)- benzyl]-methyl-amide | 384 |
| 3-28 | 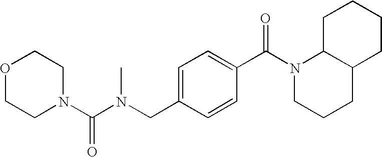 | 399.5 | Morpholine-4-carboxylic acid methyl-[4-(octahydro- quinoline-1-carbonyl)-benzyl]- amide | 400 |
| 3-29 | 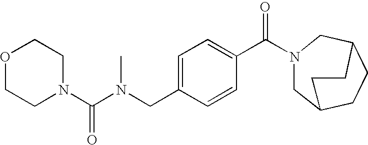 | 385.5 | Morpholine-4-carboxylic acid [4-(3-aza-bicyclo- [3.2.2]nonane-3-carbonyl)- benzyl]-methyl-amide | 386 |
| 3-30 | 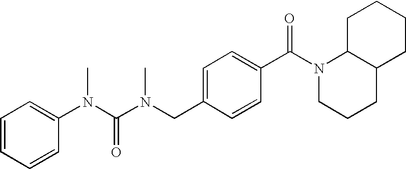 | 419.6 | 1,3-Dimethyl-3-[4-(octahydro- quinoline-1-carbonyl)-benzyl]- 1-phenyl-urea | 420 |
| 3-31 | 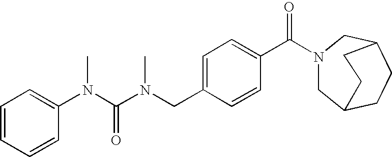 | 405.5 | 1-[4-(3-Aza-bicyclo-[3.2.2]- nonane-3-carbonyl)-benzyl]- 1,3-dimethyl-3-phenyl-urea | 406 |
| 3-32 | 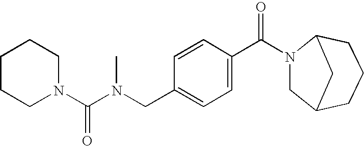 | 369.5 | Piperidine-1-carboxylic acid [4-(6-aza-bicyclo- [3.2.1]octane-6-carbonyl)- benzyl]-methyl-amide | 370 |
| 3-33 | 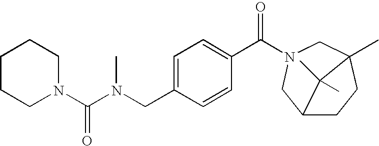 | 411.6 | Piperidine-1-carboxylic acid methyl-[4-(1,8,8-trimethyl-3- aza-bicyclo[3.2.1]octane-3- carbonyl)-benzyl]-amide | 412 |
| 3-34 | 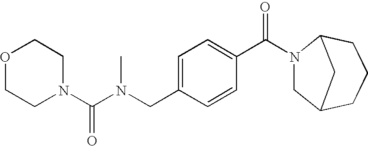 | 371.5 | Morpholine-4-carboxylic acid [4-(6-aza-bicyclo- [3.2.1]octane-6-carbonyl)- benzyl]-methyl-amide | 372 |
| 3-35 | 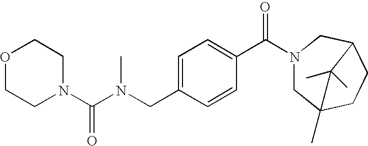 | 413.6 | Morpholine-4-carboxylic acid methyl-[4-(1,8,8-trimethyl-3- aza-bicyclo[3.2.1]octane-3- carbonyl)-benzyl]-amide | 414 |
| 3-36 | 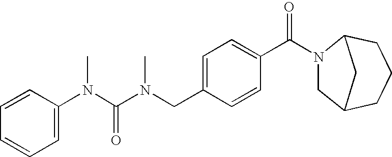 | 391.5 | 1-[4-(6-Aza-bicyclo- [3.2.1]octane-6-carbonyl)- benzyl]-1,3-dimethyl-3-phenyl- urea | 392 |
| 3-37 | 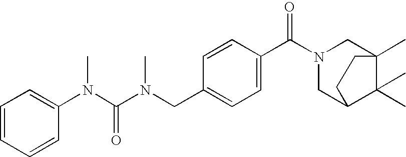 | 433.6 | 1,3-Dimethyl-1-phenyl-3-[4- (1,8,8-trimethyl-3-aza-bicyclo- [3.2.1]octane-3-carbonyl)- benzyl]-urea | 434 |
| 3-38 | 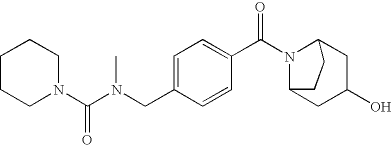 | 385.5 | Piperidine-1-carboxylic acid [4-(3-hydroxy-8-aza- bicyclo[3.2.1]-octane-8- carbonyl)-benzyl]-methyl- amide | 386 |
| 3-39 | 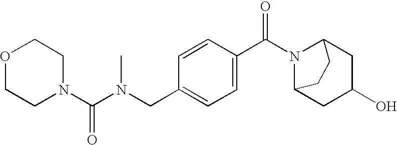 | 387.5 | Morpholine-4-carboxylic acid [4-(3-hydroxy-8-aza-bicyclo- [3.2.1]octane-8-carbonyl)- benzyl]-methyl-amide | 388 |
| 3-40 | 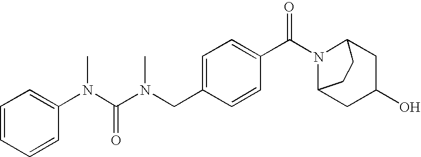 | 407.5 | 1-[4-(3-Hydroxy-8-aza-bicyclo- [3.2.1]octane-8-carbonyl)- benzyl]-1,3-di-methyl-3- phenyl-urea | 408 |
| 3-41 | 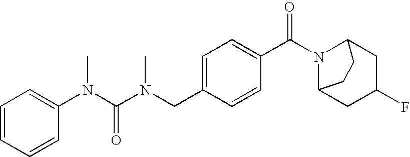 | 409.5 | 1-[4-(3-Fluoro-8-aza-bicyclo- [3.2.1]octane-8-carbonyl)- benzyl]-1,3-di-methyl-3- phenyl-urea | 410 |
| 3-42 | 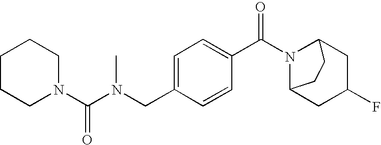 | 387.5 | Piperidine-1-carboxylic acid [4-(3-fluoro-8-aza- bicyclo[3.2.1]octane-8- carbonyl)-benzyl]-methyl- amide | 388 |
| 3-43 | 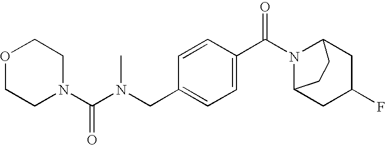 | 389.5 | Morpholine-4-carboxylic acid [4-(3-fluoro-8-aza- bicyclo[3.2.1]-octane-8- carbonyl)-benzyl]-methyl- amide | 390 |
| 3-44 | 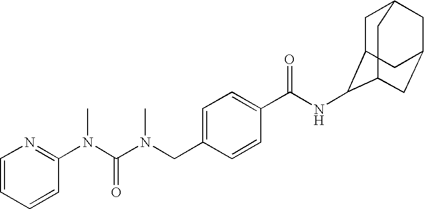 | 432.6 | N-Adamantan-2-yl-4-(1,3- dimethyl-3-pyridin-2-yl- ureidomethyl)-benzamide | 433 |
| 3-45 | 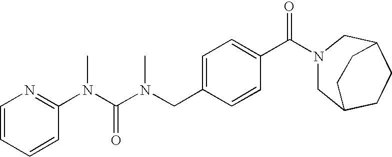 | 406.5 | 1-[4-(3-Aza-bicyclo[3.2.2]- nonane-3-carbonyl)-benzyl]- 1,3-dimethyl-3-pyridin-2-yl- urea | 407 |
| 3-46 | 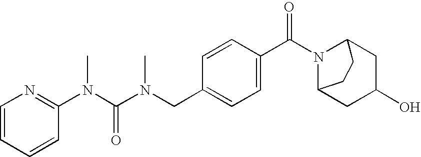 | 408.5 | 1-[4-(3-Hydroxy-8-aza-bicyclo- [3.2.1]octane-8-carbonyl)- benzyl]-1,3-dimethyl-3-pyridin- 2-yl-urea | 409 |
| 3-47 | 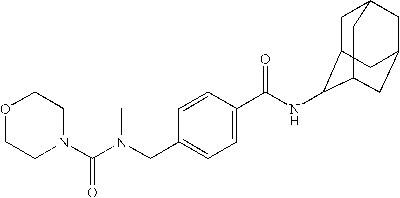 | 411.5 | Morpholine-4-carboxylic acid [4-(adamantan-2- ylcarbamoyl)-benzyl]-methyl- amide | 412 |
| 3-48 | 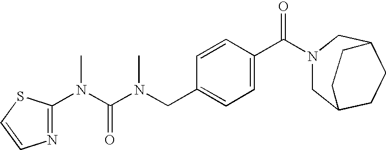 | 412.6 | 1-[4-(3-Aza-bicyclo- [3.2.2]nonane-3-carbonyl)- benzyl]-1,3-dimethyl-3-thiazol- 2-yl-urea | 413 |
| 3-49 | 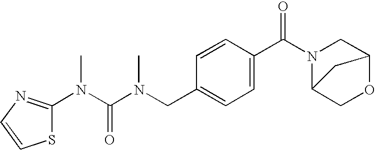 | 386.5 | 1,3-Dimethyl-3-[4-(2-oxa-5- aza-bicyclo[2.2.1]heptane-5- carbonyl)-benzyl]-1-thiazol-2- yl-urea | 387 |
| 3-50 | 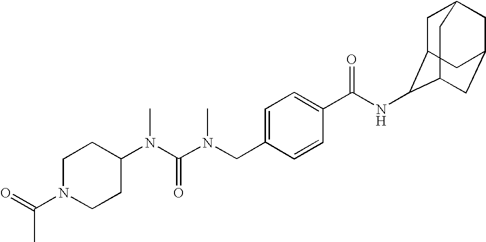 | 480.7 | 4-[3-(1-Acetyl-piperidin-4-yl)- 1,3-dimethyl-ureidomethyl]-N- adamantan-2-yl-benzamide | 481 |
| 3-51 | 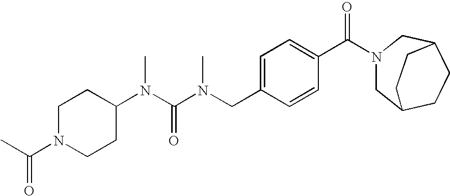 | 454.6 | 1-(1-Acetyl-piperidin-4-yl)-3-[4- (3-aza-bicyclo[3.2.2]nonane-3- carbonyl)-benzyl]-1,3- dimethyl-urea | 455 |
| 3-52 | 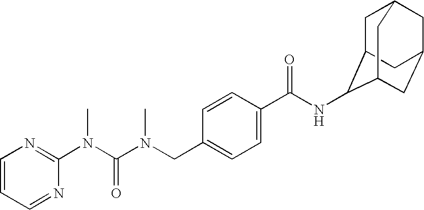 | 433.6 | N-Adamantan-2-yl-4-(1,3- dimethyl-3-pyrimidin-2-yl- ureidomethyl)-benzamide | 434 |
| 3-53 | 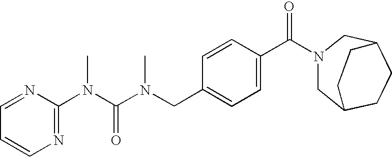 | 407.5 | 1-[4-(3-Aza-bicyclo- [3.2.2]nonane-3-carbonyl)- benzyl]-1,3-dimethyl-3- pyrimidin-2-yl-urea | 408 |
| 3-54 | 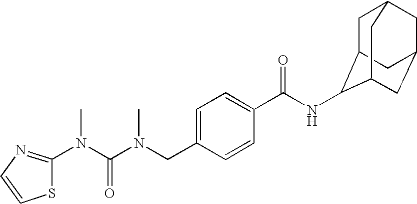 | 438.6 | N-Adamantan-2-yl-4-(1,3- dimethyl-3-thiazol-2-yl- ureidomethyl)-benzamide | 439 |
| 3-55 | 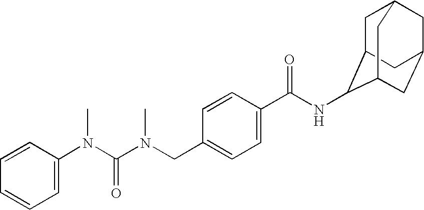 | 431.6 | N-Adamantan-2-yl-4-(1,3- dimethyl-3-phenyl-ureido- methyl)-benzamide | 432 |
| 3-56 | 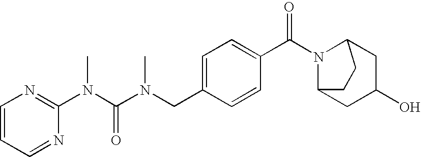 | 409.5 | 1-[4-(3-Hydroxy-8-aza- bicyclo[3.2.1]octane-8- carbonyl)-benzyl]-1,3- dimethyl-3-pyrimidin-2-yl-urea | 410 |
| 3-57 | 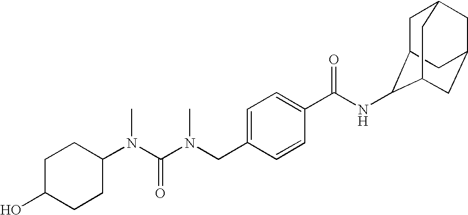 | 453.6 | N-Adamanta-2-yl-4-[3-(4- hydroxy-cyclohexyl)-1,3- dimethyl-ureidomethyl]- benzamide | 454 |
| 3-58 | 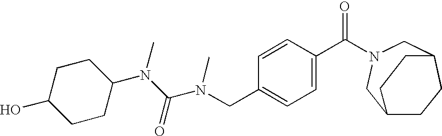 | 427.6 | 1-[4-(3-Aza-bicyclo[3.2.2]- nonane-3-carbonyl)-benzyl]-3- (4-hydroxy-cyclohexyl)-1,3- dimethyl-urea | 428 |
| 3-59 | 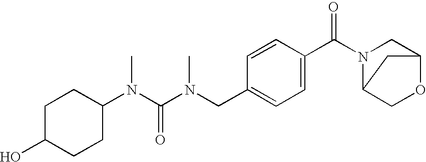 | 401.5 | 1-(4-Hydroxy-cyclohexyl)-1,3- dimethyl-3-[4-(2-oxa-5-aza- bicyclo[2.2.1]heptane-5- carbonyl)-benzyl]-urea | 402 |
| 3-60 | 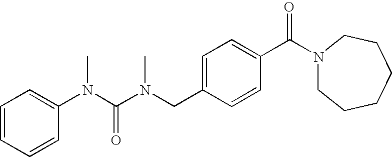 | 379.51 | 1-[4-(Azepane-1-carbonyl)- benzyl]-1,3-dimethyl-3-phenyl- urea | 380 |
| 3-61 | 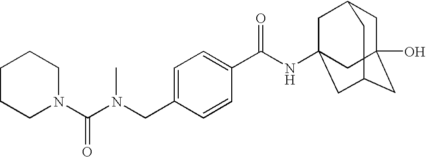 | 425.58 | Piperidine-1-carboxylic acid [4-(3-hydroxy-adamantan-1-yl- carbamoyl)-benzyl]-methyl- amide | 426 |
| 3-62 | 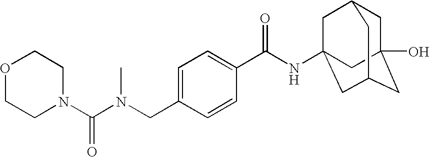 | 427.55 | Morpholine-4-carboxylic acid [4-(3-hydroxy-adamantan-1-yl- carbamoyl)-benzyl]-methyl- amide | 428 |
| 3-63 | 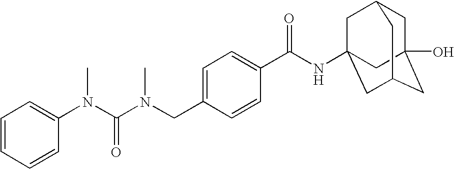 | 447.58 | 4-(1,3-Dimethyl-3-phenyl- ureidomethyl)-N-(3-hydroxy- adamantan-1-yl)-benzamide | 448 |
| 3-64 | 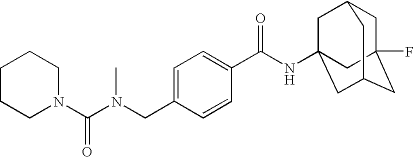 | 427.54 | Piperidine-1-carboxylic acid [4-(3-fluoro-adamantan-1-yl- carbamoyl)-benzyl]-methyl- amide | 428 |
| 3-65 | 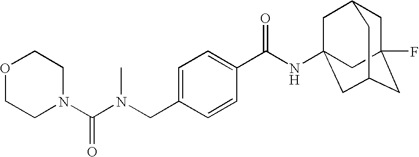 | 429.54 | Morpholine-4-carboxylic acid [4-(3-fluoro-adamantan-1-yl- carbamoyl)-benzyl]-methyl- amide | 430 |
| 3-66 | 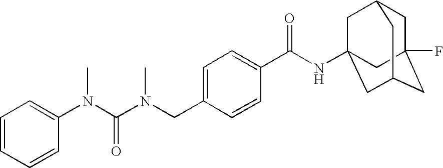 | 449.57 | 4-(1,3-Dimethyl-3-phenyl- ureidomethyl)-N-(3-fluoro- adamantan-1-yl)-benzamide | 450 |
| 3-67 | 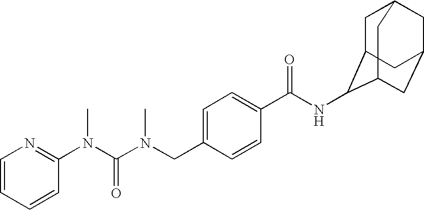 | 432.57 | N-Adamantan-2-yl-4-(1,3- dimethyl-3-pyridin-2-yl- ureidomethyl)-benzamide | 433 |
| 3-68 | 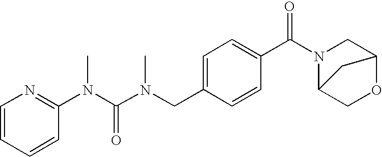 | 380.45 | 1,3-Dimethyl-3-[4-(2-oxa-5- aza-bicyclo[2.2.1]heptane-5- carbonyl)-benzyl]-1-pyridin-2- yl-urea | 381 |
| 3-69 | 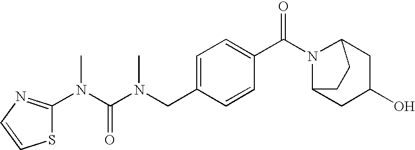 | 414.53 | 1-[4-(3-Hydroxy-8-aza- bicyclo[3.2.1]octane-8- carbonyl)-benzyl]-1,3- dimethyl-3-thiazol-2-yl-urea | 415 |
| 3-70 | 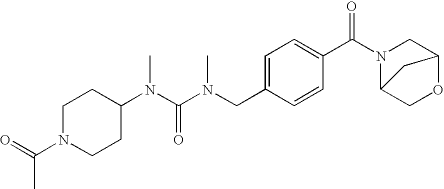 | 428.54 | 1-(1-Acetyl-piperidin-4-yl)-1,3- dimeth- yl-3-[4-(2-oxa-5-aza- icyclo[2.2.1]hept- ane-5-carbonyl)- benzyl]-urea | 429 |
| 3-71 | 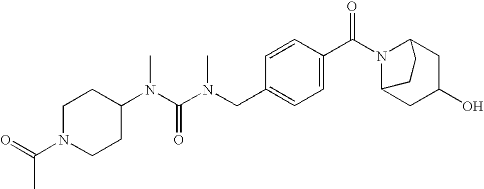 | 456.59 | 1-(1-Acetyl-piperidin-4-yl)-3-[4- (3-hydroxy-8-aza-bicyclo- [3.2.1]-octane-8-carbonyl)- benzyl]-1,3-dimethyl-urea | 457 |
| 3-72 | 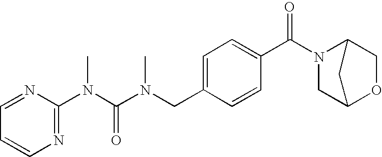 | 381.44 | 1,3-Dimethyl-3-[4-(2-oxa-5- aza-bicyclo[2.2.1]heptane-5- arbonyl)-benzyl]-1-pyrimidin-2- yl-urea | 382 |
| 3-73 | 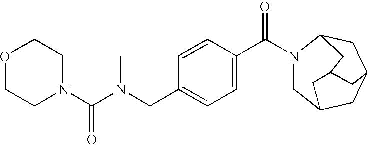 | 411.55 | Morpholine-4-carboxylic acid [4-(4-aza-tricyclo[4.3.1.1*3,8*]- undecane-4-carbonyl)-benzyl]- methyl-amide | 412 |
| 3-74 | 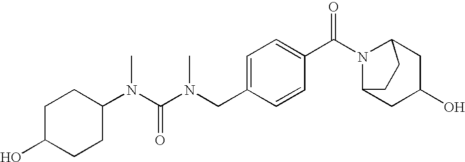 | 429.56 | 1-[4-(3-Hydroxy-8-aza- bicyclo[3.2.1]octane-8- carbonyl)-benzyl]-3-(4- hydroxy-cyclohexyl)-1,3- dimethyl-urea | 430 |
| 3-75 | 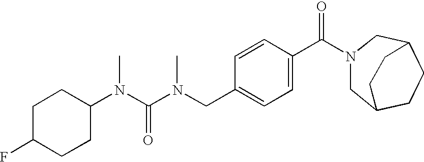 | 429.58 | 1-[4-(3-Aza-bicyclo- [3.2.2]nonane-3-carbonyl)- benzyl]-3-(4-fluoro-cyclo- hexyl)-1,3-dimethyl-urea | 430 |
| 3-76 | 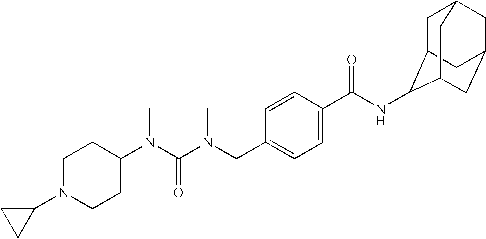 | 478.68 | N-Adamantan-2-yl-4-[3-(1- cyclopropyl-piperidin-4-yl)-1,3- dimethyl-ureidomethyl]- benzamide | 479 |
| 3-77 | 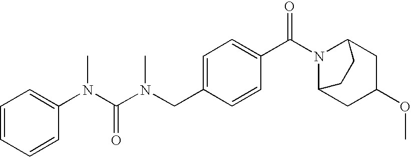 | 421.54 | 1-[4-(3-Methoxy-8-aza- bicyclo[3.2.1]octane-8- carbonyl)-benzyl]-1,3- dimethyl-3-phenyl-urea | 422 |

### Example 4-1

### General Procedure (E)

### 1-Methyl-3-[4-(1,3,3-trimethyl-6-aza-bicyclo[3.2.1]octane-6-carbonyl)-benzyl]-imidazolidin-2-one

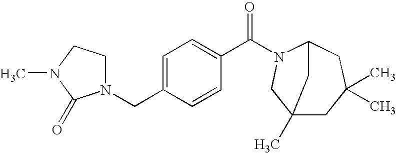

### Step A

### (2-Hydroxy-ethyl)-[4-(1,3,3-trimethyl-6-aza-bicyclo[3.2.1]octane-6-carbonyl)-benzyl]-carbamic Acid Tert-butyl Ester

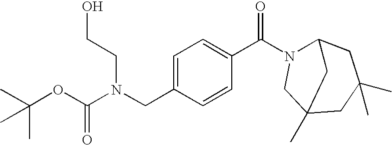

To a solution of [4-(1,3,3-trimethyl-6-aza-bicyclo[3.2.1]octane-6-carbonyl)-benzyl]-carbamic acid tert-butyl ester (1.7 g, 4.4 mmol, Example 1) in DMF (50 mL) was added with stirring sodium hydride (211 mg, 8.8 mmol, 60% in mineral oil) and the mixture was stirred for 15 min. at ambient temperature. To the resulting mixture was added (2-bromo-ethoxymethyl)-benzene (1.1 mL, 5.28 mmol). The reaction mixture was stirred for 16 h. at ambient temperature and quenched by addition of saturated aqueous ammonium chloride (50 mL) followed by water (50 mL). The mixture was extracted with Et₂O (2×100 mL) and the combined organic phases were washed with saturated aqueous ammonium chloride (2×100 mL), dried (Na₂SO₄) and the solvent evaporated. The residue was purified using silicagel column chromatography (Flash 40) affording 1.4 g of (2-benzyloxy-ethyl)-[4-(1,3,3-trimethyl-6-aza-bicyclo[3.2.1]octane-6-carbonyl)-benzyl]-carbamic acid tert-butyl ester as an oil.

HPLC-MS (Method Z1): m/z=521 (M+1); tᵣ=2.67 min.

To the above benzyl ether (1.4 g, 2.69 mmol) dissolved in EtOH (50 mL) was added 10% Pd/C (750 mg, 50% water) and the resulting mixture was hydrogenated at 1 atm. until 1 eqv. of H₂ was used. The mixture was filtered and the solvent evaporated affording 0.9 g (47%) of (2-hydroxy-ethyl)-[4-(1,3,3-trimethyl-6-aza-bicyclo[3.2.1]octane-6-carbonyl)-benzyl]-carbamic acid tert-butyl ester as a solid.

¹H NMR (400 MHz, CDCl₃) δ 0.93 (d, 3H), 1.03 (d, 3H), 1.13 (d, 3H), 1.17-1.79 (m, 15H), 2.23 (m, 0.5H), 3.15 (m, 0.5H), 3.26 (m, 0.5H), 3.41 (bs, 3H), 3.59 (d, 0.5H), 3.71 (m, 2H), 3.97 (t, 0.5H), 4.50 (m, 2H), 4.61 (m, 0.5H), 7.27 (m, 2H), 7.42 (t, 2H). HPLC-MS (Method Z1): m/z=431 (M+1); tᵣ=1.99 min.

### Step B

### {4-[(2-Methylamino-ethylamino)-methyl]-phenyl}-(1,3,3-trimethyl-6-aza-bicyclo[3.2.1]oct-6-yl)-methanone

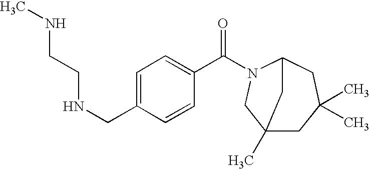

To a ice-water cooled solution of the above tert-butyl ester (300 mg, 0.696 mmol), TEA (0.3 mL, 2.3 mmol) in DCM (40 mL) was added with stirring methanesulfonyl chloride (135 μL, 1.72 mmol) and the mixture was stirred for 1 h at ambient temperature. Methylamine (5 mL, 33% in EtOH) was added and the mixture was stirred for 16 h at ambient temperature. The solvent was evaporated and to the residue was added water (50 m) and AcOEt (50 mL). The organic phase was separated and evaporated and to the residue was added DCM (20 mL) followed by TFA (10 mL). The resulting mixture was stirred for 4 h at ambient temperature and the solvent evaporated. The residue was purified using preparative HPLC (Method Z4): Amount isolated=175 mg (44%) of {4-[(2-methylamino-ethylamino)-methyl]-phenyl}-(1,3,3-trimethyl-6-aza-bicyclo[3.2.1]oct-6-yl)-methanone as an oil.

¹H NMR (400 MHz, CDCl₃) δ 0.93 (d, 3H), 1.02 (d, 3H), 1.12 (d, 3H), 1.15-1.46 (m, 3H), 1.56 (m, 1H), 1.75 (m, 1H), 2.16 (d, 0.5H), 2.25 (m, 0.5H), 2.42 (s, 3H), 2.74 (m, 4H), 3.15-3.29 (m, 1.5H), 3.60 (d, 0.5H), 3.82 (d, 2H), 3.98 (m, 0.5H), 4.60 (m, 0.5H), 7.38 (m, 4H). HPLC-MS (Method Z1): m/z=344 (M+1); tᵣ=1.09 min.

### Step C

To a solution of the above amide (50 mg, 0.146 mmol) in DCM (10 mL) was added phosgene (0.1 mL, 0.29 mmol, 30% in toluene) and the mixture was stirred for 30 min. at ambient temperature. The solvent was evaporated and the residue was purified using preparative HPLC (Method Z4): Amount isolated=11 mg (21%) of the title compound as an oil.

¹H NMR (400 MHz, CDCl₃) δ 0.93 (d, 3H), 1.02 (d, 3H), 1.13 (d, 3H), 1.15-1.60 (m, 4.5H), 1.75 (m, 1.5H), 2.23 (m, 0.5H), 2.84 (s, 3H), 3.16 (m, 2H), 3.28 (m, 3H), 3.58 (d, 0.5H), 3.97 (t, 0.5H), 4.39 (d, 2H), 4.60 (m, 0.5H), 7.29 (dd, 2H), 7.40 (t, 2H). HPLC-MS (Method Z1): m/z=370 (M+1); tᵣ=1.71 min.

The following compound was made as outlined in general method E above.

| | | | | LC/ |
|---|---|---|---|---|
| Ex. | Structure | MW | IUPAC Name | MS |
| 4-2 | 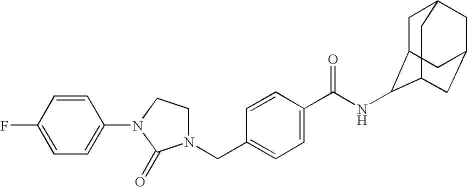 | 447.56 | N-Adamantan-2-yl-4-[3-(4- fluoro-phenyl)-2-oxo- imidazolidin-1-ylmethyl]- benzamide | 448 |
| 4-3 | 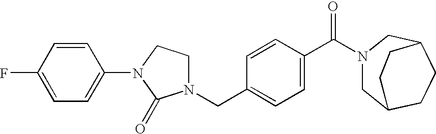 | 421.52 | 1-[4-(3-Aza-bicyclo[3.2.2]- nonane-3-carbonyl)- benzyl]-3-(4-fluoro-phenyl)- imidazolidin-2-one | 422 |
| 4-4 | 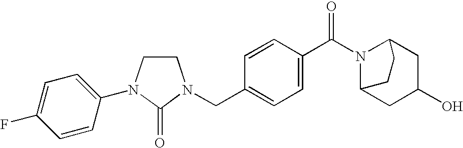 | 423.49 | 1-(4-Fluoro-phenyl)-3-[4-(3- hydroxy-8-aza-bicyclo- [3.2.1]octane-8-carbonyl)- benzyl]-imidazolidin-2-one | 424 |
| 4-5 | 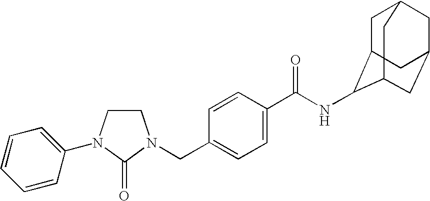 | 429.57 | N-Adamantan-2-yl-4-(2- oxo-3-phenyl-imidaozlidin- 1-ylmethyl)-benzamide | 430 |

### Example 5-1

### General Procedure (F)

### [4-(1,1-Dioxo-isothiazolidin-2-ylmethyl)-phenyl]-(1,3,3-trimethyl-6-aza-bicyclo[3.2.1]oct-6-yl)-methanone

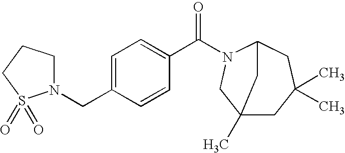

To a mixture of (4-aminomethyl-phenyl)-(1,3,3-trimethyl-6-aza-bicyclo[3.2.1]oct-6-yl)-methanone (100 mg, 0.349 mmol), TEA (100 μL, 0.698 mmol) and DCM (20 mL) was added 3-chloro-propane-1-sulfonyl chloride (51 μL, 0.419 mmol) and the mixture was stirred for 2 h at ambient temperature. The solvent was evaporated and the residues dissolved in dry THF (20 mL). Sodium hydride (25 mg, 1.05 mmol, 60% in mineral oil) was added and the mixture stirred for 90 min. at reflux temperature. The cooled mixture was quenched with water (100 μL) and evaporated. The residue was purified using preparative HPLC (Method Z4): Amount isolated=39 mg (29%) of the title compound as an oil

¹H NMR (400 MHz, CDCl₃) δ 0.93 (d, 3H), 1.03 (d, 3H), 1.13 (d, 3H), 1.17-1.60 (m, 4.5H), 1.71-1.79 (m, 2H), 2.23 (m, 0.5H), 2.32 (m, 2H), 3.09-3.29 (m, 4.5H), 3.60 (d, 0.5H), 3.97 (t, 0.5H), 4.20 (d, 2H), 4.61 (m, 0.5H), 7.38-7.45 (m, 4H). HPLC-MS (Method Z1): m/z=391 (M+1); tᵣ=1.8 min.

### Example 6-1

### General Procedure (G)

### [4-(1,1-Dioxo-2H-1,2,5-thiadiazolidin-2-ylmethyl)-phenyl]-(1,3,3-trimethyl-6-aza-bicyclo[3.2.1]oct-6-yl)-methanone

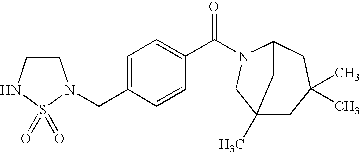

### Step A

### 1-tert-Butyloxycarbonyl-3-[4-(1,3,3-trimethyl-6-aza-bicyclo[3.2.1]octane-6-carbonyl)-benzyl]-sulfamide

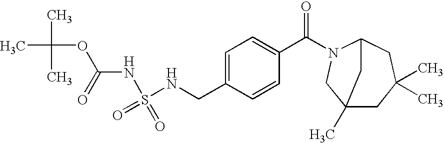

To an ice water cooled solution of chlorosulfonyl isocyanate (360 μL, 4.19 mmol) in DCM (20 mL) was added tert-butanol (400 μL, 4.19 mmol) and the mixture was stirred for 30 min. at ambient temperature. The resulting mixture was added to a solution of (4-aminomethyl-phenyl)-(1,3,3-trimethyl-6-aza-bicyclo[3.2.1]oct-6-yl)-methanone (1.2 g, 4.19 mmol), TEA (1.7 mL, 12.57 mmol) in DCM (25 mL) at 0° C. The mixture was stirred for 2 h allowing it to reach room temperature before it was washed with water (25 mL), dried (Na₂SO₄) and evaporated which afforded 1.8 g (92%) of 1-tert-butyloxycarbonyl-3-[4-(1,3,3-trimethyl-6-aza-bicyclo[3.2.1]octane-6-carbonyl)-benzyl]-sulfamide as an oil.

¹H NMR (400 MHz, CDCl₃) δ 0.93 (d, 3H), 1.03 (s, 3H), 1.12 (s, 3H), 1.15-1.59 (m, 13.5H), 1.76 (m, 1H), 2.23 (m, 0.5H), 3.13-3.28 (m, 1.5H), 3.58 (d, 0.5H), 3.96 (m, 0.5H), 4.11 (s, 2H), 4.59 (m, 0.5H), 5.75 (bs, 1H), 7.39 (m, 4H), 9.0 (bs, 1H). HPLC-MS (Method Z1): m/z=466 (M+1); tᵣ=1.98 min.

### Step B

### 1,1-Dioxo-5-[4-(1,3,3-trimethyl-6-aza-bicyclo[3.2.1]octane-6-carbonyl)-benzyl]-1,2,5-thiadiazolidine-2-carboxylic Acid Tert-butyl Ester

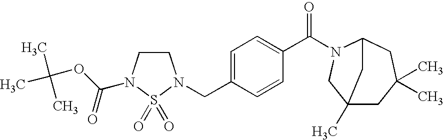

To a mixture of 1-tert-butyloxycarbonyl-3-[4-(1,3,3-trimethyl-6-aza-bicyclo[3.2.1]octane-6-carbonyl)-benzyl]-sulfamide (0.8 g, 1.72 mmol), 2-chloro-ethanol (130 μL, 1.89 mmol), triphenylphosphine (0.7 g, 2.58 mmol) in THF (50 mL) was added DIAD (0.5 mL, 2.58 mmol) and the mixture stirred for 1 h. The solvent was evaporated and the residue purified using silicagel column chromatography (Flash 40) and a mixture of AcOEt-Heptane (1:1) as eluent afforded crude 1-tert-butyloxycarbonyl-1-(2-hydroxy ethyl)-3-[4-(1,3,3-trimethyl-6-aza-bicyclo[3.2.1]octane-6-carbonyl)-benzyl]-sulfamide which was used in the next step without further purification.

HPLC-MS (Method Z1): m/z=528 (M+1).

To a solution of the above 1-tert-butyloxycarbonyl-1-(2-hydroxy ethyl)-3-[4-(1,3,3-trimethyl-6-aza-bicyclo[3.2.1]octane-6-carbonyl)-benzyl]-sulfamide in DMSO (15 mL) was added K₂CO₃ (0.36 g, 2.58 mmol) and the mixture was stirred for 2 at ambient temperature. Water (25 mL) was added and the resulting mixture was extracted with AcOEt (2×25 mL). The combined organic phases were evaporated and the residue purified using silicagel column chromatography (Flash 40) and first a mixture of AcOEt-Heptane (1:1) followed by a mixture of AcOEt-Heptane (4:1) as eluents which afforded 250 mg (30%) of 1,1-dioxo-5-[4-(1,3,3-trimethyl-6-aza-bicyclo[3.2.1]octane-6-carbonyl)-benzyl]-1,2,5-thiadiazolidine-2-carboxylic acid tert-butyl ester as an oil.

HPLC-MS (Method Z1): m/z=492 (M+1); tᵣ=2.24 min (100% ELS).

### Step C

### [4-(1,1-Dioxo-[1,2,5]thiadiazolidin-2-ylmethyl)-phenyl]-(1,3,3-trimethyl-6-aza-bicyclo[3.2.1]oct-6-yl)-methanone

To a solution of 1,1-dioxo-5-[4-(1,3,3-trimethyl-6-aza-bicyclo[3.2.1]octane-6-carbonyl)-benzyl]-1,2,5-thiadiazolidine-2-carboxylic acid tert-butyl ester (0.25 g, 0.51 mmol) in DCM (10 mL) was added TFA (5 mL) and the mixture was stirred for 16 at ambient temperature. The mixture was evaporated and the residue purified using preparative HPLC (Method Z4): Amount isolated=190 mg (95%) of the title compound as an oil

¹H NMR (400 MHz, CDCl₃) δ 0.94 (d, 3H), 1.04 (s, 3H), 1.13 (s, 3H), 1.18-1.60 (m, 4.5H), 1.77 (m, 1H), 2.23 (m, 0.5H), 3.15-3.30 (m, 3.5H), 3.44 (t, 2H), 3.60 (d, 0.5H), 3.97 (t, 0.5H), 4.18 (m, 2H), 4.61 (m, 0.5H), 5.01 (bs, 1H), 7.43 (m, 4H). HPLC-MS (Method Z1): m/z=392 (M+1); tᵣ=1.73 min (100% ELS).

### Example 6-2

### General Procedure (G)

### [4-(5-Methyl-1,1-dioxo-1,2,5-thiadiazolidin-2-ylmethyl)-phenyl]-(1,3,3-trimethyl-6-aza-bicyclo[3.2.1]oct-6-yl)-methanone

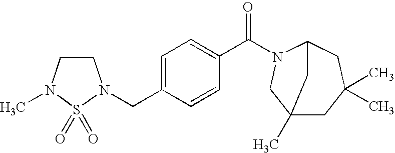

To a mixture of [4-(1,1-dioxo-2H-1,2,5-thiadiazolidin-2-ylmethyl)-phenyl]-(1,3,3-trimethyl-6-aza-bicyclo[3.2.1]oct-6-yl)-methanone (60 mg, 0.15 mmol), K₂CO₃ (30 mg, 0.31 mmol) in DMSO (4 mL) was added iodomethane (13 μL, 0.31 mmol). The mixture was stirred for 1 h at ambient temperature and evaporated. The residue was purified using preparative HPLC (Method Z4): Amount isolated=45 mg (73%) of the title compound as an oil

¹H NMR (400 MHz, CDCl₃) δ 0.94 (d, 3H), 1.04 (s, 3H), 1.13 (s, 3H), 1.18-1.61 (m, 4.5H), 1.77 (m, 1H), 2.23 (m, 0.5H), 2.79 (s, 3H), 3.15-3.20 (m, 2H), 3.23-3.30 (m, 3.5H), 3.61 (d, 0.5H), 3.98 (t, 0.5H), 4.24 (d, 2H), 4.61 (m, 0.5H), 7.42 (m, 4H). HPLC-MS (Method Z1): m/z=406 (M+1); tᵣ=1.86 min (100% ELS).

The following compounds were made as outlined in general method G above.

| | | | | LC/ |
|---|---|---|---|---|
| Ex. | Structure | MW | IUPAC Name | MS |
| 6-3 | 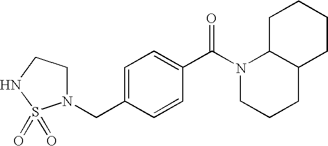 | 377.5 | (Octahydro-quinolin-1-yl)- [4-(1,1-dioxo-1,2,5-thia- diazolidin-2-ylmethyl)- phenyl]-methanone | 378 |
| 6-4 | 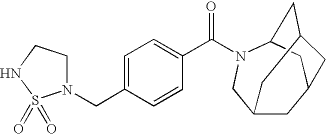 | 389.5 | (4-Aza-tricyclo[4.3.1.1^{3,8}]- undec-4-yl)-[4-(1,1-dioxo- 1,2,5-thiadiazolidin-2-yl- methyl)-phenyl]-methanone | 390 |
| 6-5 | 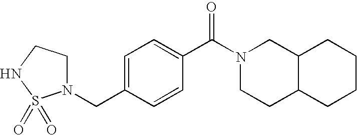 | 377.5 | (Octahydro-isoquinolin-2- yl)-[4-(1,1-dioxo-1,2,5- thiadiazolidin-2-ylmethyl)- phenyl]-methanone | 378 |
| 6-6 | 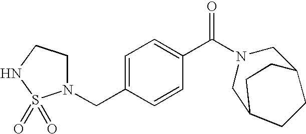 | 363.5 | (3-Aza-bicyclo[3.2.2]non-3- yl)-[4-(1,1-dioxo-1,2,5- thiadiazolidin-2-ylmethyl)- phenyl]-methanone | 364 |
| 6-7 | 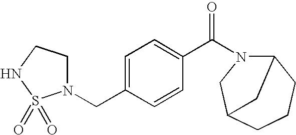 | 349.5 | (6-Aza-bicyclo[3.2.1]oct-6- yl)-[4-(1,1-dioxo-1,2,5- thiadiazolidin-2-ylmethyl)- phenyl]-methanone | 350 |
| 6-8 | 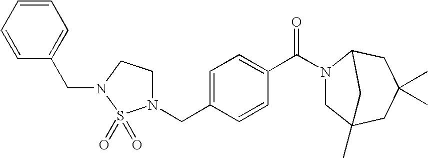 | 481.6 | [4-(-5-Benzyl-1,1-dioxo- 1,2,5-thiadiazolidin-2- ylmethyl)-phenyl]-(1,3,3- trimethyl-6-aza-bicyclo- [3.2.1]oct-6-yl)-methanone | 482 |
| 6-9 | 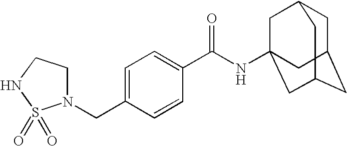 | 389.52 | N-Adamantan-1-yl-4-(1,1- dioxo--[1,2,5]thiadiazolidin- 2-ylmethyl)-benzamide | 390 |
| 6-10 | 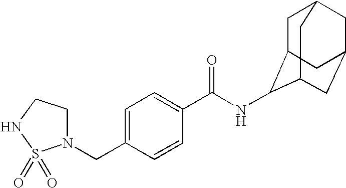 | 389.52 | N-Adamantan-2-yl-4-(1,1- dioxo--[1,2,5]thiadiazolidin- 2-ylmethyl)-benzamide | 390 |
| 6-11 | 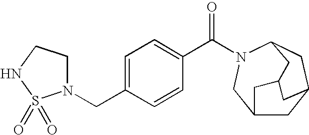 | 389.52 | (4-Azatricyclo[4.3.1.1*3,8*]- undec-4-yl)-[4-(1,1-dioxo- [1,2,5]thiadiazolidin-2-yl- methyl)-phenyl]-methanone | 390 |
| 6-12 | 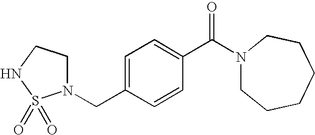 | 337.44 | Azepan-1-yl-[4-(1,1-dioxo- [1,2,5]thiadiazolidin-2-yl- methyl)-phenyl]-methanone | 338 |
| 6-13 | 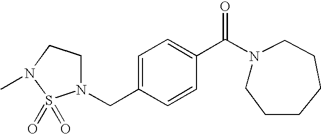 | 351.47 | Azepan-1-yl-[4-(5-methyl- 1,1-dioxo- [1,2,5]thiadiazolidin-2- ylmethyl)-phenyl]- methanone | 352 |
| 6-14 | 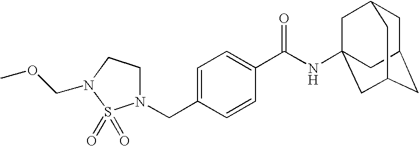 | 433.57 | N-Adamantan-1-yl-4-(5- methoxymethyl-1,1-dioxo- [1,2,5]thiadiazolidin-2- ylmethyl)-benzamide | 434 |
| 6-15 | 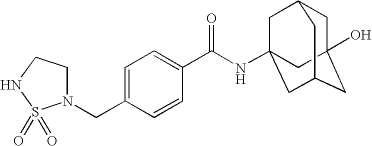 | 405.52 | 4-(1,1-Dioxo-[1,2,5]thia diazolidin-2-ylmethyl)-N-(3- hydroxy-adamantan-1-yl)- benzamide | 406 |
| 6-16 | 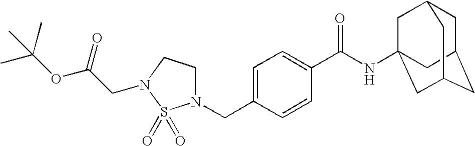 | 503.67 | {5-[4-(Adamantan-1-yl- carbamoyl)-benzyl]-1,1- dioxo-[1,2,5]thiadiazolidin- 2-yl}-acetic acid tert-butyl ester | 504 |

### Pharmacological Methods

11βHSD1 Enzyme Assay

Materials

3H-cortisone and anti-rabbit Ig coated scintillation proximity assay (SPA) beads were purchased from Amersham Pharmacia Biotech, P-NADPH was from Sigma and rabbit anticortisol antibodies were from Fitzgerald. An extract of yeast transformed with h-11βHSD1 (Hult et al., FEBS Lett., 441, 25 (1998)) was used as the source of enzyme. The test compounds were dissolved in DMSO (10 mM). All dilutions were performed in a buffer containing 50 mM TRIS-HCl (Sigma Chemical Co), 4 mM EDTA (Sigma Chemical Co), 0.1% BSA (Sigma Chemical Co), 0.01% Tween-20 (Sigma Chemical Co) and 0.005% bacitracin (Novo Nordisk A/S), pH=7.4. Optiplate 96 wells plates were supplied by Packard. The amount of 3H-cortisol bound to the SPA beads was measured on TopCount NXT, Packard.

Methods

h-11βHSD1, 120 nM 3H-cortisone, 4 mM β3-NADPH, antibody (1:200), serial dilutions of test compound and SPA particles (2 mg/well) were added to the wells. The reaction was initiated by mixing the different components and was allowed to proceed under shaking for 60 min at 30° C. The reaction was stopped be the addition of 10 fold excess of a stopping buffer containing 500 μM carbenoxolone and 1 μM cortisone. Data was analysed using GraphPad Prism software.

TABLE 1
Inhibition of 11βHSD1 by compounds of the invention

| | h-11βHSD1 |
|---|---|
| | IC₅₀ values |
| Example No. | (nM) |
| 1-28 | 19 |
| 2-5 | 10 |
| 3-8 | 500 |

While the invention has been described and illustrated with reference to certain pre-ferred embodiments thereof, those skilled in the art will appreciate that various changes, modifications, and substitutions can be made therein without departing from the spirit and scope of the present invention. Likewise, the specific pharmacological responses observed may vary according to and depending on the particular active compound selected or whether there are present pharmaceutical carriers, as well as the type of formulation and mode of administration employed, and such expected variations or differences in the results are contemplated in accordance with the objects and practices of the present invention. Accordingly, the invention is not to be limited as by the appended claims.

The features disclosed in the foregoing description and/or in the claims may both separately and in any combination thereof be material for realising the invention in diverse forms thereof.

Preferred Features of the Invention:

1. A substituted amide, a prodrug thereof, or a salt thereof with a pharmaceutically acceptable acid or base, or any optical isomer or mixture of optical isomers, including a racemic mixture or any tautomeric forms, wherein the compound is of formula I:

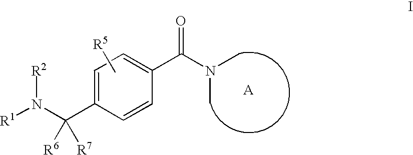

wherein:
R¹ is selected from H, R⁸(C═O)—, R⁹S(O)ₙ—, R¹⁰R¹¹NC(═Y)—, and R¹⁰R¹¹NS(O)ₙ—;
R² is selected from H, C₁-C₆alkyl, and C₃-C₆cycloalkyl;
alternatively, R¹ and R², together with the nitrogen to which they are attached, form a 3-12 membered saturated or partially saturated monocyclic or bicyclic ring consisting of the shown nitrogen, 2-10 carbon atoms, and 0-2 additional heteroatoms selected from nitrogen, oxygen, and S(O)ₘ, wherein this ring is substituted with 0-3 groups selected from C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸;
Ring A is a 5-12 membered saturated or partially saturated bicyclic or tricyclic ring consisting of the shown nitrogen, 4-10 carbon atoms and from 0 to 2 additional heteroatoms selected from nitrogen, oxygen, and S(O)ₘ;
Ring A is substituted with 0-3 groups selected from C₁-C₈alkyl, halo, OH, oxo, cyano, C₁-C₆alkyloxy, C₁-C₆alkyloxyC₁-C₆alkyl or C₁-C₆alkylcarbonyl, wherein each alkyl group is substituted with 0-3 R¹⁸;
R⁵ is selected from H, C₁-C₆alkyl, C₃-C₆cycloalkyl, halo, OH, and cyano;
R⁶ and R⁷ are independently selected from H, C₁-C₆alkyl, F, trihalomethyl, and trihalomethoxy;
alternatively, R⁶ and R⁷, together with the carbon atom to which they are attached, form a 3-8 membered saturated or partially saturated monocyclic ring consisting of the shown carbon atom, 2-5 additional carbon atoms, and 0-2 heteroatoms selected from nitrogen, oxygen, and S(O)ₘ, wherein this ring is substituted with 0-3 groups selected from halo, trihalomethyl, OH, C₁-C₆alkyl, oxo, and C₁-C₆alkyloxy;
R⁸ is selected from C₁-C₈alkyl, C₂-C₈alkenyl, aryl, hetaryl, arylC₁-C₆alkyl, hetarylC₁-C₆alkyl, C₃-C₁₀cycloalkyl, 3-10 membered hetcycloalkyl, aryloxyC₁-C₆alkyl, hetaryloxyC₁-C₆alkyl, arylC₁-C₆alkyloxyC₁-C₆alkyl, and hetarylC₁-C₆alkyloxyC₁-C₆alkyl, wherein each of the alkyl/alkyl, alkenyl, aryl, hetaryl, cycloalkyl, and hetcycloalkyl groups are independently substituted with 0-3 R¹⁹;
R⁹ is selected from C₁-C₈alkyl, C₂-C₈alkenyl, aryl, hetaryl, arylC₁-C₆alkyl, hetarylC₁-C₆alkyl, C₃-C₁₀cycloalkyl, 3-10 membered hetcycloalkyl, aryloxyC₁-C₆alkyl, and arylC₁-C₆alkyloxyC₁-C₆alkyl, wherein each of the alkyl/alkyl, alkenyl, aryl, hetaryl, cycloalkyl, and hetcycloalkyl groups are independently substituted with 0-3 R²⁰;
R¹⁰ and R¹¹ are independently selected from H, C₁-C₈alkyl, C₃-C₁₀cycloalkyl, 3-10 membered hetcycloalkyl, aryl, hetaryl, arylC₁-C₆alkyl, and hetarylC₁-C₆alkyl, wherein each of the alkyl/alkyl, cycloalkyl, hetcycloalkyl, aryl, and hetaryl groups are independently substituted with 0-3 R²¹;
alternatively, R¹⁰ and R¹¹, together with the nitrogen to which they are attached, form a 5-12 membered saturated or partially saturated monocyclic, bicyclic, or tricyclic ring consisting of the shown nitrogen atom, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkyl, hetarylC₁-C₆alkyl, hydroxy, oxo, COOH, C₁-C₆alkyloxy, arylC₁-C₆alkyloxy, hetarylC₁-C₆alkyloxy, C₁-C₆alkyloxyC₁-C₆alkyl, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkyl-carboxy, and hetarylC₁-C₆alkylcarboxy;
R¹² is selected from OH, C₁-C₈alkyl, C₃-C₁₀cycloalkyl, 3-10 membered hetcycloalkyl, trihalomethyl, C₁-C₈alkyloxy, aryl, arylC₁-C₆alkyl, hetaryl, hetarylC₁-C₆alkyl, aryloxy, hetaryloxy, and NR¹³R¹⁴;
R¹³ and R¹⁴ are independently selected from H, C₁-C₈alkyl, C₃-C₁₀cycloalkyl, aryl, hetaryl, arylC₁-C₆alkyl, and hetarylC₁-C₆alkyl, wherein each of the alkyl/alkyl, cycloalkyl, aryl, and hetaryl groups are independently substituted with 0-3 R²²;
alternatively, R¹³ and R¹⁴, together with the nitrogen to which they are attached, form a 5-12 membered saturated or partially saturated monocyclic, bicyclic, or tricyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkyl, hetarylC₁-C₆alkyl, OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkylcarboxy, and hetarylC₁-C₆alkylcarboxy;
R¹⁵ is selected from H, C₁-C₆alkyl, and C₃-C₆cycloalkyl;
R¹⁶ and R¹⁷ are independently selected from H, C₁-C₈alkyl, C₃-C₁₀cycloalkyl, halo, OH, cyano, —C(═O)R¹², —S(O)ₙR¹², —S(O)ₙNR¹³R¹⁴, —N(R¹³)S(O)ₙR¹², C₁-C₈alkyl, aryl, and hetaryl, wherein the alkyl and cycloalkyl groups are independently substituted with 0-3 R²²;
R¹³ is selected from halo, OH, oxo, COOH, cyano C₁-C₆alkyloxy, C₃-C₁₀cycloalkyloxy, aryloxy, hetaryloxy, hetarylthio and arylC₁-C₆alkyloxy;
R¹⁹, R²⁰ and R²¹ are independently selected from H, halo, OH, oxo, cyano, C₁-C₈alkyl, C₃-C₁₀cycloalkyl, 3-10 membered hetcycloalkyl, trihalomethyl, trihalomethyloxy, methylendioxo, dihalo-methylenedioxo, C₃-C₆spirocycloalkyl, C₁-C₆alkyloxy, aryl, hetaryl, arylC₁-C₆alkyl, hetarylC₁-C₆alkyl, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, and —C(═NR¹⁶)NR¹⁷;
R²² is selected from H, OH, oxo, halo, cyano, nitro, C₁-C₆alkyl, C₁-C₆alkyloxy, NR²³R²⁴, methylendioxo, dihalomethylendioxo, trihalomethyl, and trihalomethyloxy;
R²³ and R²⁴ are independently selected from H, C₁-C₈alkyl, and arylC₁-C₆alkyl;
m is selected from 0, 1, and 2;
n is selected from 1 and 2;
Y is selected from O and S;
or a salt thereof with a pharmaceutically acceptable acid or base, or any optical isomer or mixture of optical isomers, including a racemic mixture, or any tautomeric forms.
2. A compound of clause 1 wherein:
R¹ is selected from R⁸(C═O)—, R⁹S(O)₂—, R¹⁰R¹¹NC(═O)—, and R¹⁰R¹¹NS(O)₂—;
R² is C₁-C₄alkyl;
alternatively, R¹ and R², together with the nitrogen to which they are attached, form a 5-6 membered saturated ring consisting of the shown nitrogen, 2-4 carbon atoms, and 0-2 additional heteroatoms selected from nitrogen, oxygen, and S(O)ₘ, wherein this ring is substituted with 0-2 groups selected from C₁-C₈alkyl, aryl, hetaryl, arylC₁-C₆alkyl, hetarylC₁-C₆alkyl, —C(═O)R¹², —S(O)ₙR¹², —S(═O)ₙNR¹³R¹⁴, —N(R¹³)S(O)ₙR¹², OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸;
Ring A is an 8-11 membered saturated or partially saturated bicyclic or tricyclic ring consisting of the shown nitrogen, 5-10 carbon atoms and from 0 to 1 additional heteroatoms selected from nitrogen, oxygen, and S(O)ₘ;
Ring A is substituted with 0-3 groups selected from C₁-C₄alkyl, halo, OH, oxo, cyano, C₁-C₄alkyloxy, C₁-C₄alkyloxyC₁-C₄alkyl or C₁-C₄alkylcarbonyl, wherein each alkyl/alkyl group is substituted with 0-1 R¹⁸;

### R⁵ is H;

R⁶ and R⁷ are independently selected from H and C₁-C₄alkyl; and,
n is 2.
3. A compound of clause 1 wherein:
R⁸ is selected from C₁-C₆alkyl, C₂-C₆alkenyl, aryl, hetaryl, arylC₁-C₄alkyl, hetarylC₁-C₄alkyl, C₃-C₆cycloalkyl, 3-6 membered hetcycloalkyl, aryloxyC₁-C₄alkyl, and hetaryloxyC₁-C₄alkyl, wherein each of the alkyl/alkyl, alkenyl, aryl, hetaryl, cycloalkyl, and hetcycloalkyl groups are independently substituted with 0-2 R¹⁹;
R⁹ is selected from C₁-C₆alkyl, C₂-C₆alkenyl, aryl, hetaryl, arylC₁-C₄alkyl, hetarylC₁-C₄alkyl, C₃-C₆cycloalkyl, 3-6 membered hetcycloalkyl, and aryloxyC₁-C₄alkyl, wherein each of the alkyl/alkyl, alkenyl, aryl, hetaryl, cycloalkyl, and hetcycloalkyl groups are independently substituted with 0-2 R²⁰;
R¹⁰ and R¹¹ are independently selected from H, C₃-C₆cycloalkyl, 3-6 membered hetcycloalkyl, aryl, and hetaryl, wherein each of the cycloalkyl, hetcycloalkyl, aryl, and hetaryl groups are independently substituted with 0-3 R²¹;
alternatively, R¹⁰ and R¹¹, together with the nitrogen to which they are attached, form a 5-6 membered saturated or partially saturated monocyclic ring consisting of the shown nitrogen atom, 4-5 carbon atoms, and 0-1 additional heteroatoms selected from nitrogen, oxygen, and S(O)ₘ, wherein this ring is substituted with 0-2 groups selected from C₁-C₈alkyl, aryl, hetaryl, hydroxy, oxo, COOH, C₁-C₆alkyloxy, arylC₁-C₆alkyloxy, hetarylC₁-C₆alkyloxy, and C₁-C₆alkylcarbonyl;
R¹² is selected from OH, C₁-C₄alkyl, C₃-C₆cycloalkyl, 3-10 membered hetcycloalkyl, trihalomethyl, C₁-C₄alkyloxy, aryl, arylC₁-C₄alkyl, hetaryl, hetarylC₁-C₄alkyl, aryloxy, and hetaryloxy;
R¹⁹, R²⁰ and R²¹ are independently selected from H, halo, OH, oxo, cyano, C₁-C₆alkyl, C₃-C₆cycloalkyl, 3-6 membered hetcycloalkyl, trihalomethyl, trihalomethyloxy, dihalo-methylenedioxo, C₁-C₄alkyloxy, aryl, hetaryl, arylC₁-C₄alkyl, hetarylC₁-C₄alkyl, —C(═O)R¹, —S(O)ₙR¹², and —S(O)ₙNR¹³R¹⁴; and,
n is 2.
4. A compound of clause 1 wherein the compound is of formula Ia:

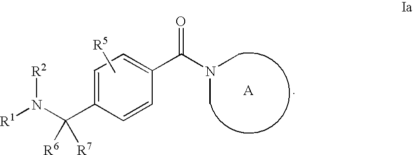

5. A compound of clause 1 wherein the compound is of formula Ib:

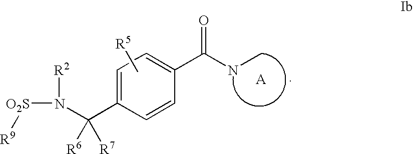

6. A compound of clause 1 wherein the compound is of formula Ic:

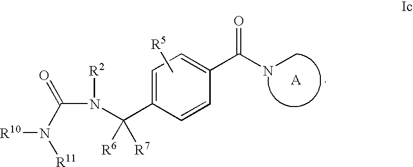

7. A compound of clause 1 wherein the compound is of formula Id:

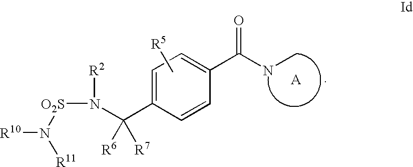

8. A compound of clause 1 wherein
R¹ and R², together with the nitrogen to which they are attached, form a 5-12 membered saturated or partially saturated monocyclic or bicyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸.
9. A compound of clause 1 wherein:
Ring A is selected from:

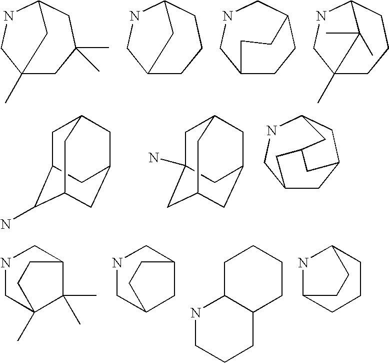

Ring A is substituted with 0-2 R²⁵; and,
R²⁵ is selected from C₁-C₈alkyl, halo, hydroxy, oxo, cyano, C(═O)R¹², and C₁-C₆alkyloxy, wherein R¹² is as defined above.
10. A compound of clause 1 wherein:
Ring A is selected from:

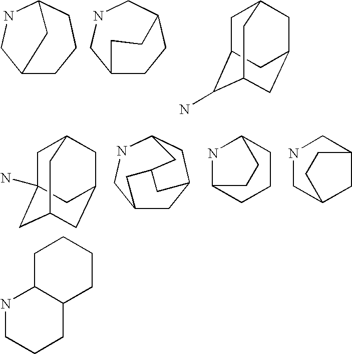

Ring A is substituted with 0-2 R²⁵; and,
R²⁵ is selected from C₁-C₈alkyl, halo, hydroxy, oxo, cyano, and C₁-C₆alkyloxy.
11. A compound of clause 1 wherein:
ring A is

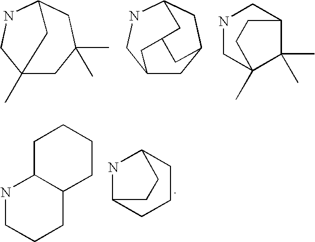

Ring A is substituted with 0-2 R²⁵; and,
R²⁵ is selected from C₁-C₈alkyl, halo, hydroxy, oxo, cyano, and C₁-C₆alkyloxy.
12. A compound of clause 1 wherein the compound is selected from the group:
N-Methyl-N-[4-(1,3,3-trimethyl-6-aza-bicyclo[3.2.1]octane-6-carbonyl)-benzyl]-acetamide
N-Methyl-N-[4-(1,3,3-trimethyl-6-aza-bicyclo[3.2.1]octane-6-carbonyl)-benzyl]-isobutyramide
Cyclopentanecarboxylic acid methyl-[4-(1,3,3-trimethyl-6-aza-bicyclo[3.2.1]octane-6-carbonyl)-benzyl]-amide
Cyclohexanecarboxylic acid methyl-[4-(1,3,3-trimethyl-6-aza-bicyclo[3.2.1]octane-6-carbonyl)-benzyl]-amide
Piperidine-1-carboxylic acid methyl-[4-(1,3,3-trimethyl-6-aza-bicyclo[3.2.1]octane-6-carbonyl)-benzyl]-amide
N-Methyl-N-[4-(1,3,3-trimethyl-6-aza-bicyclo[3.2.1]octane-6-carbonyl)-benzyl]-benzamide
1-Acetyl-piperidine-4-carboxylic acid methyl-[4-(1,3,3-trimethyl-6-aza-bicyclo[3.2.1]octane-6-carbonyl)-benzyl]-amide
1-Acetyl-piperidine-3-carboxylic acid methyl-[4-(1,3,3-trimethyl-6-aza-bicyclo[3.2.1]octane-6-carbonyl)-benzyl]-amide
Cyclopentanecarboxylic acid ethyl-[4-(1,3,3-trimethyl-6-aza-bicyclo[3.2.1]octane-6-carbonyl)-benzyl]-amide
Morpholine-4-carboxylic acid methyl-[4-(1,3,3-trimethyl-6-aza-bicyclo[3.2.1]octane-6-carbonyl)-benzyl]-amide
2,2-N-Trimethyl-N-[4-(1,3,3-trimethyl-6-aza-bicyclo[3.2.1]octane-6-carbonyl)-benzyl]-propionamide
Tetrahydro-furan-3-carboxylic acid methyl-[4-(1,3,3-trimethyl-6-aza-bicyclo[3.2.1]octane-6-carbonyl)-benzyl]-amide
N-Methyl-4-trifluoromethoxy-N-[4-(1,3,3-trimethyl-6-aza-bicyclo[3.2.1]octane-6-carbonyl)-benzyl]-benzamide
Thiophene-2-carboxylic acid methyl-[4-(1,3,3-trimethyl-6-aza-bicyclo[3.2.1]octane-6-carbonyl)-benzyl]-amide
Furan-2-carboxylic acid methyl-[4-(1,3,3-trimethyl-6-aza-bicyclo[3.2.1]octane-6-carbonyl)-benzyl]-amide
3-Chloro-4-(propane-2-sulfonyl)-thiophene-2-carboxylic acid methyl-[4-(1,3,3-trimethyl-6-aza-bicyclo[3.2.1]octane-6-carbonyl)-benzyl]-amide
6-Chloro-N-methyl-N-[4-(1,3,3-trimethyl-6-aza-bicyclo[3.2.1]octane-6-carbonyl)-benzyl]-nicotinamide
5-Methyl-isoxazole-3-carboxylic acid methyl-[4-(1,3,3-trimethyl-6-aza-bicyclo[3.2.1]octane-6-carbonyl)-benzyl]-amide
3,3,N-Trimethyl-N-[4-(1,3,3-trimethyl-6-aza-bicyclo[3.2.1]octane-6-carbonyl)-benzyl]-butyramide
3-Cyano-N-methyl-N-[4-(1,3,3-trimethyl-6-aza-bicyclo[3.2.1]octane-6-carbonyl)-benzyl]-benzamide
N-Methyl-2-phenoxy-N-[4-(1,3,3-trimethyl-6-aza-bicyclo[3.2.1]octane-6-carbonyl)-benzyl]-acetamide
N-Methyl-N-[4-(1,3,3-trimethyl-6-aza-bicyclo[3.2.1]octane-6-carbonyl)-benzyl]-malonamic acid methyl ester
3-Methyl-but-2-enoic acid methyl-[4-(1,3,3-trimethyl-6-aza-bicyclo[3.2.1]octane-6-carbonyl)-benzyl]-amide
N-Methyl-2-phenyl-N-[4-(1,3,3-trimethyl-6-aza-bicyclo[3.2.1]octane-6-carbonyl)-benzyl]-acetamide
1-Trifluoromethyl-cyclobutanecarboxylic acid methyl-[4-(1,3,3-trimethyl-6-aza-bicyclo[3.2.1]octane-6-carbonyl)-benzyl]-amide
3,5-Dimethoxy-N-methyl-N-[4-(1,3,3-trimethyl-6-aza-bicyclo[3.2.1]octane-6-carbonyl)-benzyl]-benzamide
4-Methanesulfonyl-N-methyl-N-[4-(1,3,3-trimethyl-6-aza-bicyclo[3.2.1]octane-6-carbonyl)-benzyl]-benzamide
N-Methyl-3-trifluoromethoxy-N-[4-(1,3,3-trimethyl-6-aza-bicyclo[3.2.1]octane-6-carbonyl)-benzyl]-benzamide
2,2-Difluoro-1,3-benzodioxole-4-carboxylic acid methyl-[4-(1,3,3-trimethyl-6-aza-bicyclo[3.2.1]octane-6-carbonyl)-benzyl]-amide
N-Methyl-6-morpholin-4-yl-N-[4-(1,3,3-trimethyl-6-aza-bicyclo[3.2.1]octane-6-carbonyl)-benzyl]-nicotinamide
N-Methyl-4-(2,2,2-trifluoro-acetyl)-N-[4-(1,3,3-trimethyl-6-aza-bicyclo[3.2.1]octane-6-carbonyl)-benzyl]-benzamide
N-[4-(1,3,3-Trimethyl-6-aza-bicyclo[3.2.1]octane-6-carbonyl)-benzyl]-benzamide
N-Methyl-N-[4-(1,3,3-trimethyl-6-aza-bicyclo[3.2.1]octane-6-carbonyl)-benzyl]-isophthalamic acid
2,3-Dihydro-benzofuran-7-carboxylic acid methyl-[4-(1,3,3-trimethyl-6-aza-bicyclo[3.2.1]octane-6-carbonyl)-benzyl]-amide
3-Acetyl-N-methyl-N-[4-(1,3,3-trimethyl-6-aza-bicyclo[3.2.1]octane-6-carbonyl)-benzyl]-benzamide
1,1,3-Trimethyl-3-[4-(1,3,3-trimethyl-bicyclo[3.2.1]octane-6-carbonyl)-benzyl]-sulphonylurea
N-Methyl-N-[4-(1,3,3-trimethyl-6-aza-bicyclo[3.2.1]octane-6-carbonyl)-benzyl]-methanesulfonamide
2,2,2-Trifluoro-ethanesulfonic acid methyl-[4-(1,3,3-trimethyl-6-aza-bicyclo[3.2.1]octane-6-carbonyl)-benzyl]-amide
N-Methylphenyl-N-[4-(1,3,3-trimethyl-6-aza-bicyclo[3.2.1]octane-6-carbonyl)-benzyl]-methanesulfonamide
Trifluoro-N-isopropyl-N-[4-(1,3,3-trimethyl-6-aza-bicyclo[3.2.1]octane-6-carbonyl)-benzyl]-methanesulfonamide
N-Cyclopropyl-trifluoro-N-[4-(1,3,3-trimethyl-6-aza-bicyclo[3.2.1]octane-6-carbonyl)-benzyl]-methanesulfonamide
N-Ethyl-trifluoro-N-[4-(1,3,3-trimethyl-6-aza-bicyclo[3.2.1]octane-6-carbonyl)-benzyl]-methanesulfonamide
Trifluoro-N-methyl-N-[4-(1,3,3-trimethyl-6-aza-bicyclo[3.2.1]octane-6-carbonyl)-benzyl]-methanesulfonamide
3-Benzoyl-1-methyl-1-[4-(1,3,3-trimethyl-6-aza-bicyclo[3.2.1]octane-6-carbonyl)-benzyl]-urea
3-Cyclohexyl-1-methyl-1-[4-(1,3,3-trimethyl-6-aza-bicyclo[3.2.1]octane-6-carbonyl)-benzyl]-urea
3-(4-methyl-phenyl)sulfonyl-1-methyl-1-[4-(1,3,3-tri-methyl-6-aza-bicyclo[3.2.1]octane-6-carbonyl)-benzyl]-urea
1,3-Dimethyl-3-phenyl-1-[4-(1,3,3-trimethyl-6-aza-bicyclo[3.2.1]octane-6-carbonyl)-benzyl]-urea
3-(2,3-Dihydro-1,4-benzodioxin-2-ylmethyl)-1-methyl-1-[4-(1,3,3-trimethyl-6-aza-bicyclo[3.2.1]octane-6-carbonyl)-benzyl]-urea
3-(3-Methoxy-benzyl)-1-methyl-1-[4-(1,3,3-trimethyl-6-aza-bicyclo[3.2.1]octane-6-carbonyl)-benzyl]-urea
3-(1,1-Dioxo-tetrahydro-thiophen-3-yl)-1-methyl-1-[4-(1,3,3-trimethyl-6-aza-bicyclo[3.2.1]octane-6-carbonyl)-benzyl]-urea
1-Methyl-3-(tetrahydro-pyran-4-yl)-1-[4-(1,3,3-trimethyl-6-aza-bicyclo[3.2.1]-octane-6-carbonyl)-benzyl]-urea
{1,3-Dimethyl-3-[4-(1,3,3-trimethyl-6-aza-bicyclo[3.2.1]octane-6-carbonyl)-benzyl]-ureido}-acetic acid methyl ester
1-Methyl-3-(5-trifluoromethyl-1,3,4-thiadiazol-2-yl)-1-[4-(1,3,3-trimethyl-6-aza-bicyclo[3.2.1]octane-6-carbonyl)-benzyl]-urea
2-{3-Methyl-3-[4-(1,3,3-trimethyl-6-aza-bicyclo[3.2.1]octane-6-carbonyl)-benzyl]-ureido}-benzoic acid methyl ester
3-{3-Methyl-3-[4-(1,3,3-trimethyl-6-aza-bicyclo[3.2.1]octane-6-carbonyl)-benzyl]-ureido}-benzoic acid ethyl ester
1-Methyl-3-(3-ethylsulfanyl-phenyl)-1-[4-(1,3,3-trimethyl-6-aza-bicyclo-[3.2.1]-octane-6-carbonyl)-benzyl]-urea
1-Methyl-3-(4-ethylsulfanyl-phenyl)-1-[4-(1,3,3-trimethyl-6-aza-bicyclo-[3.2.1]-octane-6-carbonyl)-benzyl]-urea
3-(4-Benzyloxy-phenyl)-1-methyl-1-[4-(1,3,3-trimethyl-6-aza-bicyclo[3.2.1]octane-6-carbonyl)-benzyl]-urea
1-Methyl-3-(4-trifluoro-methylsulfanyl-phenyl)-1-[4-(1,3,3-trimethyl-6-aza-bicyclo[3.2.1]octane-6-carbonyl)-benzyl]-urea
3-(4-Acetyl-phenyl)-1-methyl-1-[4-(1,3,3-tri-methyl-6-aza-bicyclo[3.2.1]octane-6-carbonyl)-benzyl]-urea
3-(3-Acetyl-phenyl)-1-methyl-1-[4-(1,3,3-tri-methyl-6-aza-bicyclo[3.2.1]octane-6-carbonyl)-benzyl]-urea
3-(3-Cyano-phenyl)-1-methyl-1-[4-(1,3,3-tri-methyl-6-aza-bicyclo[3.2.1]octane-6-carbonyl)-benzyl]-urea
1-Methyl-3-(4-trifluoro-methyl-phenyl)-1-[4-(1,3,3-trimethyl-6-aza-bicyclo-[3.2.1]octane-6-carbonyl)-benzyl]-urea
3-(4-Methoxy-benzyl)-1-methyl-1-[4-(1,3,3-tri-methyl-6-aza-bicyclo[3.2.1]octane-6-carbonyl)-benzyl]-urea
1-Methyl-3-(2,2,4,4-tetrafluoro-4H-benzo-[1,3]dioxin-6-yl)-1-[4-(1,3,3-trimethyl-6-aza-bicyclo-[3.2.1]octane-6-carbonyl)-benzyl]-urea
1-Methyl-3-(4-trifluoro-methoxy-phenyl)-1-[4-(1,3,3-trimethyl-6-aza-bicyclo[3.2.1]-octane-6-carbonyl)-benzyl]-urea
1-Methyl-3-[4-(2,2,2-trifluoro-acetyl)-cyclohexyl]-1-[4-(1,3,3-trimethyl-6-aza-bicyclo[3.2.1]octane-6-carbonyl)-benzyl]-urea
1-(4-Acetyl-phenyl)-1,3-dimethyl-3-[4-(1,3,3-tri-methyl-6-aza-bicyclo[3.2.1]octane-6-carbonyl)-benzyl]-urea
1-Phenyl-3-{1-[4-(1,3,3-trimethyl-6-aza-bicyclo-[3.2.1]octane-6-carbonyl)-phenyl]-cyclopropyl}-urea
Piperidine-1-carboxylic acid methyl-[4-(octahydro-quinoline-1-carbonyl)-benzyl]-amide
Piperidine-1-carboxylic acid [4-(3-aza-bicyclo-[3.2.2]nonane-3-carbonyl)-benzyl]-methyl-amide
Morpholine-4-carboxylic acid methyl-[4-(octahydro-quinoline-1-carbonyl)-benzyl]-amide
Morpholine-4-carboxylic acid [4-(3-aza-bicyclo-[3.2.2]nonane-3-carbonyl)-benzyl]-methyl-amide
1,3-Dimethyl-3-[4-(octahydro-quinoline-1-carbonyl)-benzyl]-1-phenyl-urea
1-[4-(3-Aza-bicyclo-[3.2.2]nonane-3-carbonyl)-benzyl]-1,3-dimethyl-3-phenyl-urea
Piperidine-1-carboxylic acid [4-(6-aza-bicyclo-[3.2.1]octane-6-carbonyl)-benzyl]-methyl-amide
Piperidine-1-carboxylic acid methyl-[4-(1,8,8-trimethyl-3-aza-bicyclo[3.2.1]octane-3-carbonyl)-benzyl]-amide
Morpholine-4-carboxylic acid [4-(6-aza-bicyclo-[3.2.1]octane-6-carbonyl)-benzyl]-methyl-amide
Morpholine-4-carboxylic acid methyl-[4-(1,8,8-trimethyl-3-aza-bicyclo[3.2.1]-octane-3-carbonyl)-benzyl]-amide
1-[4-(6-Aza-bicyclo-[3.2.1]octane-6-carbonyl)-benzyl]-1,3-dimethyl-3-phenyl-urea
1,3-Dimethyl-1-phenyl-3-[4-(1,8,8-trimethyl-3-aza-bicyclo[3.2.1]octane-3-carbonyl)-benzyl]-urea
Piperidine-1-carboxylic acid [4-(3-hydroxy-8-aza-bicyclo[3.2.1]octane-8-carbonyl)-benzyl]-methyl-amide
Morpholine-4-carboxylic acid [4-(3-hydroxy-8-aza-bicyclo[3.2.1]octane-8-carbonyl)-benzyl]-methyl-amide
1-[4-(3-Hydroxy-8-aza-bicyclo[3.2.1]octane-8-carbonyl)-benzyl]-1,3-di-methyl-3-phenyl-urea
1-[4-(3-Fluoro-8-aza-bicyclo[3.2.1]octane-8-carbonyl)-benzyl]-1,3-di-methyl-3-phenyl-urea
Piperidine-1-carboxylic acid [4-(3-fluoro-8-aza-bicyclo[3.2.1]octane-8-carbonyl)-benzyl]-methyl-amide
Morpholine-4-carboxylic acid [4-(3-fluoro-8-aza-bicyclo[3.2.1]octane-8-carbonyl)-benzyl]-methyl-amide
N-Adamantan-2-yl-4-(1,3-dimethyl-3-pyridin-2-yl-ureidomethyl)-benzamide
1-[4-(3-Aza-bicyclo[3.2.2]nonane-3-carbonyl)-benzyl]-1,3-dimethyl-3-pyridin-2-yl-urea
1-[4-(3-Hydroxy-8-aza-bicyclo[3.2.1]octane-8-carbonyl)-benzyl]-1,3-dimethyl-3-pyridin-2-yl-urea
Morpholine-4-carboxylic acid [4-(adamantan-2-ylcarbamoyl)-benzyl]-methyl-amide
1-[4-(3-Aza-bicyclo[3.2.2]nonane-3-carbonyl)-benzyl]-1,3-dimethyl-3-thiazol-2-yl-urea
1,3-Dimethyl-3-[4-(2-oxa-5-aza-bicyclo[2.2.1]heptane-5-carbonyl)-benzyl]-1-thiazol-2-yl-urea
4-[3-(1-Acetyl-piperidin-4-yl)-1,3-dimethyl-ureidomethyl]-N-adamantan-2-yl-benzamide
1-(1-Acetyl-piperidin-4-yl)-3-[4-(3-aza-bicyclo[3.2.2]nonane-3-carbonyl)-benzyl]-1,3-dimethyl-urea
N-Adamantan-2-yl-4-(1,3-dimethyl-3-pyrimidin-2-yl-ureidomethyl)-benzamide
1-[4-(3-Aza-bicyclo[3.2.2]nonane-3-carbonyl)-benzyl]-1,3-dimethyl-3-pyrimidin-2-yl-urea
N-Adamantan-2-yl-4-(1,3-dimethyl-3-thiazol-2-yl-ureidomethyl)-benzamide
N-Adamantan-2-yl-4-(1,3-dimethyl-3-phenyl-ureidomethyl)-benzamide
1-[4-(3-Hydroxy-8-aza-bicyclo[3.2.1]octane-8-carbonyl)-benzyl]-1,3-dimethyl-3-pyrimidin-2-yl-urea
N-Adamantan-2-yl-4-[3-(4-hydroxy-cyclohexyl)-1,3-dimethyl-ureidomethyl]-benzamide
1-[4-(3-Aza-bicyclo[3.2.2]nonane-3-carbonyl)-benzyl]-3-(4-hydroxy-cyclohexyl)-1,3-dimethyl-urea
1-(4-Hydroxy-cyclohexyl)-1,3-dimethyl-3-[4-(2-oxa-5-aza-bicyclo[2.2.1]heptane-5-carbonyl)-benzyl]-urea
1-Methyl-3-[4-(1,3,3-trimethyl-6-aza-bicyclo[3.2.1]octane-6-carbonyl)-benzyl]-imidazolidin-2-one
[4-(1,1-Dioxo-isothiazolidin-2-ylmethyl)-phenyl]-(1,3,3-trimethyl-6-aza-bicyclo[3.2.1]-oct-6-yl)-methanone
[4-(1,1-Dioxo-1,2,5-thiadiazolidin-2-ylmethyl)-phenyl]-(1,3,3-trimethyl-bicyclo[3.2.1]oct-6-yl)-methanone
[4-(5-Methyl-1,1-dioxo-1,2,5-thiadiazolidin-2-ylmethyl)-phenyl]-(1,3,3-trimethyl-bicyclo[3.2.1]oct-6-yl)-methanone
(Octahydro-quinolin-1-yl)-[4-(1,1-dioxo-1,2,5-thiadiazolidin-2-ylmethyl)-phenyl]-methanone
(4-Aza-tricyclo[4.3.1.1^{3,8}]-undec-4-yl)-[4-(1,1-dioxo-1,2,5-thiadiazolidin-2-ylmethyl)-phenyl]-methanone
(Octahydro-isoquinolin-2-yl)-[4-(1,1-dioxo-1,2,5-thiadiazolidin-2-ylmethyl)-phenyl]-methanone
(3-Aza-bicyclo[3.2.2]non-3-yl)-[4-(1,1-dioxo-1,2,5-thiadiazolidin-2-ylmethyl)-phenyl]-methanone
(6-Aza-bicyclo[3.2.1]oct-6-yl)-[4-(1,1-dioxo-1,2,5-thiadiazolidin-2-ylmethyl)-phenyl]-methanone
[4-(5-Benzyl-1,1-dioxo-[1,2,5]thiadiazolidin-2-ylmethyl)-phenyl]-(1,3,3-trimethyl-6-aza-bicyclo[3.2.1]oct-6-yl)-methanone
   or a salt thereof with a pharmaceutically acceptable acid or base, or any optical isomer or mixture of optical isomers, including a racemic mixture, or any tautomeric forms.
   13. The compound according to any of the clauses 1-12, which is an agent useful for the treatment of conditions, disorders, or diseases wherein a modulation or an inhibition of the activity of 11βHSD1 is beneficial.
   14. The compound according to clause 13, wherein the conditions, disorders, and diseases that are influenced by intracellular glucocorticoid levels.
   15. The compound according to clause 13, wherein the conditions, disorders, or diseases are selected from metabolic syndrome, insulin resistance, dyslipidemia, hypertension, obesity, type 2 diabetes, impaired glucose tolerance (IGT), impaired fasting glucose (IFG), progression from IGT to type 2 diabetes, progression of metabolic syndrome into type 2 diabetes, diabetic late complications, neurodegenerative and psychiatric disorders, and the adverse effects of glucocorticoid receptor agonist treatment or therapy.
   16. A pharmaceutical composition comprising, as an active ingredient, at least one compound according to any one of the clauses 1-12 together with one or more pharmaceutically acceptable carriers or excipients.
   17. The pharmaceutical composition according to clause 16 which is suitable for oral, nasal, buccal, transdermal, pulmonal, or parenteral administration.
   18. Use of a substituted amide, a prodrug thereof, or a salt thereof with a pharmaceutically acceptable acid or base, or any optical isomer or mixture of optical isomers, including a racemic mixture or any tautomeric forms, wherein the substituted amide or a prodrug thereof is of formula I:

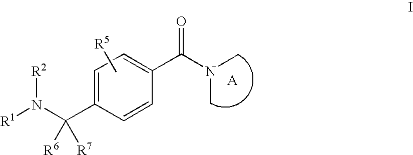

wherein:
R¹ is selected from H, R⁸(C═O)—, R⁹S(O)ₙ—, R¹⁰R¹¹NC(═Y)—, and R¹⁰R¹¹NS(O)ₙ—;
R² is selected from H, C₁-C₆alkyl, and C₃-C₆cycloalkyl;
alternatively, R¹ and R², together with the nitrogen to which they are attached, form a 3-12 membered saturated or partially saturated monocyclic or bicyclic ring consisting of the shown nitrogen, 2-10 carbon atoms, and 0-2 additional heteroatoms selected from nitrogen, oxygen, and S(O)ₘ, wherein this ring is substituted with 0-3 groups selected from C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸;
Ring A is a 5-12 membered saturated or partially saturated bicyclic or tricyclic ring consisting of the shown nitrogen, 4-10 carbon atoms and from 0 to 2 additional heteroatoms selected from nitrogen, oxygen, and S(O)ₘ;
Ring A is substituted with 0-3 groups selected from C₁-C₈alkyl, halo, OH, oxo, cyano, C₁-C₆alkyloxy, C₁-C₆alkyloxyC₁-C₆alkyl or C₁-C₆alkylcarbonyl, wherein each alkyl group is substituted with 0-3 R¹⁸;
R⁵ is selected from H, C₁-C₆alkyl, C₃-C₆cycloalkyl, halo, OH, and cyano;
R⁶ and R⁷ are independently selected from H, C₁-C₆alkyl, F, trihalomethyl, and trihalomethoxy;
alternatively, R⁶ and R⁷, together with the carbon atom to which they are attached, form a 3-8 membered saturated or partially saturated monocyclic ring consisting of the shown carbon atom, 2-5 additional carbon atoms, and 0-2 heteroatoms selected from nitrogen, oxygen, and S(O)ₘ, wherein this ring is substituted with 0-3 groups selected from halo, trihalomethyl, OH, C₁-C₆alkyl, oxo, and C₁-C₆alkyloxy;
R⁸ is selected from C₁-C₈alkyl, C₂-C₈alkenyl, aryl, hetaryl, arylC₁-C₆alkyl, hetarylC₁-C₆alkyl, C₃-C₁₀cycloalkyl, 3-10 membered hetcycloalkyl, aryloxyC₁-C₆alkyl, hetaryloxyC₁-C₆alkyl, arylC₁-C₆alkyloxyC₁-C₆alkyl, and hetarylC₁-C₆alkyloxyC₁-C₆alkyl, wherein each of the alkyl/alkyl, alkenyl, aryl, hetaryl, cycloalkyl, and hetcycloalkyl groups are independently substituted with 0-3 R¹⁹;
R⁹ is selected from C₁-C₈alkyl, C₂-C₈alkenyl, aryl, hetaryl, arylC₁-C₆alkyl, hetarylC₁-C₆alkyl, C₃-C₁₀cycloalkyl, 3-10 membered hetcycloalkyl, aryloxyC₁-C₆alkyl, and arylC₁-C₆alkyloxyC₁-C₆alkyl, wherein each of the alkyl/alkyl, alkenyl, aryl, hetaryl, cycloalkyl, and hetcycloalkyl groups are independently substituted with 0-3 R²⁰;
R¹⁰ and R¹¹ are independently selected from H, C₁-C₈alkyl, C₃-C₁₀cycloalkyl, 3-10 membered hetcycloalkyl, aryl, hetaryl, arylC₁-C₆alkyl, and hetarylC₁-C₆alkyl, wherein each of the alkyl/alkyl, cycloalkyl, hetcycloalkyl, aryl, and hetaryl groups are independently substituted with 0-3 R²¹;
alternatively, R¹⁰ and R¹¹, together with the nitrogen to which they are attached, form a 5-12 membered saturated or partially saturated monocyclic, bicyclic, or tricyclic ring consisting of the shown nitrogen atom, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkyl, hetarylC₁-C₆alkyl, hydroxy, oxo, COOH, C₁-C₆alkyloxy, arylC₁-C₆alkyloxy, hetarylC₁-C₆alkyloxy, C₁-C₆alkyloxyC₁-C₆alkyl, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkyl-carboxy, and hetarylC₁-C₆alkylcarboxy;
R¹² is selected from OH, C₁-C₈alkyl, C₃-C₁₀cycloalkyl, 3-10 membered hetcycloalkyl, trihalomethyl, C₁-C₈alkyloxy, aryl, arylC₁-C₆alkyl, hetaryl, hetarylC₁-C₆alkyl, aryloxy, hetaryloxy, and NR¹³R¹⁴;
R¹³ and R¹⁴ are independently selected from H, C₁-C₈alkyl, C₃-C₁₀cycloalkyl, aryl, hetaryl, arylC₁-C₆alkyl, and hetarylC₁-C₆alkyl, wherein each of the alkyl/alkyl, cycloalkyl, aryl, and hetaryl groups are independently substituted with 0-3 R²²;
alternatively, R¹³ and R¹⁴, together with the nitrogen to which they are attached, form a 5-12 membered saturated or partially saturated monocyclic, bicyclic, or tricyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkyl, hetarylC₁-C₆alkyl, OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkylcarboxy, and hetarylC₁-C₆alkylcarboxy;
R¹⁵ is selected from H, C₁-C₆alkyl, and C₃-C₆cycloalkyl;
R¹⁶ and R¹⁷ are independently selected from H, C₁-C₈alkyl, C₃-C₁₀cycloalkyl, halo, OH, cyano, —C(═O)R¹², —S(O)ₙR¹², —S(O)ₙNR¹³R¹⁴, —N(R¹³)S(O)ₙR¹², C₁-C₈alkyl, aryl, and hetaryl, wherein the alkyl and cycloalkyl groups are independently substituted with 0-3 R²²;
R¹³ is selected from halo, OH, oxo, COOH, cyano C₁-C₆alkyloxy, C₃-C₁₀cycloalkyloxy, aryloxy, hetaryloxy, hetarylthio and arylC₁-C₆alkyloxy;
R¹⁹, R²⁰ and R²¹ are independently selected from H, halo, OH, oxo, cyano, C₁-C₈alkyl, C₃-C₁₀cycloalkyl, 3-10 membered hetcycloalkyl, trihalomethyl, trihalomethyloxy, methylendioxo, dihalo-methylenedioxo, C₃-C₆spirocycloalkyl, C₁-C₆alkyloxy, aryl, hetaryl, arylC₁-C₆alkyl, hetarylC₁-C₆alkyl, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, and —C(═NR¹⁶)NR¹⁷;
R²² is selected from H, OH, oxo, halo, cyano, nitro, C₁-C₆alkyl, C₁-C₆alkyloxy, NR²³R²⁴, methylendioxo, dihalomethylendioxo, trihalomethyl, and trihalomethyloxy;
R²³ and R²⁴ are independently selected from H, C₁-C₈alkyl, and arylC₁-C₆alkyl;
m is selected from 0, 1, and 2;
n is selected from 1 and 2;
Y is selected from O and S;
or a salt thereof with a pharmaceutically acceptable acid or base, or any optical isomer or mixture of optical isomers, including a racemic mixture, or any tautomeric forms.
19. The use according to clause 18 wherein:
R¹ is selected from H, R⁸(C═O)—, R⁹S(O)ₙ—, R¹⁰R¹¹NC(═Y)—, and R¹⁰R¹¹NS(O)ₙ—;
R² is selected from H, C₁-C₆alkyl, and C₃-C₆cycloalkyl;
alternatively, R¹ and R², together with the nitrogen to which they are attached, form a 3-12 membered saturated or partially saturated monocyclic or bicyclic ring consisting of the shown nitrogen, 2-10 carbon atoms, and 0-2 additional heteroatoms selected from nitrogen, oxygen, and S(O)ₘ, wherein this ring is substituted with 0-3 groups selected from C₁-C₈alkyl, C₃-C₆spirocycloalkyl, 3-6 membered spirohetcycloalkyl, aryl, hetaryl, arylC₁-C₆alkylene, hetarylC₁-C₆alkylene, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each aryl/hetaryl group is substituted with 0-3 R¹⁸;
Ring A is a 5-12 membered saturated or partially saturated bicyclic or tricyclic ring consisting of the shown nitrogen, 4-10 carbon atoms and from 0 to 2 additional heteroatoms selected from nitrogen, oxygen, and S(O)ₘ;
Ring A is substituted with 0-3 groups selected from C₁-C₈alkyl, halo, OH, oxo, cyano, C₁-C₆alkyloxy, C₁-C₆alkyloxyC₁-C₆alkylene or C₁-C₆alkylcarbonyl, wherein each alkyl/alkylene group is substituted with 0-3 R¹⁸;
R⁵ is selected from H, C₁-C₆alkyl, C₃-C₆cycloalkyl, halo, OH, and cyano;
R⁶ and R⁷ are independently selected from H, C₁-C₆alkyl, F, trihalomethyl, and trihalomethoxy;
alternatively, R⁶ and R⁷, together with the carbon atom to which they are attached, form a 3-8 membered saturated or partially saturated monocyclic ring consisting of the shown carbon atom, 2-5 additional carbon atoms, and 0-2 heteroatoms selected from nitrogen, oxygen, and S(O)ₘ, wherein this ring is substituted with 0-3 groups selected from halo, trihalomethyl, OH, C₁-C₆alkyl, oxo, and C₁-C₆alkyloxy;
R⁸ is selected from C₁-C₈alkyl, C₂-C₈alkenyl, aryl, hetaryl, arylC₁-C₆alkylene, hetarylC₁-C₆alkylene, C₃-C₁₀cycloalkyl, 3-10 membered hetcycloalkyl, aryloxyC₁-C₆alkylene, hetaryloxyC₁-C₆alkylene, arylC₁-C₆alkyloxyC₁-C₆alkylene, and hetarylC₁-C₆alkyloxyC₁-C₆alkylene, wherein each of the alkyl/alkylene, alkenyl, aryl, hetaryl, cycloalkyl, and hetcycloalkyl groups are independently substituted with 0-3 R¹⁹;
R⁹ is selected from C₁-C₈alkyl, C₂-C₈alkenyl, aryl, hetaryl, arylC₁-C₆alkylene, hetarylC₁-C₆alkylene, C₃-C₁₀cycloalkyl, 3-10 membered hetcycloalkyl, aryloxyC₁-C₆alkylene, and arylC₁-C₆alkyloxyC₁-C₆alkylene, wherein each of the alkyl/alkylene, alkenyl, aryl, hetaryl, cycloalkyl, and hetcycloalkyl groups are independently substituted with 0-3 R²⁰;
R¹⁰ and R¹¹ are independently selected from H, C₁-C₈alkyl, C₃-C₁₀cycloalkyl, 3-10 membered hetcycloalkyl, aryl, hetaryl, arylC₁-C₆alkylene, and hetarylC₁-C₆alkylene, wherein each of the alkyl/alkylene, cycloalkyl, hetcycloalkyl, aryl, and hetaryl groups are independently substituted with 0-3 R²¹;
alternatively, R¹⁰ and R¹¹, together with the nitrogen to which they are attached, form a 5-12 membered saturated or partially saturated monocyclic, bicyclic, or tricyclic ring consisting of the shown nitrogen atom, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkylene, hetarylC₁-C₆alkylene, hydroxy, oxo, COOH, C₁-C₆alkyloxy, arylC₁-C₆alkyloxy, hetarylC₁-C₆alkyloxy, C₁-C₆alkyloxyC₁-C₆alkylene, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkyl-carboxy, and hetarylC₁-C₆alkylcarboxy;
R¹² is selected from OH, C₁-C₈alkyl, C₃-C₁₀cycloalkyl, 3-10 membered hetcycloalkyl, trihalomethyl, C₁-C₈alkyloxy, aryl, arylC₁-C₆alkylene, hetaryl, hetarylC₁-C₆alkylene, aryloxy, hetaryloxy, and NR¹³R¹⁴;
R¹³ and R¹⁴ are independently selected from H, C₁-C₈alkyl, C₃-C₁₀cycloalkyl, aryl, hetaryl, arylC₁-C₆alkylene, and hetarylC₁-C₆alkylene, wherein each of the alkyl/alkylene, cycloalkyl, aryl, and hetaryl groups are independently substituted with 0-3 R²²;
alternatively, R¹³ and R¹⁴, together with the nitrogen to which they are attached, form a 5-12 membered saturated or partially saturated monocyclic, bicyclic, or tricyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkylene, hetarylC₁-C₆alkylene, OH, oxo, C₁-C₆alkyloxy, arylC₁-C₆alkyloxy, hetarylC₁-C₆alkyloxy, C₁-C₆alkyloxyC₁-C₆alkylene, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkylcarboxy, and hetarylC₁-C₆alkylcarboxy;
R¹⁵ is selected from H, C₁-C₆alkyl, and C₃-C₆cycloalkyl;
R¹⁶ and R¹⁷ are independently selected from H, C₁-C₈alkyl, C₃-C₁₀cycloalkyl, halo, OH, cyano, —C(═O)R¹², —S(O)ₙR¹², —S(O)ₙNR¹³R¹⁴, —N(R¹³)S(O)ₙR¹², C₁-C₈alkyl, aryl, and hetaryl, wherein the alkyl and cycloalkyl groups are independently substituted with 0-3 R²²;
R¹⁸ is selected from halo, OH, oxo, and cyano;
R¹⁹, R²⁰ and R²¹ are independently selected from H, halo, OH, oxo, cyano, C₁-C₈alkyl, C₃-C₁₀cycloalkyl, 3-10 membered hetcycloalkyl, trihalomethyl, trihalomethyloxy, methylendioxo, dihalo-methylenedioxo, C₃-C₆spirocycloalkyl, C₁-C₆alkyloxy, aryl, hetaryl, arylC₁-C₆alkylene, hetarylC₁-C₆alkylene, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, and —C(═NR¹⁶)NR¹⁷;
R²² is selected from H, OH, oxo, halo, cyano, nitro, C₁-C₆alkyl, C₁-C₆alkyloxy, NR²³R²⁴, methylendioxo, dihalomethylendioxo, trihalomethyl, and trihalomethyloxy;
R²³ and R²⁴ are independently selected from H, C₁-C₈alkyl, and arylC₁-C₆alkylene;
m is selected from 0, 1, and 2;
n is selected from 1 and 2;
Y is selected from O and S;
20. The use according to clause 18 wherein:
R¹ is selected from R⁸(C═O)—, R⁹S(O)₂—, R¹⁰R¹¹NC(═O)—, and R¹⁰R¹¹NS(O)₂—;
R² is C₁-C₄alkyl;
alternatively, R¹ and R², together with the nitrogen to which they are attached, form a 5-6 membered saturated ring consisting of the shown nitrogen, 2-4 carbon atoms, and 0-2 additional heteroatoms selected from nitrogen, oxygen, and S(O)ₘ, wherein this ring is substituted with 0-2 groups selected from C₁-C₈alkyl, aryl, hetaryl, arylC₁-C₆alkyl, hetarylC₁-C₆alkyl, —C(═O)R¹², —S(O)ₙR¹², —S(═O)ₙNR¹³R¹⁴, —N(R¹³)S(O)ₙR¹², OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸;
Ring A is an 8-11 membered saturated or partially saturated bicyclic or tricyclic ring consisting of the shown nitrogen, 5-10 carbon atoms and from 0 to 1 additional heteroatoms selected from nitrogen, oxygen, and S(O)ₘ;
Ring A is substituted with 0-3 groups selected from C₁-C₄alkyl, halo, OH, oxo, cyano, C₁-C₄alkyloxy, C₁-C₄alkyloxyC₁-C₄alkyl or C₁-C₄alkylcarbonyl, wherein each alkyl/alkyl group is substituted with 0-1 R¹⁸;

### R⁵ is H;

R⁶ and R⁷ are independently selected from H and C₁-C₄alkyl; and,
n is 2.
21. The use according to clause 19 wherein:
R¹ is selected from R⁸(C═O)—, R⁹S(O)₂—, R¹⁰R¹¹NC(═O)—, and R¹¹R¹¹NS(O)₂—;
R² is C₁-C₄alkyl;
alternatively, R¹ and R², together with the nitrogen to which they are attached, form a 5-6 membered saturated ring consisting of the shown nitrogen, 2-4 carbon atoms, and 0-2 additional heteroatoms selected from nitrogen, oxygen, and S(O)ₘ, wherein this ring is substituted with 0-2 groups selected from C₁-C₈alkyl, aryl, hetaryl, arylC₁-C₆alkylene, hetarylC₁-C₆alkylene, —C(═O)R¹², —S(O)ₙR¹², —S(═O)ₙNR¹³R¹⁴, —N(R¹³)S(O)ₙR¹², OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each aryl/hetaryl group is substituted with 0-3 R¹⁸;
Ring A is an 8-11 membered saturated or partially saturated bicyclic or tricyclic ring consisting of the shown nitrogen, 5-10 carbon atoms and from 0 to 1 additional heteroatoms selected from nitrogen, oxygen, and S(O)ₘ;
Ring A is substituted with 0-3 groups selected from C₁-C₄alkyl, halo, OH, oxo, cyano, C₁-C₄alkyloxy, C₁-C₄alkyloxyC₁-C₄alkylene or C₁-C₄alkylcarbonyl, wherein each alkyl/alkylene group is substituted with 0-1 R¹⁸;

### R⁵ is H;

R⁶ and R⁷ are independently selected from H and C₁-C₄alkyl; and,
n is 2.
22. The use according to clause 18 wherein:
R⁸ is selected from C₁-C₆alkyl, C₂-C₆alkenyl, aryl, hetaryl, arylC₁-C₄alkyl, hetarylC₁-C₄alkyl, C₃-C₆cycloalkyl, 3-6 membered hetcycloalkyl, aryloxyC₁-C₄alkyl, and hetaryloxyC₁-C₄alkyl, wherein each of the alkyl/alkyl, alkenyl, aryl, hetaryl, cycloalkyl, and hetcycloalkyl groups are independently substituted with 0-2 R¹⁹;
R⁹ is selected from C₁-C₆alkyl, C₂-C₆alkenyl, aryl, hetaryl, arylC₁-C₄alkyl, hetarylC₁-C₄alkyl, C₃-C₆cycloalkyl, 3-6 membered hetcycloalkyl, and aryloxyC₁-C₄alkyl, wherein each of the alkyl/alkyl, alkenyl, aryl, hetaryl, cycloalkyl, and hetcycloalkyl groups are independently substituted with 0-2 R²⁰1
R¹⁰ and R¹¹ are independently selected from H, C₃-C₆cycloalkyl, 3-6 membered hetcycloalkyl, aryl, and hetaryl, wherein each of the cycloalkyl, hetcycloalkyl, aryl, and hetaryl groups are independently substituted with 0-3 R²¹;
alternatively, R¹⁰ and R¹¹, together with the nitrogen to which they are attached, form a 5-6 membered saturated or partially saturated monocyclic ring consisting of the shown nitrogen atom, 4-5 carbon atoms, and 0-1 additional heteroatoms selected from nitrogen, oxygen, and S(O)ₘ, wherein this ring is substituted with 0-2 groups selected from C₁-C₈alkyl, aryl, hetaryl, hydroxy, oxo, COOH, C₁-C₆alkyloxy, arylC₁-C₆alkyloxy, hetarylC₁-C₆alkyloxy, and C₁-C₆alkylcarbonyl;
R¹² is selected from OH, C₁-C₄alkyl, C₃-C₆cycloalkyl, 3-10 membered hetcycloalkyl, trihalomethyl, C₁-C₄alkyloxy, aryl, arylC₁-C₄alkyl, hetaryl, hetarylC₁-C₄alkyl, aryloxy, and hetaryloxy;
R¹⁹, R²⁰ and R²¹ are independently selected from H, halo, OH, oxo, cyano, C₁-C₆alkyl, C₃-C₆cycloalkyl, 3-6 membered hetcycloalkyl, trihalomethyl, trihalomethyloxy, dihalo-methylenedioxo, C₁-C₄alkyloxy, aryl, hetaryl, arylC₁-C₄alkyl, hetarylC₁-C₄alkyl, —C(═O)R¹², —S(O)ₙR¹², and —S(O)ₙNR¹³R¹⁴; and,
n is 2.
23. The use according to clause 19 wherein:
R³ is selected from C₁-C₆alkyl, C₂-C₆alkenyl, aryl, hetaryl, arylC₁-C₄alkylene, hetarylC₁-C₄alkylene, C₃-C₆cycloalkyl, 3-6 membered hetcycloalkyl, aryloxyC₁-C₄alkylene, and hetaryloxyC₁-C₄alkylene, wherein each of the alkyl/alkylene, alkenyl, aryl, hetaryl, cycloalkyl, and hetcycloalkyl groups are independently substituted with 0-2 R¹⁹;
R⁹ is selected from C₁-C₆alkyl, C₂-C₆alkenyl, aryl, hetaryl, arylC₁-C₄alkylene, hetarylC₁-C₄alkylene, C₃-C₆cycloalkyl, 3-6 membered hetcycloalkyl, and aryloxyC₁-C₄alkylene, wherein each of the alkyl/alkylene, alkenyl, aryl, hetaryl, cycloalkyl, and hetcycloalkyl groups are independently substituted with 0-2 R²⁰;
R¹⁰ and R¹¹ are independently selected from H, C₃-C₆cycloalkyl, 3-6 membered hetcycloalkyl, aryl, and hetaryl, wherein each of the cycloalkyl, hetcycloalkyl, aryl, and hetaryl groups are independently substituted with 0-3 R²¹;
alternatively, R¹⁰ and R¹¹, together with the nitrogen to which they are attached, form a 5-6 membered saturated or partially saturated monocyclic ring consisting of the shown nitrogen atom, 4-5 carbon atoms, and 0-1 additional heteroatoms selected from nitrogen, oxygen, and S(O)ₘ, wherein this ring is substituted with 0-2 groups selected from C₁-C₈alkyl, aryl, hetaryl, hydroxy, oxo, COOH, C₁-C₆alkyloxy, arylC₁-C₆alkyloxy, hetarylC₁-C₆alkyloxy, and C₁-C₆alkylcarbonyl;
R¹² is selected from OH, C₁-C₄alkyl, C₃-C₆cycloalkyl, 3-10 membered hetcycloalkyl, trihalomethyl, C₁-C₄alkyloxy, aryl, arylC₁-C₄alkylene, hetaryl, hetarylC₁-C₄alkylene, aryloxy, and hetaryloxy;
R¹⁹, R²⁰ and R²¹ are independently selected from H, halo, OH, oxo, cyano, C₁-C₆alkyl, C₃-C₆cycloalkyl, 3-6 membered hetcycloalkyl, trihalomethyl, trihalomethyloxy, di-halo-methylenedioxo, C₁-C₄alkyloxy, aryl, hetaryl, arylC₁-C₄alkylene, hetarylC₁-C₄alkylene, —C(═O)R¹², —S(O)ₙR¹², and —S(O)ₙNR¹³R¹⁴; and,
n is 2.
24. The use according to clause 18 wherein the substituted amide or prodrug thereof is of formula Ia:

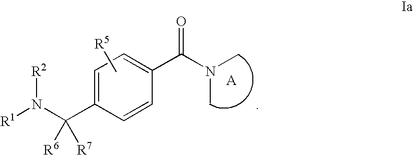

25. The use according to clause 18 wherein the substituted amide or prodrug thereof is of formula Ib:

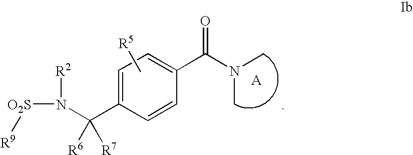

26. The use according to clause 18 wherein the substituted amide or prodrug thereof is of formula Ic:

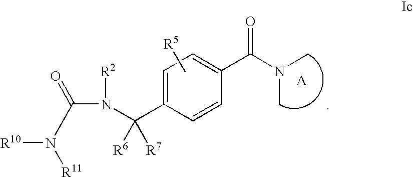

27. The use according to clause 18 wherein the substituted amide or prodrug thereof is of formula Id:

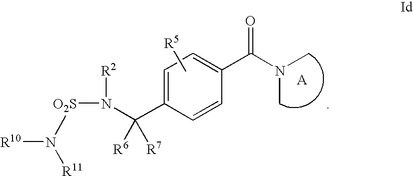

28. The use according to clause 19 wherein the substituted amide or prodrug thereof is of formula Ie:

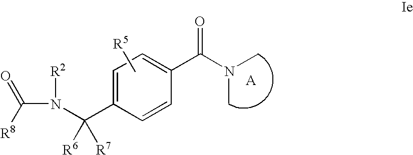

29. The use according to clause 18 wherein:
R¹ and R², together with the nitrogen to which they are attached, form a 5-12 membered saturated or partially saturated monocyclic or bicyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹²—S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸.
30. The use according to clause 19 wherein:
R¹ and R², together with the nitrogen to which they are attached, form a 5-12 membered saturated or partially saturated monocyclic or bicyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, C₃-C₆spirocycloalkyl, 3-6 membered spirohetcycloalkyl, aryl, hetaryl, arylC₁-C₆alkylene, hetarylC₁-C₆alkylene, —C(═O)R¹², —S(O)ₙR¹², —S(O)ₙNR¹³R¹⁴, —N(R¹³)S(O)R¹², —N(R¹⁵)C(═Y)NR¹³R¹⁴, —C(═NR¹⁶)NR¹⁷, OH, oxo, C₁-C₆alkyloxy, arylC₁-C₆alkyl-oxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each aryl/hetaryl group is substituted with 0-3 R¹⁸.
31. The use according to anyone of the clauses 18-30 wherein:
Ring A is selected from:

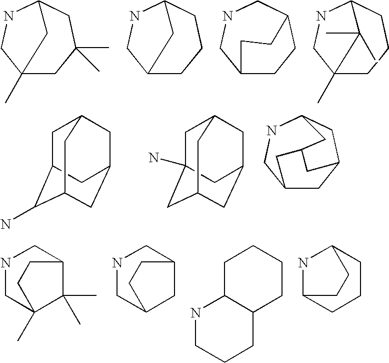

Ring A is substituted with 0-2 R²⁵; and,
R²⁵ is selected from C₁-C₈alkyl, halo, hydroxy, oxo, cyano, C(═O)R¹², and C₁-C₆alkyloxy, wherein R¹² is as defined above.
32. The use according to anyone of the clauses 18-30 wherein:
Ring A is selected from:

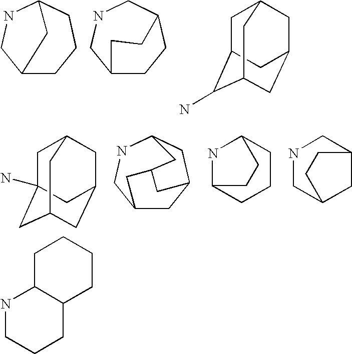

Ring A is substituted with 0-2 R²⁵; and,
R²⁵ is selected from C₁-C₈alkyl, halo, hydroxy, oxo, cyano, and C₁-C₆alkyloxy.
33. The use according to anyone of the clauses 18-30 wherein:
Ring A is selected from:

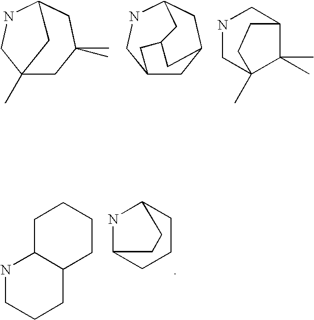

ring A is substituted with 0-2 R²⁵; and,
R²⁵ is selected from C₁-C₈alkyl, halo, hydroxy, oxo, cyano, and C₁-C₆alkyloxy.
34. The use according to clause 18 or 19 wherein the substituted amide or a prodrug thereof is of the selected from the group of clause 12.
35. The use according to any of the clauses 18-34 for the preparation of a pharmaceutical composition for the treatment of conditions, disorders, or diseases wherein a modulation or an inhibition of the activity of 11βHSD1 is beneficial.
36. The use according to clause 35, wherein the conditions, disorders, and diseases that are influenced by intracellular glucocorticoid levels.
37. The use according to clause 35, wherein the conditions, disorders, or diseases are selected from metabolic syndrome, insulin resistance, dyslipidemia, hypertension, obesity, type 2 diabetes, impaired glucose tolerance (IGT), impaired fasting glucose (IFG), the progression from IGT to type 2 diabetes, the progression of the metabolic syndrome into type 2 diabetes, diabetic late complications, neurodegenerative and psychiatric disorders, and the adverse effects of glucocorticoid receptor agonist treatment or therapy.
38. The use according to any of the clauses 18-37 wherein the pharmaceutical composition is suitable for a route of administration selected from oral, nasal, buccal, transdermal, pulmonal, and parenteral.
39. A method for the treatment of conditions, disorders, or diseases wherein a modulation or an inhibition of the activity of 11βHSD1 is beneficial, the method comprising administering to a subject in need thereof an effective amount of a compound according to any of clauses 1-12.
40. The method according to clause 39, wherein the conditions, disorders, and diseases that are influenced by intracellular glucocorticoid levels.
41. The method according to clause 39, wherein the conditions, disorders, or diseases are selected from metabolic syndrome, insulin resistance, dyslipidemia, hypertension, obesity, type 2 diabetes, impaired glucose tolerance (IGT), impaired fasting glucose (IFG), progression from IGT to type 2 diabetes, progression of metabolic syndrome into type 2 diabetes, diabetic late complications, neurodegenerative and psychiatric disorders, and the adverse effects of glucocorticoid receptor agonist treatment or therapy.
42. The method according to any one of clauses 40-41, wherein the administering is via a route selected from oral, nasal, buccal, transdermal, pulmonal, and parenteral.

1. A compound of the formula I: 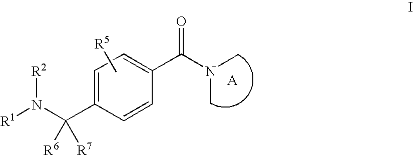
wherein:
R¹ is selected from H, R⁸(C═O)—, R⁹S(O)ₙ—, R¹⁰R¹¹NC(═Y)—, and R¹⁰R¹¹NS(O)ₙ—;
R² is selected from H, C₁-C₆alkyl, and C₃-C₆cycloalkyl;
alternatively, R¹ and R², together with the nitrogen to which they are attached, form a 3-12 membered saturated or partially saturated monocyclic or bicyclic ring consisting of the shown nitrogen, 2-10 carbon atoms, and 0-2 additional heteroatoms selected from nitrogen, oxygen, and S(O)ₘ, wherein this ring is substituted with 0-3 groups selected from C₁-C₈alkyl, C₃-C₁₀cycloalkyl, C₃-C₁₀hetcycloalkyl, C₃-C₆spirocycloalkyl, 3-6 membered spirohetcycloalkyl, aryl, hetaryl, arylC₁-C₆alkyl, hetarylC₁-C₆alkyl, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, —C(═NR¹⁶)NR¹⁷, OH, oxo, C₁-C₆alkyloxy, aryl C₁-C₆alkyl-oxy, hetaryl C₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹³;
Ring A is a 5-12 membered saturated or partially saturated bicyclic or tricyclic ring consisting of the shown nitrogen, 4-10 carbon atoms and from 0 to 2 additional heteroatoms selected from nitrogen, oxygen, and S(O)ₘ;
Ring A is substituted with 0-3 groups selected from C₁-C₈alkyl, halo, OH, oxo, cyano, C₁-C₆alkyloxy, C₁-C₆alkyloxyC₁-C₆alkyl or C₁-C₆alkylcarbonyl, wherein each alkyl group is substituted with 0-3 R¹¹;
R⁵ is selected from H, C₁-C₆alkyl, C₃-C₆cycloalkyl, halo, OH, and cyano;
R⁶ and R⁷ are independently selected from H, C₁-C₆alkyl, F, trihalomethyl, and trihalomethoxy;
alternatively, R⁶ and R⁷, together with the carbon atom to which they are attached, form a 3-8 membered saturated or partially saturated monocyclic ring consisting of the shown carbon atom, 2-5 additional carbon atoms, and 0-2 heteroatoms selected from nitrogen, oxygen, and S(O)ₘ, wherein this ring is substituted with 0-3 groups selected from halo, trihalomethyl, OH, C₁-C₆alkyl, oxo, and C₁-C₆alkyloxy;
R⁸ is selected from C₁-C₈alkyl, C₂-C₈alkenyl, aryl, hetaryl, arylC₁-C₆alkyl, hetarylC₁-C₆alkyl, C₃-C₁₀cycloalkyl, 3-10 membered hetcycloalkyl, aryloxyC₁-C₆alkyl, hetaryloxyC₁-C₆alkyl, arylC₁-C₆alkyloxyC₁-C₆alkyl, and hetarylC₁-C₆alkyloxyC₁-C₆alkyl, wherein each of the alkyl/alkyl, alkenyl, aryl, hetaryl, cycloalkyl, and hetcycloalkyl groups are independently substituted with 0-3 R¹⁹;
R⁹ is selected from C₁-C₈alkyl, C₂-C₈alkenyl, aryl, hetaryl, arylC₁-C₆alkyl, hetarylC₁-C₆alkyl, C₃-C₁₀cycloalkyl, 3-10 membered hetcycloalkyl, aryloxyC₁-C₆alkyl, and arylC₁-C₆alkyloxyC₁-C₆alkyl, wherein each of the alkyl/alkyl, alkenyl, aryl, hetaryl, cycloalkyl, and hetcycloalkyl groups are independently substituted with 0-3 R²⁰;
R¹⁰ and R¹¹ are independently selected from H, C₁-C₈alkyl, C₃-C₁₀cycloalkyl, 3-10 membered hetcycloalkyl, aryl, hetaryl, arylC₁-C₆alkyl, and hetarylC₁-C₆alkyl, wherein each of the alkyl/alkyl, cycloalkyl, hetcycloalkyl, aryl, and hetaryl groups are independently substituted with 0-3 R²¹;
alternatively, R¹⁰ and R¹¹, together with the nitrogen to which they are attached, form a 5-12 membered saturated or partially saturated monocyclic, bicyclic, or tricyclic ring consisting of the shown nitrogen atom, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkyl, hetarylC₁-C₆alkyl, hydroxy, oxo, COOH, C₁-C₆alkyloxy, arylC₁-C₆alkyloxy, hetarylC₁-C₆alkyloxy, C₁-C₆alkyloxyC₁-C₆alkyl, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkyl-carboxy, and hetarylC₁-C₆alkylcarboxy;
R¹² is selected from OH, C₁-C₈alkyl, C₃-C₁₀cycloalkyl, 3-10 membered hetcycloalkyl, trihalomethyl, C₁-C₈alkyloxy, aryl, arylC₁-C₆alkyl, hetaryl, hetarylC₁-C₆alkyl, aryloxy, hetaryloxy, and NR¹³R¹⁴;
R¹³ and R¹⁴ are independently selected from H, C₁-C₈alkyl, C₃-C₁₀cycloalkyl, aryl, hetaryl, arylC₁-C₆alkyl, and hetarylC₁-C₆alkyl, wherein each of the alkyl/alkyl, cycloalkyl, aryl, and hetaryl groups are independently substituted with 0-3 R²²;
alternatively, R¹³ and R¹⁴, together with the nitrogen to which they are attached, form a 5-12 membered saturated or partially saturated monocyclic, bicyclic, or tricyclic ring consisting of the shown nitrogen, 4-10 carbon atoms, and 0-2 additional heteroatoms selected from nitrogen, oxygen, and S(O)ₘ, wherein this ring is substituted with 0-3 groups selected from C₁-C₈alkyl, aryl, hetaryl, arylC₁-C₆alkyl, hetarylC₁-C₆alkyl, OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarbonyl, arylcarbonyl, hetarylcarbonyl, arylC₁-C₆alkylcarbonyl, hetarylC₁-C₆alkylcarbonyl, C₁-C₆alkylcarboxy, arylcarboxy, hetarylcarboxy, arylC₁-C₆alkylcarboxy, and hetarylC₁-C₆alkylcarboxy;
R¹⁵ is selected from H, C₁-C₆alkyl, and C₃-C₆cycloalkyl;
R¹⁶ and R¹⁷ are independently selected from H, C₁-C₈alkyl, C₃-C₁₀cycloalkyl, halo, OH, cyano, —C(═O)R¹², —S(O)ₙR¹², —S(O)ₙNR¹³R¹⁴, —N(R¹³)S(O)ₙR¹², C₁-C₈alkyl, aryl, and hetaryl, wherein the alkyl and cycloalkyl groups are independently substituted with 0-3 R²²;
R¹⁸ is selected from halo, OH, oxo, COOH, cyano C₁-C₆alkyloxy, C₃-C₁₀cycloalkyloxy, aryloxy, hetaryloxy, hetarylthio and arylC₁-C₆alkyloxy;
R¹⁹, R²⁰ and R²¹ are independently selected from H, halo, OH, oxo, cyano, C₁-C₈alkyl, C₃-C₁₀cycloalkyl, 3-10 membered hetcycloalkyl, trihalomethyl, trihalomethyloxy, methylendioxo, dihalo-methylenedioxo, C₃-C₆spirocycloalkyl, C₁-C₆alkyloxy, aryl, hetaryl, arylC₁-C₆alkyl, hetarylC₁-C₆alkyl, —C(═O)R¹², —S(O)ₙR¹², —S(O)ₙNR¹³R¹⁴, —N(R¹³)S(O)ₙR¹², —N(R¹⁵)C(═Y)NR¹³R¹⁴, and —C(═NR¹⁶)NR¹⁷;
R²² is selected from H, OH, oxo, halo, cyano, nitro, C₁-C₆alkyl, C₁-C₆alkyloxy, NR²³R²⁴ methylendioxo, dihalomethylendioxo, trihalomethyl, and trihalomethyloxy;
R²³ and R²⁴ are independently selected from H, C₁-C₈alkyl, and arylC₁-C₆alkyl;
m is selected from 0, 1, and 2;
n is selected from 1 and 2;
Y is selected from O and S;
or a salt thereof with a pharmaceutically acceptable acid or base, or any optical isomer or mixture of optical isomers, including a racemic mixture, or any tautomeric forms.

2. A compound according to claim 1 wherein:
R¹ is selected from R⁸(C═O)—, R⁹S(O)₂—, R¹⁰R¹¹NC(═O)—, and R¹⁰R¹¹NS(O)₂—;
R² is C₁-C₄alkyl;
alternatively, R¹ and R², together with the nitrogen to which they are attached, form a 5-6 membered saturated ring consisting of the shown nitrogen, 2-4 carbon atoms, and 0-2 additional heteroatoms selected from nitrogen, oxygen, and S(O)ₘ, wherein this ring is substituted with 0-2 groups selected from C₁-C₈alkyl, aryl, hetaryl, arylC₁-C₆alkyl, hetarylC₁-C₆alkyl, —C(═O)R¹², —S(O)ₙR¹², —S(═O)ₙNR¹³R¹⁴, —N(R¹³)S(O)ₙR¹², OH, oxo, C₁-C₆alkyloxy, arylC₁-C₆alkyloxy, hetarylC₁-C₆alkyloxy, C₁-C₆alkyloxyC₁-C₆alkyl, C₁-C₆alkylcarboxy, arylcarboxy, hetarylcarboxy, arylC₁-C₆alkylcarboxy, and hetarylC₁-C₆alkylcarboxy, wherein each alkyl and aryl/hetaryl group is substituted with 0-3 R¹⁸;
Ring A is an 8-11 membered saturated or partially saturated bicyclic or tricyclic ring consisting of the shown nitrogen, 5-10 carbon atoms and from 0 to 1 additional heteroatoms selected from nitrogen, oxygen, and S(O)ₘ;
Ring A is substituted with 0-3 groups selected from C₁-C₄alkyl, halo, OH, oxo, cyano, C₁-C₄alkyloxy, C₁-C₄alkyloxyC₁-C₄alkyl or C₁-C₄alkylcarbonyl, wherein each alkyl/alkyl group is substituted with 0-1 R¹⁸;
R⁵ is H;
R⁶ and R⁷ are independently selected from H and C₁-C₄alkyl; and,
n is 2.

3. A compound according to claim 1 wherein:
R¹ is selected from R⁸(C═O)—, R⁹S(O)₂—, R¹⁰R¹¹NC(═O)—, and R¹⁰R¹¹NS(O)₂—;
R² is selected from H, C₁-C₄alkyl and C₃-C₆cycloalkyl;
Ring A is an 8-11 membered saturated or partially saturated bicyclic or tricyclic ring consisting of the shown nitrogen, 5-10 carbon atoms and from 0 to 1 additional heteroatoms selected from nitrogen, oxygen, and S(O)ₘ;
Ring A is substituted with 0-3 groups selected from C₁-C₄alkyl, halo, OH, oxo, cyano, C₁-C₄alkyloxy, C₁-C₄alkyloxyC₁-C₄alkyl or C₁-C₄alkylcarbonyl, wherein each alkyl/alkyl group is substituted with 0-1 R¹⁸;
R⁵ is H;
R⁶ and R⁷ are independently selected from H and C₁-C₄alkyl; and,
n is 2.

4. A compound according to claim 1 wherein:
R⁸ is selected from C₁-C₆alkyl, C₂-C₆alkenyl, aryl, hetaryl, arylC₁-C₄alkyl, hetarylC₁-C₄alkyl, C₃-C₆cycloalkyl, 3-6 membered hetcycloalkyl, aryloxyC₁-C₄alkyl, and hetaryloxyC₁-C₄alkyl, wherein each of the alkyl/alkyl, alkenyl, aryl, hetaryl, cycloalkyl, and hetcycloalkyl groups are independently substituted with 0-2 R¹⁹;
R⁹ is selected from C₁-C₆alkyl, C₂-C₆alkenyl, aryl, hetaryl, arylC₁-C₄alkyl, hetarylC₁-C₄alkyl, C₃-C₆cycloalkyl, 3-6 membered hetcycloalkyl, and aryloxyC₁-C₄alkyl, wherein each of the alkyl/alkyl, alkenyl, aryl, hetaryl, cycloalkyl, and hetcycloalkyl groups are independently substituted with 0-2 R²⁰;
R¹⁰ and R¹¹ are independently selected from H, C₃-C₆cycloalkyl, 3-6 membered hetcycloalkyl, aryl, and hetaryl, wherein each of the cycloalkyl, hetcycloalkyl, aryl, and hetaryl groups are independently substituted with 0-3 R²¹;
alternatively, R¹⁰ and R¹¹, together with the nitrogen to which they are attached, form a 5-6 membered saturated or partially saturated monocyclic ring consisting of the shown nitrogen atom, 4-5 carbon atoms, and 0-1 additional heteroatoms selected from nitrogen, oxygen, and S(O)ₘ, wherein this ring is substituted with 0-2 groups selected from C₁-C₈alkyl, aryl, hetaryl, hydroxy, oxo, COOH, C₁-C₆alkyloxy, arylC₁-C₆alkyloxy, hetarylC₁-C₆alkyloxy, and C₁-C₆alkylcarbonyl;
R¹² is selected from OH, C₁-C₄alkyl, C₃-C₆cycloalkyl, 3-10 membered hetcycloalkyl, trihalomethyl, C₁-C₄alkyloxy, aryl, arylC₁-C₄alkyl, hetaryl, hetarylC₁-C₄alkyl, aryloxy, and hetaryloxy;
R¹⁹, R²⁰ and R²¹ are independently selected from H, halo, OH, oxo, cyano, C₁-C₆alkyl, C₃-C₆cycloalkyl, 3-6 membered hetcycloalkyl, trihalomethyl, trihalomethyloxy, dihalo-methylenedioxo, C₁-C₄alkyloxy, aryl, hetaryl, arylC₁-C₄alkyl, hetarylC₁-C₄alkyl, —C(═O)R¹², —S(O)ₙR¹², and —S(O)ₙNR¹³R¹⁴; and,
n is 2.

5. A compound according to claim 1 wherein the compound is of formula Ia:

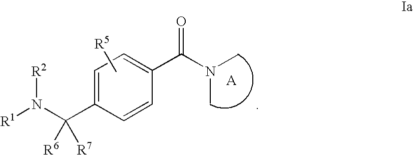

6. A compound according to claim 1 wherein the compound is of formula Ib:

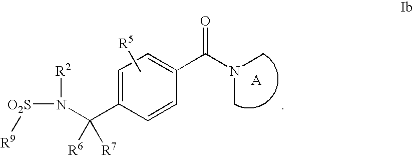

7. A compound according to claim 1 wherein the compound is of formula Ic:

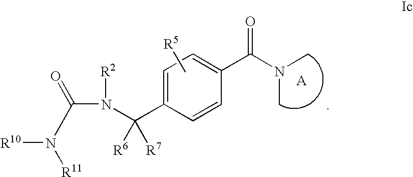

8. A compound according to claim 1 wherein the compound is of formula Id:

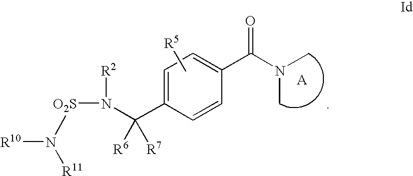

9. A compound according to claim 1 wherein the compound is of formula Ie:

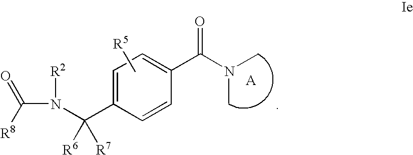

10. A compound according to claim 1 wherein:
Ring A is selected from: 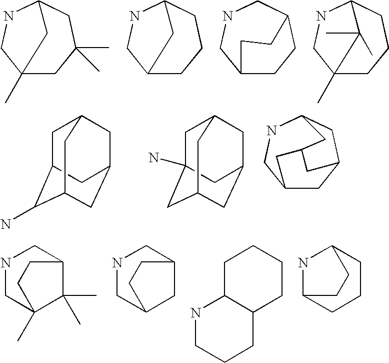
Ring A is substituted with 0-2 R²⁵; and,
R²⁵ is selected from C₁-C₈alkyl, halo, hydroxy, oxo, cyano, C(═O)R¹², and C₁-C₆alkyloxy, wherein R¹² is as defined above.

11. A compound according to claim 1 wherein:
ring A is 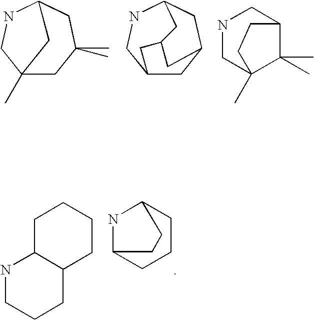
Ring A is substituted with 0-2 R²⁵; and,
R²⁵ is selected from C₁-C₈alkyl, halo, hydroxy, oxo, cyano, and C₁-C₆alkyloxy.

12. A compound according to claim 1 wherein the compound is selected from the group:
N-Methyl-N-[4-(1,3,3-trimethyl-6-aza-bicyclo[3.2.1]octane-6-carbonyl)-benzyl]-acetamide
N-Methyl-N-[4-(1,3,3-trimethyl-6-aza-bicyclo[3.2.1]octane-6-carbonyl)-benzyl]-isobutyramide
Cyclopentanecarboxylic acid methyl-[4-(1,3,3-trimethyl-6-aza-bicyclo[3.2.1]octane-6-carbonyl)-benzyl]-amide
Cyclohexanecarboxylic acid methyl-[4-(1,3,3-trimethyl-6-aza-bicyclo[3.2.1]octane-6-carbonyl)-benzyl]-amide
Piperidine-1-carboxylic acid methyl-[4-(1,3,3-trimethyl-6-aza-bicyclo[3.2.1]octane-6-carbonyl)-benzyl]-amide
N-Methyl-N-[4-(1,3,3-trimethyl-6-aza-bicyclo[3.2.1]octane-6-carbonyl)-benzyl]-benzamide
1-Acetyl-piperidine-4-carboxylic acid methyl-[4-(1,3,3-trimethyl-6-aza-bicyclo[3.2.1]-octane-6-carbonyl)-benzyl]-amide
1-Acetyl-piperidine-3-carboxylic acid methyl-[4-(1,3,3-trimethyl-6-aza-bicyclo[3.2.1]-octane-6-carbonyl)-benzyl]-amide
Cyclopentanecarboxylic acid ethyl-[4-(1,3,3-trimethyl-6-aza-bicyclo[3.2.1]octane-6-carbonyl)-benzyl]-amide
Morpholine-4-carboxylic acid methyl-[4-(1,3,3-trimethyl-6-aza-bicyclo[3.2.1]octane-6-carbonyl)-benzyl]-amide
2,2-N-Trimethyl-N-[4-(1,3,3-trimethyl-6-aza-bicyclo[3.2.1]octane-6-carbonyl)-benzyl]-propionamide
Tetrahydro-furan-3-carboxylic acid methyl-[4-(1,3,3-trimethyl-6-aza-bicyclo[3.2.1]-octane-6-carbonyl)-benzyl]-amide
N-Methyl-4-trifluoromethoxy-N-[4-(1,3,3-trimethyl-6-aza-bicyclo[3.2.1]octane-6-carbonyl)-benzyl]-benzamide
Thiophene-2-carboxylic acid methyl-[4-(1,3,3-trimethyl-6-aza-bicyclo[3.2.1]octane-6-carbonyl)-benzyl]-amide
Furan-2-carboxylic acid methyl-[4-(1,3,3-trimethyl-6-aza-bicyclo[3.2.1]octane-6-carbonyl)-benzyl]-amide
3-Chloro-4-(propane-2-sulfonyl)-thiophene-2-carboxylic acid methyl-[4-(1,3,3-trimethyl-6-aza-bicyclo[3.2.1]octane-6-carbonyl)-benzyl]-amide
6-Chloro-N-methyl-N-[4-(1,3,3-trimethyl-6-aza-bicyclo[3.2.1]octane-6-carbonyl)-benzyl]-nicotinamide
5-Methyl-isoxazole-3-carboxylic acid methyl-[4-(1,3,3-trimethyl-6-aza-bicyclo[3.2.1]-octane-6-carbonyl)-benzyl]-amide
3,3,N-Trimethyl-N-[4-(1,3,3-trimethyl-6-aza-bicyclo[3.2.1]octane-6-carbonyl)-benzyl]-butyramide
3-Cyano-N-methyl-N-[4-(1,3,3-trimethyl-6-aza-bicyclo[3.2.1]octane-6-carbonyl)-benzyl]-benzamide
N-Methyl-2-phenoxy-N-[4-(1,3,3-trimethyl-6-aza-bicyclo[3.2.1]octane-6-carbonyl)-benzyl]-acetamide
N-Methyl-N-[4-(1,3,3-trimethyl-6-aza-bicyclo[3.2.1]octane-6-carbonyl)-benzyl]-malonamic acid methyl ester
3-Methyl-but-2-enoic acid methyl-[4-(1,3,3-trimethyl-6-aza-bicyclo[3.2.1]octane-6-carbonyl)-benzyl]-amide
N-Methyl-2-phenyl-N-[4-(1,3,3-trimethyl-6-aza-bicyclo[3.2.1]octane-6-carbonyl)-benzyl]-acetamide
1-Trifluoromethyl-cyclobutanecarboxylic acid methyl-[4-(1,3,3-trimethyl-6-aza-bicyclo[3.2.1]octane-6-carbonyl)-benzyl]-amide
3,5-Dimethoxy-N-methyl-N-[4-(1,3,3-trimethyl-6-aza-bicyclo[3.2.1]octane-6-carbonyl)-benzyl]-benzamide
4-Methanesulfonyl-N-methyl-N-[4-(1,3,3-trimethyl-6-aza-bicyclo[3.2.1]octane-6-carbonyl)-benzyl]-benzamide
N-Methyl-3-trifluoromethoxy-N-[4-(1,3,3-trimethyl-6-aza-bicyclo[3.2.1]octane-6-carbonyl)-benzyl]-benzamide
2,2-Difluoro-1,3-benzodioxole-4-carboxylic acid methyl-[4-(1,3,3-trimethyl-6-aza-bicyclo[3.2.1]octane-6-carbonyl)-benzyl]-amide
N-Methyl-6-morpholin-4-yl-N-[4-(1,3,3-trimethyl-6-aza-bicyclo[3.2.1]octane-6-carbonyl)-benzyl]-nicotinamide
N-Methyl-4-(2,2,2-trifluoro-acetyl)-N-[4-(1,3,3-trimethyl-6-aza-bicyclo[3.2.1]octane-6-carbonyl)-benzyl]-benzamide
N-[4-(1,3,3-Trimethyl-6-aza-bicyclo[3.2.1]octane-6-carbonyl)-benzyl]-benzamide
N-Methyl-N-[4-(1,3,3-trimethyl-6-aza-bicyclo[3.2.1]octane-6-carbonyl)-benzyl]-isophthalamic acid
2,3-Dihydro-benzofuran-7-carboxylic acid methyl-[4-(1,3,3-trimethyl-6-aza-bicyclo-[3.2.1]octane-6-carbonyl)-benzyl]-amide
3-Acetyl-N-methyl-N-[4-(1,3,3-trimethyl-6-aza-bicyclo[3.2.1]octane-6-carbonyl)-benzyl]-benzamide
1,1,3-Trimethyl-3-[4-(1,3,3-trimethyl-bicyclo[3.2.1]octane-6-carbonyl)-benzyl]-sulphonylurea
N-Methyl-N-[4-(1,3,3-trimethyl-6-aza-bicyclo[3.2.1]octane-6-carbonyl)-benzyl]-methanesulfonamide
2,2,2-Trifluoro-ethanesulfonic acid methyl-[4-(1,3,3-trimethyl-6-aza-bicyclo[3.2.1]-octane-6-carbonyl)-benzyl]-amide
N-Methylphenyl-N-[4-(1,3,3-trimethyl-6-aza-bicyclo[3.2.1]octane-6-carbonyl)-benzyl]-methanesulfonamide
Trifluoro-N-isopropyl-N-[4-(1,3,3-trimethyl-6-aza-bicyclo[3.2.1]octane-6-carbonyl)-benzyl]-methanesulfonamide
N-Cyclopropyl-trifluoro-N-[4-(1,3,3-trimethyl-6-aza-bicyclo[3.2.1]octane-6-carbonyl)-benzyl]-methanesulfonamide
N-Ethyl-trifluoro-N-[4-(1,3,3-trimethyl-6-aza-bicyclo[3.2.1]octane-6-carbonyl)-benzyl]-methanesulfonamide
Trifluoro-N-methyl-N-[4-(1,3,3-trimethyl-6-aza-bicyclo[3.2.1]octane-6-carbonyl)-benzyl]-methanesulfonamide
3-Benzoyl-1-methyl-1-[4-(1,3,3-trimethyl-6-aza-bicyclo[3.2.1]octane-6-carbonyl)-benzyl]-urea
3-Cyclohexyl-1-methyl-1-[4-(1,3,3-trimethyl-6-aza-bicyclo[3.2.1]octane-6-carbonyl)-benzyl]-urea
3-(4-methyl-phenyl)sulfonyl-1-methyl-1-[4-(1,3,3-tri-methyl-6-aza-bicyclo[3.2.1]octane-6-carbonyl)-benzyl]-urea
1,3-Dimethyl-3-phenyl-1-[4-(1,3,3-trimethyl-6-aza-bicyclo[3.2.1]octane-6-carbonyl)-benzyl]-urea
3-(2,3-Dihydro-1,4-benzodioxin-2-ylmethyl)-1-methyl-1-[4-(1,3,3-trimethyl-6-aza-bicyclo[3.2.1]octane-6-carbonyl)-benzyl]-urea
3-(3-Methoxy-benzyl)-1-methyl-1-[4-(1,3,3-trimethyl-6-aza-bicyclo[3.2.1]octane-6-carbonyl)-benzyl]-urea
3-(1,1-Dioxo-tetrahydro-thiophen-3-yl)-1-methyl-1-[4-(1,3,3-trimethyl-6-aza-bicyclo[3.2.1]octane-6-carbonyl)-benzyl]-urea
1-Methyl-3-(tetrahydro-pyran-4-yl)-1-[4-(1,3,3-trimethyl-6-aza-bicyclo[3.2.1]-octane-6-carbonyl)-benzyl]-urea
{1,3-Dimethyl-3-[4-(1,3,3-trimethyl-6-aza-bicyclo[3.2.1]octane-6-carbonyl)-benzyl]-ureido}-acetic acid methyl ester
1-Methyl-3-(5-trifluoromethyl-1,3,4-thiadiazol-2-yl)-1-[4-(1,3,3-trimethyl-6-aza-bicyclo[3.2.1]octane-6-carbonyl)-benzyl]-urea
2-{3-Methyl-3-[4-(1,3,3-trimethyl-6-aza-bicyclo[3.2.1]octane-6-carbonyl)-benzyl]-ureido}-benzoic acid methyl ester
3-{3-Methyl-3-[4-(1,3,3-trimethyl-6-aza-bicyclo[3.2.1]octane-6-carbonyl)-benzyl]-ureido}-benzoic acid ethyl ester
1-Methyl-3-(3-ethylsulfanyl-phenyl)-1-[4-(1,3,3-trimethyl-6-aza-bicyclo-[3.2.1]-octane-6-carbonyl)-benzyl]-urea
1-Methyl-3-(4-ethylsulfanyl-phenyl)-1-[4-(1,3,3-trimethyl-6-aza-bicyclo-[3.2.1]-octane-6-carbonyl)-benzyl]-urea
3-(4-Benzyloxy-phenyl)-1-methyl-1-[4-(1,3,3-trimethyl-6-aza-bicyclo[3.2.1]octane-6-carbonyl)-benzyl]-urea
1-Methyl-3-(4-trifluoro-methylsulfanyl-phenyl)-1-[4-(1,3,3-trimethyl-6-aza-bicyclo-[3.2.1]octane-6-carbonyl)-benzyl]-urea
3-(4-Acetyl-phenyl)-1-methyl-1-[4-(1,3,3-tri-methyl-6-aza-bicyclo[3.2.1]octane-6-carbonyl)-benzyl]-urea
3-(3-Acetyl-phenyl)-1-methyl-1-[4-(1,3,3-tri-methyl-6-aza-bicyclo[3.2.1]octane-6-carbonyl)-benzyl]-urea
3-(3-Cyano-phenyl)-1-methyl-1-[4-(1,3,3-tri-methyl-6-aza-bicyclo[3.2.1]octane-6-carbonyl)-benzyl]-urea
1-Methyl-3-(4-trifluoro-methyl-phenyl)-1-[4-(1,3,3-trimethyl-6-aza-bicyclo-[3.2.1]-octane-6-carbonyl)-benzyl]-urea
3-(4-Methoxy-benzyl)-1-methyl-1-[4-(1,3,3-tri-methyl-6-aza-bicyclo[3.2.1]octane-6-carbonyl)-benzyl]-urea
1-Methyl-3-(2,2,4,4-tetrafluoro-4H-benzo-[1,3]dioxin-6-yl)-1-[4-(1,3,3-trimethyl-6-aza-bicyclo-[3.2.1]octane-6-carbonyl)-benzyl]-urea
1-Methyl-3-(4-trifluoro-methoxy-phenyl)-1-[4-(1,3,3-trimethyl-6-aza-bicyclo[3.2.1]-octane-6-carbonyl)-benzyl]-urea
1-Methyl-3-[4-(2,2,2-trifluoro-acetyl)-cyclohexyl]-1-[4-(1,3,3-trimethyl-6-aza-bicyclo[3.2.1]octane-6-carbonyl)-benzyl]-urea
1-(4-Acetyl-phenyl)-1,3-dimethyl-3-[4-(1,3,3-tri-methyl-6-aza-bicyclo[3.2.1]octane-6-carbonyl)-benzyl]-urea
1-Phenyl-3-{1-[4-(1,3,3-trimethyl-6-aza-bicyclo-[3.2.1]octane-6-carbonyl)-phenyl]-cyclopropyl}-urea
Piperidine-1-carboxylic acid methyl-[4-(octahydro-quinoline-1-carbonyl)-benzyl]-amide
Piperidine-1-carboxylic acid [4-(3-aza-bicyclo-[3.2.2]nonane-3-carbonyl)-benzyl]-methyl-amide
Morpholine-4-carboxylic acid methyl-[4-(octahydro-quinoline-1-carbonyl)-benzyl]-amide
Morpholine-4-carboxylic acid [4-(3-aza-bicyclo-[3.2.2]nonane-3-carbonyl)-benzyl]-methyl-amide
1,3-Dimethyl-3-[4-(octahydro-quinoline-1-carbonyl)-benzyl]-1-phenyl-urea
1-[4-(3-Aza-bicyclo-[3.2.2]nonane-3-carbonyl)-benzyl]-1,3-dimethyl-3-phenyl-urea
Piperidine-1-carboxylic acid [4-(6-aza-bicyclo-[3.2.1]octane-6-carbonyl)-benzyl]-methyl-amide
Piperidine-1-carboxylic acid methyl-[4-(1,8,8-trimethyl-3-aza-bicyclo[3.2.1]octane-3-carbonyl)-benzyl]-amide
Morpholine-4-carboxylic acid [4-(6-aza-bicyclo-[3.2.1]octane-6-carbonyl)-benzyl]-methyl-amide
Morpholine-4-carboxylic acid methyl-[4-(1,8,8-trimethyl-3-aza-bicyclo[3.2.1]-octane-3-carbonyl)-benzyl]-amide
1-[4-(6-Aza-bicyclo-[3.2.1]octane-6-carbonyl)-benzyl]-1,3-dimethyl-3-phenyl-urea
1,3-Dimethyl-1-phenyl-3-[4-(1,8,8-trimethyl-3-aza-bicyclo[3.2.1]octane-3-carbonyl)-benzyl]-urea
Piperidine-1-carboxylic acid [4-(3-hydroxy-8-aza-bicyclo[3.2.1]octane-8-carbonyl)-benzyl]-methyl-amide
Morpholine-4-carboxylic acid [4-(3-hydroxy-8-aza-bicyclo[3.2.1]octane-8-carbonyl)-benzyl]-methyl-amide
1-[4-(3-Hydroxy-8-aza-bicyclo[3.2.1]octane-8-carbonyl)-benzyl]-1,3-di-methyl-3-phenyl-urea
1-[4-(3-Fluoro-8-aza-bicyclo[3.2.1]octane-8-carbonyl)-benzyl]-1,3-di-methyl-3-phenyl-urea
Piperidine-1-carboxylic acid [4-(3-fluoro-8-aza-bicyclo[3.2.1]octane-8-carbonyl)-benzyl]-methyl-amide
Morpholine-4-carboxylic acid [4-(3-fluoro-8-aza-bicyclo[3.2.1]octane-8-carbonyl)-benzyl]-methyl-amide
N-Adamantan-2-yl-4-(1,3-dimethyl-3-pyridin-2-yl-ureidomethyl)-benzamide
1-[4-(3-Aza-bicyclo[3.2.2]nonane-3-carbonyl)-benzyl]-1,3-dimethyl-3-pyridin-2-yl-urea
1-[4-(3-Hydroxy-8-aza-bicyclo[3.2.1]octane-8-carbonyl)-benzyl]-1,3-dimethyl-3-pyridin-2-yl-urea
Morpholine-4-carboxylic acid [4-(adamantan-2-ylcarbamoyl)-benzyl]-methyl-amide
1-[4-(3-Aza-bicyclo[3.2.2]nonane-3-carbonyl)-benzyl]-1,3-dimethyl-3-thiazol-2-yl-urea
1,3-Dimethyl-3-[4-(2-oxa-5-aza-bicyclo[2.2.1]heptane-5-carbonyl)-benzyl]-1-thiazol-2-yl-urea
4-[3-(1-Acetyl-piperidin-4-yl)-1,3-dimethyl-ureidomethyl]-N-adamantan-2-yl-benzamide
1-(1-Acetyl-piperidin-4-yl)-3-[4-(3-aza-bicyclo[3.2.2]nonane-3-carbonyl)-benzyl]-1,3-dimethyl-urea
N-Adamantan-2-yl-4-(1,3-dimethyl-3-pyrimidin-2-yl-ureidomethyl)-benzamide
1-[4-(3-Aza-bicyclo[3.2.2]nonane-3-carbonyl)-benzyl]-1,3-dimethyl-3-pyrimidin-2-yl-urea
N-Adamantan-2-yl-4-(1,3-dimethyl-3-thiazol-2-yl-ureidomethyl)-benzamide
N-Adamantan-2-yl-4-(1,3-dimethyl-3-phenyl-ureidomethyl)-benzamide
1-[4-(3-Hydroxy-8-aza-bicyclo[3.2.1]octane-8-carbonyl)-benzyl]-1,3-dimethyl-3-pyrimidin-2-yl-urea
N-Adamantan-2-yl-4-[3-(4-hydroxy-cyclohexyl)-1,3-dimethyl-ureidomethyl]-benzamide
1-[4-(3-Aza-bicyclo[3.2.2]nonane-3-carbonyl)-benzyl]-3-(4-hydroxy-cyclohexyl)-1,3-dimethyl-urea
1-(4-Hydroxy-cyclohexyl)-1,3-dimethyl-3-[4-(2-oxa-5-aza-bicyclo[2.2.1]heptane-5-carbonyl)-benzyl]-urea
1-Methyl-3-[4-(1,3,3-trimethyl-6-aza-bicyclo[3.2.1]octane-6-carbonyl)-benzyl]-imidazo-lidin-2-one
[4-(1,1-Dioxo-isothiazolidin-2-ylmethyl)-phenyl]-(1,3,3-trimethyl-6-aza-bicyclo[3.2.1]-oct-6-yl)-methanone
[4-(1,1-Dioxo-1,2,5-thiadiazolidin-2-ylmethyl)-phenyl]-(1,3,3-trimethyl-bicyclo[3.2.1]oct-6-yl)-methanone
[4-(5-Methyl-1,1-dioxo-1,2,5-thiadiazolidin-2-ylmethyl)-phenyl]-(1,3,3-trimethyl-bicyclo[3.2.1]oct-6-yl)-methanone
(Octahydro-quinolin-1-yl)-[4-(1,1-dioxo-1,2,5-thiadiazolidin-2-ylmethyl)-phenyl]-methanone
(4-Aza-tricyclo[4.3.1.1^{3,8}]-undec-4-yl)-[4-(1,1-dioxo-1,2,5-thiadiazolidin-2-ylmethyl)-phenyl]-methanone
(Octahydro-isoquinolin-2-yl)-[4-(1,1-dioxo-1,2,5-thiadiazolidin-2-ylmethyl)-phenyl]-methanone
(3-Aza-bicyclo[3.2.2]non-3-yl)-[4-(1,1-dioxo-1,2,5-thiadiazolidin-2-ylmethyl)-phenyl]-methanone
(6-Aza-bicyclo[3.2.1]oct-6-yl)-[4-(1,1-dioxo-1,2,5-thiadiazolidin-2-ylmethyl)-phenyl]-methanone
[4-(5-Benzyl-1,1-dioxo-[1,2,5]thiadiazolidin-2-ylmethyl)-phenyl]-(1,3,3-trimethyl-6-aza-bicyclo[3.2.1]oct-6-yl)-methanone
or a salt thereof with a pharmaceutically acceptable acid or base, or any optical isomer or mixture of optical isomers, including a racemic mixture, or any tautomeric forms.

13. A compound according to claim 1 wherein the compound is selected from the group:
[4-(1-Amino-cyclopropyl)-phenyl]-(1,3,3-trimethyl-6-aza-bicyclo[3.2.1]oct-6-yl)-methanone
Piperidine-3-carboxylic acid methyl-[4-(1,3,3-trimethyl-6-aza-bicyclo[3.2.1]octane-6-carbonyl)-benzyl]-amide
N-Methyl-N-[4-(1,3,3-tri-methyl-6-aza-bicyclo[3.2.1]-octane-6-carbonyl)-benzyl]-butyramide
N-Methyl-N-[4-(octahydro-quinoline-1-carbonyl)-benzyl]-benzamide
N-[4-(3-Aza-bicyclo[3.2.2]-nonane-3-carbonyl)-benzyl]-N-methyl-benzamide
3-Cyano-N-methyl-N-[4-(octahydro-quinoline-1-carbonyl)-benzyl]-benzamide
N-[4-(3-Azabicyclo[3.2.2]-nonane-3-carbonyl)-benzyl]-3-cyano-N-methyl-benzamide
3-Fluoro-N-methyl-N-[4-(octahydro-quinoline-1-carbonyl)-benzyl]-benzamide
N-[4-(3-Aza-bicyclo[3.2.2]-nonane-3-carbonyl)-benzyl]-3-fluoro-N-methyl-benzamide
N-[4-(Azepane-1-carbonyl)-benzyl]-3-fluoro-N-methyl-benzamide
N-[4-(Azepane-1-carbonyl)-benzyl]-N-methyl-benzamide
N-[4-(Azepane-1-carbonyl)-benzyl]-3-cyano-N-methyl-benzamide
Piperidine-1-carboxylic acid [4-(azepane-1-carbonyl)-benzyl]-methyl-amide
Morpholine-4-carboxylic acid [4-(azepane-1-carbonyl)-benzyl]-methyl-amide
N-[4-(Octahydro-quinoline-1-carbonyl)-benzyl]-benzamide
N-[4-(3-Azabicyclo[3.2.2]-nonane-3-carbonyl)-benzyl]-benzamide
N-[4-(Azepane-1-carbonyl)-benzyl]-benzamide
N-[4-(6-Aza-bicyclo[3.2.1]-octane-6-carbonyl)-benzyl]-N-methyl-benzamide
4-[(Benzoyl-methyl-amino)-methyl]-N-(3-hydroxy-adamantan-1-yl)-benzamide
N-[4-(6-Aza-bicyclo[3.2.1]-octane-6-carbonyl)-benzyl]-3-cyano-N-methyl-benzamide
4-[(3-Cyano-benzoyl-methyl-amino)-methyl]-N-(3-hydroxy-adamantan-1-yl)-benzamide
N-[4-(6-Aza-bicyclo[3.2.1]-octane-6-carbonyl)-benzyl]-3-fluoro-N-methyl-benzamide
4-[(3-Fluoro-benzoyl-methyl-amino)-methyl]-N-(3-hydroxy-adamantan-1-yl)-benzamide
1-Acetyl-piperidine-4-carboxylic acid methyl-[4-(octahydro-quinoline-1-carbonyl)-benzyl]-amide
N-[4-(3-Hydroxy-8-aza-bicyclo[3.2.1]octane-8-carbonyl)-benzyl]-N-methyl-benzamide
3-Fluoro-N-methyl-N-[4-(1,8,8-trimethyl-3-aza-bicyclo[3.2.1]octane-3-carbonyl)-benzyl]-benzamide
3-Fluoro-N-[4-(3-hydroxy-8-aza-bicyclo[3.2.1]octane-8-carbonyl)-benzyl]-N-methyl-benzamide
N-Methyl-N-[4-(1,8,8-trimethyl-3-aza-bicyclo[3.2.1]-octane-3-carbonyl)-benzyl]-benzamide
N-[4-(6-Aza-bicyclo[3.2.1]-octane-6-carbonyl)-benzyl]-benzamide
N-[4-(1,8,8-Trimethyl-3-aza-bicyclo[3.2.1]octane-3-carbonyl)-benzyl]-benzamide
N-[4-(3-Hydroxy-8-aza-bicyclo[3.2.1]octane-8-carbonyl)-benzyl]-benzamide
1-Acetyl-piperidine-4-carboxylic acid [4-(azepane-1-carbonyl)-benzyl]-methyl-amide
4-(Benzoylamino-methyl)-N-(3-hydroxy-adamantan-1-yl)-benzamide
3-Cyano-N-methyl-N-[4-(1,8,8-trimethyl-3-aza-bi-cyclo[3.2.1]octane-3-car-bonyl)-benzyl]-benzamide
3-Cyano-N-[4-(3-fluoro-8-aza-bicyclo[3.2.1]octane-8-carbonyl)-benzyl]-N-methyl-benzamide
3-Fluoro-N-[4-(3-fluoro-8-aza-bicyclo[3.2.1]octane-8-carbonyl)-benzyl]-N-methyl-benzamide
4-(3-Fluoro-benzoylamino-methyl)-N-methyl-N-(3-fluoro-adamantan-1-yl)-benzamide
4-(3-Cyano-benzoylamino-methyl)-N-methyl-N-(3-fluoro-adamantan-1-yl)-benzamide
1-Acetyl-piperidine-4-carboxylic acid methyl-[4-(1,8,8-trimethyl-3-aza-bicyclo[3.2.1]-octane-3-carbonyl)-benzyl]-amide
N-[4-(3-Fluoro-8-aza-bicyclo[3.2.1]octane-8-carbonyl)-benzyl]-N-methyl-benzamide
4-[(Benzoyl-methyl-amino)-methyl]-N-(3-fluoro-adamantan-1-yl)-benzamide
3-Cyano-N-[4-(3-hydroxy-8-aza-bicyclo[3.2.1]octane-8-carbonyl)-benzyl]-N-methyl-benzamide
4-(Benzoylamino-methyl)-N-(3-fluoro-adamantan-1-yl)-benzamide
1-Acetyl-piperidine-4-carboxylic acid [4-(3-aza-bicyclo[3.2.2]nonane-3-carbonyl)-benzyl]-methyl-amide
N-[4-(3-Fluoro-8-aza-bicyclo[3.2.1]octane-8-carbonyl)-benzyl]-benzamide
4-(3-Cyano-benzoylamino-methyl)-N-(adamantan-2-yl)-benzamide
4-(3-Fluoro-benzoylamino-methyl)-N-(adamantan-2-yl)-benzamide
N-[4-(4-Azatricyclo-[4.3.1.1*3,8*]undecane-4-carbonyl)-benzyl]-3-fluoro-N-methyl-benzamide
N-{1-[4-(1,3,3-Trimethyl-6-aza-bicyclo[3.2.1]octane-6-carbonyl)-phenyl]-cyclo-propyl}-benzamide
N-{1-[4-(1,3,3-Trimethyl-6-aza-bicyclo[3.2.1]octane-6-carbonyl)-phenyl]-cyclo-propyl}-acetamide
4-Methanesulfonyl-N-{1-[4-(1,3,3-trimethyl-6-aza-bicyclo[3.2.1]octane-6-carbonyl)-phenyl]-cyclo-propyl}-benzamide
N-Methyl-N-{1-[4-(1,3,3-trimethyl-6-aza-bicyclo-[3.2.1]octane-6-carbonyl)-phenyl]-cyclo-propyl}-benzamide
N-Methyl-N-{1-[4-(1,3,3-trimethyl-6-aza-bicyclo-[3.2.1]octane-6-carbonyl)-phenyl]-cyclo-propyl}-acetamide
4-Methanesulfonyl-N-methyl-N-{1-[4-(1,3,3-trimethyl-6-aza-bicyclo[3.2.1]octane-6-carbonyl)-phenyl]-cyclo-propyl}-benzamide
N-[4-(Azepane-1-carbonyl)-benzyl]-N-methyl-methane-sulfonamide
N-[4-(3-Aza-bicyclo[3.2.2]-nonane-3-carbonyl)-benzyl]-N-methyl-methane-sulfonamide
N-Methyl-N-[4-(1,8,8-tri-methyl-3-aza-bicyclo[3.2.1]-octane-3-carbonyl)-benzyl]-methane-sulfonamide
N-[4-(6-Aza-bicyclo[3.2.1]-octane-6-carbonyl)-benzyl]-N-methyl-methane-sulfonamide
N-[4-(3-Hydroxy-8-aza-bicyclo[3.2.1]octane-8-carbonyl)-benzyl]-N-methyl-methanesulfonamide
N-Methyl-N-[4-(octahydro-quinoline-1-carbonyl)-benzyl]-methanesulfon-amide
N-(3-Hydroxy-adamantan-1-yl)-4-[(methanesulfonyl-methyl-amino)-methyl]-benzamide
N-(3-Fluoro-adamantan-1-yl)-4-[(methanesulfonyl-methyl-amino)-methyl]-benzamide
N-[4-(3-Fluoro-8-aza-bicyclo[3.2.1]octane-8-carbonyl)-benzyl]-N-methyl-methane-sulfonamide
N-Adamantan-2-yl-4-[(methanesulfonyl-methyl-amino)-methyl]-benzamide
N-(4-{1-[4-(1,3,3-Trimethyl-6-aza-bicyclo[3.2.1]octane-6-carbonyl)-phenyl]-cyclo-propylsulfamoyl}-phenyl)-acetamide
4-Chloro-N-{1-[4-(1,3,3-tri-methyl-6-aza-bicyclo-[3.2.1]octane-6-carbonyl)-phenyl]-cyclo-propyl}-benzene-sulfonamide
1-Methyl-1H-imidazole-4-sulfonic acid {1-[4-(1,3,3-trimethyl-6-aza-bicyclo-[3.2.1]-octane-6-carbonyl)-phenyl]-cyclopropyl}-amide
N-{1-[4-(1,3,3-Trimethyl-6-aza-bicyclo[3.2.1]octane-6-carbonyl)-phenyl]-cyclo-propyl}-ethanesulfonamide
1-[4-(Azepane-1-carbonyl)-benzyl]-1,3-dimethyl-3-phenyl-urea
Piperidine-1-carboxylic acid [4-(3-hydroxy-adamantan-1-yl-carbamoyl)-benzyl]-methyl-amide
Morpholine-4-carboxylic acid [4-(3-hydroxy-adamantan-1-yl-carbamoyl)-benzyl]-methyl-amide
4-(1,3-Dimethyl-3-phenyl-ureidomethyl)-N-(3-hydroxy-adamantan-1-yl)-benzamide
Piperidine-1-carboxylic acid [4-(3-fluoro-adamantan-1-yl-carbamoyl)-benzyl]-methyl-amide
Morpholine-4-carboxylic acid [4-(3-fluoro-adamantan-1-yl-carbamoyl)-benzyl]-methyl-amide
4-(1,3-Dimethyl-3-phenyl-ureidomethyl)-N-(3-fluoro-adamantan-1-yl)-benzamide
N-Adamantan-2-yl-4-(1,3-dimethyl-3-pyridin-2-yl-ureidomethyl)-benzamide
1,3-Dimethyl-3-[4-(2-oxa-5-aza-bicyclo[2.2.1]heptane-5-carbonyl)-benzyl]-1-pyridin-2-yl-urea
1-[4-(3-Hydroxy-8-aza-bicyclo[3.2.1]octane-8-carbonyl)-benzyl]-1,3-dimethyl-3-thiazol-2-yl-urea
1-(1-Acetyl-piperidin-4-yl)-1,3-dimethyl-3-[4-(2-oxa-5-aza-bicyclo[2.2.1]heptane-5-carbonyl)-benzyl]-urea
1-(1-Acetyl-piperidin-4-yl)-3-[4-(3-hydroxy-8-aza-bicyclo[3.2.1]-octane-8-carbonyl)-benzyl]-1,3-dimethyl-urea
1,3-Dimethyl-3-[4-(2-oxa-5-aza-bicyclo[2.2.1]heptane-5-carbonyl)-benzyl]-1-pyrimidin-2-yl-urea
Morpholine-4-carboxylic acid [4-(4-aza-tricyclo[4.3.1.1*3,8*]-undecane-4-carbonyl)-benzyl]-methyl-amide
1-[4-(3-Hydroxy-8-aza-bicyclo[3.2.1]octane-8-carbonyl)-benzyl]-3-(4-hydroxy-cyclohexyl)-1,3-dimethyl-urea
1-[4-(3-Aza-bicyclo[3.2.2]nonane-3-carbonyl)-benzyl]-3-(4-fluoro-cyclohexyl)-1,3-dimethyl-urea
N-Adamantan-2-yl-4-[3-(1-cyclopropyl-piperidin-4-yl)-1,3-dimethyl-ureidomethyl]-benzamide
1-[4-(3-Methoxy-8-aza-bicyclo[3.2.1]octane-8-carbonyl)-benzyl]-1,3-dimethyl-3-phenyl-urea
N-Adamantan-2-yl-4-[3-(4-fluoro-phenyl)-2-oxo-imidazolidin-1-ylmethyl]-benzamide
1-[4-(3-Aza-bicyclo[3.2.2]nonane-3-carbonyl)-benzyl]-3-(4-fluoro-phenyl)-imidazolidin-2-one
1-(4-Fluoro-phenyl)-3-[4-(3-hydroxy-8-aza-bicyclo[3.2.1]octane-8-carbonyl)-benzyl]-imidazolidin-2-one
N-Adamantan-2-yl-4-(2-oxo-3-phenyl-imidazolidin-1-ylmethyl)-benzamide
N-Adamantan-1-yl-4-(1,1-dioxo-[1,2,5]thiadiazolidin-2-ylmethyl)-benzamide
N-Adamantan-2-yl-4-(1,1-dioxo-[1,2,5]thiadiazolidin-2-ylmethyl)-benzamide
(4-Azatricyclo[4.3.1.1*3,8*]-undec-4-yl)-[4-(1,1-dioxo-[1,2,5]thiadiazolidin-2-ylmethyl)-phenyl]-methanone
Azepan-1-yl-[4-(1,1-dioxo-[1,2,5]thiadiazolidin-2-ylmethyl)-phenyl]-methanone
Azepan-1-yl-[4-(5-methyl-1,1-dioxo-[1,2,5]thiadiazolidin-2-ylmethyl)-phenyl]-methanone
N-Adamantan-1-yl-4-(5-methoxymethyl-1,1-dioxo-[1,2,5]thiadiazolidin-2-ylmethyl)-benzamide
4-(1,1-Dioxo-[1,2,5]thiadiazolidin-2-ylmethyl)-N-(3-hydroxy-adamantan-1-yl)-benzamide
{5-[4-(Adamantan-1-yl-carbamoyl)-benzyl]-1,1-dioxo-[1,2,5]thiadiazolidin-2-yl}-acetic acid tert-butyl ester
or a salt thereof with a pharmaceutically acceptable acid or base, or any optical isomer or mixture of optical isomers, including a racemic mixture, or any tautomeric forms.

14. The compound according to claim 1, which is an agent useful for the treatment of conditions, disorders, or diseases wherein a modulation or an inhibition of the activity of 11βHSD1 is beneficial.

15. The compound according to claim 14, wherein the conditions, disorders, and diseases are influenced by intracellular glucocorticoid levels.

16. The compound according to claim 14 wherein the conditions, disorders, or diseases are selected from metabolic syndrome, insulin resistance, dyslipidemia, hypertension, obesity, type 2 diabetes, impaired glucose tolerance (IGT), impaired fasting glucose (IFG), progression from IGT to type 2 diabetes, progression of metabolic syndrome into type 2 diabetes, diabetic late complications, neurodegenerative and psychiatric disorders, and the adverse effects of glucocorticoid receptor agonist treatment or therapy.

17. A pharmaceutical composition comprising, as an active ingredient, at least one compound according to claim 1 together with one or more pharmaceutically acceptable carriers or excipients.

18. The pharmaceutical composition according to claim 17, which is suitable for oral, nasal, buccal, transdermal, pulmonal, or parenteral administration.

19. (canceled)

20. (canceled)

21. (canceled)

22. (canceled)

23. A method for the treatment of conditions, disorders, or diseases wherein a modulation or an inhibition of the activity of 11βHSD1 is beneficial, the method comprising administering to a subject in need thereof an effective amount of a compound according to claim 1.

24. The method according to claim 23, wherein the conditions, disorders, and diseases are influenced by intracellular glucocorticoid levels.

25. The method according to claim 23 wherein the conditions, disorders, or diseases are selected from metabolic syndrome, insulin resistance, dyslipidemia, hypertension, obesity, type 2 diabetes, impaired glucose tolerance (IGT), impaired fasting glucose (IFG), progression from IGT to type 2 diabetes, progression of metabolic syndrome into type 2 diabetes, diabetic late complications, neurodegenerative and psychiatric disorders, and the adverse effects of glucocorticoid receptor agonist treatment or therapy.

26. The method according to claim 23 wherein the administering is via a route selected from oral, nasal, buccal, transdermal, pulmonal, and parenteral.
